(19) United States
(12) Patent Application Publication
(10) Pub. No.: US 2025/0154110 A1
(43) Pub. Date: May 15, 2025

(54) CLAUDIN INHIBITORS AND METHODS OF USE THEREOF

(71) Applicants: The University of Chicago, Chicago, IL (US); The Board of Trustees of the University of Illinois, Urbana, IL (US)

(72) Inventors: Christopher R. Weber, Oak Park, IL (US); Le Shen, Chicago, IL (US); Fatemeh Khalili-Araghi, Chicago, IL (US)

(73) Assignees: The University of Chicago, Chicago, IL (US); The Board of Trustees of the University of Illinois, Urbana, IL (US)

(21) Appl. No.: 18/715,299

(22) PCT Filed: Dec. 2, 2022
(86) PCT No.: PCT/US2022/051692

§ 371 Date: May 31, 2024

Related U.S. Application Data
(60) Provisional application No. 63/285,837, filed on Dec. 3, 2021.
(60) Provisional application No. 63/285,853, filed on Dec. 3, 2021.

(51) Int. Cl. C07D 239/06; A61K 31/165; A61K 31/17; A61K 31/343; A61K 31/4178; A61K 31/506; A61K 31/517; A61K 31/536; C07D 233/24

(52) U.S. Cl. CPC C07D 239/06; A61K 31/165; A61K 31/17; A61K 31/343; A61K 31/4178; A61K 31/506; A61K 31/517; A61K 31/536; C07D 233/24

(57) ABSTRACT

Provided is a method of treating a disorder mediated by claudin-2 and/or claudin-15, particularly an intestinal disorder, such as colitis or enteritis. The method comprises administering to the subject an effective amount of a compound of formula (I), formula (II), or otherwise as described herein or a pharmaceutically acceptable salt thereof. In another aspect, also provided are compounds of formula (Ia) and pharmaceutically acceptable salts thereof.

### CROSS-REFERENCE TO PRIOR APPLICATIONS

This application claims benefit to U.S. Provisional Patent Application Nos. 63/285,837 and 63/285,853, each filed Dec. 3, 2021, both of which are hereby incorporated by reference in their entireties.

### STATEMENT REGARDING FEDERALLY SPONSORED RESEARCH OR DEVELOPMENT

This invention was made with Government support under grant number 1 R01 DK131542-01 awarded by the National Institutes of Health. The Government has certain rights in this invention.

### BACKGROUND

Intestinal diseases, including inflammatory bowel disease (IBD), celiac disease, and infectious colitis are associated with epithelial barrier dysfunction that contribute to diarrhea and nutrient malabsorption and affect millions of Americans. IBD alone has emerged as a global disease with an estimated worldwide incidence of over 5 million people. Tight junctions seal spaces between epithelial cells and maintain barrier function by controlling paracellular flux. The claudin family of tight junction proteins is critical in defining the tight junction barrier to ions and small molecules. In the gastrointestinal tract, cation selective claudins (e.g., claudin-2 and -15) are particularly important in their role regulating paracellular sodium and water flux, and their altered expression can contribute to intestinal disease development. There continues to be a need for treatments of intestinal diseases, such as IBD, and targeting claudin flux could be one such approach.

### BRIEF SUMMARY

In an aspect, the disclosure provides a method of treating a disorder mediated by claudin-2 and/or claudin-15 in a subject comprising administering to the subject a compound of formula (I)

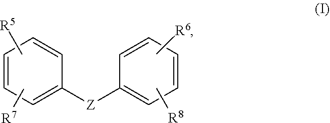

or a pharmaceutically acceptable salt thereof, wherein R⁵, R⁶, R⁷, R⁸, and Z are described herein.

In another aspect, also provided is compound of formula (I) that is a 1,3- or 1-4-substituted phenyl bridged compound of formula (Ia)

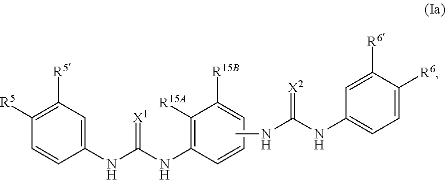

or a pharmaceutically acceptable salt thereof, wherein X¹, X², R⁵, R^{5′}, R⁶, R^{6′}, R^{15A}, and R^{15B} are described herein.

In another aspect, the disclosure provides a method of treating a disorder mediated by claudin-2 and/or claudin-15 in a subject comprising administering to the subject a compound of formula (II)

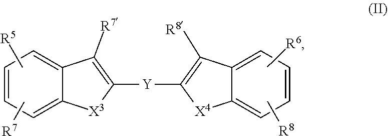

or a pharmaceutically acceptable salt thereof, wherein R⁵, R⁶, R⁷, R⁸, R^{7′}, R^{8′}, X³, X⁴, and Y are described herein.

Additional aspects are as described herein.

### BRIEF DESCRIPTION OF THE DRAWINGS

FIG. 1 is a reaction scheme of preparing building blocks 3 and 3a-1. Boc is a tert-butyloxycarbonyl protecting group; THE is tetrahydrofruan; CDI is 1,1′-carbonyldiimidazole; and DCM is dichloromethane.

FIG. 2 is a reaction scheme of preparing compound 1. TEA is triethylamine; DIEA is N,N-diisopropylethylamine; and TFA is trifluoroacetic acid.

FIG. 3 is a reaction scheme of preparing compound 5. DMAP is 4-dimethylaminopyridine.

FIG. 4 is a reaction scheme of preparing compound 4.

FIG. 5 is a reaction scheme of preparing compound 7.

FIG. 6 is a reaction scheme of preparing compound 8.

FIG. 7 is a reaction scheme of preparing compound 9.

FIG. 8 is a reaction scheme of preparing compound 21.

FIG. 9 is a reaction scheme of preparing compound 22. TCDI is 1,1′-thiocarbonyldiimidazole.

FIG. 10 is a reaction scheme of preparing compound 23.

FIG. 11 is a reaction scheme of preparing building blocks 27 and 28.

FIG. 12 is a reaction scheme of preparing compound 24.

FIG. 13 is a reaction scheme of preparing compound 25.

FIG. 14 is a reaction scheme of preparing compound 26.

FIG. 15 is a reaction scheme of preparing compound 27.

FIG. 16 is a reaction scheme of preparing compound 28.

FIG. 17 shows the approach used to assess epithelial barrier function over time after application of compounds. This is assessed as the change in transepithelial electrical resistance (TER). Representative recordings are shown for NSC 64906, NSC 76538, NSC 72574, NSC 75992, and NSC 85195 compared to vehicle (control) using Caco-2BBe epithelial cell monolayers. Note maximal TER change is achieved by 1 hour.

FIG. 18 shows the change in TER over time for the compounds NSC 80945, NSC 60765, NSC 66761, and NSC 80117 compared to vehicle (control) using MDCK I epithelial cell monolayers induced to express claudin-15. Note maximal TER change is achieved by 1 hour.

FIG. 19 is a table showing the percent change in TER after one hour, as measured in MDCK cells expressing either claudin-2 or claudin-15 and/or Caco-2 BBe cells, for exemplary compounds of the disclosure, as identified by their NSC number or compound number. The experimental approach is identical to FIGS. 17 and 18.

FIG. 20 is dose response data for NSC 53299 in MDCK I monolayers induced to express claudin-15.

FIGS. 21A-21B show representative ion selectivity measurements with permeability as a function of ion radius. FIG. 21A shows data for NSC 50464, NSC 66761, and NSC 58483 compared to vehicle (control) using MDCK I epithelial cell monolayers induced to express claudin-15. FIG. 21B shows data for NSC 60768, NSC 63672, NSC 63685, NSC 64906, NSC 66761, NSC 72574, NSC 72589, NSC 76538, NSC 76540, NSC 57144, NSC 57146, NSC 84450, and NSC 85195 compared to vehicle (control) using Caco-2 BBe monolayers.

FIG. 22 shows the change in TER over time for NSC 67736 versus vehicle (control) in mouse mucosa. One representative recording is shown on the left from before and after application of drug or vehicle at the time of the arrows. Change in TER from before and after application in multiple mice is shown on the right. The “**” refers to the p-value of the data for NSC 67736, in which P<0.01.

FIG. 23A shows mouse NaCl dilution potential measurements (ion selectivity measurement) of NSC 73446 versus vehicle (control) after apical solution was changed to Hank's buffered salt solution (HBSS) containing reduced NaCl (50% of the apical NaCl concentration balanced osmotically using mannitol).

FIG. 23B shows the percent change in the permeability of Na⁺ relative to the permeability of Cl⁻ for NSC 73446 and NSC 64906 compared to vehicle (control), as measured in small intestine and colon.

FIG. 24 provides an exemplary scheme of an organoid, first equilibrated in steady-state with Na⁺ as the predominant extracellular cation, which has been buffer exchanged with claudin-impermeant NMDG⁺, resulting in a high driving force for flux of Na⁺ and water out of the lumen of the organoid.

FIG. 25 provides the results of an organoid shrinking assay for colonoids derived from a patient with inflammatory bowel disease, which were treated with NSC 73446, as assessed using colonoid size change.

FIGS. 26A-26C show that in a molecular dynamics simulation, NSC 73446 and NSC 64906 reduce the conductance of claudin-15 to Na⁺. FIG. 26A provides a graph showing the conductance (pS) as a function of voltage for claudin-15, claudin-15 in the presence of NSC 73446, and claudin-15 in the presence of NSC 64906, as determined by all-atom molecular dynamics simulation of a claudin-15 pore in 200 mM NaCl under a voltage bias. FIG. 26B shows a snapshot of a claudin-15 pore complexed with NSC 73446, which was taken from a molecular dynamic trajectory. FIG. 26C shows a snapshot of a claudin-15 pore complexed with NSC 64906, which was taken from a molecular dynamic trajectory.

### DETAILED DESCRIPTION

In an aspect, the disclosure provides a method of treating a disorder mediated by claudin-2 and/or claudin-15 in a subject comprising administering to the subject an effective amount of a compound of formula (I)

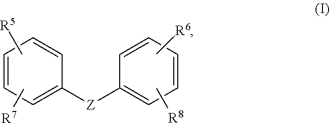

wherein
R⁵ and R⁶ are the same and are selected from

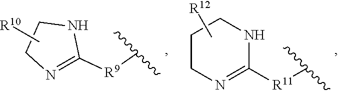

and —C(NHR¹³)—NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is alkyl;
R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
Z is selected from

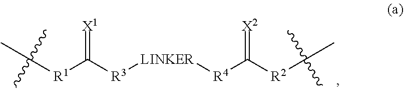

wherein
X¹ and X² are the same or different and each is O or S;
R¹ and R² are the same or different and each is —NH— or a bond;
R³ and R⁴ are the same or different and each is —NH—, —CH═CH—, —CH₂CH₂—, —NHC(O)—, —NHC(O)CH₂NHC(O)NH—, or a bond;
provided that R¹ and R³ are not both bonds and R² and R⁴ are not both bonds; and
LINKER is selected from
(i) —(CH₂)ₙ— that is optionally substituted with a hydroxy, wherein n is an integer of 1-12,
(ii) —O(CH₂)ₘO— that is optionally substituted, wherein m is an integer of 3-6,
(iii) —CH═CH—,

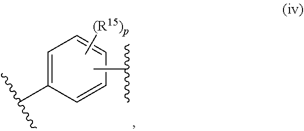

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2,

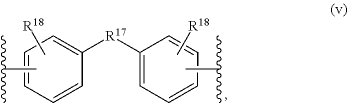

wherein R¹⁷ is selected from a bond, —CH═CH—, —N═N—, —CH₂—, —NHC(O)—, —C(O)NH—, —NHC(O)NH—, —NHC(O)—(CH₂)₁₋₄—C(O)NH—, —NHC(O)NH—(CH₂)₁₋₄—NHC(O)NH—, —NHC(O)-Ph-C(O)NH—, and —NHC(O)NH-Ph-NHC(O)NH—, and each R¹⁸ is the same or different and each is hydrogen, alkyl, alkoxy, 1,4-dihydroimidazolyl, or —R¹⁶—C(O)NH-Ph-R⁵,

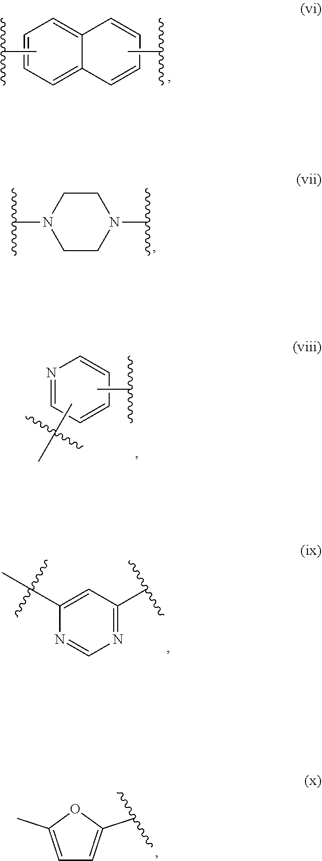

and (xi) a bond,
and

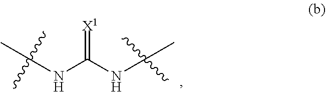

wherein X¹ is O or S;
or a pharmaceutically acceptable salt thereof to the subject.

In some aspects, the compound of formula (I) is a compound of formula (I-1):

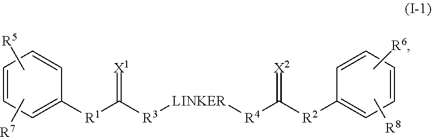

wherein
X¹ and X² are the same or different and each is O or S;
R¹ and R² are the same or different and each is —NH— or a bond;
R³ and R⁴ are the same or different and each is —NH—, —CH═CH—, —CH₂CH₂—, —NHC(O)—, —NHC(O)CH₂NHC(O)NH—, or a bond;
provided that R¹ and R³ are not both bonds and R² and R⁴ are not both bonds;
R⁵ and R⁶ are the same and are selected from

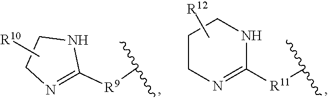

and —C(NHR¹³)═NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is alkyl;
R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
LINKER is selected from
(i) —(CH₂)ₙ— that is optionally substituted with a hydroxy, wherein n is an integer of 1-12,
(ii) —O(CH₂)ₘO— that is optionally substituted, wherein m is an integer of 3-6,
(iii) —CH═CH—,

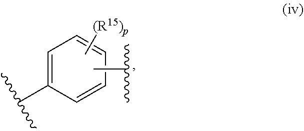

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2,

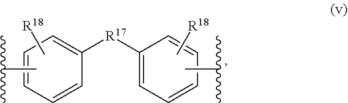

wherein R¹⁷ is selected from a bond, —CH═CH—, —N═N—, —CH₂—, —NHC(O)—, —C(O)NH—, —NHC(O)NH—, —NHC(O)—(CH₂)₁₋₄—C(O)NH—, —NHC(O)NH—(CH₂)₁₋₄—NHC(O)NH—, —NHC(O)-Ph-C(O)NH—, and —NHC(O)NH-Ph-NHC(O)NH—, and each R¹⁸ is the same or different and each is hydrogen, alkyl, alkoxy, 1,4-dihydroimidazolyl, or —R¹⁶—C(O)NH-Ph-R⁵,

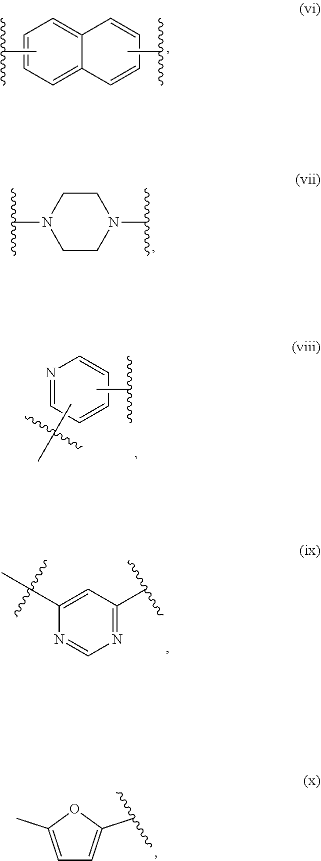

and (xi) a bond,
or a pharmaceutically acceptable salt thereof to the subject.

In other aspects, the compound of formula (I) is a compound of formula (I-2)

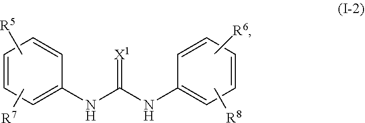

wherein
X¹ is O or S;
R⁵ and R⁶ are the same and are selected from

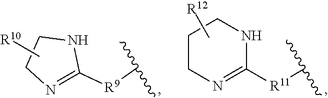

and —C(NHR¹³)—NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is alkyl; and
R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—;
or a pharmaceutically acceptable salt thereof to the subject.

In some aspects of formula (I), including formula (I-1), or a pharmaceutically acceptable salt thereof, X¹ and X² are both O. In other aspects, X¹ is S and X² is O or both X¹ and X² are S. In some aspects of formula (I), including formula (I-2), or a pharmaceutically acceptable salt thereof, X¹ is O.

In any of the foregoing aspects of the compound of formula (I), including formulas (I-1), or a pharmaceutically acceptable salt thereof, R¹ and R² are each —NH—.

In any of the foregoing aspects of the compound of formula (I), including formula (I-1), or a pharmaceutically acceptable salt thereof, R³ and R⁴ are each —NH— or a bond. Preferably, R³ and R⁴ are each —NH—.

Suitable examples of —R¹—C(═X¹)—R³— and —R⁴—C(═X²)—R²— in combination include, e.g.,

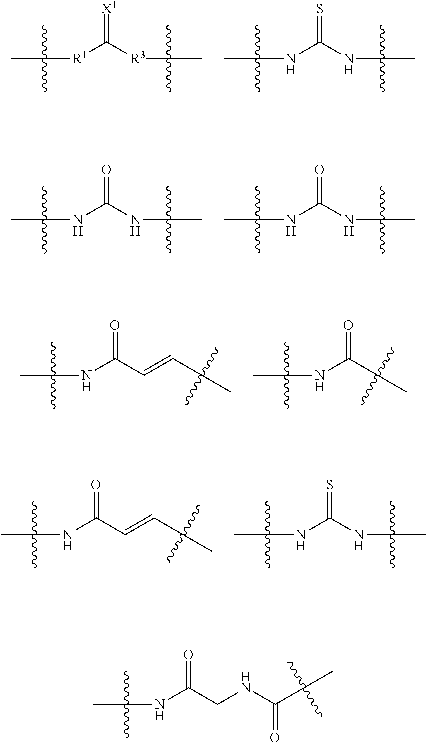

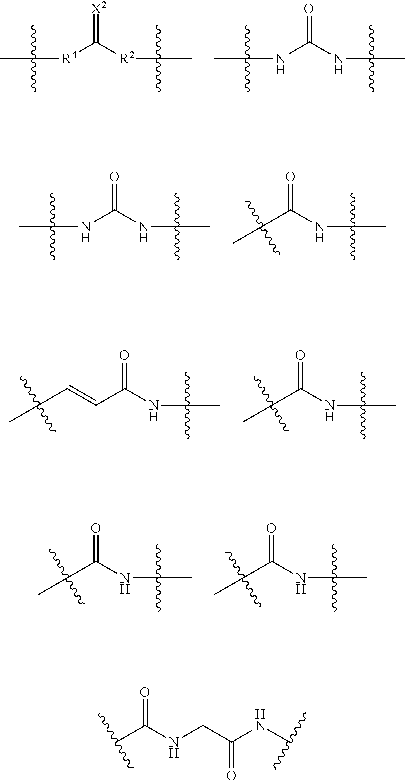

In any of the foregoing aspects of the compound of formula (I), including formulas (I-1) and (I-2), or a pharmaceutically acceptable salt thereof, R⁵ and R⁶ are both either

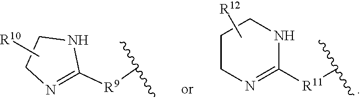

In some aspects of this aspect of R⁵ and R⁶, R⁹ and R¹¹ are both a bond and/or R¹⁰ and R¹² are both hydrogen or alkyl.

Suitable examples of compounds in which R⁵ and R⁶ are the same as each other include, e.g.,

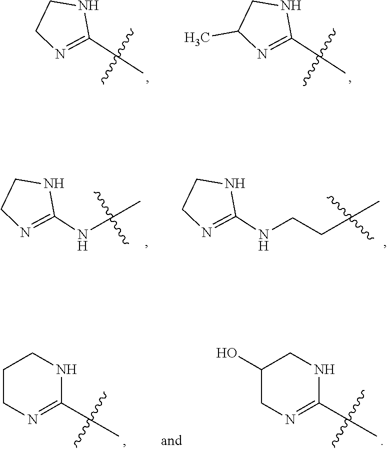

In some aspects of formula (I), including formulas (I-1) and (I-2), R⁵ and R⁶ are each

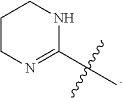 [SMILES: *C1=NCCCN1]

In any of the foregoing aspects of the compound of formula (I), including formulas (I-1) and (I-2), or a pharmaceutically acceptable salt thereof, R⁵ and R⁶ are both —C(NHR¹³)═NR¹⁴, in which R¹³ and R¹⁴ are the same or different and each is alkyl, such as C₁-C₆, preferably C₁-C₄. Specific examples include compounds in which R¹³ and R¹¹ are different, such as R¹³ is methyl and R¹⁴ is n-butyl; R¹³ is methyl and R¹⁴ is i-propyl; R¹³ is ethyl and R¹⁴ is i-propyl; or R¹³ is ethyl and R¹⁴ is n-butyl; and compounds in which R¹³ and R¹⁴ are the same and both are methyl, ethyl, n-propyl, i-propyl, n-butyl, iso-butyl, sec-butyl, or tert-butyl.

In any of the foregoing aspects of the compound of formula (I), including formula (I-1), or a pharmaceutically acceptable salt thereof, (i) R¹ and R⁵ are meta or para to each other and (ii) R² and R⁶ are meta or para to each other. In such aspects, preferably (i) R¹ and R⁵ are para to each other and (ii) R² and R⁶ are para to each other. In any of the foregoing aspects of the compound of formula (I), including formula (I-2), or a pharmaceutically acceptable salt thereof, —NH— and R⁵ are meta or para to each other and preferably para to each other.

In any of the foregoing aspects of the compound of formula (I), including formulas (I-1) and (I-2), or a pharmaceutically acceptable salt thereof, R⁷ and R⁸ are the same and each is selected from hydrogen, alkyl, amido, alkylamido, dialkylamido, and R⁵. In some preferred aspects, R⁷ and R⁸ are the same and each is hydrogen, alkylamido (e.g., —C(O)NH(C₁₋₄ alkyl)),

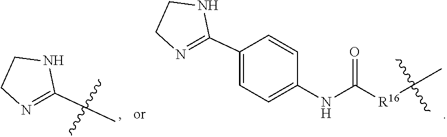

In any of the foregoing aspects of the compound of formula (I-1) or a pharmaceutically acceptable salt thereof, LINKER is selected from
(i) —(CH₂)ₙ— that is optionally substituted with a hydroxy, wherein n is an integer of 1-12,

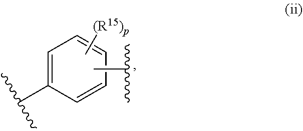

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, R¹⁶ is a bond or —NH—, and p is 0 or an integer of 1 or 2,

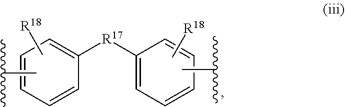

wherein R¹⁷ is selected from a bond; —CH═CH—, —N═N—, —CH₂—, —NHC(O)NH—, and —NHC(O)-Ph-C(O)NH—, and R¹⁸ is hydrogen, alkyl, or alkoxy, and

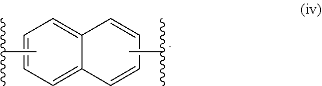

In an aspect, LINKER is —(CH₂)ₙ— in which n is an integer of 1-12 (i.e., 1, 2, 3, 4, 5, 6, 7, 8, 9, 10, 11, or 12). In a preferred aspect, n is 1, 3, 6, 7, 9, or 10. More preferably, n is 9 or 10.

In an aspect, LINKER is

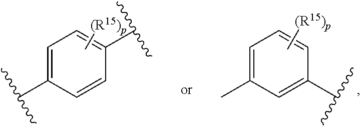

wherein
p is 0, or
p is 1 and R¹⁵ is selected from alkyl (e.g., C₁₋₄ alkyl), halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, and
R¹⁶ is a bond or —NH—.

In some aspects of when LINKER is 1,3-phenyl or 1,4-phenyl, p is 0. In other aspects of when LINKER is 1,3-phenyl or 1,4-phenyl, p is 1 and R¹⁵ is C₁₋₄ alkyl, alkoxy, chloro, bromo, iodo, nitro, amino, C₁₋₄ alkylamido, C₁₋₄ dialkylamido, or phenylamido.

Alternatively, p is 1 and R¹⁵ is —R¹⁶—C(O)NH-Ph-R⁵ to form a trimer structure. When the compound is a trimer, —R¹⁶—C(O)NH-Ph-R⁵ is the same structure as R⁵-Ph-R¹—C(═X¹)—R³—, which is the same structure as —R⁴—C(═X²)—R²-Ph-R⁶. In a preferred aspect, R⁵ and R⁶ are both 1.

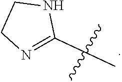 [SMILES: *C1=NCCN1]

In other aspects of when LINKER is 1,3-phenyl or 1,4-phenyl, p is 2 and both instances of R¹⁵ are C₁₋₄ alkyl or halo (e.g., Cl).

Specific examples of a phenyl-based LINKER include, e.g.,

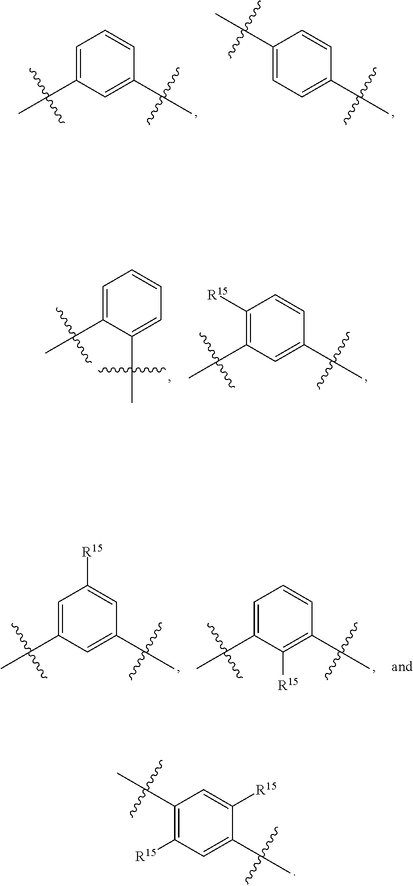

In an aspect, LINKER is

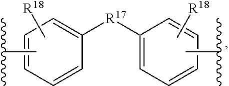

wherein R⁷ is selected from a bond; —CH₂—, or —NHC(O)NH—, and each instance of R¹⁸ is hydrogen or alkyl. Specific examples of such LINKER include, e.g.,

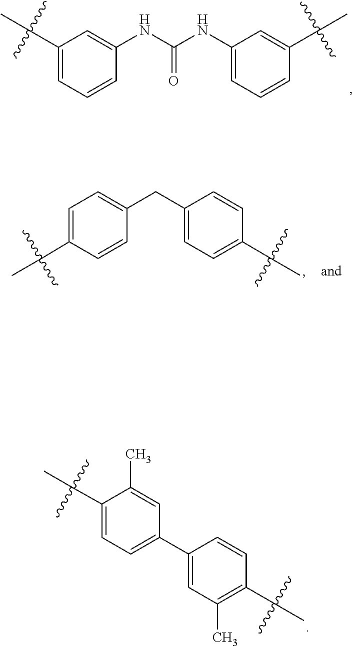 [SMILES: *c1cccc(NC(=O)Nc2cccc(*)c2)c1 ; *c1ccc(Cc2ccc(*)cc2)cc1 ; *c1ccc(-c2ccc(*)c(C)c2)cc1C]

In an aspect, LINKER is

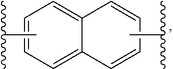

especially

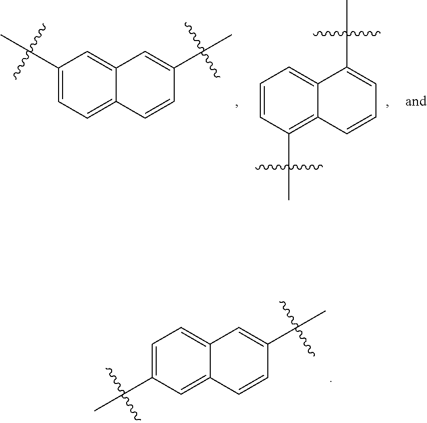 [SMILES: *c1ccc2ccc(*)cc2c1 ; *c1cccc2c(*)cccc12 ; *c1ccc2cc(*)ccc2c1]

Exemplary core structures of formula (I) include, e.g.,

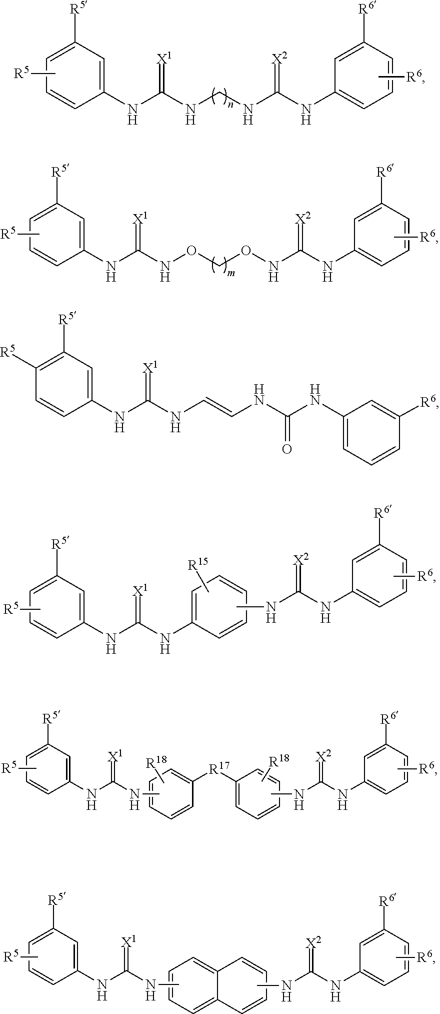

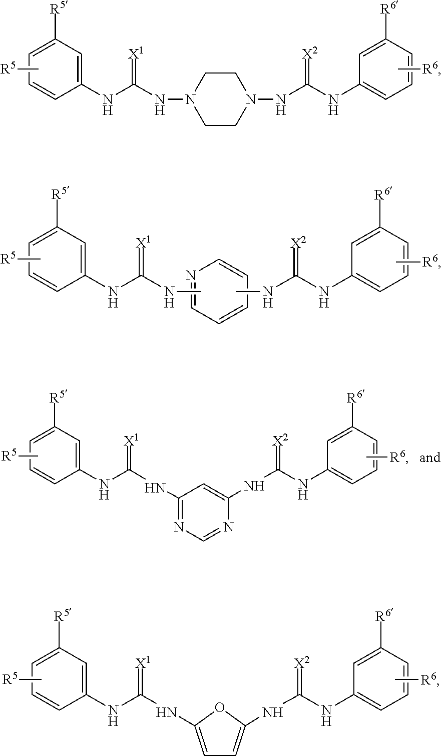

in which X¹, X², R⁵, R^{5′}, R⁶, R^{6′}, R¹, R¹⁷, R¹⁸, n, and m are described herein.

Exemplary compounds, including compounds of formulas (I-1) and (I-2), for use in the method described herein include:

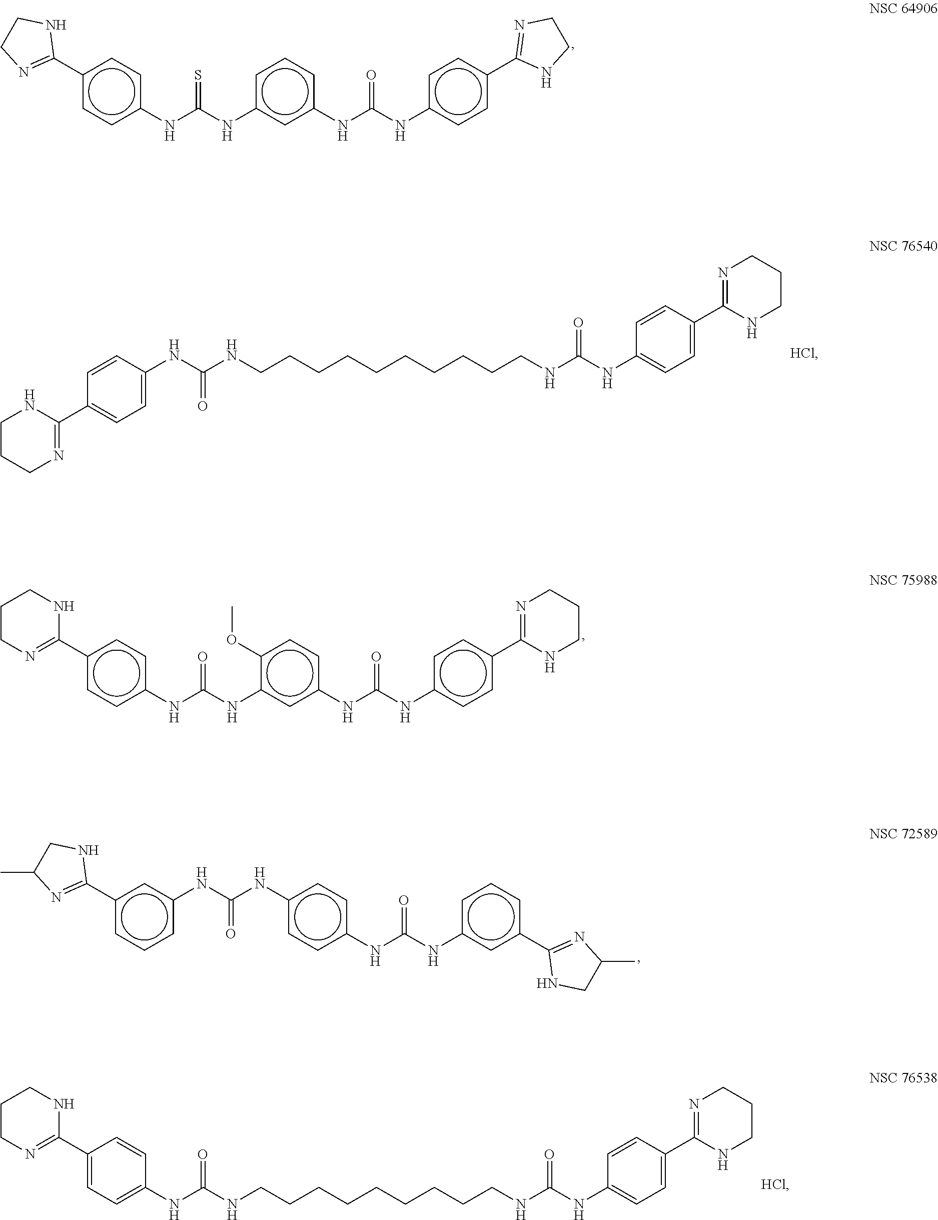

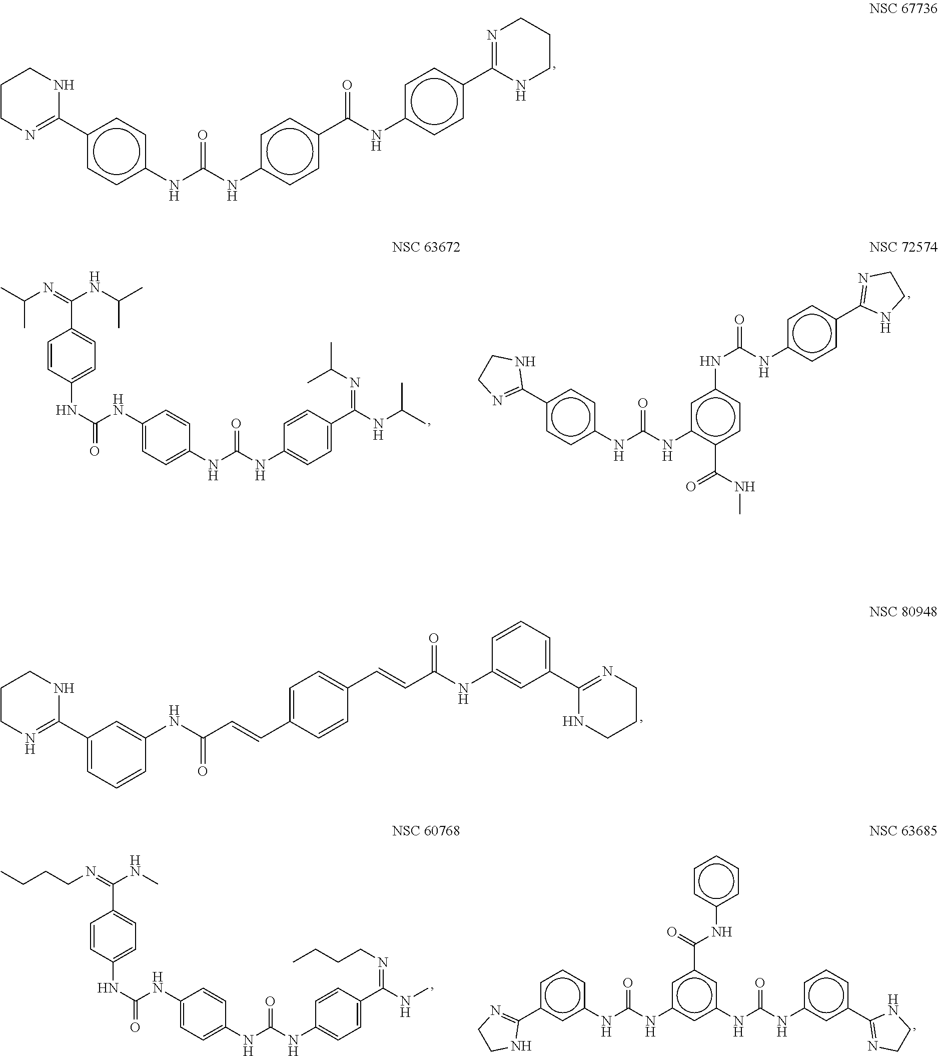

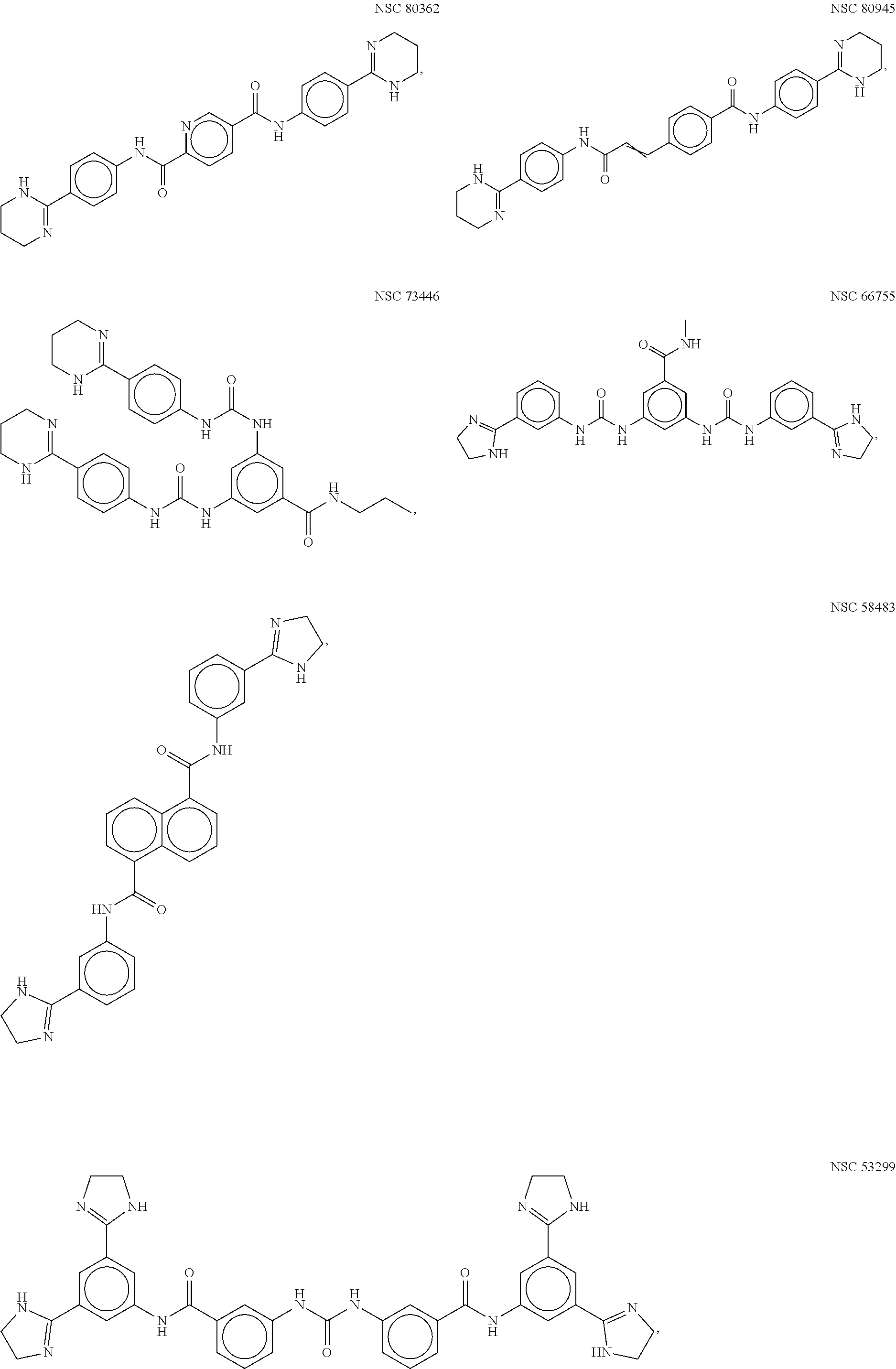

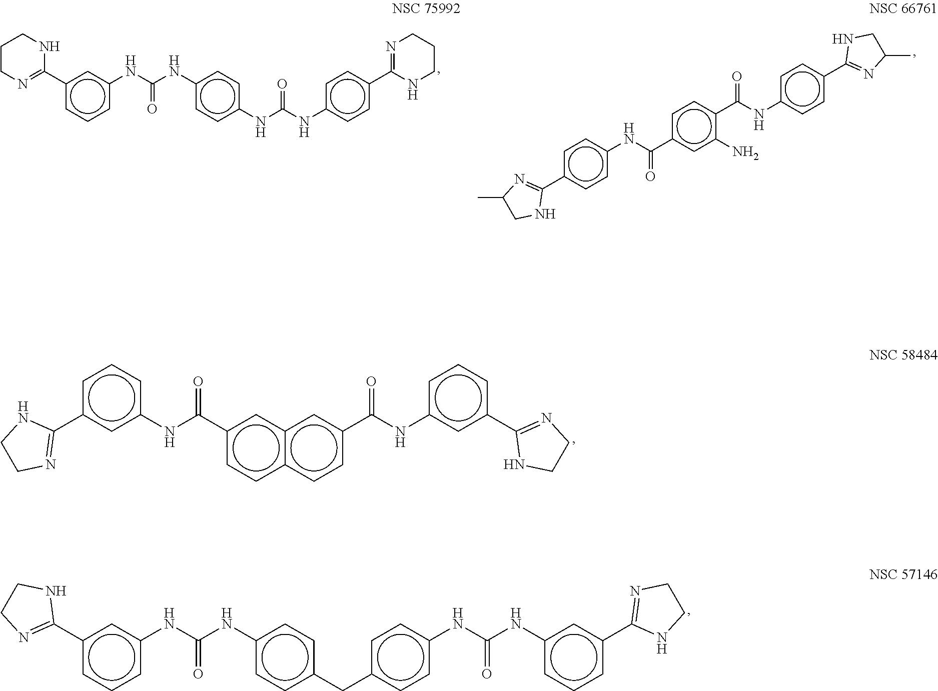

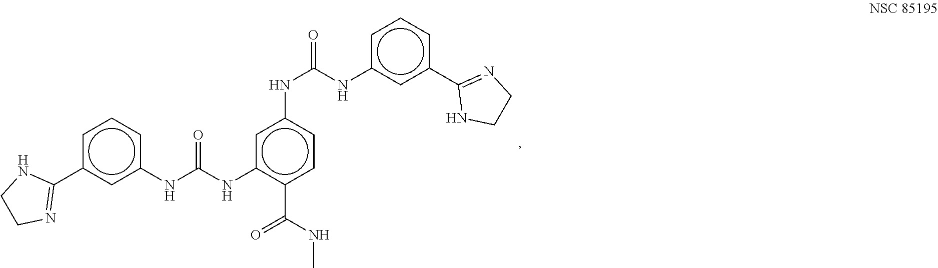

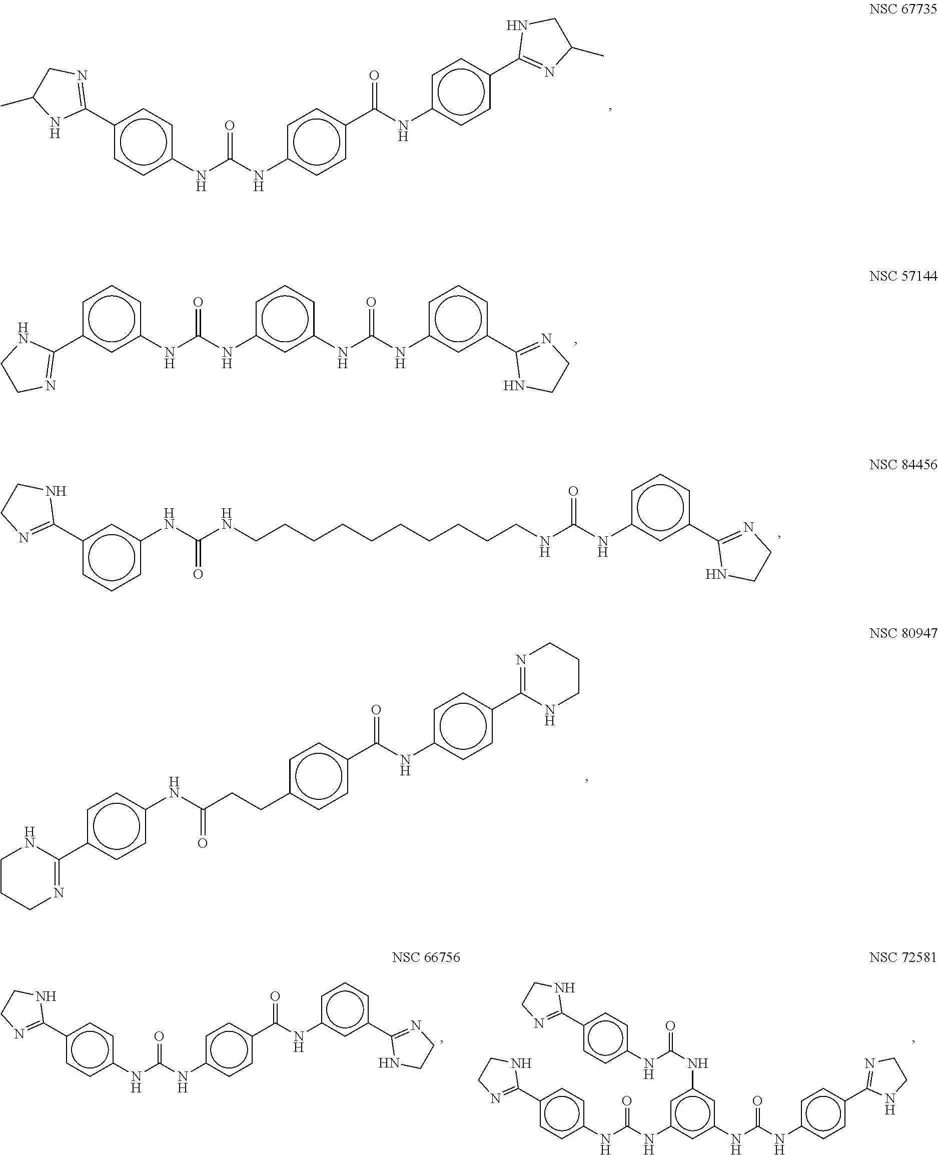

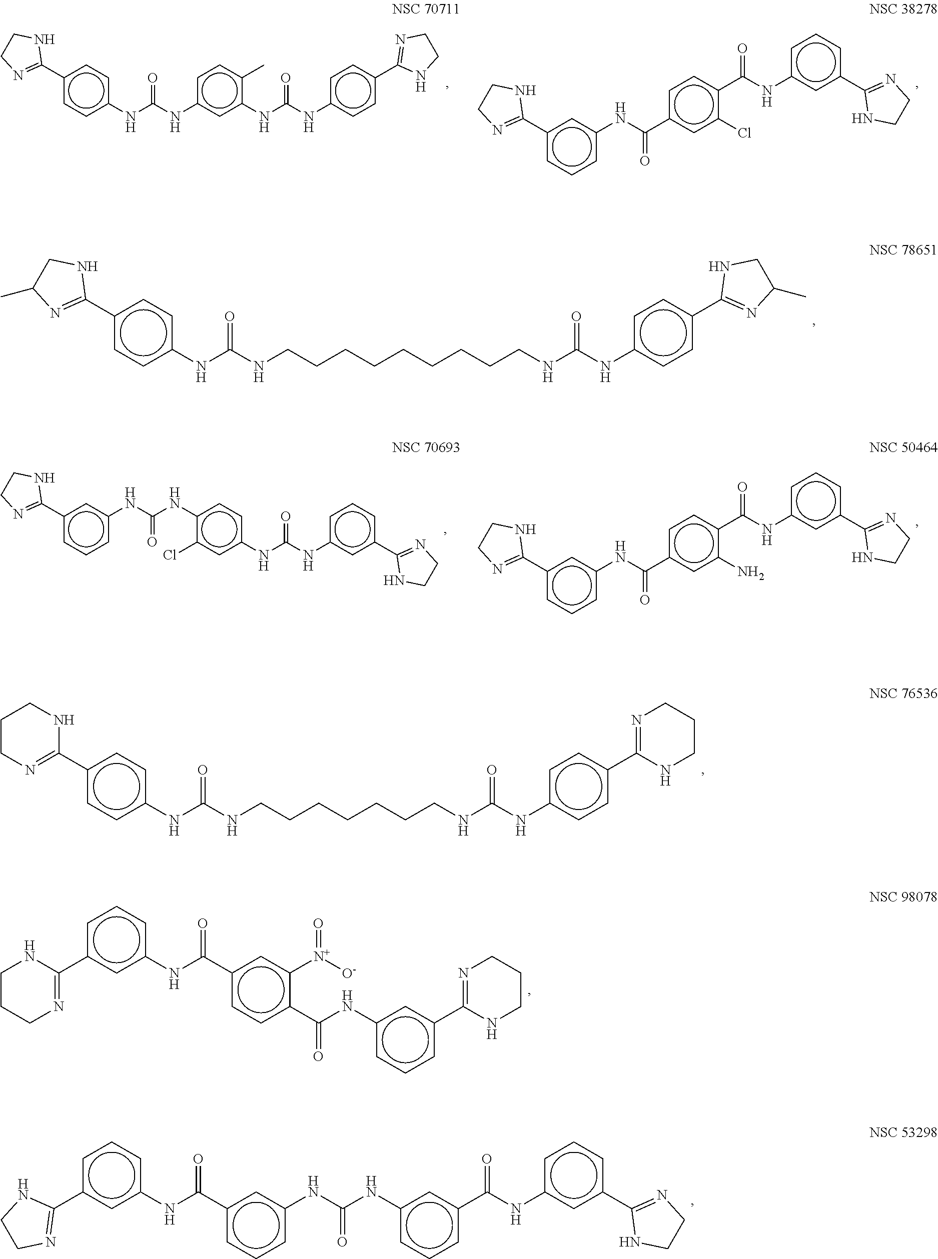

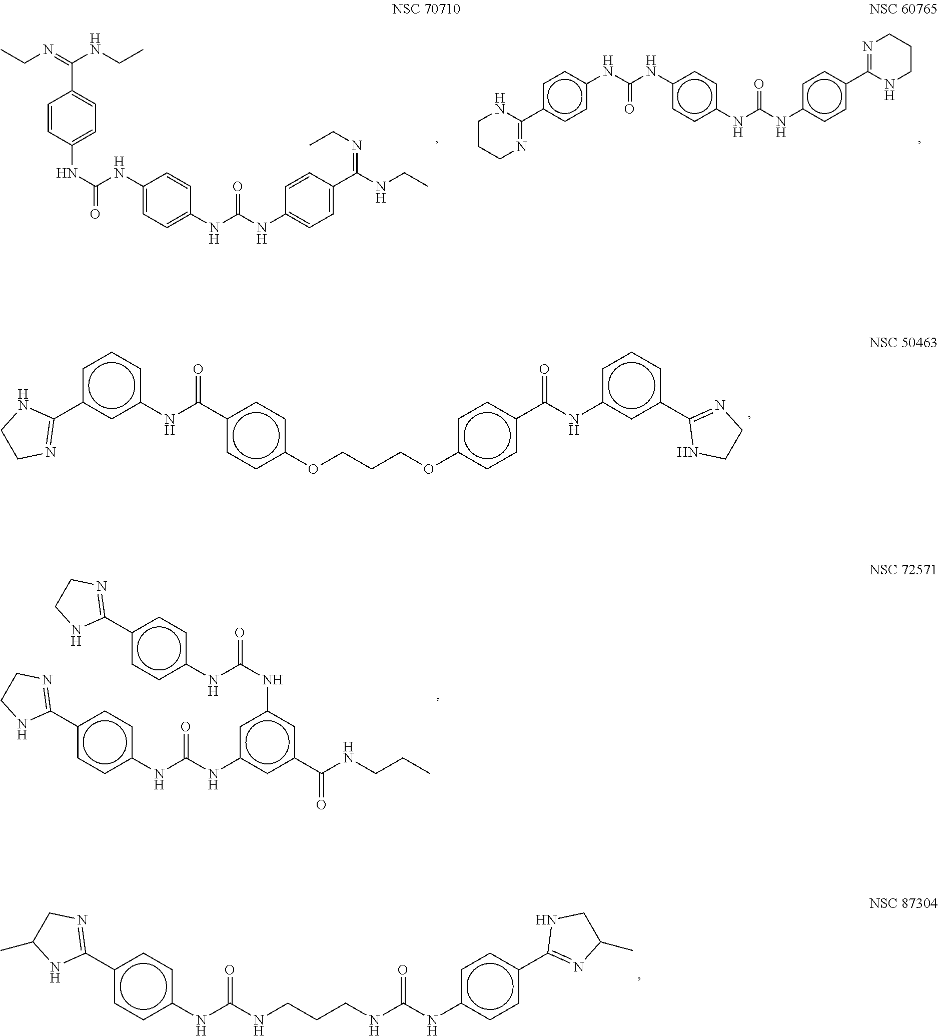

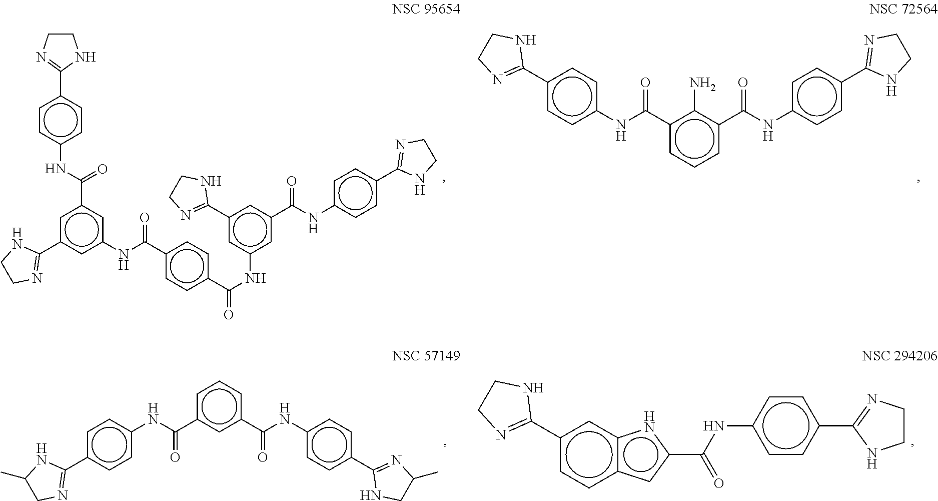

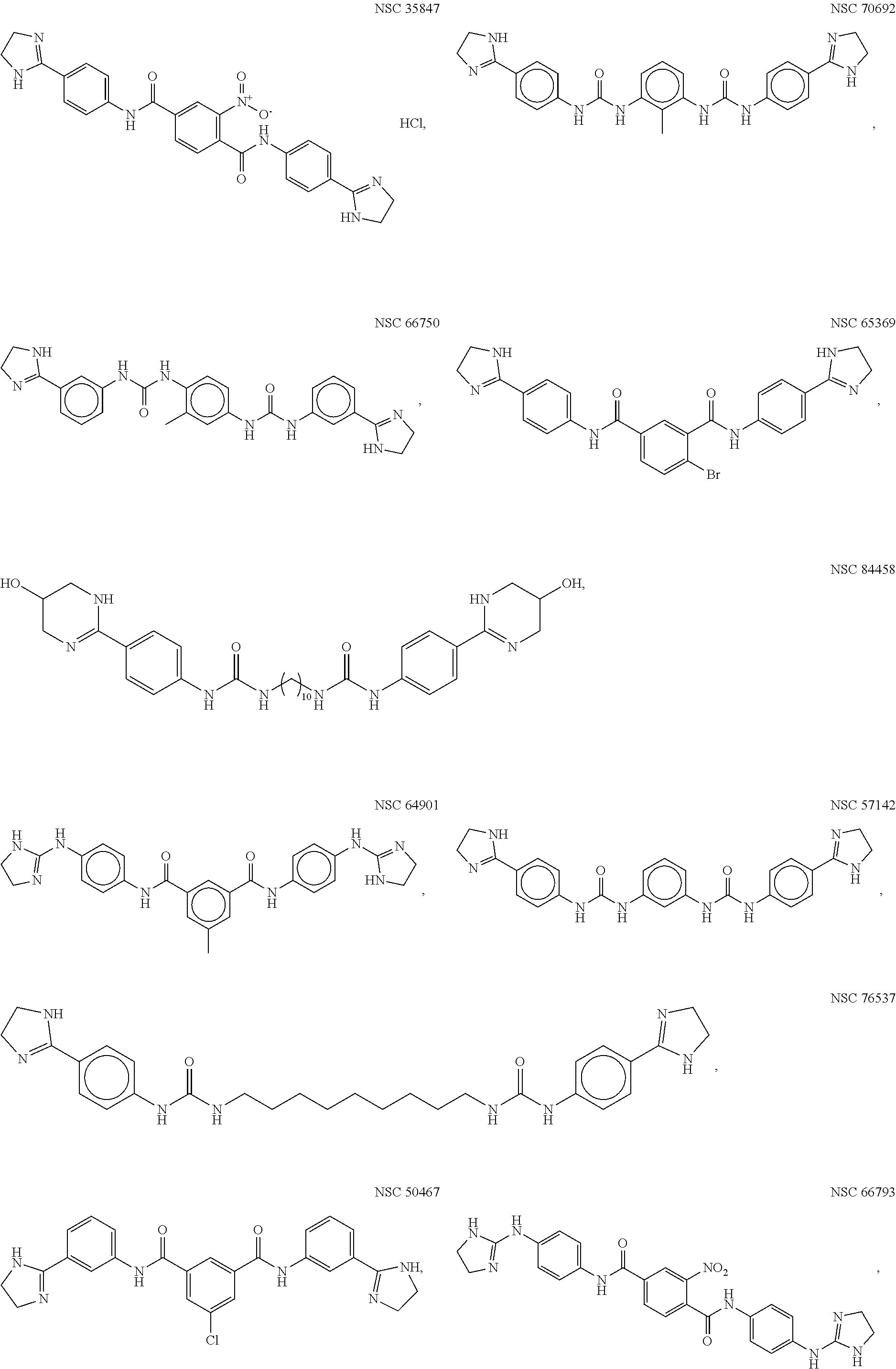

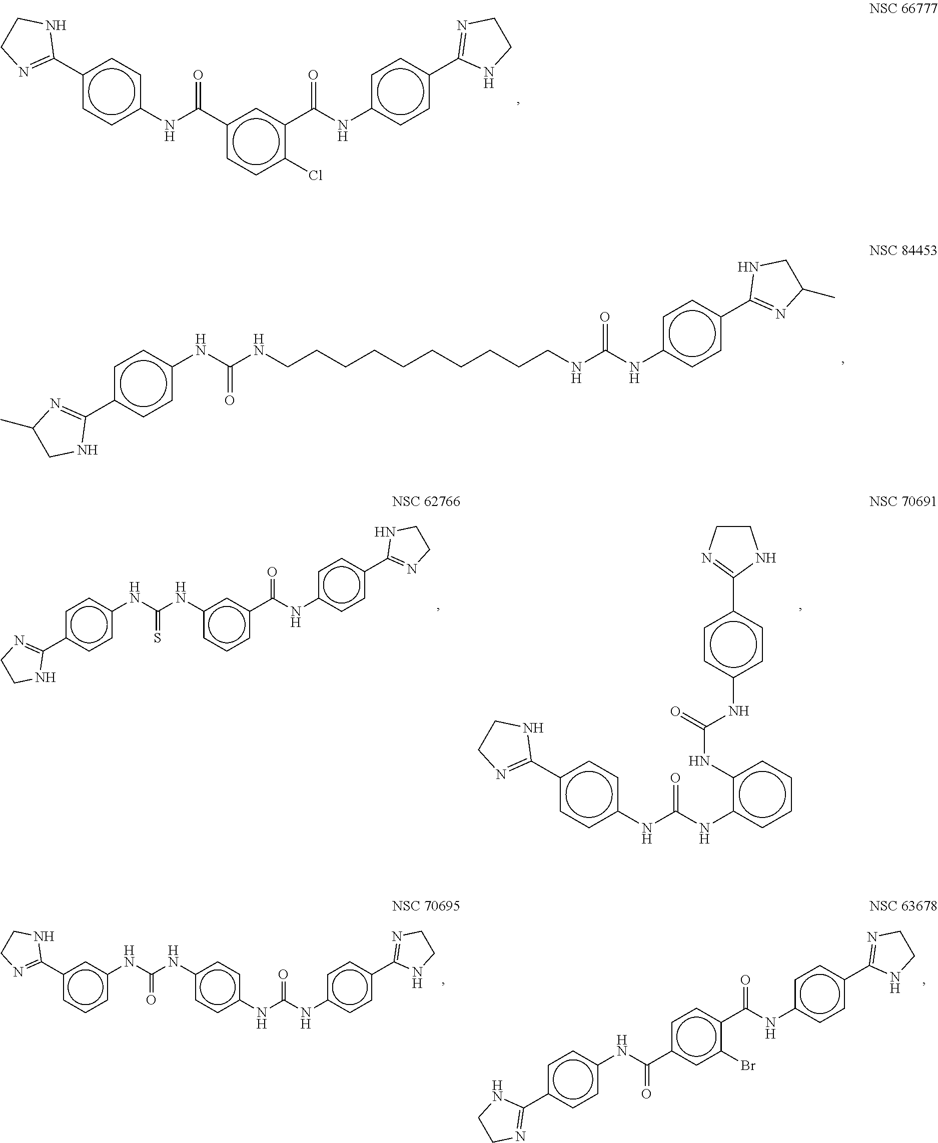

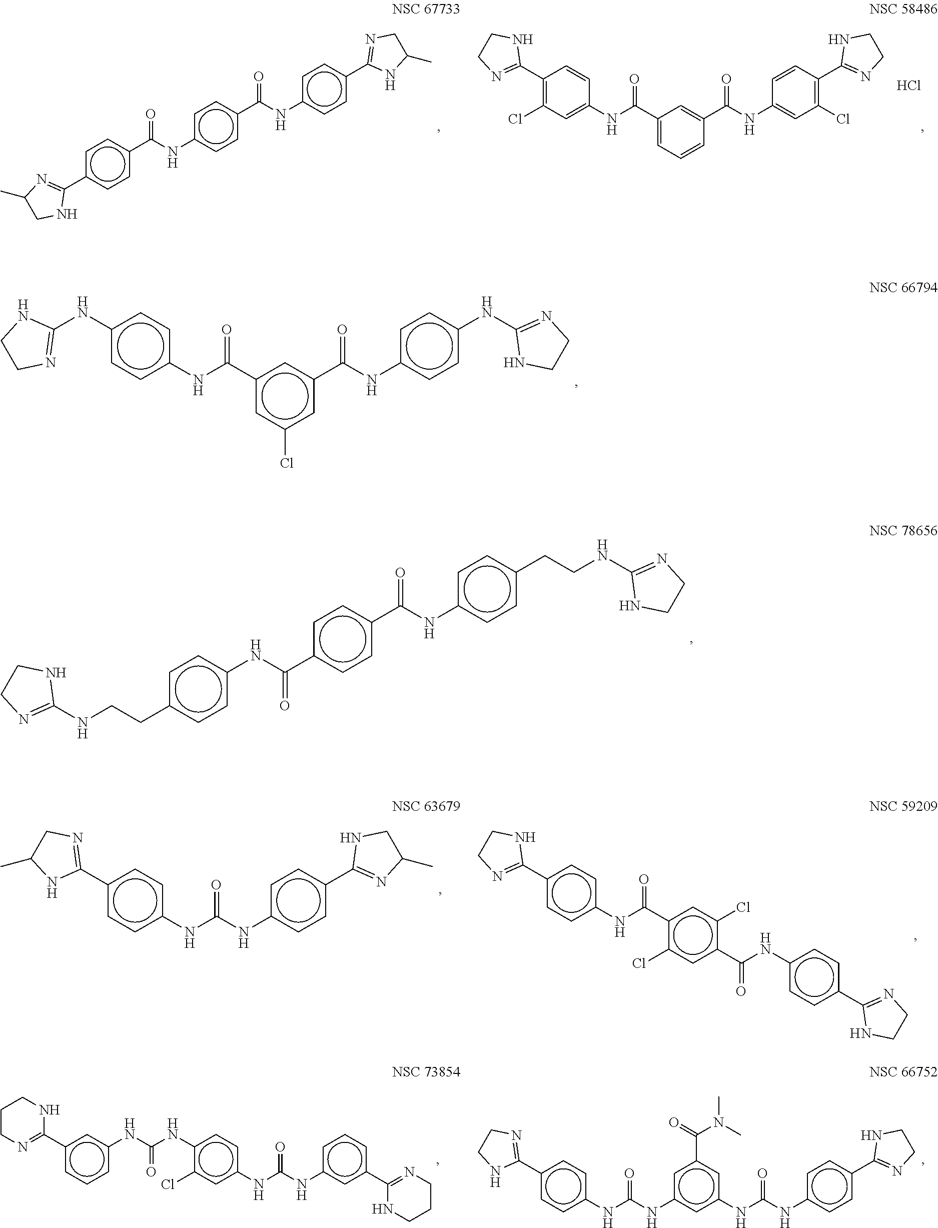

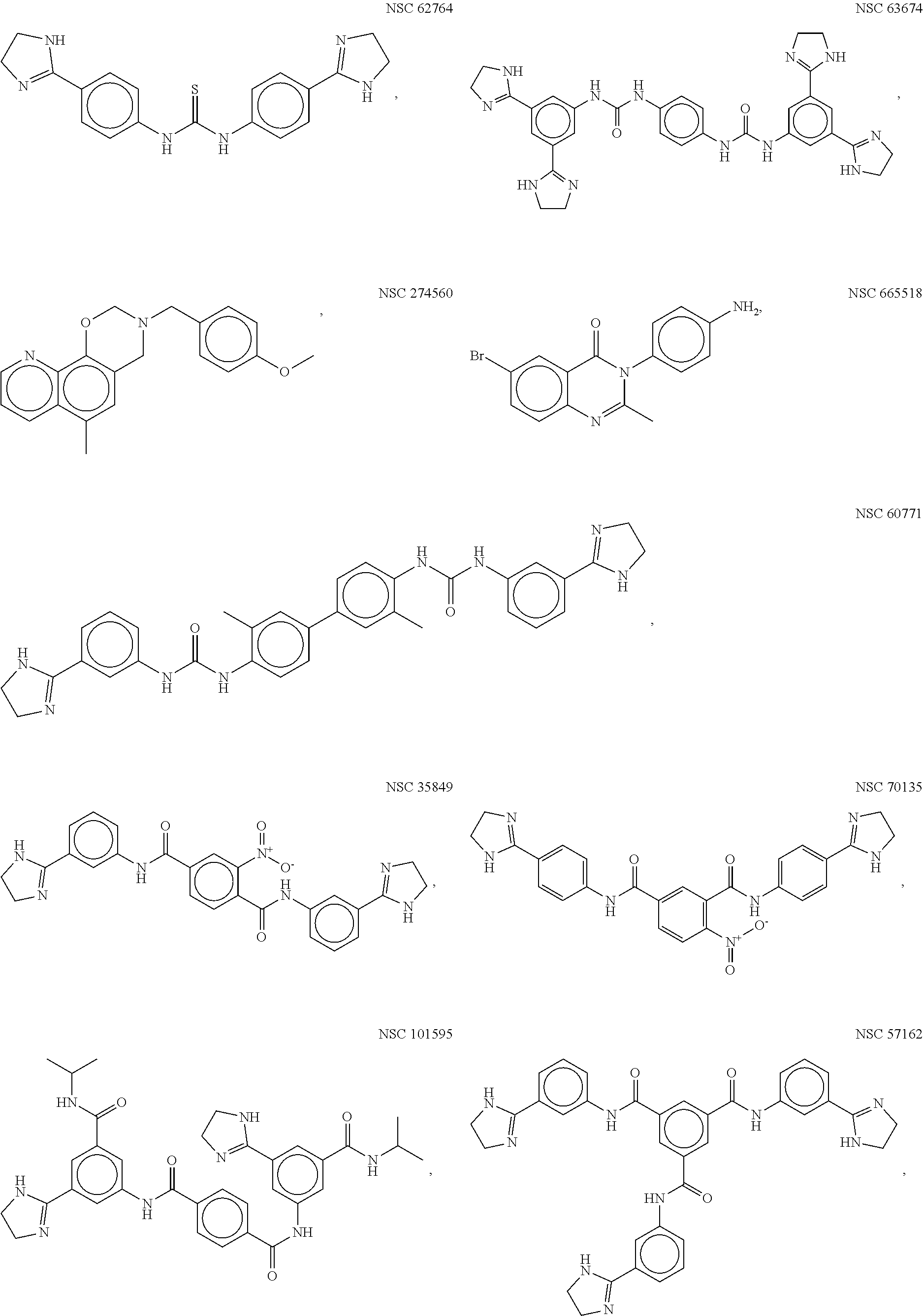

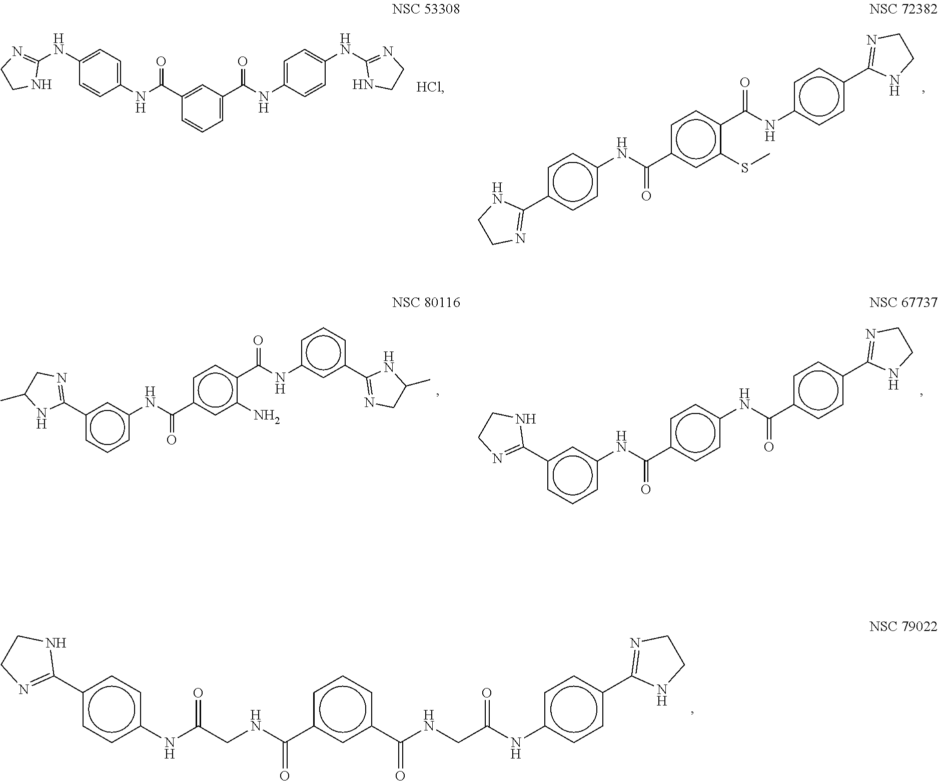

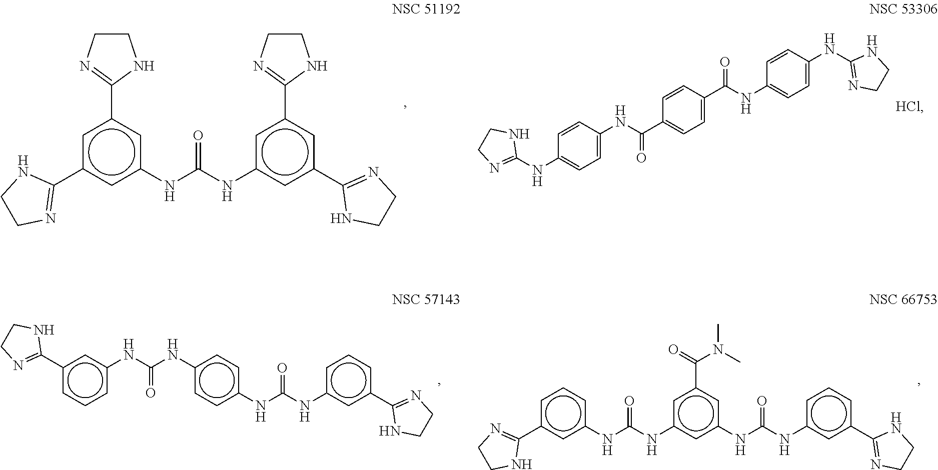

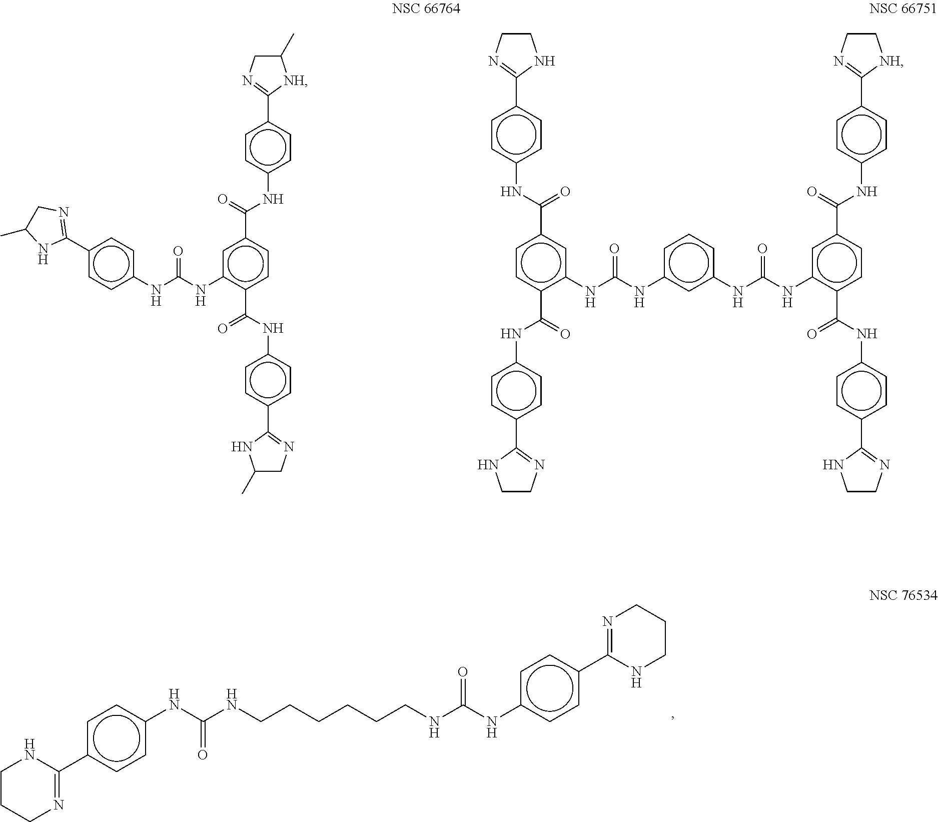

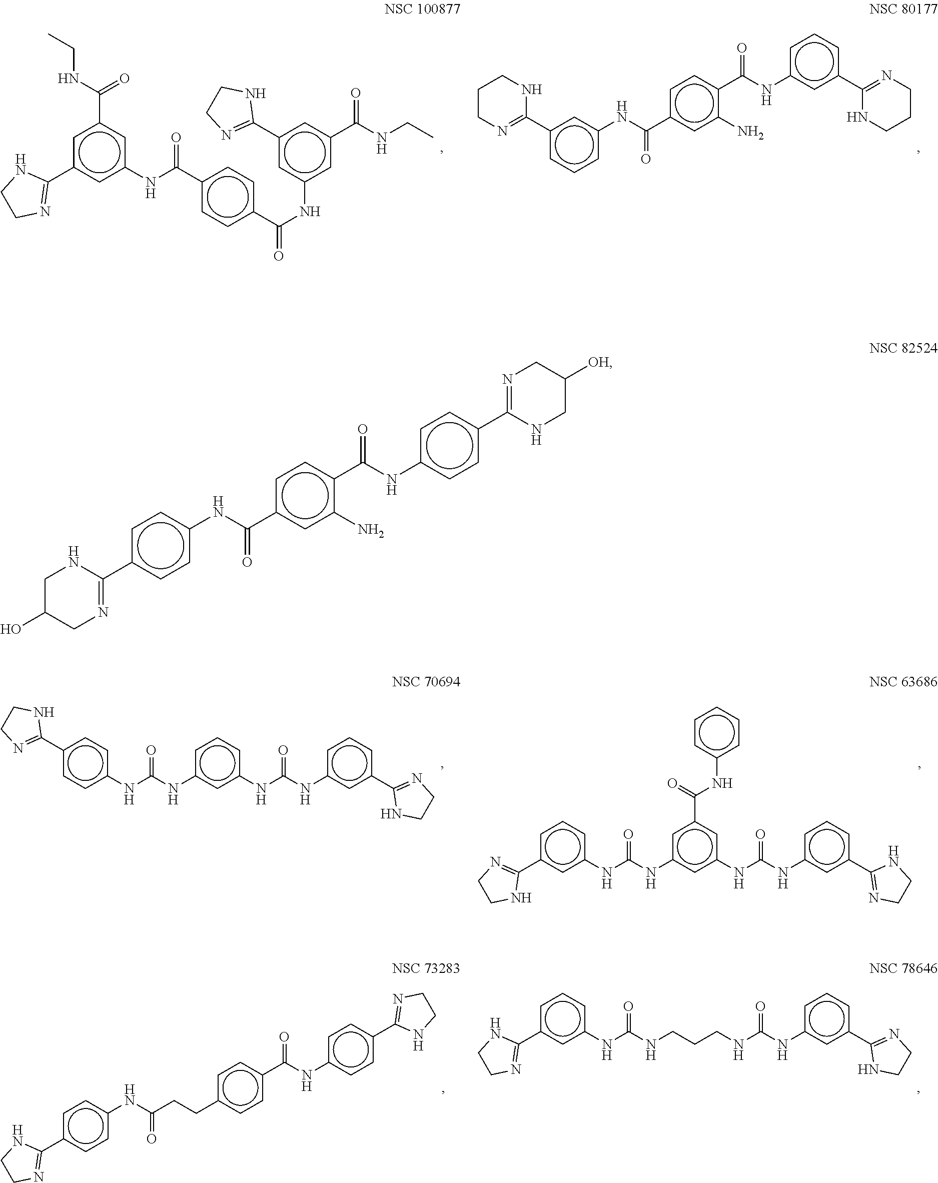

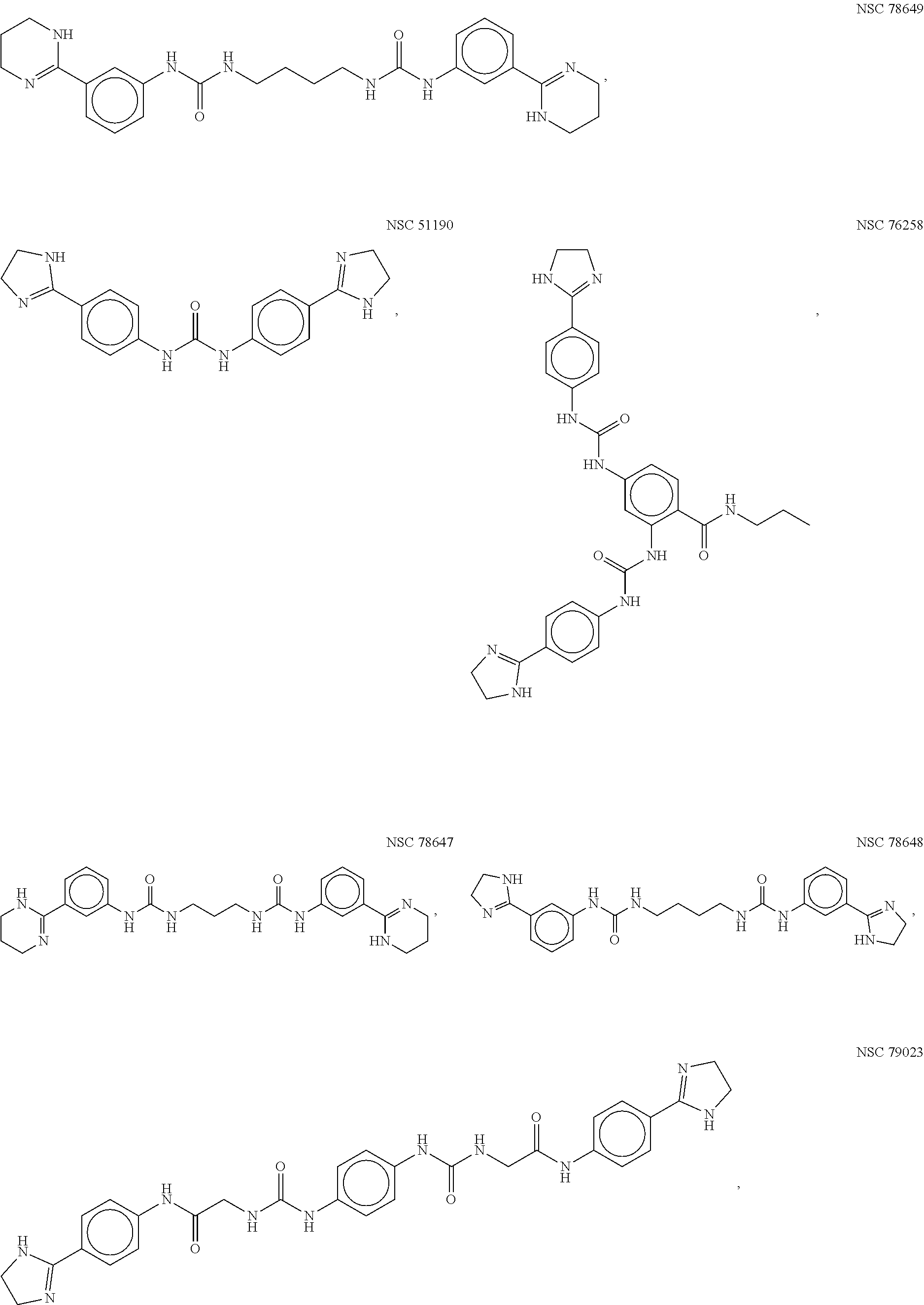

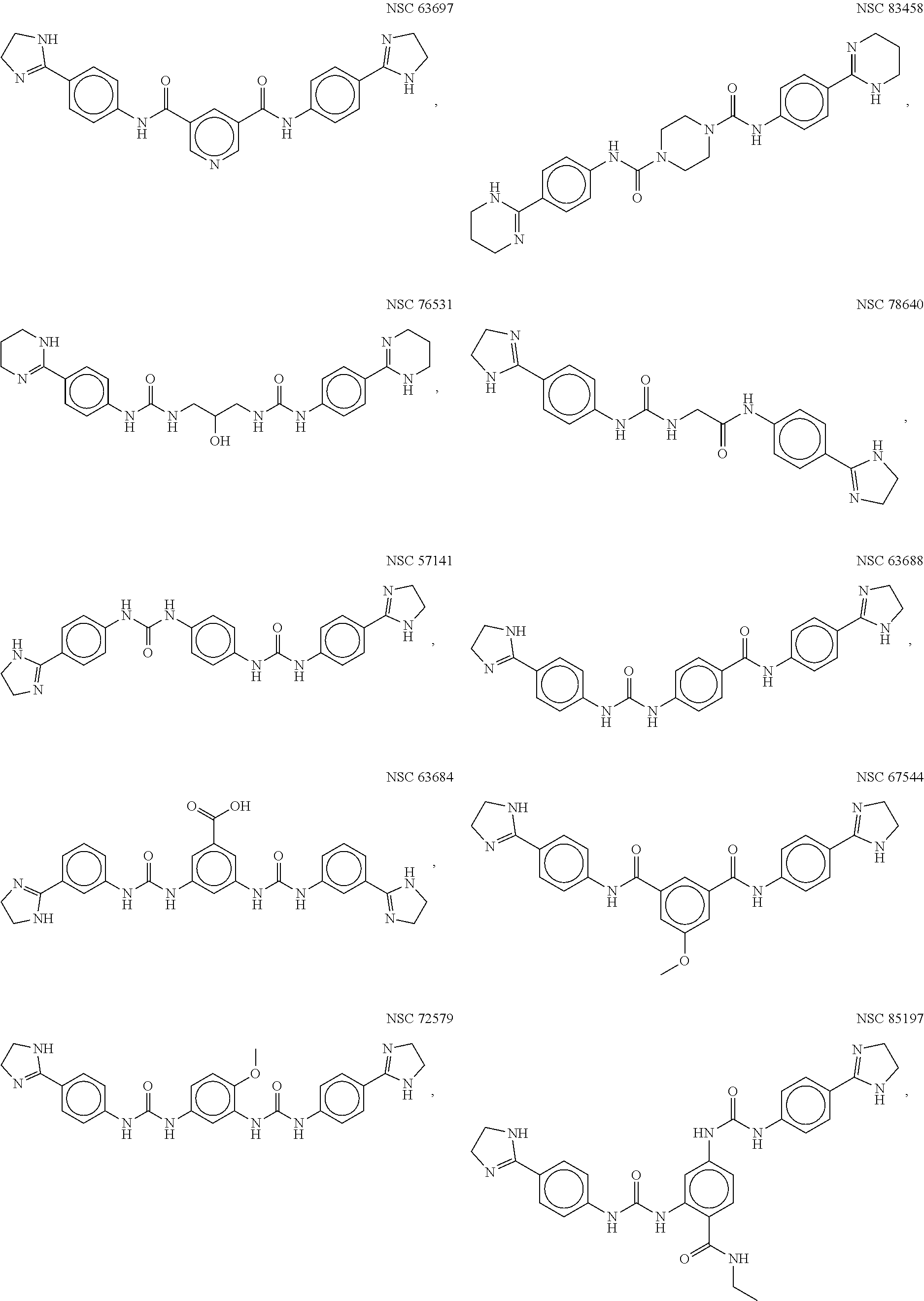

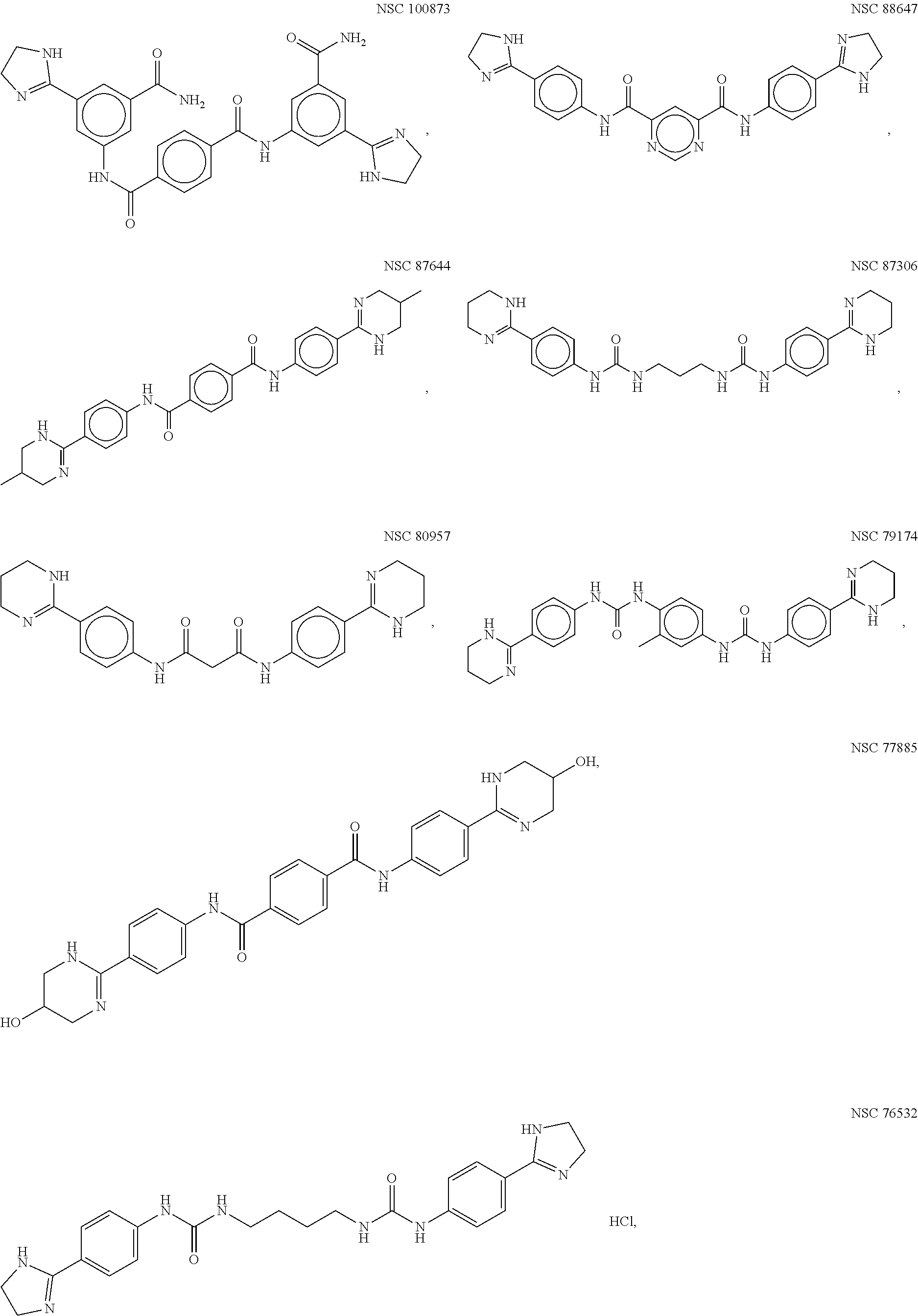

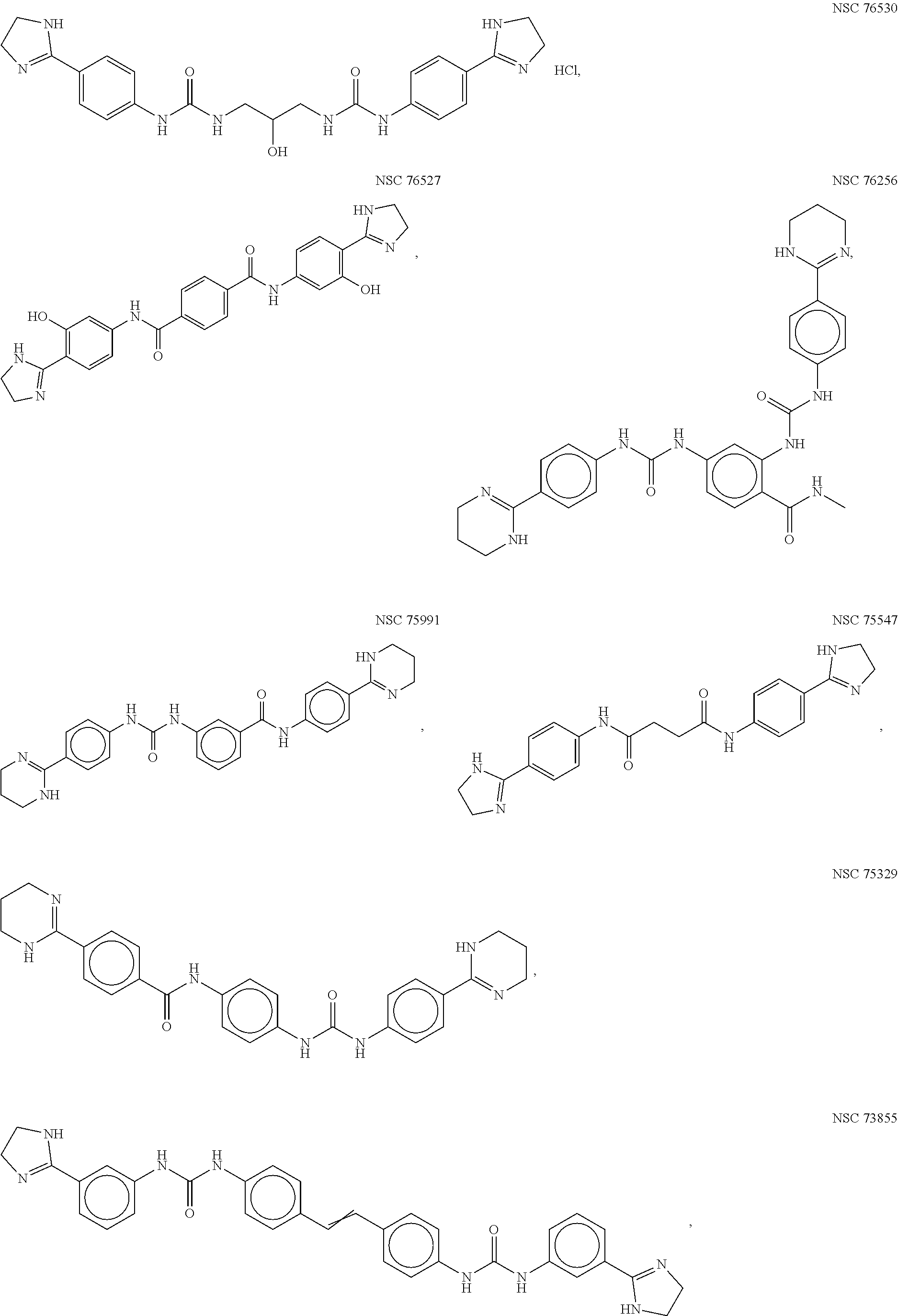

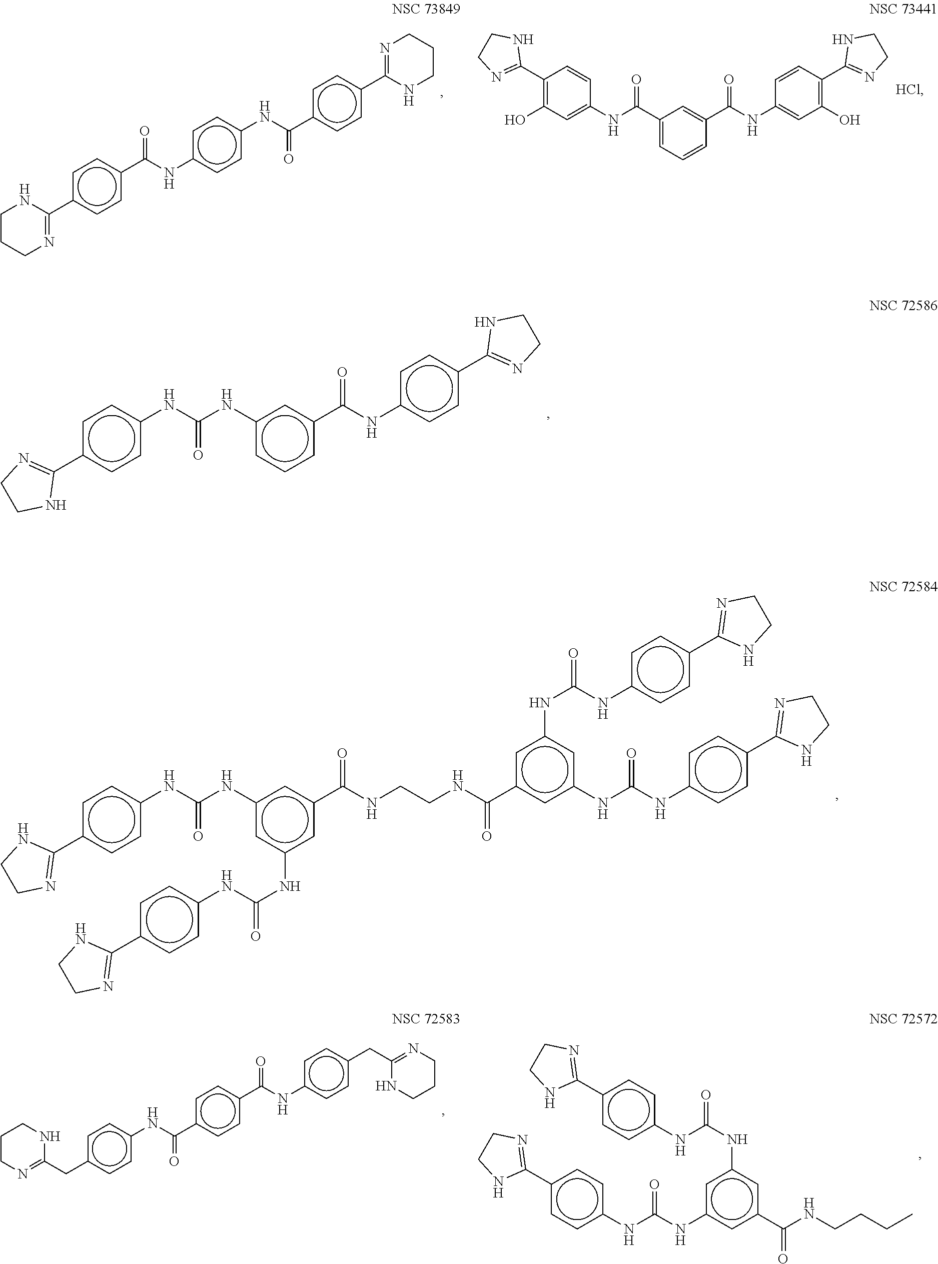

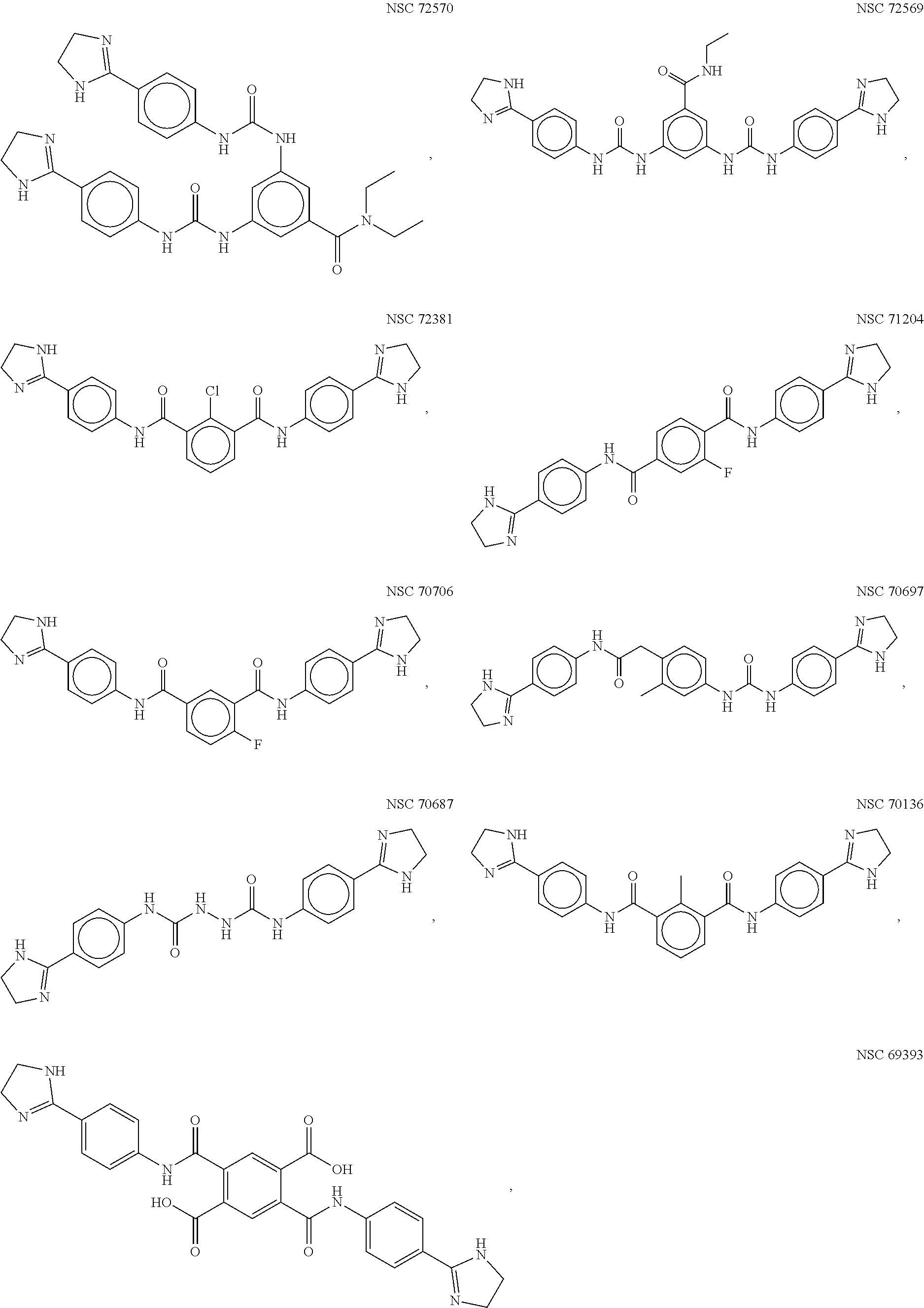

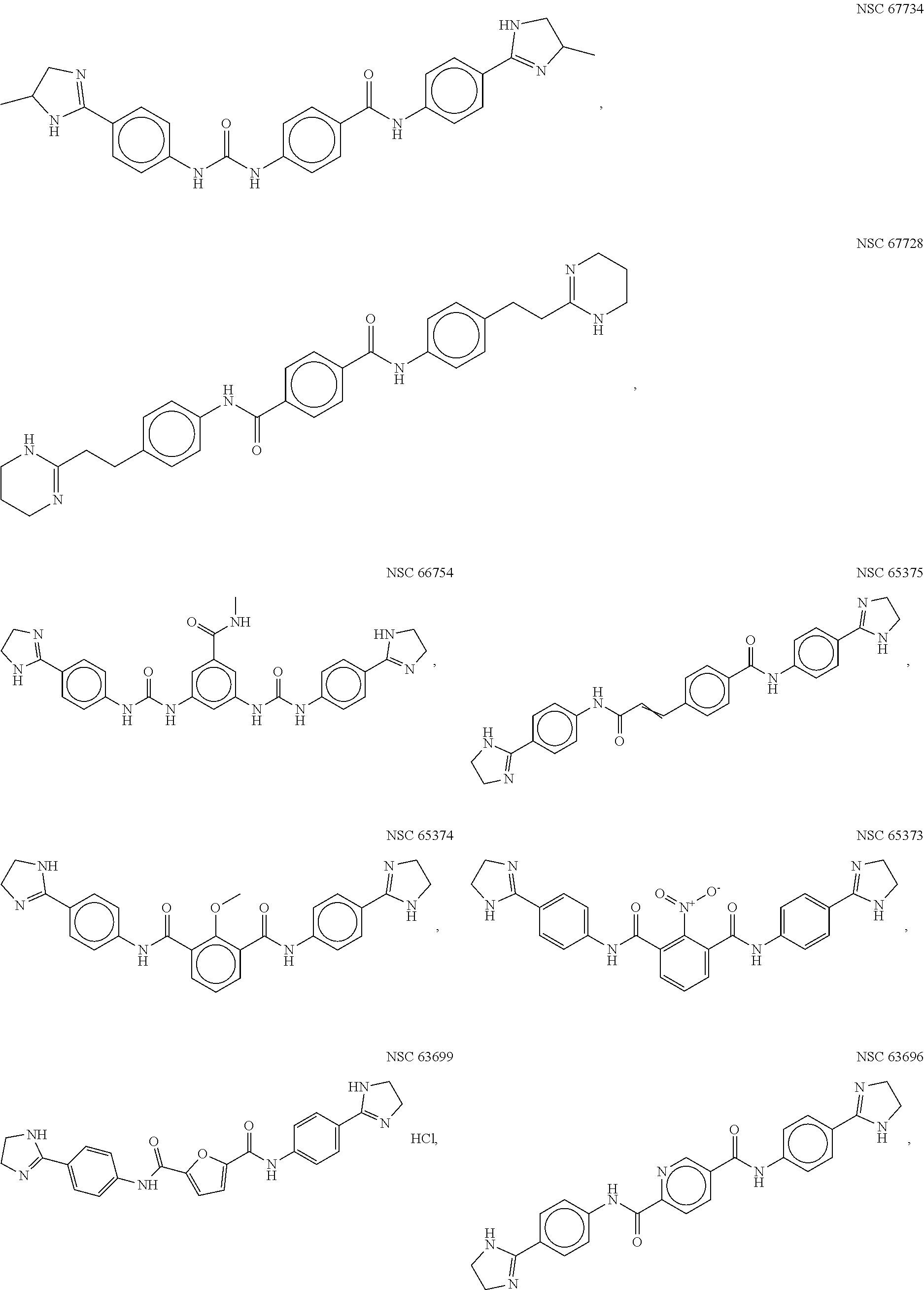

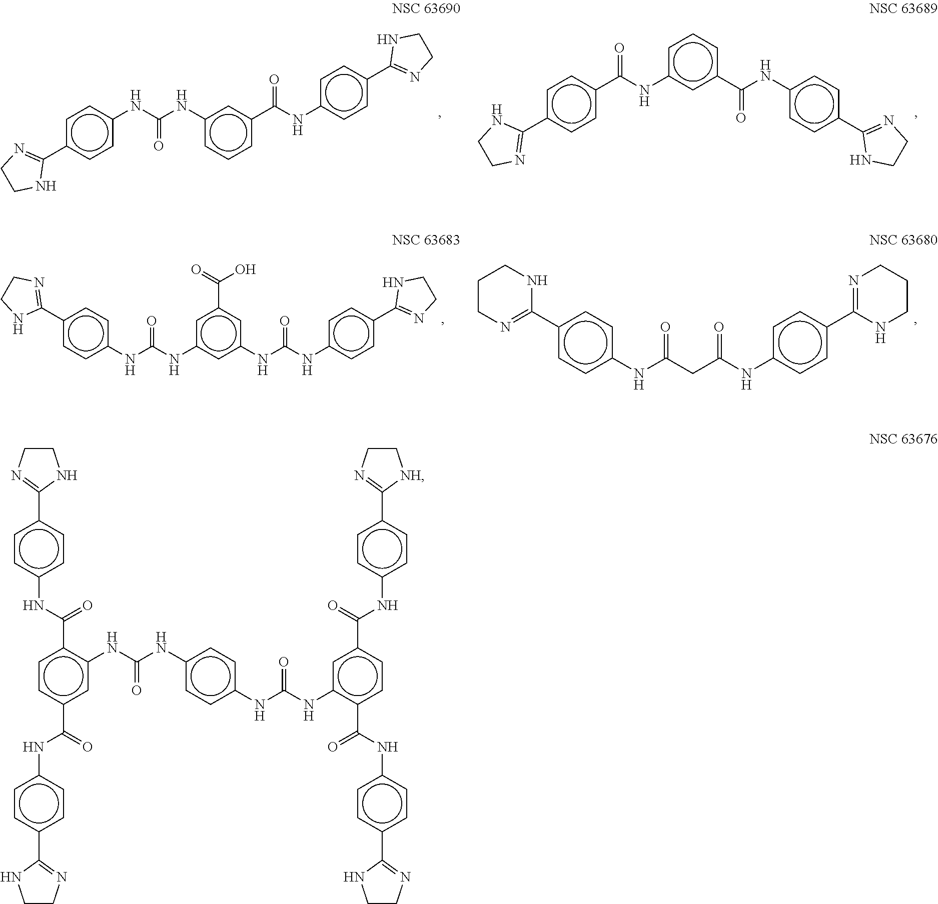

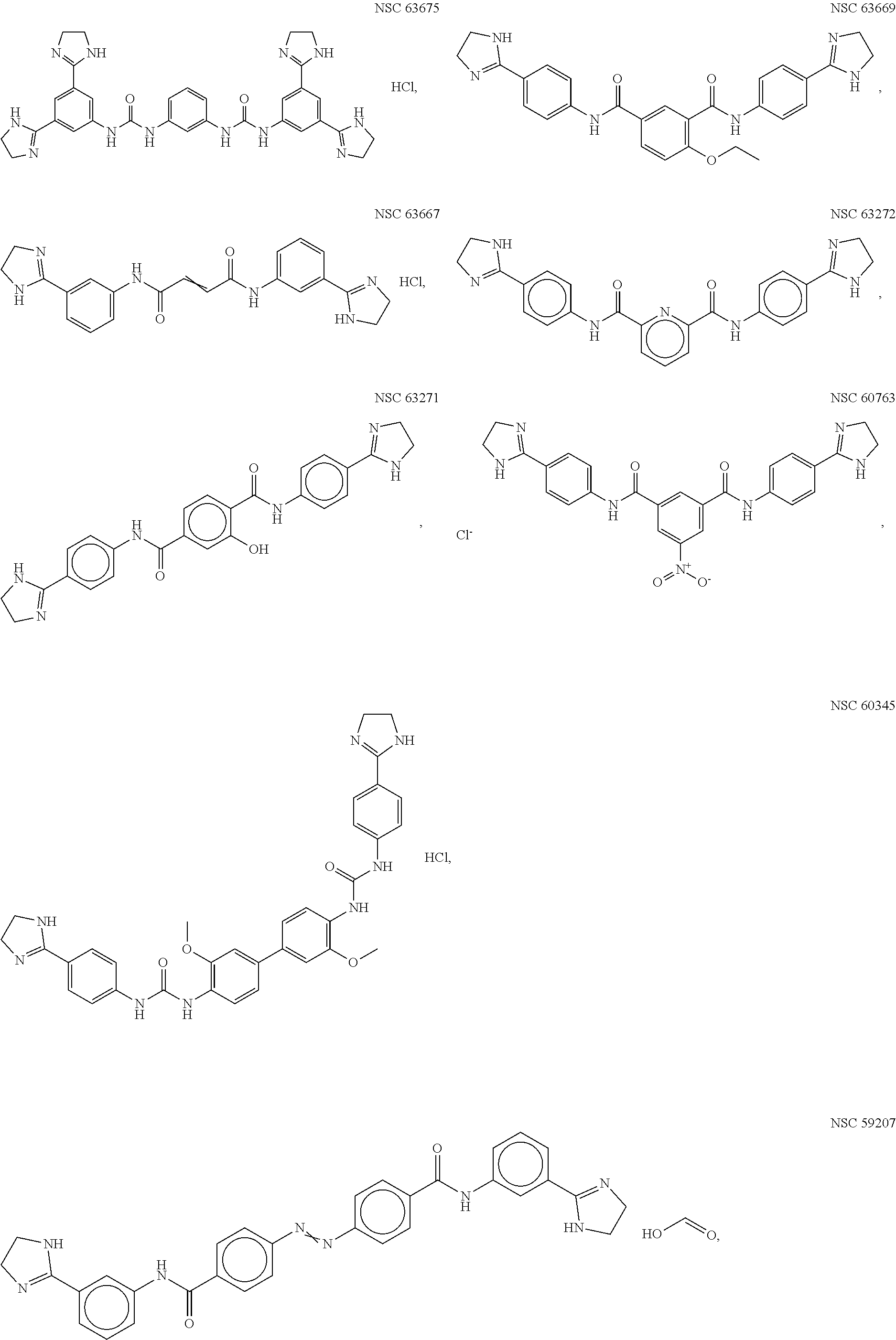

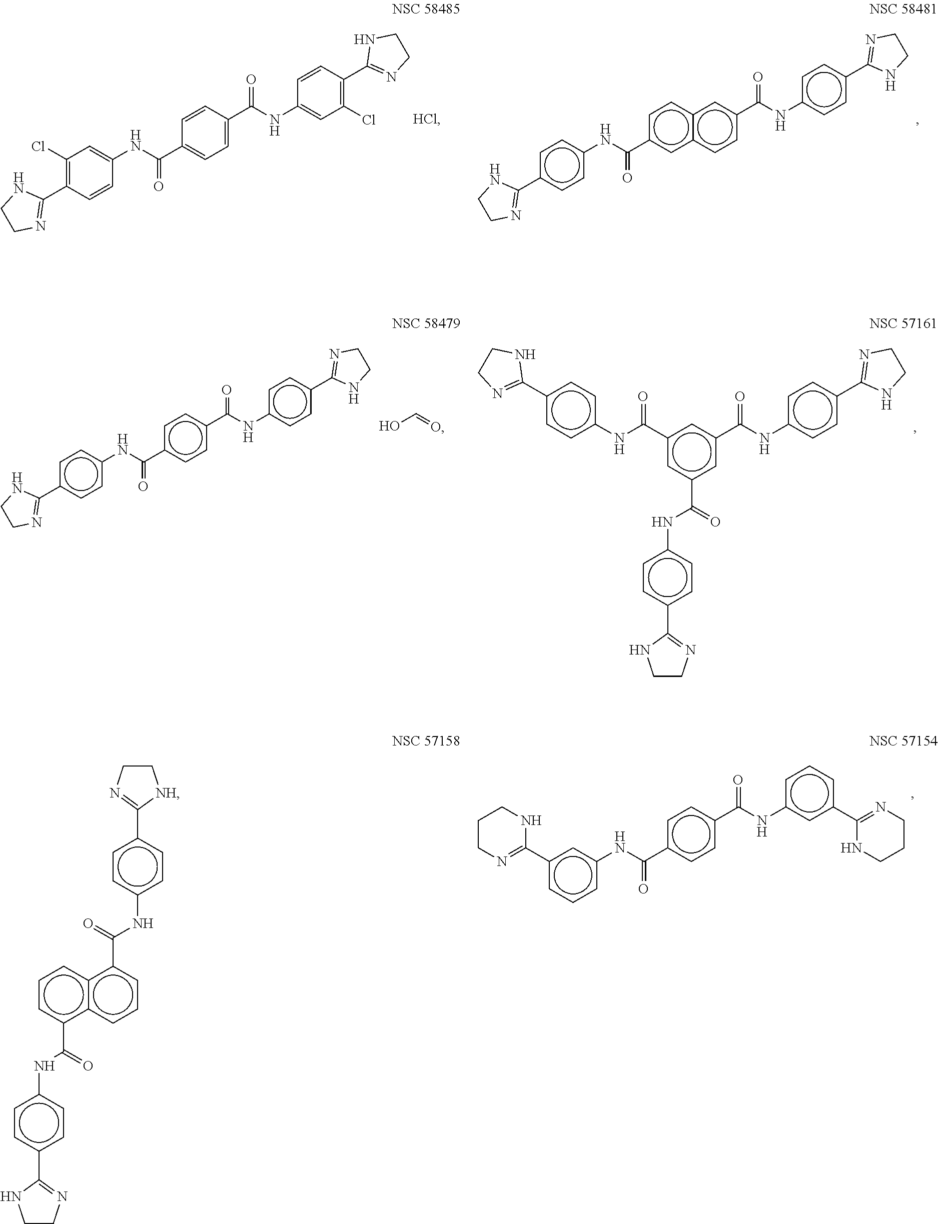

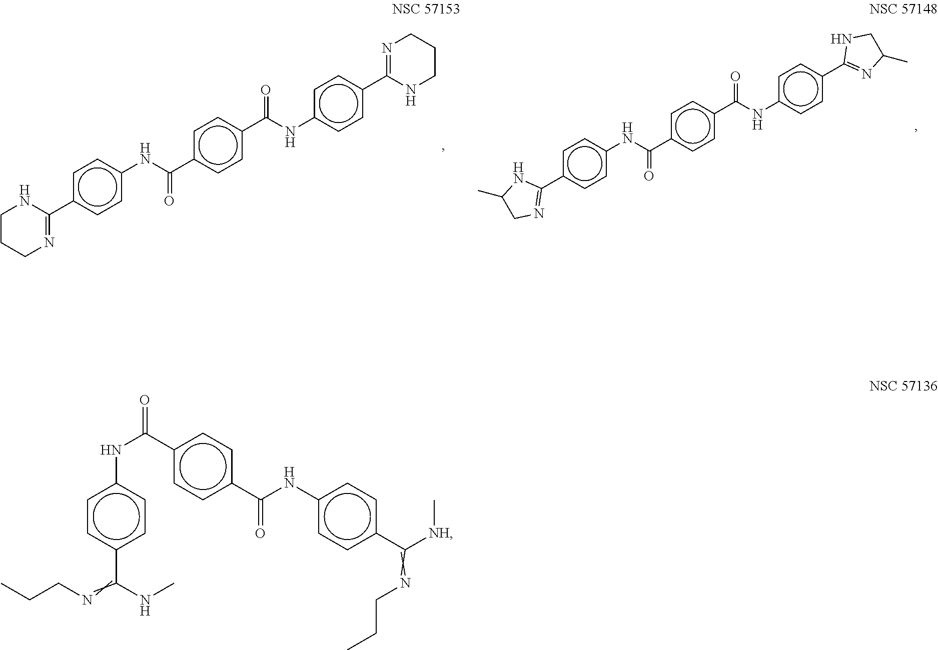

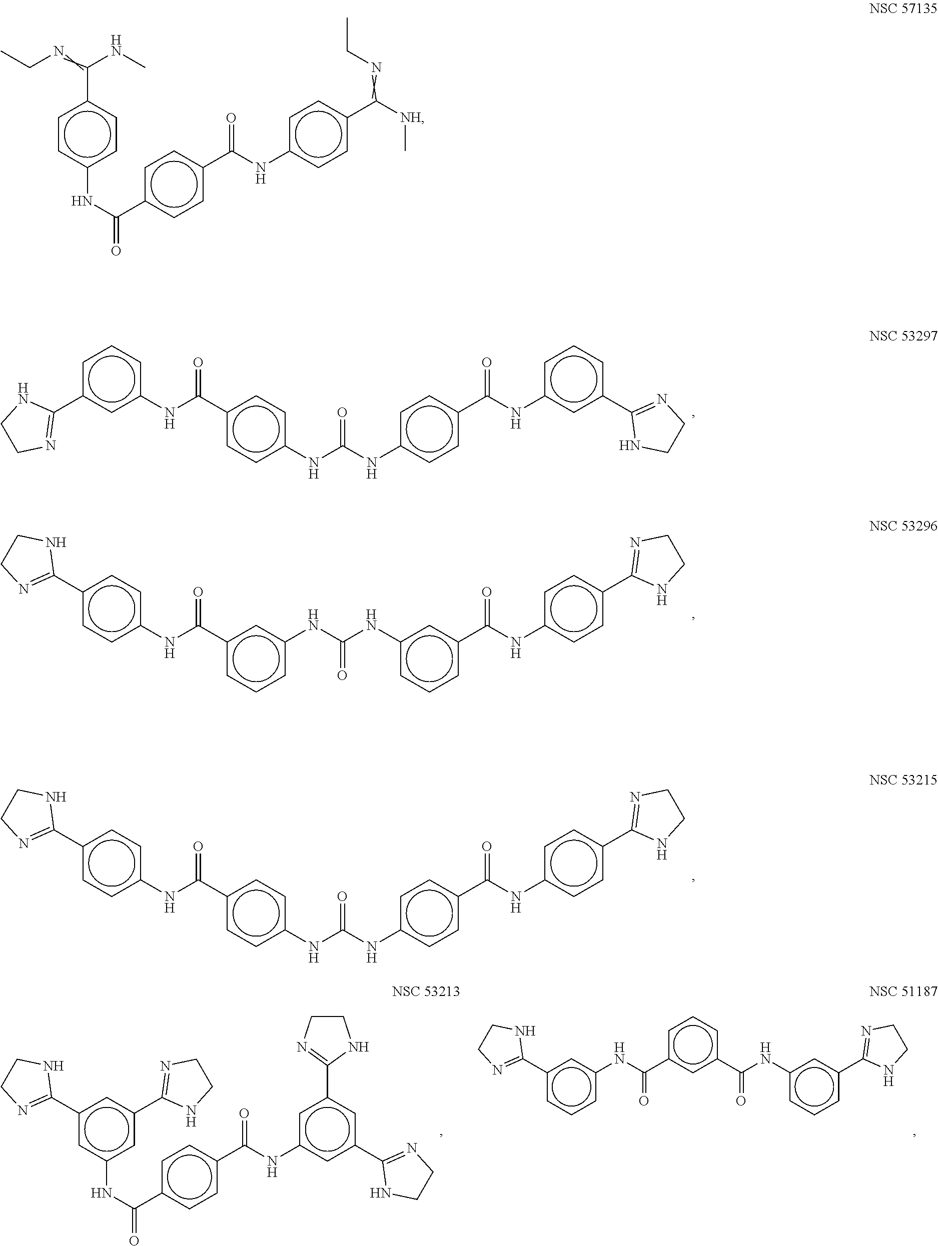

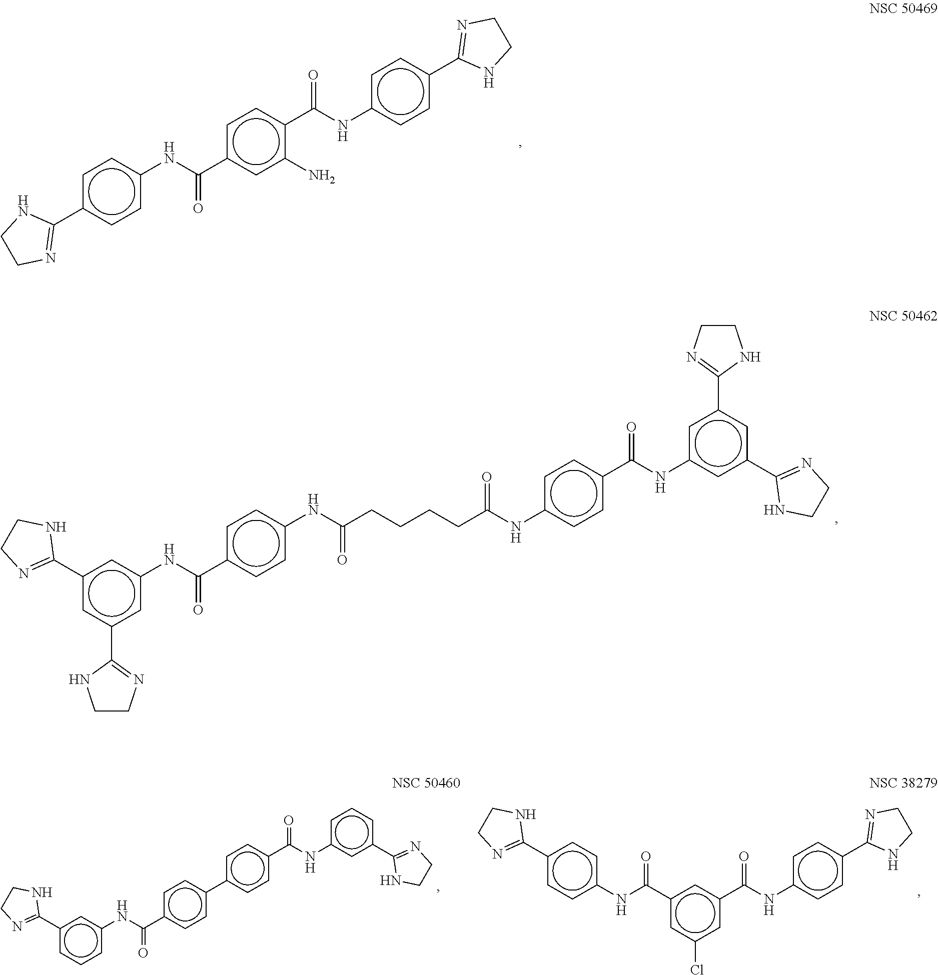

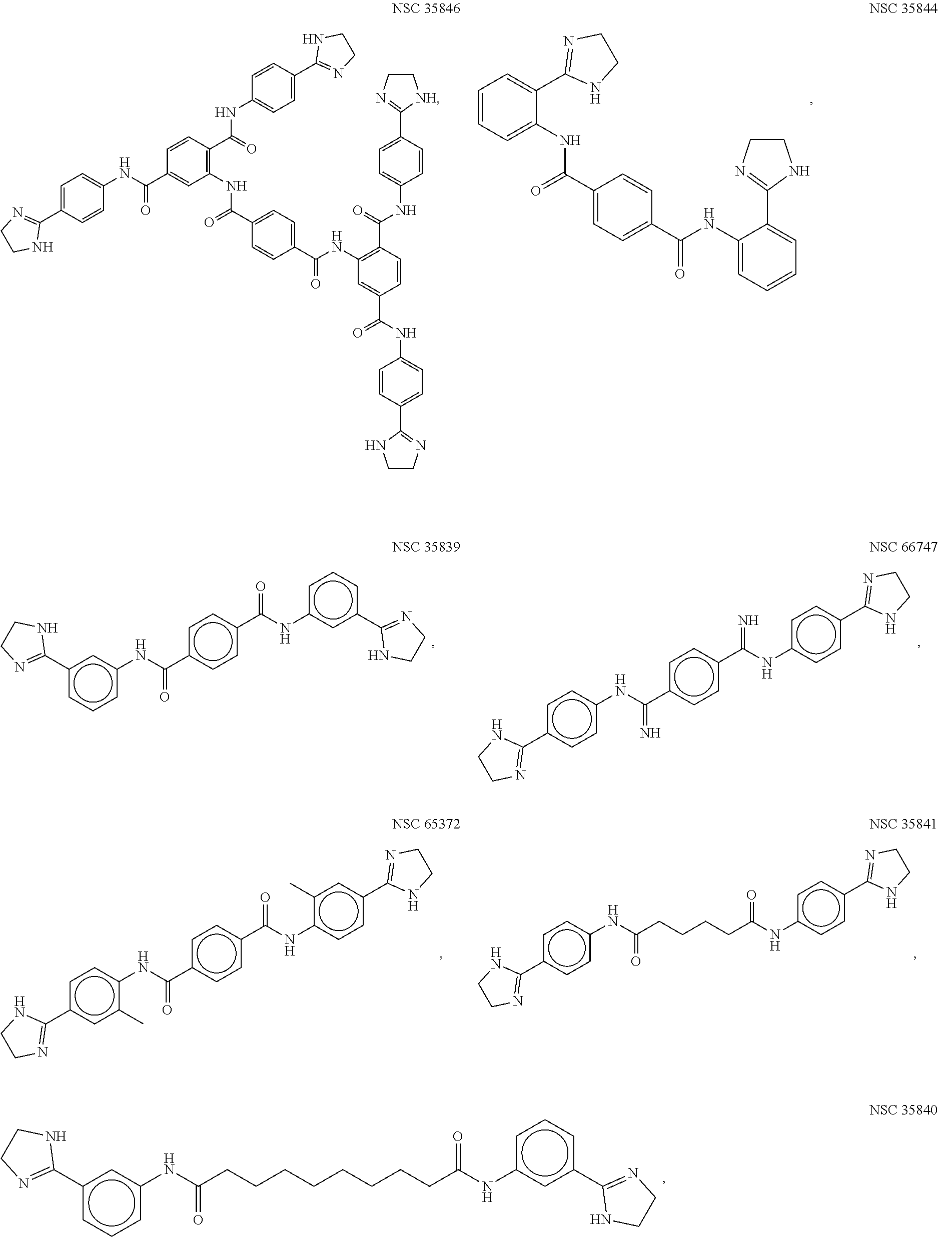

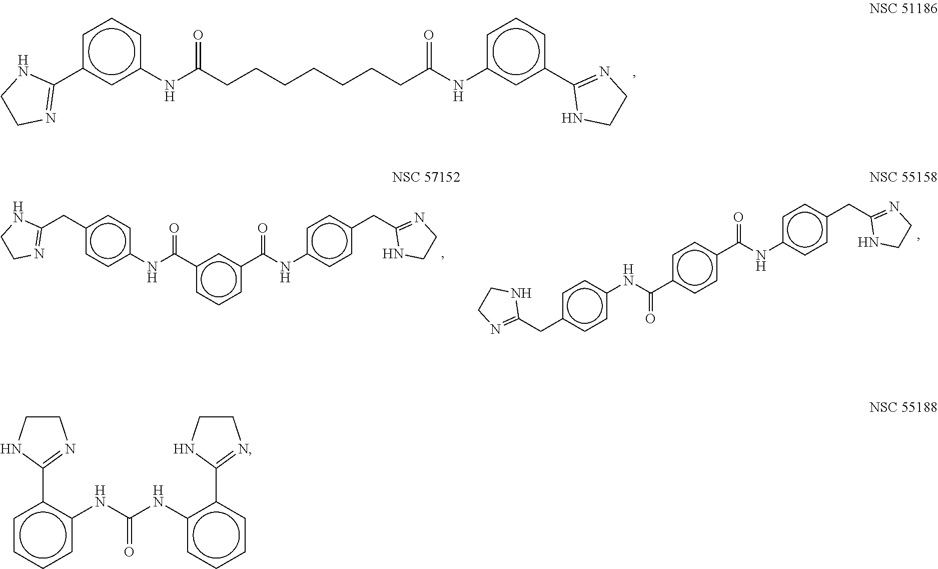

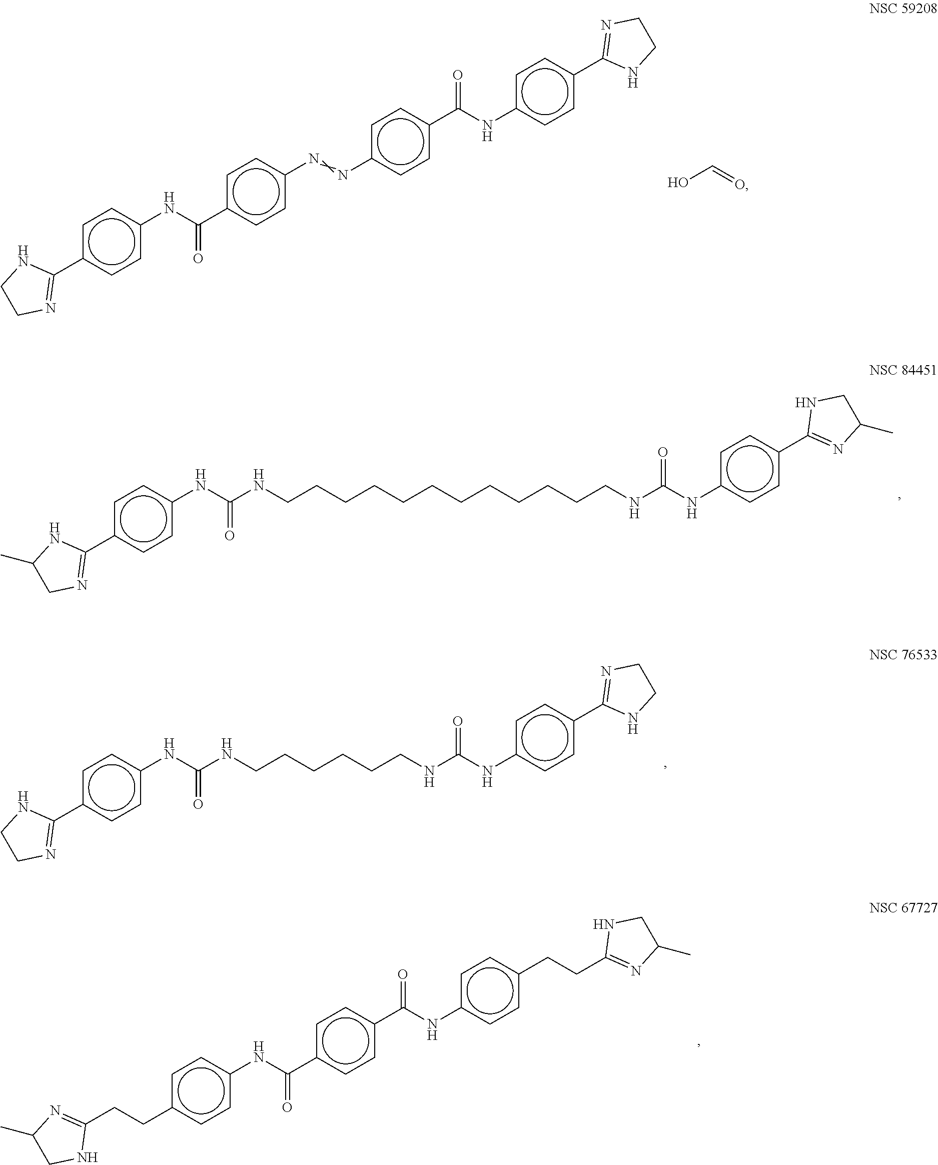

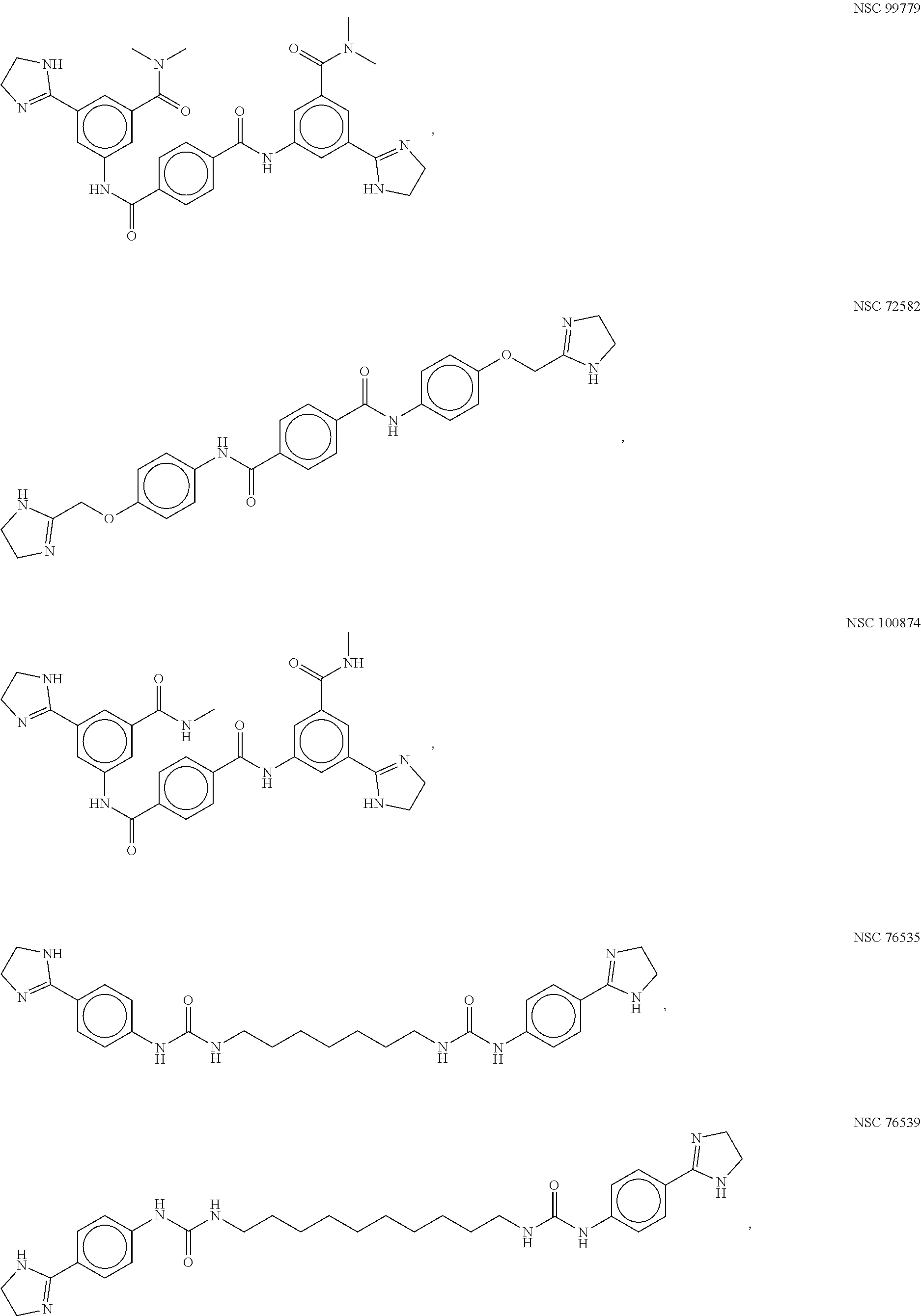

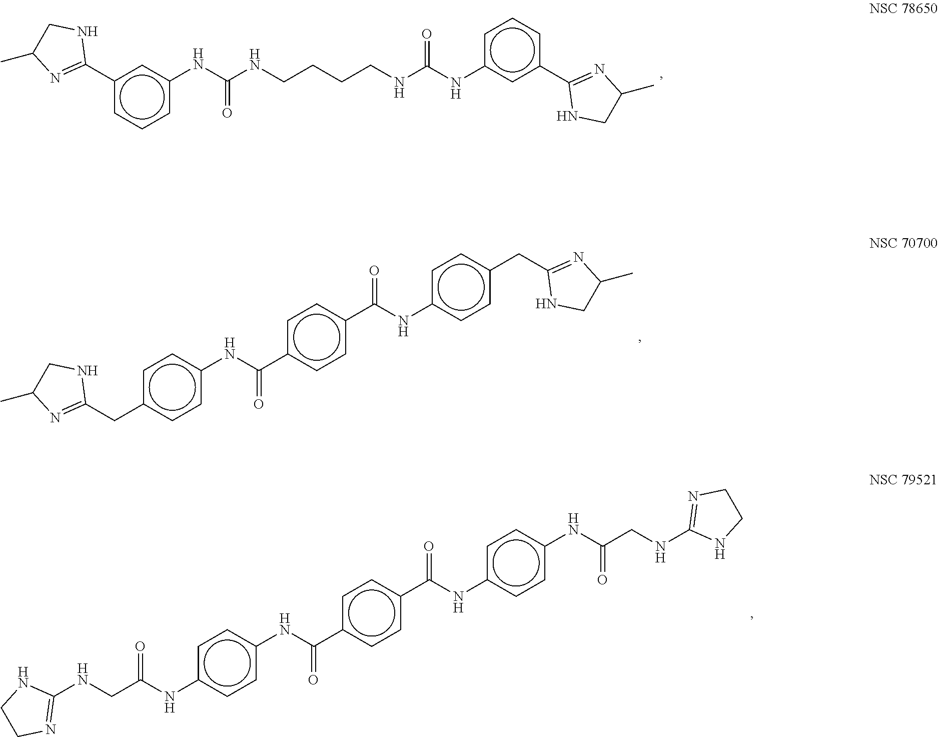

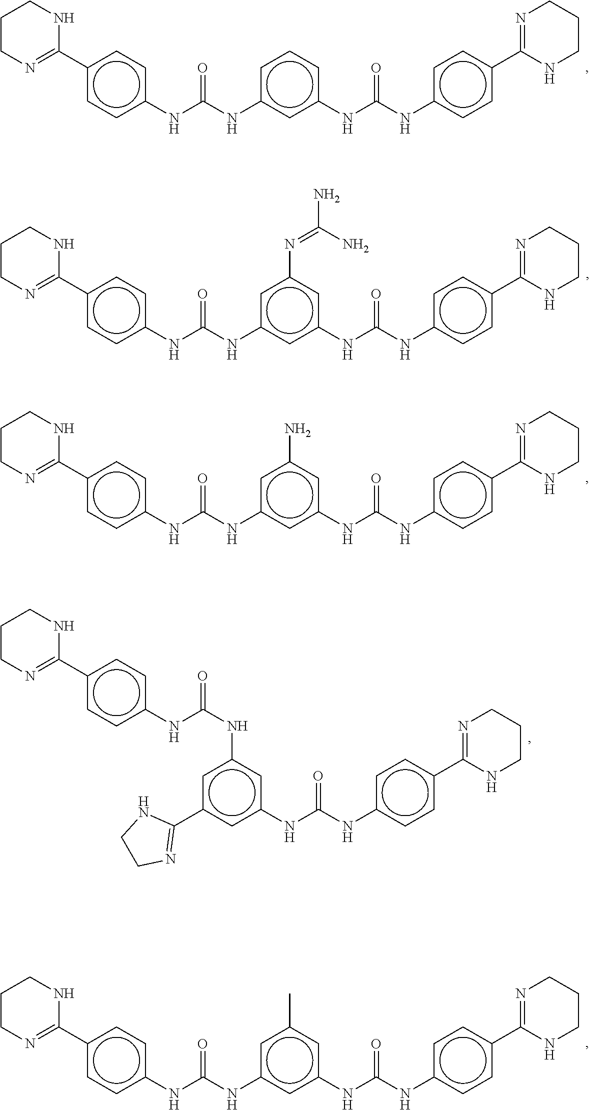

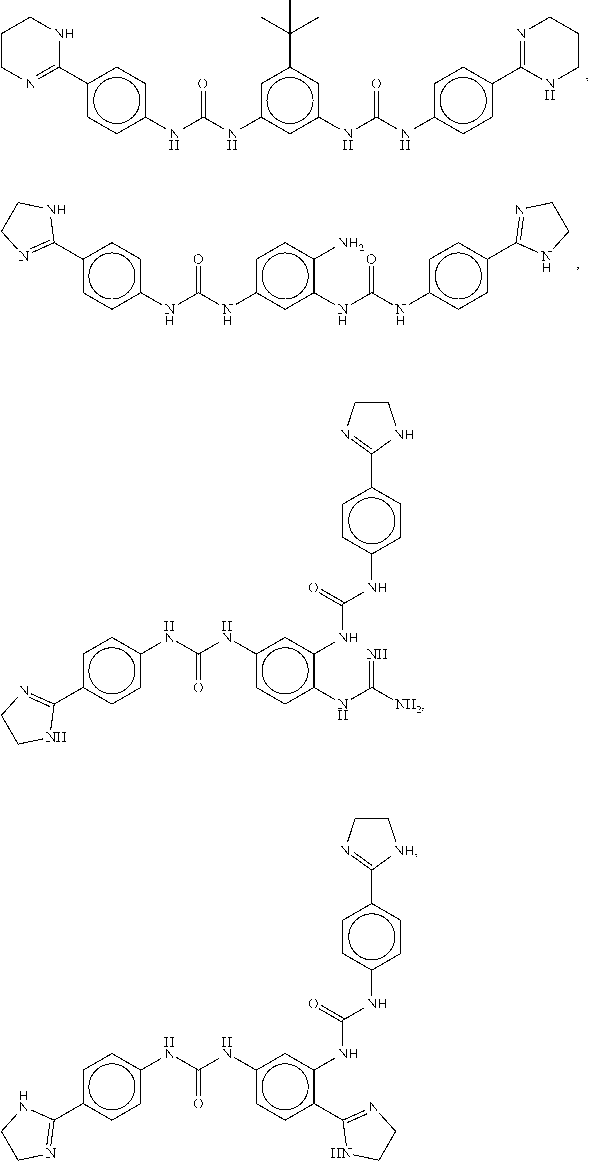

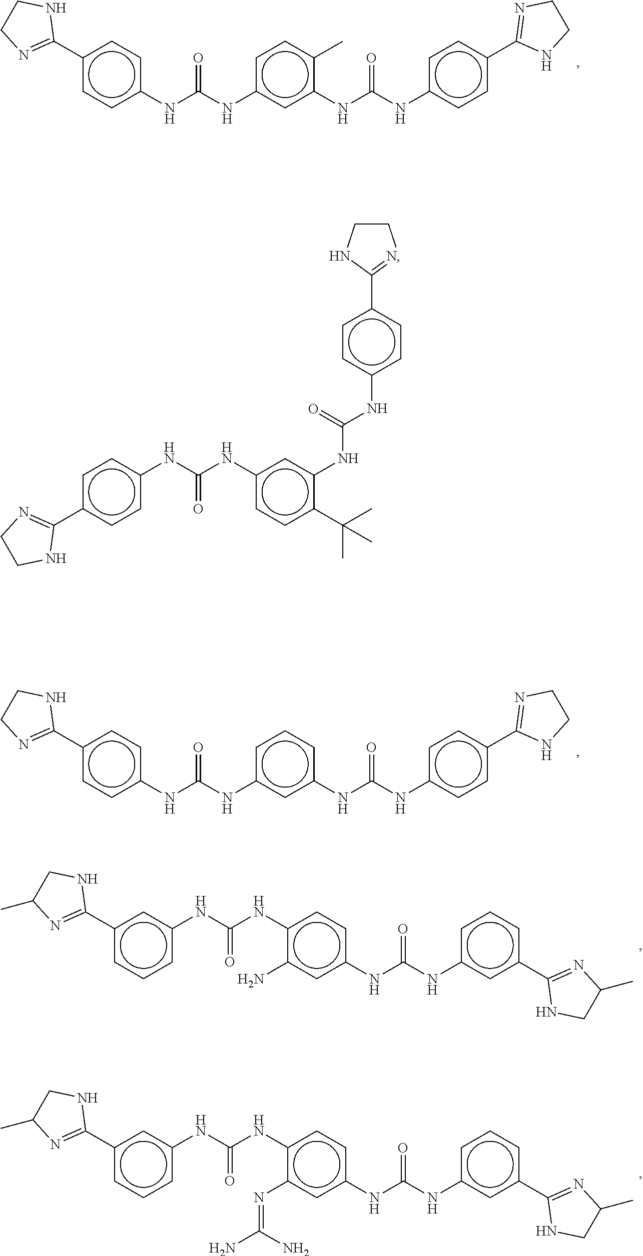

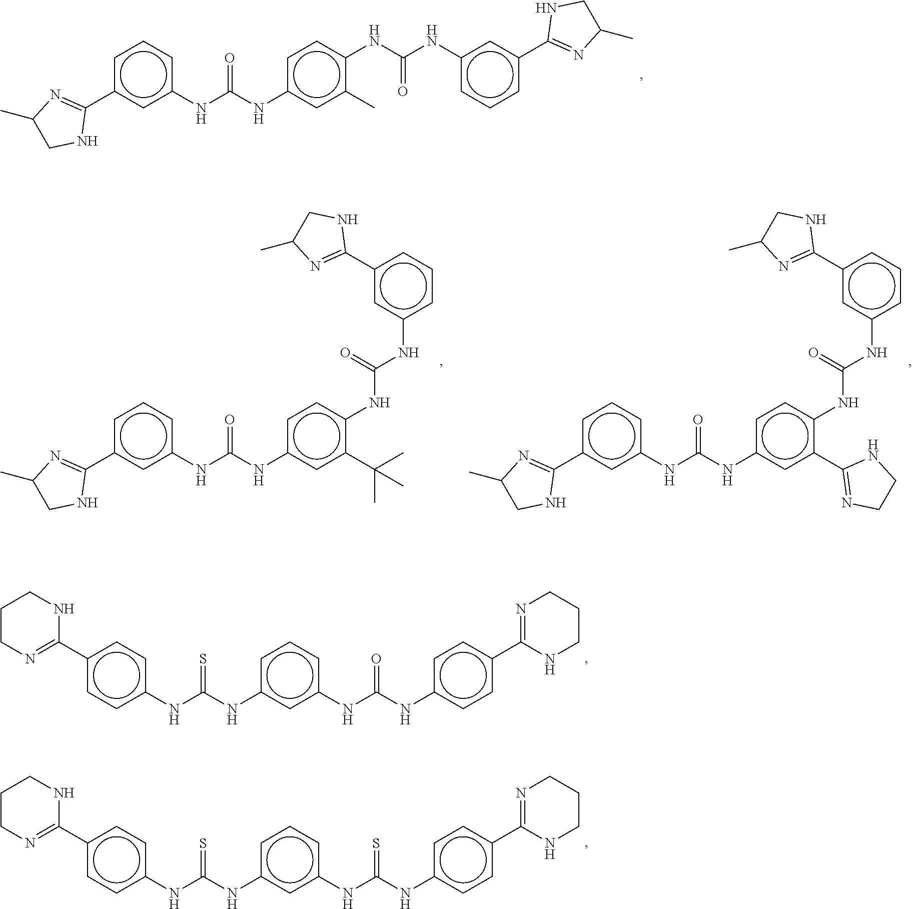

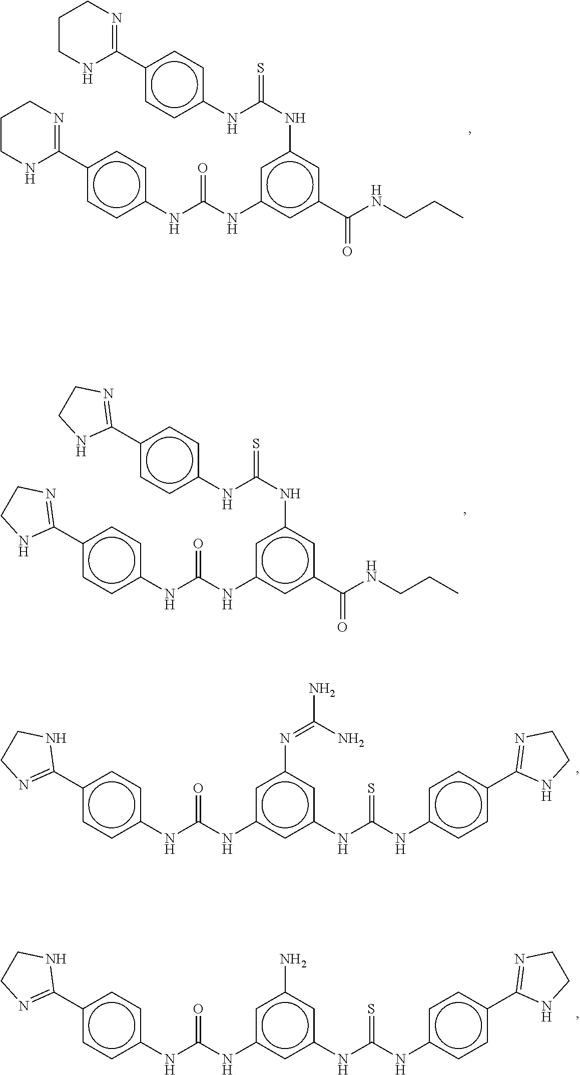

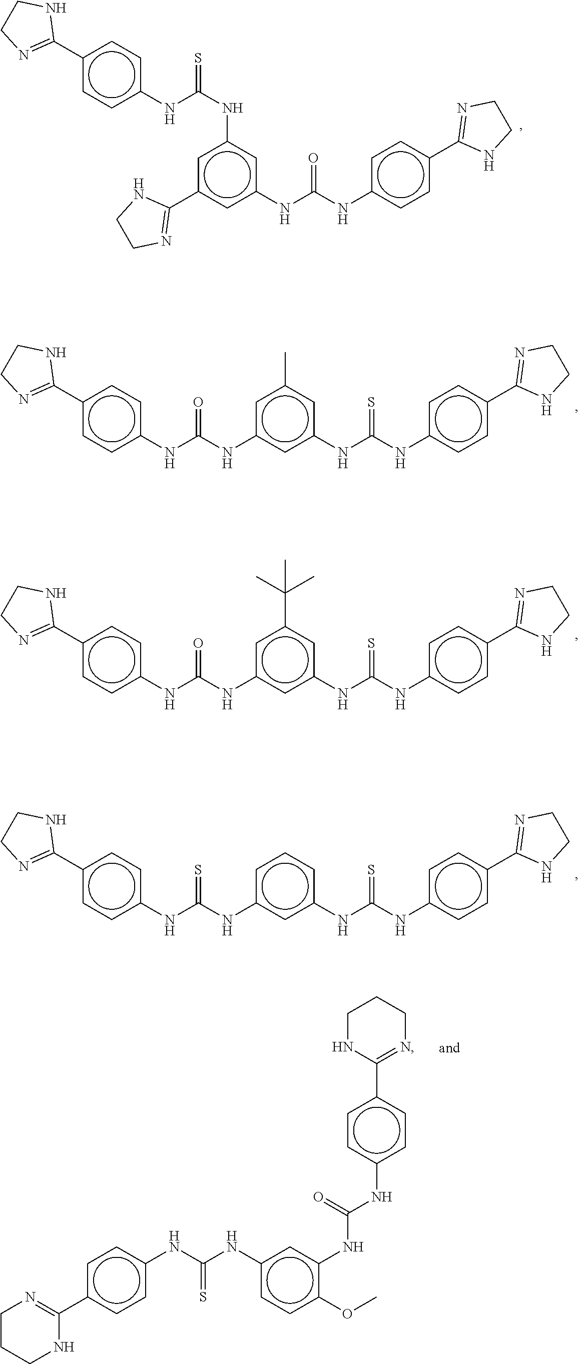

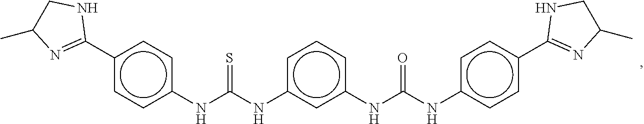

or a pharmaceutically acceptable salt thereof.

In some preferred aspects, the compound of formula (I) is selected from

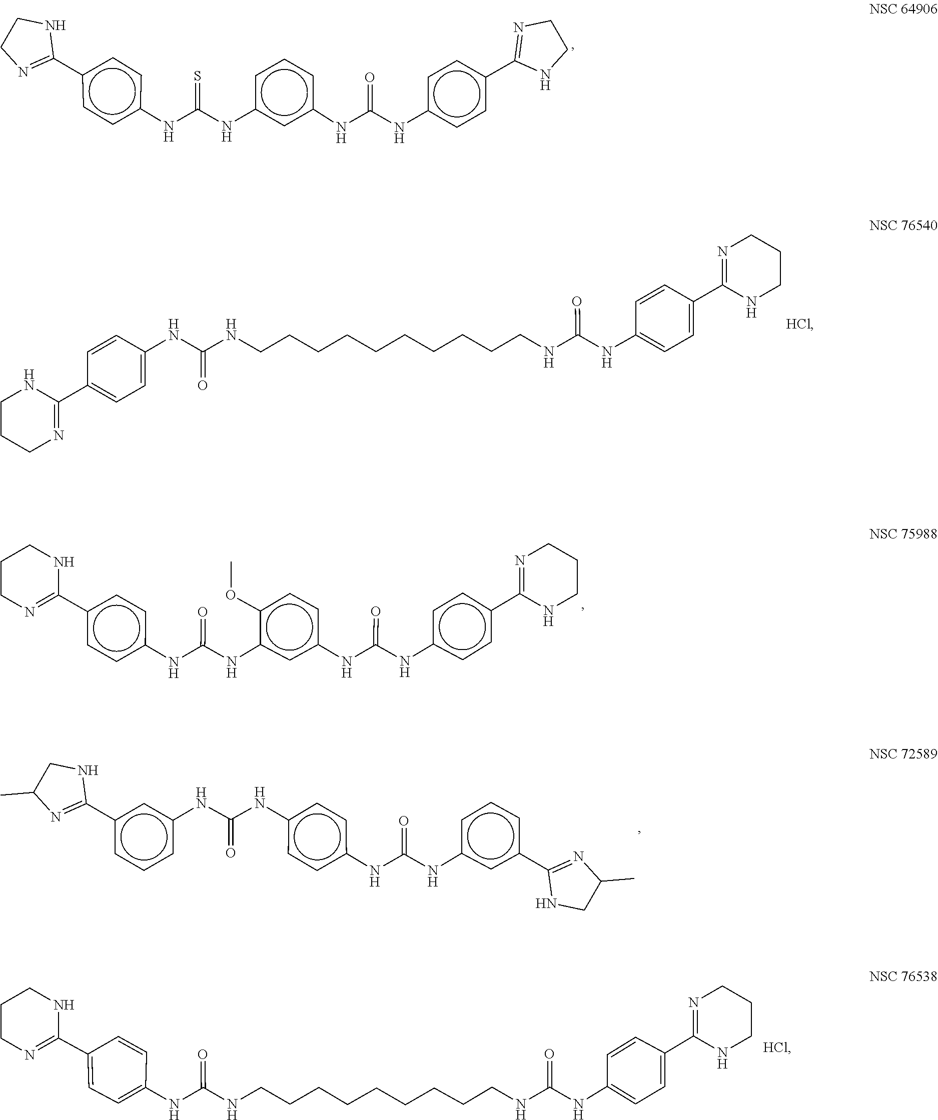

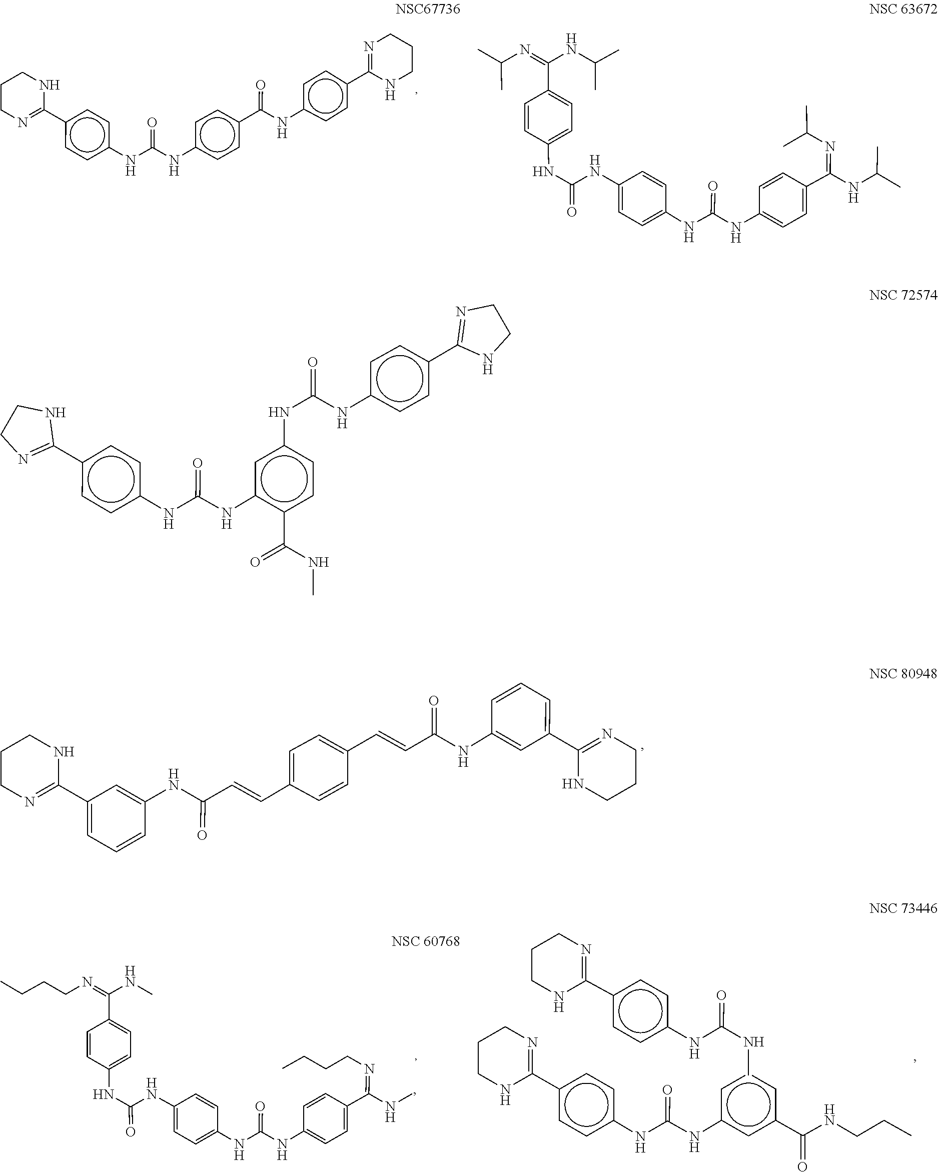

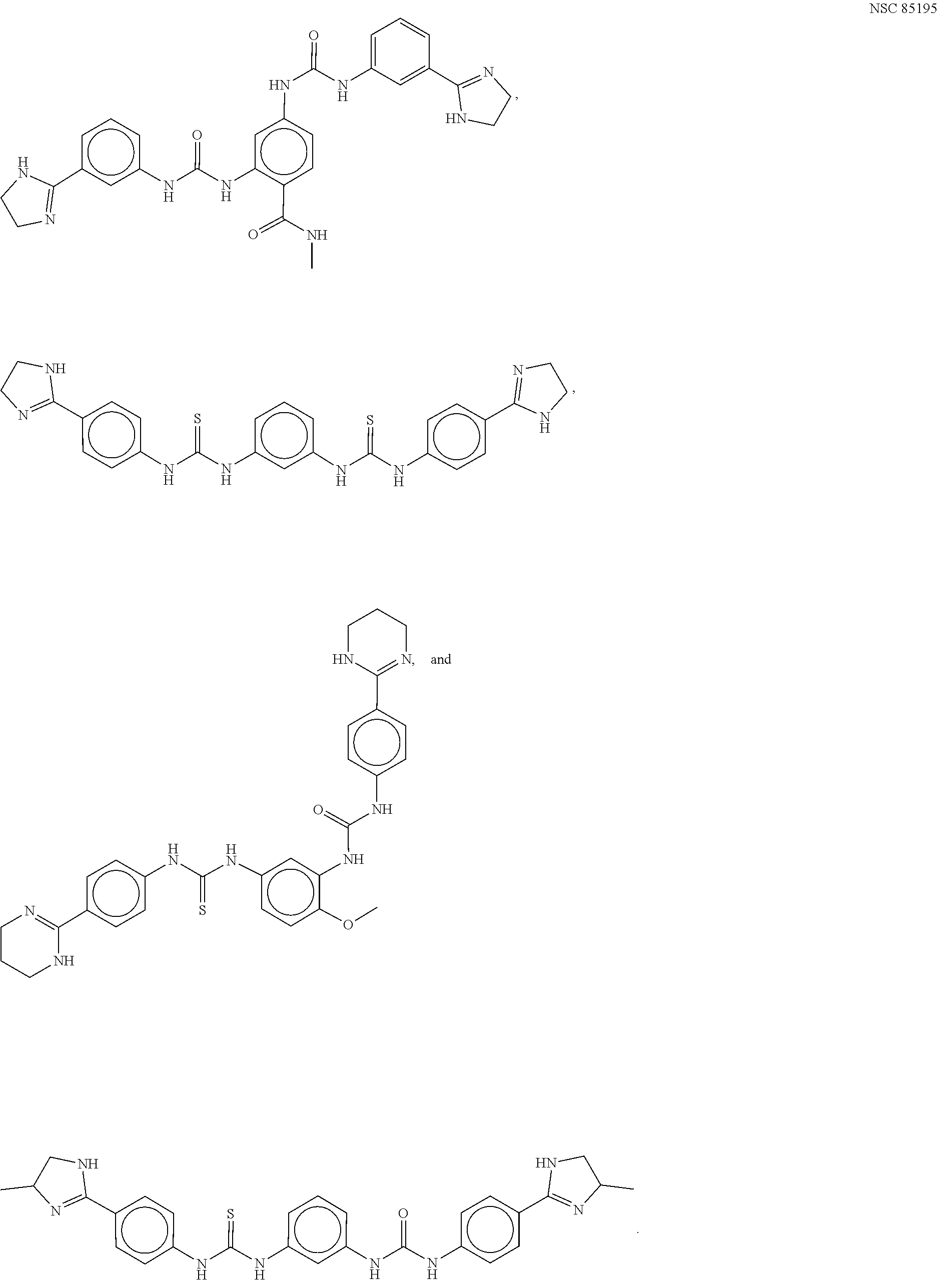

In some aspects, the disclosure provides a compound of formula (I) that is a 1,3- or 1-4-substituted phenyl bridged compound of formula (Ia)

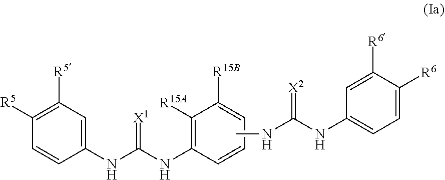

wherein
X¹ and X² are the same or different and each is O or S;
one of R⁵ and R^{5′} is

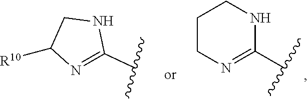

and the other is hydrogen;
one of R⁶ and R^{6′} is

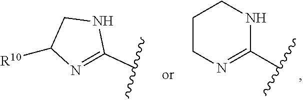

and the other is hydrogen;
wherein R⁵ and R⁶ are the same and R^{5′} and R^{6′} are the same;
R¹⁰ is hydrogen or alkyl;
R^{15A} and R^{15B} are the same or different and each is selected from hydrogen, alkyl, alkoxy, amino, alkylamido, 1,4-dihydroimidazolyl, and guanidinyl,
or a pharmaceutically acceptable salt thereof.

In some aspects, the disclosure provides a compound of formula (I) that is a 1,3- or 1-4-substituted phenyl bridged compound of formula (Ia) or a pharmaceutically acceptable salt thereof, described above, provided that the following compounds are excluded from the genus defined by formula (Ia):
(1) when the compound of formula (Ia) is a 1,4-substituted phenyl bridged compound,
X¹ and X² are both O,
R⁵ and R⁶ are both

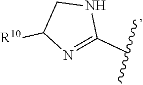

R^{5′} and R^{6′} are both hydrogen, and
R¹⁰ is hydrogen or methyl,
then R^{15A} and R^{15B} are not both hydrogen;
(2) when the compound of formula (Ia) is a 1,3- or 1,4-substituted phenyl bridged compound,
X¹ and X² are both O,
R⁵ and R⁶ are both

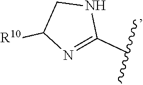

R^{5′} and R^{6′} are both hydrogen,
or
R^{5′} and R^{6′} are both

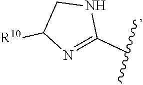

R⁵ and R⁶ are both hydrogen,
and
R¹⁰ is hydrogen,
then R^{15A} and R^{15B} are not alkylamido, R^{15A} and R^{15B} are not both hydrogen, and R^{15A} is not alkyl;
(3) when the compound of formula (Ia) is a 1,3-substituted phenyl bridged compound,
X¹ is S;
X² is O,
R⁵ and R⁶ are both

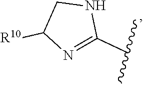

R^{5′} and R^{6′} are both hydrogen, and
R¹⁰ is hydrogen,
then R^{15A} and R^{15B} are not both hydrogen;
(4) when the compound of formula (Ia) is a 1,4-substituted phenyl bridged compound,
X¹ and X² are both O,
R⁵ and R⁶ are both

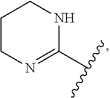 [SMILES: *C1=NCCCN1]

and
R^{5′} and R^{6′} are both hydrogen,
then R^{15A} and R^{15B} are not both hydrogen, and R^{15A} is not alkyl;
(5) when the compound of formula (Ia) is a 1,4-substituted phenyl bridged compound,
X¹ and X² are both O,
R^{5′} and R^{6′} are both

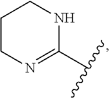 [SMILES: *C1=NCCCN1]

and
R⁵ and R⁶ are both hydrogen,
then R^{15A} is not amino; and/or
(6) when the compound of formula (Ia) is a 1,3- or 1,4-substituted phenyl bridged compound,
X¹ and X² are both O,
R⁵ and R⁶ are both

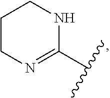 [SMILES: *C1=NCCCN1]

R^{5′} and R^{6′} are both hydrogen,
then R^{15A} and R^{15B} are not alkylamido.

In any of the foregoing aspects of the compound of formula (Ia) or a pharmaceutically acceptable salt thereof, wherein X¹ and X² are both O. In other aspects, at least one of X¹ and X² is S.

In an aspect of the compound of formula (Ia) or a pharmaceutically acceptable salt thereof, the compound is a 1,3-substituted phenyl bridged compound,
R⁵ and R⁶ are both

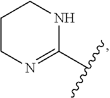 [SMILES: *C1=NCCCN1]

R^{5′} and R^{6′} are both hydrogen, and
R^{15A} is hydrogen.

In an aspect of the compound of formula (Ia) or a pharmaceutically acceptable salt thereof, the compound is a 1,3-substituted phenyl bridged compound,
R⁵ and R⁶ are both

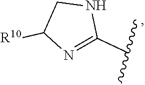

R^{5′} and R^{6′} are both hydrogen,
R¹⁰ is hydrogen, and
R^{15B} is hydrogen.

In an aspect of the compound of formula (Ia) or a pharmaceutically acceptable salt thereof, the compound is a 1,4-substituted phenyl bridged compound,
R⁵ and R⁶ are both

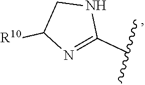

R^{5′} and R^{6′} are both hydrogen,
R¹⁰ is methyl, and
R^{15B} is hydrogen.

Exemplary compounds of formula (Ia) include, e.g.,

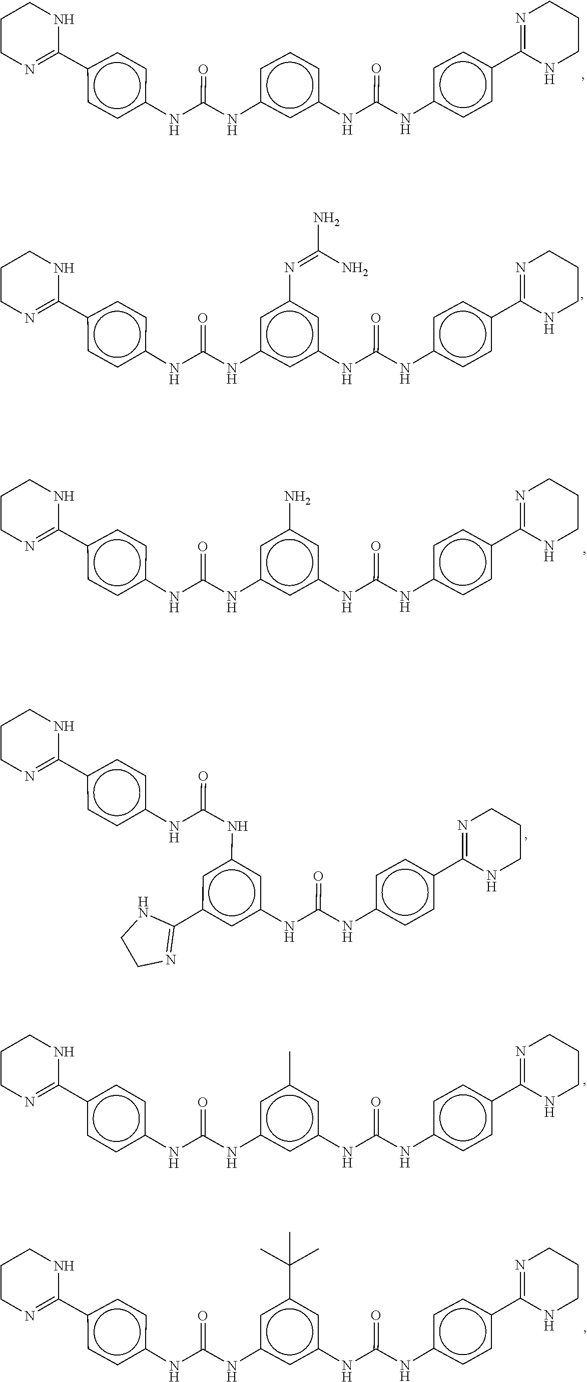

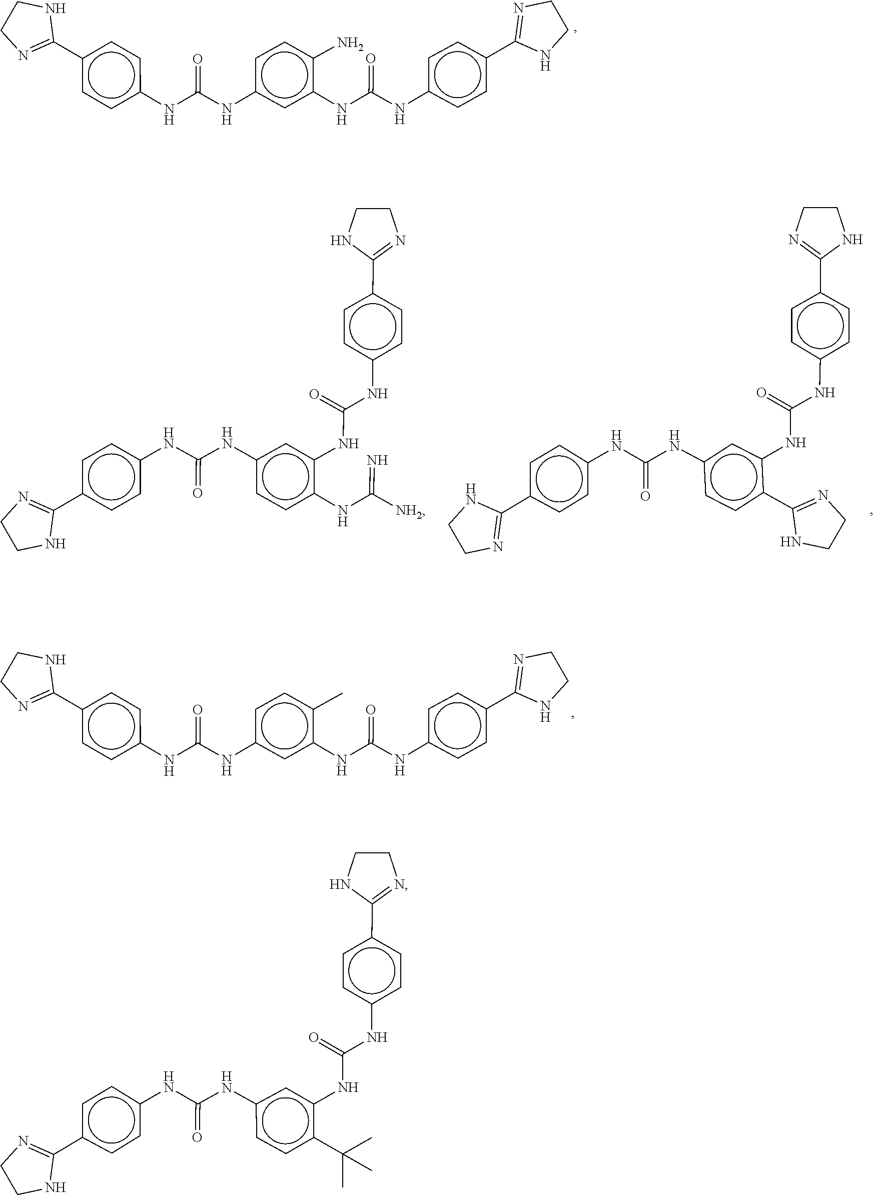

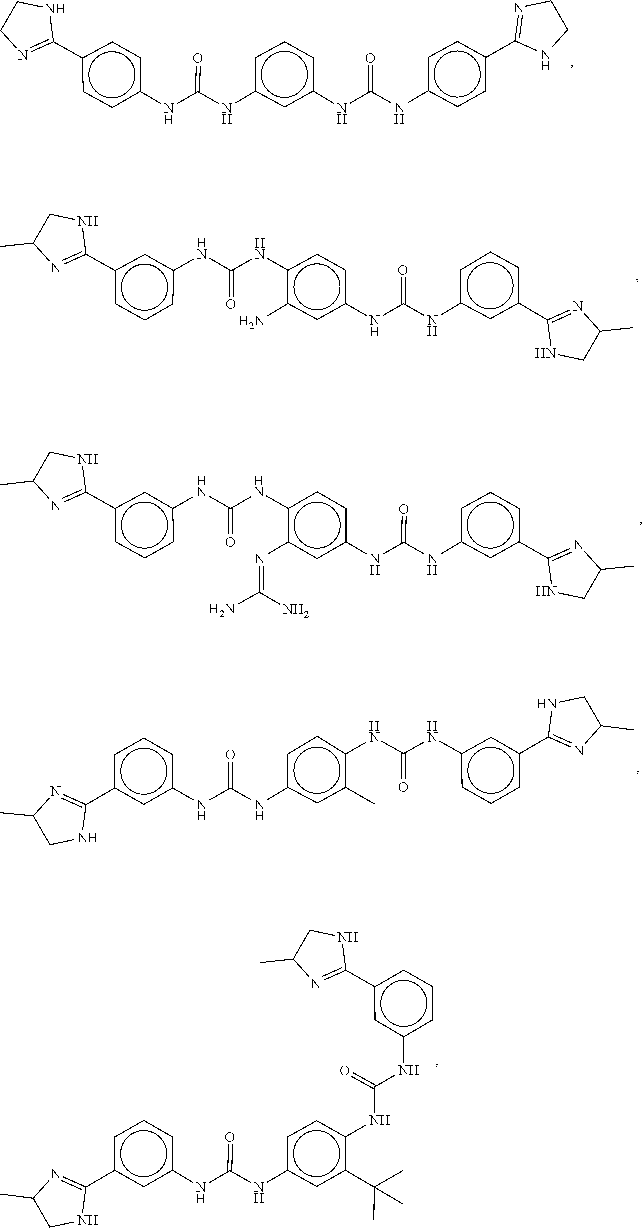

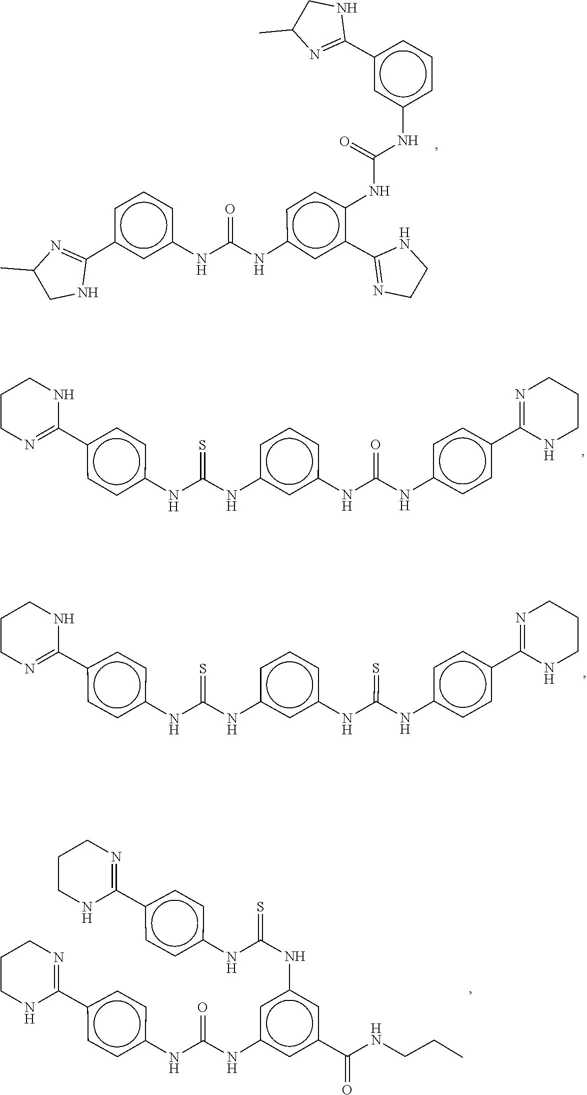

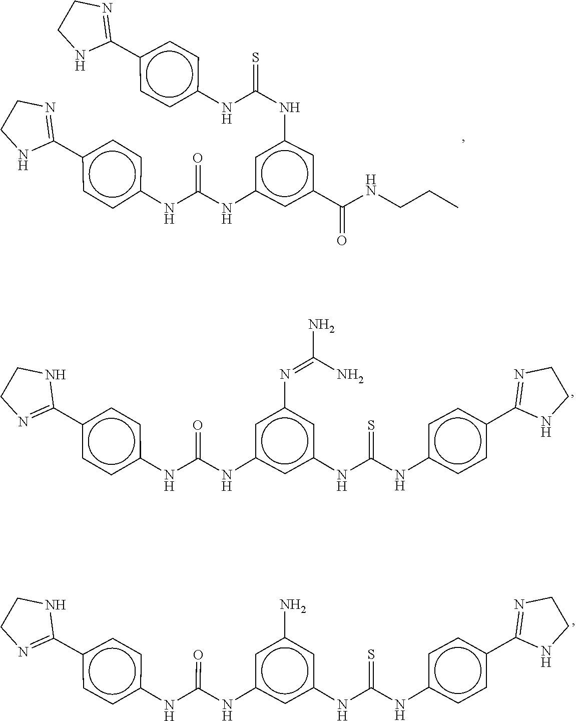

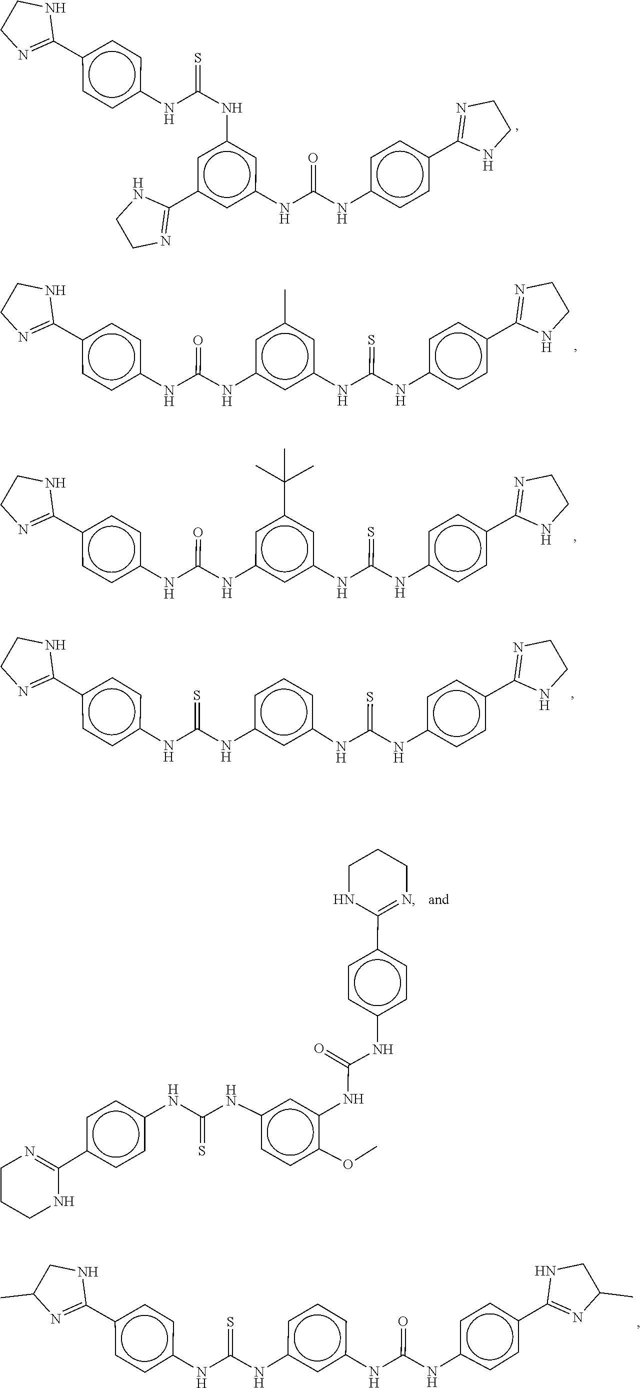

or a pharmaceutically acceptable salt thereof.

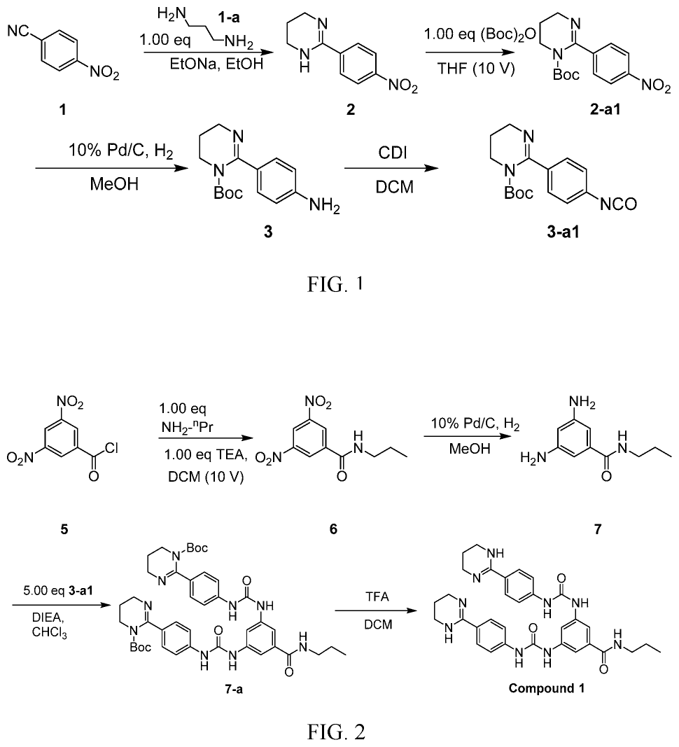

Compounds of formula (Ia) can be provided by any suitable synthetic method, including the methods set forth in FIGS. 1-16.

In another aspect, the disclosure provides a method of treating a disorder mediated by claudin-2 and/or claudin-15 in a subject comprising administering to the subject an effective amount of a compound of formula (II)

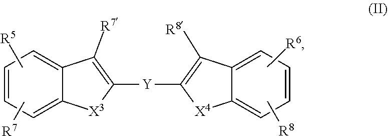

wherein
X³ and X⁴ are the same or different and each is O, S, or NH;
R⁵ and R⁶ are the same and are selected from

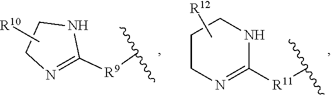

and —C(NHR¹³)═NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is hydrogen or alkyl;
R⁷, R⁸, R^{7′}, and R^{8′} are the same or different and each is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
Y is

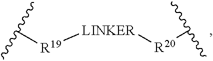

wherein
LINKER is selected from
(a) —CR²¹=CR²²—,
(b) —CR²¹₂CR²²₂—, and

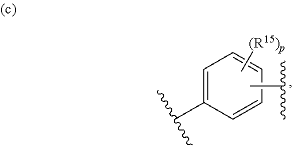

wherein each R¹⁵, R²¹, and R²² is the same or different and is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2; and
each of R¹⁹ and R²⁰ is independently selected from
(i) a bond,
(ii) —CR²¹=CR²²—,
(iii) —CR²¹₂CR²²₂—, and

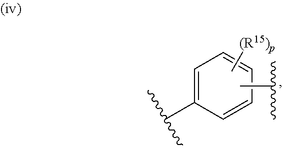

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2;
or a pharmaceutically acceptable salt thereof to the subject.

In some aspects, the compound of formula (II) is a compound of formula (II-1):

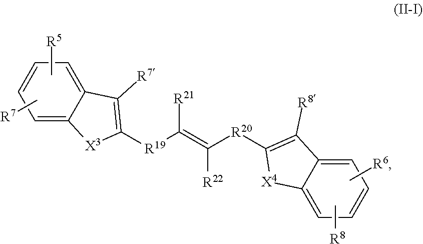

wherein
X³ and X⁴ are the same or different and each is O, S, or NH;
R⁵ and R⁶ are the same and are selected from

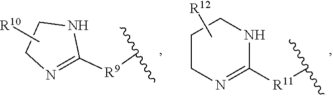

and —C(NHR¹³)—NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is hydrogen or alkyl;
R⁷, R⁸, R^{7′}, and R^{8′} are the same or different and each is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
wherein R²¹ and R²² are the same or different and are independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
each of R¹⁹ and R²⁰ is independently selected from
(i) a bond,
(ii) —CR²¹=CR²²—,
(iii) —CR²¹2CR²²₂—, and

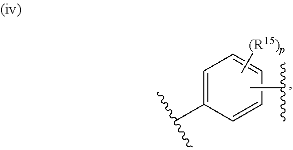

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2;
or a pharmaceutically acceptable salt thereof to the subject.

In some aspects, the compound of formula (II) is a compound of formula (II-2):

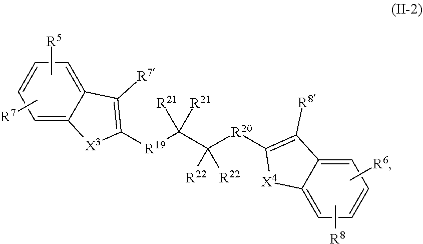

wherein
X³ and X⁴ are the same or different and each is O, S, or NH;
R⁵ and R⁶ are the same and are selected from

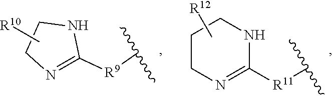

and —C(NHR¹³)═NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is hydrogen or alkyl;
R⁷, R⁸, R^{7′}, and R^{8′} are the same or different and each is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
wherein each R²¹ and R²² is the same or different and is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
each of R¹⁹ and R²⁰ is independently selected from
(i) a bond,
(ii) —CR²¹=CR²²—,
(iii) —CR²¹2CR²²₂—, and

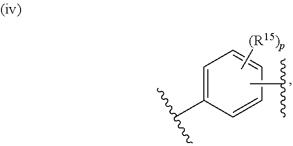

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2;
or a pharmaceutically acceptable salt thereof to the subject.

In other aspects, the compound of formula (II) is a compound of formula (II-3):

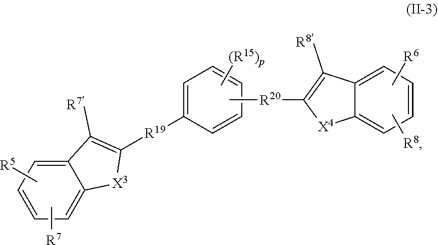

wherein
X³ and X⁴ are the same or different and each is O, S, or NH;
R⁵ and R⁶ are the same and are selected from

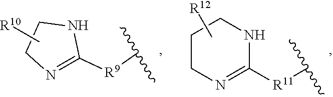

and —C(NHR¹³)═NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is hydrogen or alkyl;
R⁷, R⁸, R^{7′}, and R^{8′} are the same or different and each is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
wherein each R¹⁵ is the same or different and is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2; and
each of R¹⁹ and R²⁰ is independently selected from
(i) a bond,
(ii) —CR²¹=CR²²—,
(iii) —CR²¹2CR²²₂—, and

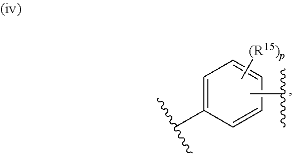

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2;
or a pharmaceutically acceptable salt thereof to the subject.

In some aspects of formula (II), including formulae (II-1), (II-2), and (II-3), or a pharmaceutically acceptable salt thereof, X³ and X⁴ are both O. In some other aspects of formula (II), including formulae (II-1), (II-2), and (II-3), or a pharmaceutically acceptable salt thereof, X³ and X⁴ are both S or both NH.

In any of the foregoing aspects of the compound of formula (II), including formulae (II-1), (II-2), and (II-3), or a pharmaceutically acceptable salt thereof, R⁵ and R⁶ are each —C(NHR¹³)═NR¹⁴, where R¹³ and R¹⁴ are the same or different and each is hydrogen or alkyl. In some other aspects of formula (II), including formulae (II-1), (II-2), and (II-3), or a pharmaceutically acceptable salt thereof, R⁵ and R⁶ are each —C(NHR¹³)═NR¹⁴, where R¹³ and R¹⁴ are both hydrogen.

In any of the foregoing aspects of the compound of formula (II), including formulae (II-1), (II-2), and (II-3), or a pharmaceutically acceptable salt thereof, R⁷, R⁸, R^{7′}, and R^{8′} are the same or different and each is independently selected from hydrogen and alkyl. In some other aspects of formula (II), including formulae (II-1), (II-2), and (II-3), or a pharmaceutically acceptable salt thereof, R⁷, R⁸, R^{7′}, and R^{8′} are each hydrogen.

In any of the foregoing aspects of the compound of formula (II), including formulae (II-1), (II-2), and (II-3), or a pharmaceutically acceptable salt thereof, the LINKER is selected from
(a) —CR²¹=CR²²—,
(b) —CR²¹2CR²²₂—, and

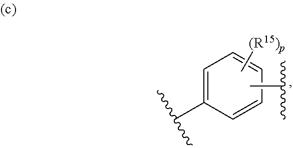

wherein each R¹⁵, R²¹, and R²² is the same or different and is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2.

In some aspects of the compound of formula (II), including formulae (II-1), (II-2), and (II-3), or a pharmaceutically acceptable salt thereof, the LINKER is —CR²¹═CR²²—, where the olefin is a cis olefin, a trans olefin, or a mixture thereof. In certain aspects of the compound of formula (II), including formulae (II-1), (II-2), and (II-3), or a pharmaceutically acceptable salt thereof, the LINKER is —CH═CH—, where the olefin is a cis olefin, a trans olefin, or a mixture thereof.

In some aspects of the compound of formula (II), including formulae (II-1), (II-2), and (II-3), or a pharmaceutically acceptable salt thereof, the LINKER is —CR²¹2CR²²₂—. In certain aspects of the compound of formula (II), including formulae (II-1), (II-2), and (II-3), or a pharmaceutically acceptable salt thereof, the LINKER is —CH₂CH₂—.

In some aspects of the compound of formula (II), including formulae (II-1), (II-2), and (II-3), or a pharmaceutically acceptable salt thereof, the LINKER is

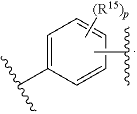

For example, in aspects of the compound of formula (II), including formulae (II-1), (II-2), and (II-3), or a pharmaceutically acceptable salt thereof, the LINKER can be

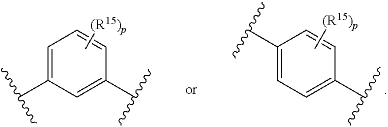

In certain of the compound of formula (II), including formulae (II-1), (II-2), and (II-3), or a pharmaceutically acceptable salt thereof, the LINKER is or

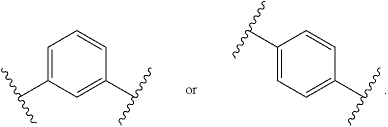 [SMILES: *c1cccc(*)c1 ; *c1ccc(*)cc1]

In any of the foregoing aspects of the compound of formula (II), including formulae (II-1), (II-2), and (II-3), or a pharmaceutically acceptable salt thereof, suitable examples of Y include, e.g.,

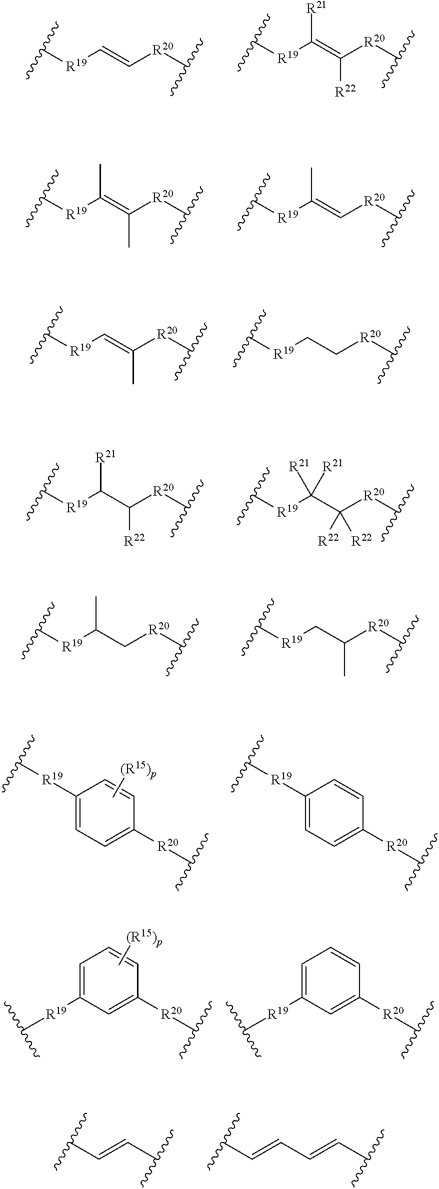

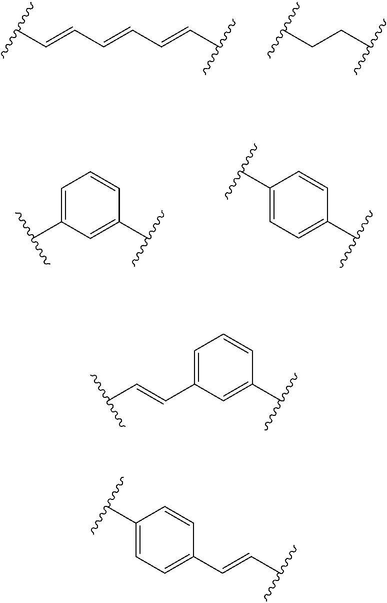

Additional definitions of variables X³, X⁴, R⁵, R⁶, R⁷, R⁸, R^{7′}, R^{8′}, R⁹, R¹⁰, R¹¹, R¹², R¹³, R¹⁴, R¹⁵, R¹⁶, R¹⁹, R²⁰, R²¹, R²², and p with respect to the compound of formula (II), including formulae (II-1), (II-2), and (II-3), or a pharmaceutically acceptable salt thereof, will be readily apparent from the disclosure here, including the definitions provided with respect to the compound of formula (I).

Exemplary core structures of formula (II) include, e.g.,

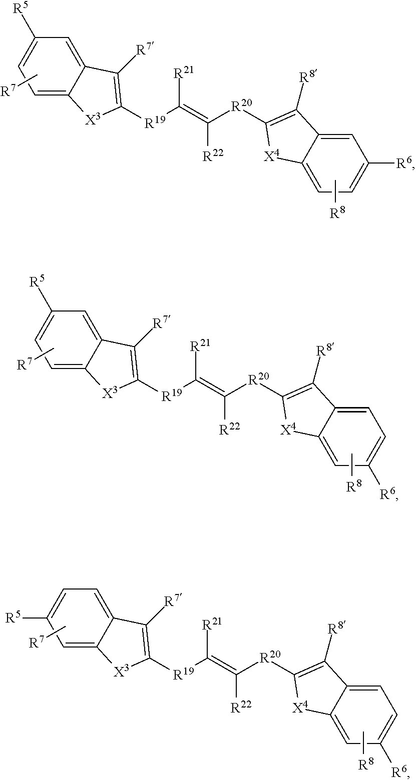

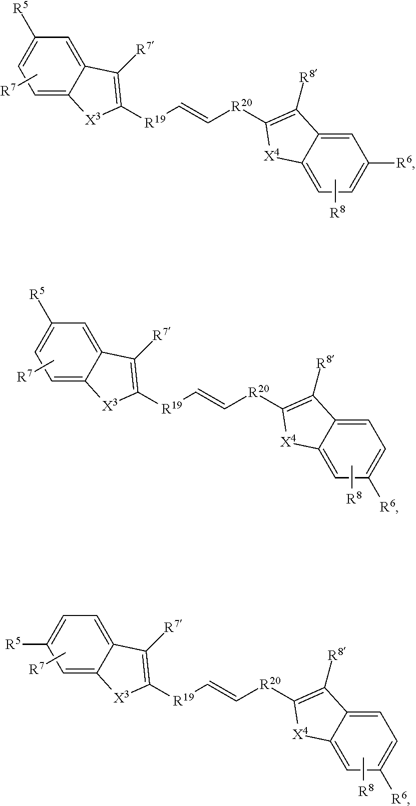

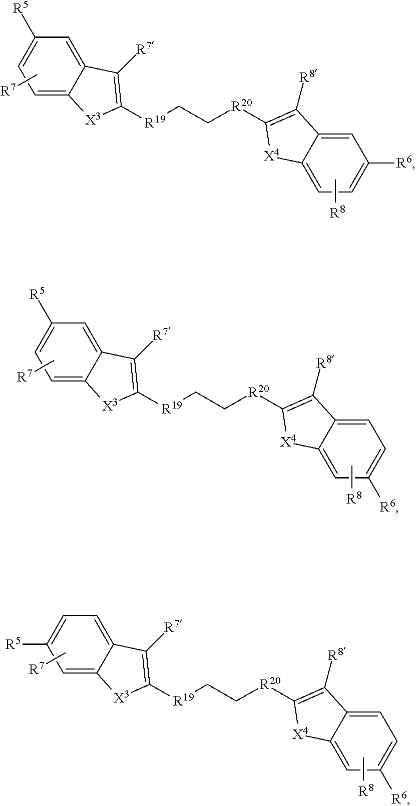

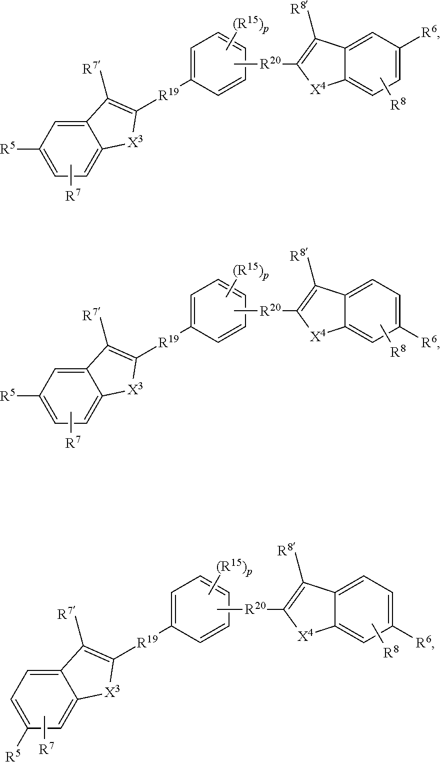

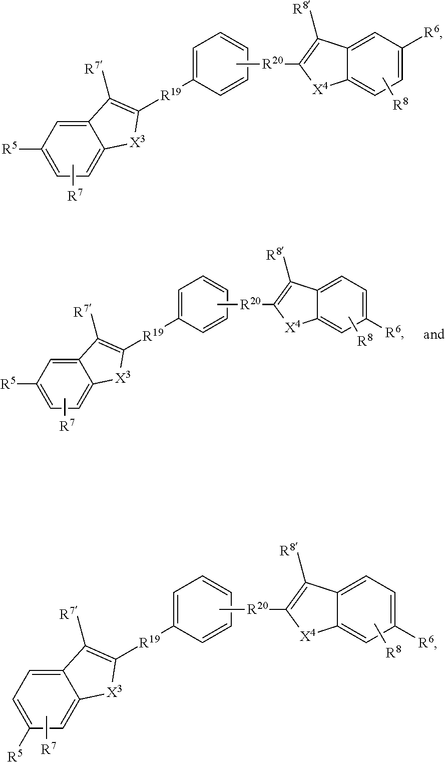

in which X³, X⁴, R⁵, R⁶, R⁷, R⁸, R^{7′}, R^{8′}, R¹⁵, R¹⁹, R²⁰, R²¹, R²², and p are described herein.

Exemplary compounds, including compounds of formula (II), for use in the method described herein include:

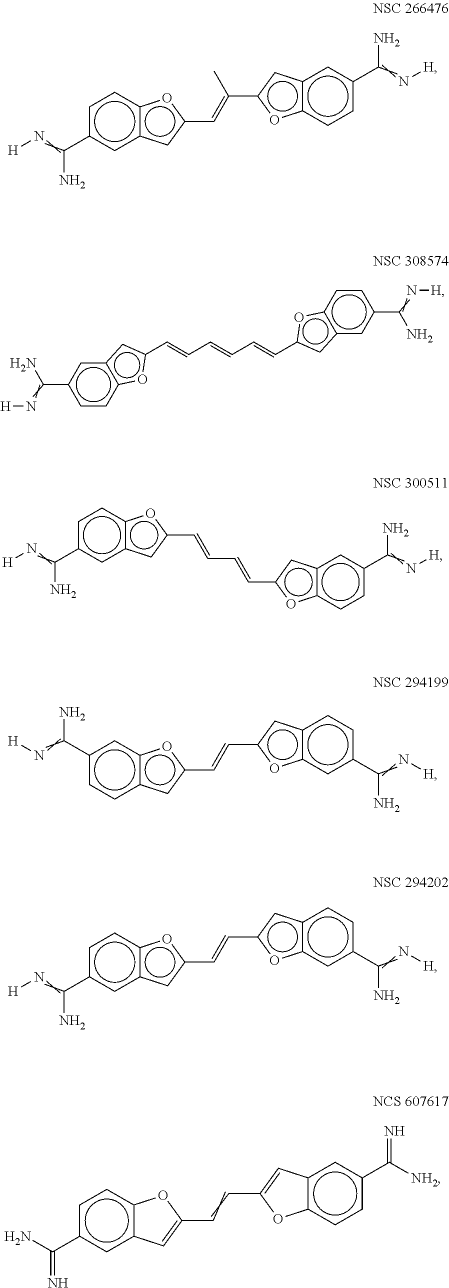

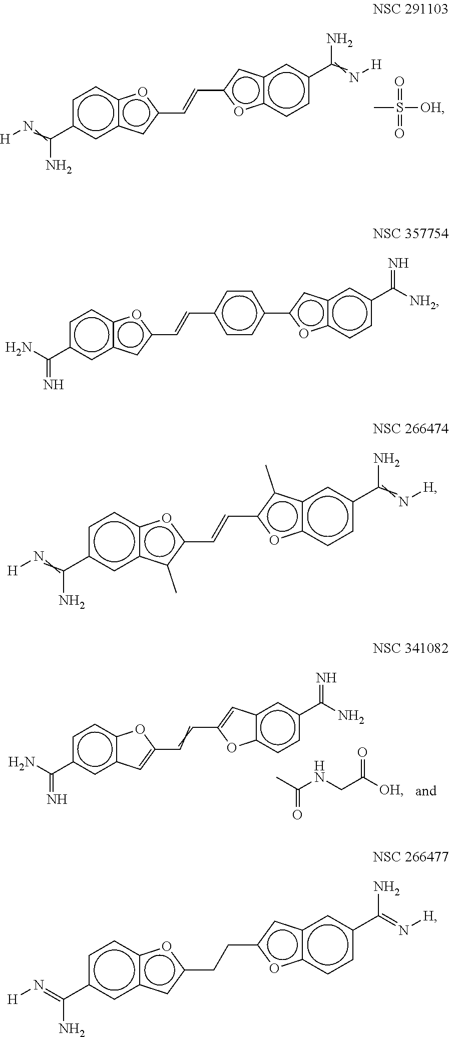

or a pharmaceutically acceptable salt thereof.

Additional exemplary compounds for use in the method described herein include:

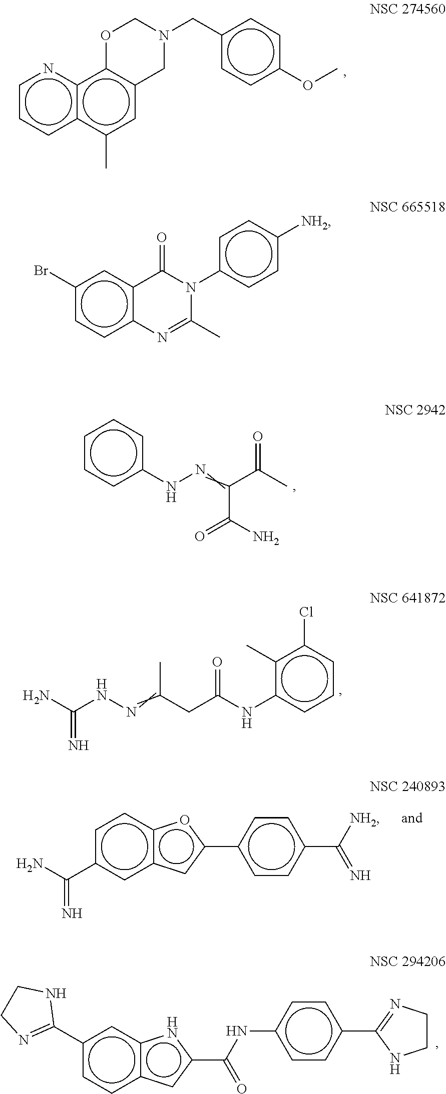

or a pharmaceutically acceptable salt thereof.

In any of the foregoing aspects of the compound of formula (I), the compound of formula (Ia), the compound of formula (II), or any other exemplary compound, where the compound is a pharmaceutically acceptable salt thereof, the compound is a pharmaceutically acceptable salt as described herein. The phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., formic acid, oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, PA, 1990, p. 1445, and Journal of Pharmaceutical Science, 66, 2-19 (1977).

In any of the aspects herein, the term “alkyl” implies a straight-chain or branched alkyl substituent containing from, for example, from about 1 to about 6 carbon atoms, e.g., from about 1 to about 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in C₃-C₆ cycloalkylalkyl, hydroxyalkyl, haloalkyl (e.g., monohaloalkyl, dihaloalkyl, and trihaloalkyl), cyanoalkyl, aminoalkyl, alkylamino, dialkylamino, alkylaminoalkyl, dialkylaminoalkyl, arylcarbonylalkyl (-(alkyl)C(O)aryl), arylalkyl, etc. The alkyl can be substituted or unsubstituted, as described herein. Even in instances in which the alkyl is an alkylene chain (e.g., —(CH₂)ₙ—, in which n is 1 to 10, 1 to 8, 1 to 6, 1 to 4, 1 to 3, 1 to 2, or 2), the alkyl group can be substituted or unsubstituted as described herein.

In any of the aspects herein, the term “alkenyl,” as used herein, means a linear alkenyl substituent containing from, for example, about 2 to about 6 carbon atoms (branched alkenyls are about 3 to about 6 carbons atoms), e.g., from about 3 to about 5 carbon atoms (branched alkenyls are about 3 to about 6 carbons atoms). In accordance with an aspect, the alkenyl group is a C₂-C₄ alkenyl. Examples of alkenyl group include ethenyl, allyl, 2-propenyl, 1-butenyl, 2-butenyl, 1-pentenyl, 2-pentenyl, 3-pentenyl, 1-hexenyl, and the like. The alkenyl can be substituted or unsubstituted, as described herein.

In any of the aspects herein, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hückel's Rule, wherein n=1, 2, or 3. This definition also applies wherever “aryl” occurs as part of a group, such as, e.g., in haloaryl (e.g., monohaloaryl, dihaloaryl, and trihaloaryl), arylalkyl, etc. The aryl can be substituted or unsubstituted, as described herein.

In any of the aspects herein, the term “heteroaryl” refers to aromatic 5 or 6 membered monocyclic groups, 9 or 10 membered bicyclic groups, and II to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)- and (1,2,4)-triazolyl, pyrazinyl, tetrazolyl, furyl, pyrrolyl, thienyl, isothiazolyl, thiazolyl, isoxazolyl, and oxadiazolyl. The heteroaryl can be substituted or unsubstituted, as described herein.

The term “heterocycloalkyl” means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocycloalkyl is a 5, 6, or 7-membered monocyclic ring and contains one, two, or three heteroatoms selected from nitrogen, oxygen, and sulfur. The heterocycloalkyl may be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl. The heterocycloalkyl can be substituted or unsubstituted, as described herein.

In any of the aspects herein, the term “hydroxy” refers to the group —OH.

In any of the aspects herein, the term “cyano” refers to the group —CN.

In any of the aspects herein, the term “nitro” refers to the group —NO₂.

In any of the aspects herein, the term “alkoxy” embraces linear or branched alkyl groups that are attached to a divalent oxygen. The alkyl group is the same as described herein.

In any of the aspects herein, the term “alkylthio” embraces linear or branched alkyl groups that are attached to a divalent sulfur. The alkyl group is the same as described herein.

In any of the aspects herein, the term “halo” refers to a halogen residue selected from fluoro, chloro, bromo, and iodo.

In any of the aspects herein, the term “carboxylate” refers to the group —C(O)OH.

In any of the aspects herein, the term “amino” refers to the group —NH₂. The term “alkylamino” refers to —NHR, whereas the term “dialkylamino” refers to —NRR′. R and R′ are the same or different and each is a substituted or unsubstituted alkyl group, as described herein.

In any of the aspects herein, the term “amido” refers to the group —C(O)NH₂, whereas “alkylamido” and “dialkylamido” refer to an amido in which one or both hydrogens are replaced with a substituted or unsubstituted alkyl group, as described herein. The term “phenylamido” refers to the group —C(O)NHPh, in which the phenyl group is an aryl group that is substituted or unsubstituted, as described herein.

In any of the aspects herein, the term “guanidinyl” refers to the group —N=(NH₂)₂.

In any of the aspects herein, any substituent that is not hydrogen (e.g., alkyl, aryl, etc.) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present disclosure. Suitable substituents include, e.g., halo, alkyl, alkenyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocycloalkyl, each of which is described herein.

In any of the aspects herein, whenever a range of the number of atoms in a structure is indicated (e.g., a C₁₋₁₂, C₁₋₈, C₁₋₆, C₁₋₄, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

The subscripts “n” and “m” represent the number of methylene (—CH₂—) repeat units. The subscript n is an integer from 1-12 (i.e., 1, 2, 3, 4, 5, 6, 7, 8, 9, 10, 11, or 12). The subscript m is an integer from 3-6 (i.e., 3, 4, 5, or 6).

The subscript “p” represents the number of R¹⁵ substituents. The subscript p can be 0 an integer of 1-2 (i.e., 1 or 2). In some aspects of formula (I) and/or formula (II), p preferably is 0 or 1.

The methods described herein comprise administering, to a subject in need thereof, a compound described herein (e.g., a compound of formula (I), (Ia), or (II)), or a pharmaceutically acceptable salt thereof in the form of a pharmaceutical composition. In particular, a pharmaceutical composition will comprise at least one compound described herein, or a pharmaceutically acceptable salt thereof and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intraperitoneal, intramuscular, intratumoral, peritumoral, intraperitoneal, intrathecal, rectal, vaginal, or aerosol formulations. In some aspects, the pharmaceutical composition is administered orally or intravenously.

In accordance with any of the aspects, the compound of formula (I), the compound of formula (Ia), the compound of formula (II), or any other exemplary compound, or a pharmaceutically acceptable salt thereof can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of formula (I), the compound of formula (Ia), the compound of formula (II), or any other exemplary compound, or a salt thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the inhibitors in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compound may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution, such as a mouthwash. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the disclosure, the vehicle for topical application to the skin can include water, buffered solutions, various alcohols, glycols such as glycerin, lipid materials such as fatty acids, mineral oils, phosphoglycerides, collagen, gelatin and silicone based materials.

The compound of formula (I), the compound of formula (Ia), the compound of formula (II), or any other exemplary compound, or a pharmaceutically acceptable salt thereof, alone or in combination with other suitable components, can be made into aerosol formulations to be administered via inhalation. These aerosol formulations can be placed into pressurized acceptable propellants, such as dichlorodifluoromethane, propane, nitrogen, and the like. The compound also may be formulated as a pharmaceutical for non-pressured preparations, such as in a nebulizer or an atomizer.

The dose administered to the subject, particularly a human and other mammals, in accordance with the present disclosure should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular inhibitor and the desired effect. It will be appreciated by one of ordinary skill in the art that various conditions or disease states may require prolonged treatment involving multiple administrations.

The inventive methods comprise administering an effective amount of a compound of formula (I), a compound of formula (Ia), a compound of formula (II), or any other exemplary compound, or a pharmaceutically acceptable salt thereof. An “effective amount” means an amount sufficient to show a meaningful benefit in an individual, e.g., mediating at least one aspect of a disorder to be treated (e.g., reducing inflammation, reducing pain, reducing and/or preventing the formation of ulcerations, increasing intestinal function, improving the condition of mucosa, or facilitating the excretion of urine and/or stool), or treatment, healing, prevention, delay of onset, halting, or amelioration of other relevant medical condition(s) associated with a particular disorder (e.g., colitis). The meaningful benefit observed in the subject can be to any suitable degree (10, 20, 30, 40, 50, 60, 70, 80, 90% or more). In some aspects, one or more symptoms of the disorder are prevented, reduced, halted, or eliminated subsequent to administration of a compound described herein (e.g., a compound of formula (I), (Ia), or (II)) or a pharmaceutically acceptable salt thereof, thereby effectively treating the disorder to at least some degree.

Effective amounts may vary depending upon the biological effect desired in the individual, condition to be treated, and/or the specific characteristics of the compound described herein (e.g., a compound of formula (I), (Ia), or (II)) or a pharmaceutically acceptable salt thereof, and the individual. In this respect, any suitable dose of the compound or a pharmaceutically acceptable salt thereof can be administered to the subject (e.g., human), according to the type of disorder or disease to be treated. Various general considerations taken into account in determining the “effective amount” are known to those of skill in the art and are described, e.g., in Brunton et al., eds., Goodman and Gilman's: The Pharmacological Bases of Therapeutics, 13th ed., McGraw Hill, 2017; and Remington: The Science and Practice of Pharmacy, 23rd Ed., Elsevier, Inc., Philadelphia, PA, 2021, each of which is herein incorporated by reference. The dose of the compound or a pharmaceutically acceptable salt thereof desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

For purposes of the present disclosure, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

Proteins called claudins form channels that control the movement of water and ions, such as sodium, in the intestines. Claudin-2 and claudin-15 are expressed in intestinal epithelial cells. Without wishing to be bound by any theory, it is envisioned that blocking claudin-2 and/or claudin-15 channels is an approach to treating intestinal disorders, such as diarrhea and colitis. Without wishing to be bound by any theory, compounds of formula (I) and compounds of formula (II) are believed to block the claudin-2 and/or -15 channel to limit the water and ion movement, thereby providing therapeutic relief of one or more symptoms of a disorder to be treated. Thus, inhibition of the claudin-2 and/or claudin-15 channel is seen as a viable treatment of certain disorders that are mediated by claudin-2 and/or claudin-15, such as intestinal disorders. Accordingly, a compound as described herein, or a pharmaceutically acceptable salt thereof, can be administered to a subject in need thereof to treat a disorder mediated by claudin-2 and/or claudin-15 such that the disorder is treated by inhibiting claudin-2 and/or claudin-15. In some aspects of the method, the compound of formula (I) to be used is a compound of formula (Ia) or a pharmaceutically acceptable salt thereof. The type of disorder is not particularly limited, but in certain aspects, the disorder includes, e.g., colitis (including ulcerative colitis), Crohn's disease, enteritis, gastroenteritis, inflammatory bowel disease (IBD), irritable bowel syndrome (IBS), celiac disease, enteropathies (autoimmune- or drug induced), pouchitis, or diarrhea. In some aspects, the disorder has altered claudin-2 and/or claudin-15 function.

In certain aspects of the method, the compound described herein (e.g., a compound of formula (I), (Ia), or (II)) or a pharmaceutically acceptable salt thereof can be co-administered with one or more (e.g., 1, 2, 3, etc.) anti-inflammatory agents. The anti-inflammatory agent can be from any suitable class, such as a nonsteroidal anti-inflammatory drug (NSAID) (e.g., a nonselective COX inhibitor, a selective COX2 inhibitor). Examples of anti-inflammatory agents include, for example, celecoxib, etoricoxib, valdecoxib, aspirin, diclofenac, ibuprofen, flurbiprofen, indomethacin, ketoprofen, ketorolac, diflunisal, naproxen, phenylbutazone, piroxicam, sulindac, mefenamic acid, etodolac, meloxicam, nambumetone, oxaprozin, tolmetin, acetaminophen, duloxetine, and a combination thereof.

In certain aspects of the method, the compound described herein (e.g., a compound of formula (I), (Ia), or (II)) or a pharmaceutically acceptable salt thereof can be co-administered with one or more (e.g., 1, 2, 3, etc.) agents for treating an intestinal disorder (e.g., ulcerative colitis, IBS). The agent for treating an intestinal disorder can be from any suitable class, such as an aminosalicylate (e.g., sulfasalazine, mesalamine, olsalazine, balsalazide), a corticosteroid (e.g., prednisone, prednisolone, methylprednisolone, budesonide), an immunomodulator (e.g., azathioprine, 6-mercaptopurine, cyclosporine, tacrolimus), an antispasmodic, an antidepressant, other small molecules (e.g., ozanimod, tofacitinib, pregabalin, gabapentin, alosetron, eluxadoline, rifaximin, lubiprostone, linaclotide, tegaserod, alosetron, asimadoline,), monoclonal antibody therapeutic agents (e.g., adalimumab, golimumab, infliximab, ustekinumab, vedolizumab), a probiotic (e.g., Lactobacillus, Bifidobacterium, Enterococcus, Streptococcus, and/or Leuconostoc), or a combination thereof. Examples of an antispasmodic include, for example, an anticholinergic agent (e.g., hyoscyamine, dicyclomine (dicycloverine)), a direct smooth muscle relaxant (e.g., cimetropium, mebeverine, otilonium, pinaverium bromide, trimebutine), and peppermint oil. Examples of the antidepressant include, for example, a tricyclic antidepressant (e.g., imipramine, despiramine, amitriptyline, nortiptyline), a selective serotonin reuptake inhibitor (SSRI) antidepressant (e.g., fluoxetine, paroxetine, citalopram, escitalopram, sertraline), a serotonin-norepinephrine reuptake inhibitor (SNRI) antidepressant (e.g., venlafaxine, duloxetine, desvenlavaxine, milnacipram), bupropion, mirtazipine, and trazodone.

The compound or a pharmaceutically acceptable salt thereof can be administered before, concurrently with, or after administration of an anti-inflammatory agent or additional agent for treating an intestinal disorder. One or more than one, e.g., two or more, thre or more, etc. anti-inflammatory agents and/or agents for treating an intestinal disorder can be administered. Accordingly, the present disclosure is directed to a pharmaceutical composition comprising a pharmaceutically acceptable carrier and a combination of the compound described herein (e.g., a compound of formula (I), (Ia), or (II)) or a pharmaceutically acceptable salt thereof and at least one anti-inflammatory agent and/or agent for treating an intestinal disorder.

In an aspect, a compound described herein (e.g., a compound of formula (I), (Ia), or (II)) inhibits claudin-2, claudin-15, or both. In a preferred aspect, the compound inhibits at least claudin-2. In a preferred aspect, the compound inhibits at least claudin-15. In a preferred aspect, the compound inhibits claudin-2 and claudin-15. In an aspect, a compound of formula (I), a compound of formula (Ia), or a compound of formula (II) is selective for claudin-2 and/or claudin-15 relative to other claudin channels. For example, the compound can be at least 2 times (e.g., at least 3 times, at least 4 times, at least 5 times, at least 6 time, at least 8 times, at least 10 times, at least 15 times, at least 20 times, at least 30 times, at least 40 times, at least 50 times, at least 60 times, or at least 100 times) more selective for claudin-2 and/or claudin-15 compared to one or more other claudin channels. In another aspect, a compound of formula (I), a compound of formula (Ia), or a compound of formula (II) is selective for claudin-2 relative to claudin-15, e.g., the compound is at least 1.5 times, at least 2 times, at least 2.5 times, at least 3 times, at least 3.5 times, or at least 4 times more selective for claudin-2 relative to claudin-15 at the same dose. In another aspect, a compound of formula (I), a compound of formula (Ia), or a compound of formula (II) is selective for claudin-15 relative to claudin-2, e.g., the compound is at least 1.5 times, at least 2 times, at least 2.5 times, at least 3 times, at least 3.5 times, or at least 4 times more selective for claudin-15 relative to claudin-2 at the same dose. In another aspect, a compound of formula (I), a compound of formula (Ia), or a compound of formula (II) is not selective for claudin-15 or claudin-2 relative to other claudins. In another aspect, a compound of formula (I) or a compound of formula (II) is equally effective for claudin-2 and claudin-15.

Accordingly, the disclosure is further directed to a method of inhibiting claudin-2 and/or claudin-15 activity in a cell comprising contacting a compound described herein (e.g., a compound of formula (I), (Ia), or (II)), or a pharmaceutically acceptable salt thereof to a cell, whereby the activity of claudin-2 and/or claudin-15 is inhibited. The claudin-2 and/or claudin-15 activity can be measured by any method, including the assays described herein. The cell can be from any suitable tissue, including stomach tissue and intestinal tissue, including the small intestine and colon.

The disclosure is further illustrated by the following aspects.

Aspect (1) A method of treating a disorder mediated by claudin-2 and/or claudin-15 in a subject comprising administering to the subject an effective amount of a compound of formula (I)

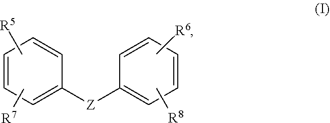

wherein
R⁵ and R⁶ are the same and are selected from

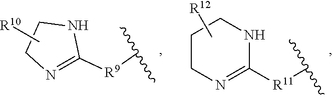

and —C(N³)═NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is alkyl;
R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
Z is selected from

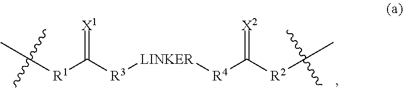

wherein
X¹ and X² are the same or different and each is O or S;
R¹ and R² are the same or different and each is —NH— or a bond;
R³ and R⁴ are the same or different and each is —NH—, —CH═CH—, —CH₂CH₂—, —NHC(O)—, —NHC(O)CH₂NHC(O)NH—, or a bond;
provided that R¹ and R³ are not both bonds and R² and R⁴ are not both bonds; and
LINKER is selected from
(i) —(CH₂)ₙ— that is optionally substituted with a hydroxy, wherein n is an integer of 1-12,
(ii) —O(CH₂)ₘO— that is optionally substituted, wherein m is an integer of 3-6,
(iii) —CH═CH—,

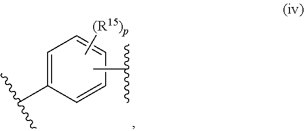

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2,

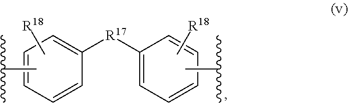

wherein R¹⁷ is selected from a bond, —CH═CH—, —N═N—, —CH₂—, —NHC(O)—, —C(O)NH—, —NHC(O)NH—, —NHC(O)—(CH₂)₁₋₄—C(O)NH—, —NHC(O)NH—(CH₂)₁₋₄—NHC(O)NH—, —NHC(O)-Ph-C(O)NH—, and —NHC(O)NH-Ph-NHC(O)NH—, and each R¹⁸ is the same or different and each is hydrogen, alkyl, alkoxy, 1,4-dihydroimidazolyl, or —R¹⁶—C(O)NH-Ph-R⁵,

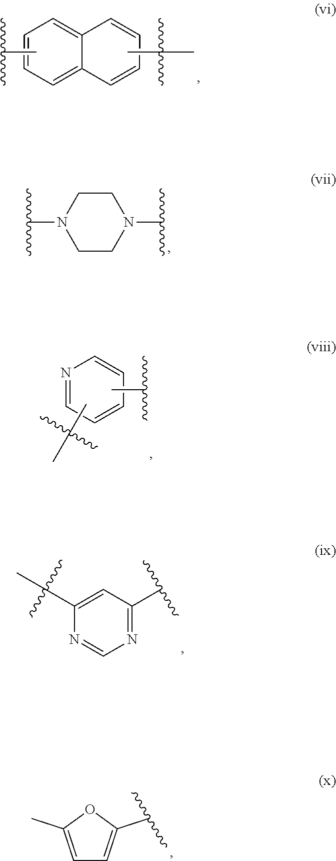

and (xi) a bond,
and

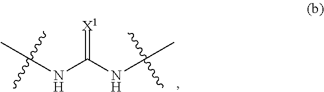

wherein X¹ is O or S;
   or a pharmaceutically acceptable salt thereof to the subject.

Aspect (2) The method of aspect (1), wherein the compound of formula (I) is a compound of formula (I-1)

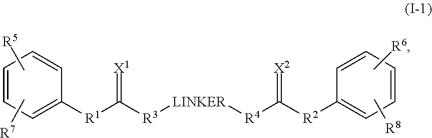

wherein
X¹ and X² are the same or different and each is 0 or S;
R¹ and R² are the same or different and each is —NH— or a bond;
R³ and R⁴ are the same or different and each is —NH—, —CH═CH—, —CH₂CH₂—, —NHC(O)—, —NHC(O)CH₂NHC(O)NH—, or a bond;
provided that R¹ and R³ are not both bonds and R² and R⁴ are not both bonds;
R⁵ and R⁶ are the same and are selected from

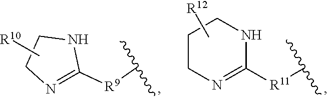

and —C(NHR¹³)═NR¹⁴;
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is alkyl;
R⁷ and R⁸ are the same or different and each is selected from hydrogen, alkyl, halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, and R¹⁶ is a bond or —NH—; and
LINKER is selected from
—(CH₂)ₙ— that is optionally substituted with a hydroxy, wherein n is an integer of 1-12,
—O(CH₂)ₘO— that is optionally substituted, wherein m is an integer of 3-6,
—CH═CH—,

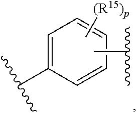

wherein each R¹⁵ is the same or different and is selected from alkyl, halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2,

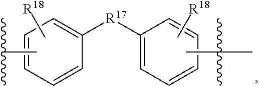

wherein R¹⁷ is selected from a bond, —CH═CH—, —N═N—, —CH₂—, —NHC(O)—, —C(O)NH—, —NHC(O)NH—, —NHC(O)—(CH₂)₁₋₄—C(O)NH—, —NHC(O)NH—(CH₂)₁₋₄—NHC(O)NH—, —NHC(O)-Ph-C(O)NH—, and —NHC(O)NH-Ph-NHC(O)NH—, and each R¹⁸ is the same or different and each is hydrogen, alkyl, alkoxy, 1,4-dihydroimidazolyl, or —R¹⁶—C(O)NH-Ph-R⁵,

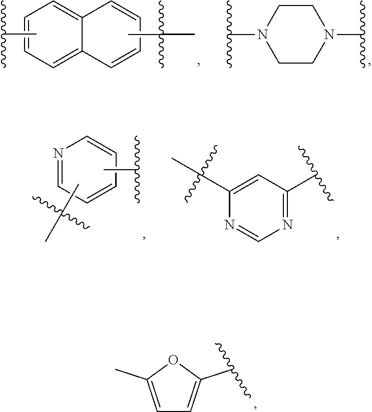

and a bond,
or a pharmaceutically acceptable salt thereof to the subject.

Aspect (3) The method of aspect (1) or (2), wherein in the compound of formula (I) or a pharmaceutically acceptable salt thereof, X¹ and X² are both O.

Aspect (4) The method of any one of aspect (1)-(3), wherein in the compound of formula (I) or a pharmaceutically acceptable salt thereof, R¹ and R² are each —NH—.

Aspect (5) The method of any one of aspects (1)-(4), wherein in the compound of formula (I) or a pharmaceutically acceptable salt thereof, R³ and R⁴ are each —NH— or a bond.

Aspect (6) The method of aspect (1), wherein the compound of formula (I) is a compound of formula (I-2)

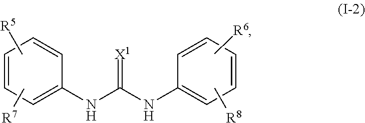

wherein
X¹ is O or S;
R⁵ and R⁶ are the same and are selected from

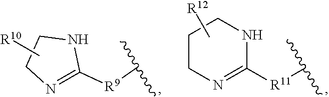

and —C(NHR¹³)NR¹⁴
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is alkyl; and
   R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; or a pharmaceutically acceptable salt thereof to the subject.

Aspect (7) The method of any one of aspects (1)-(6), wherein in the compound of formula (I) or a pharmaceutically acceptable salt thereof, R⁵ and R⁶ are both

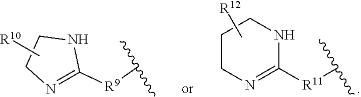

Aspect (8) The method of aspect (7) wherein in the compound of formula (I) or a pharmaceutically acceptable salt thereof, R⁹ and R¹¹ are both a bond.

Aspect (9) The method of aspect (8), wherein in the compound of formula (I) or a pharmaceutically acceptable salt thereof, R¹⁰ and R¹² are both hydrogen.

Aspect (10) The method of any one of aspects (1)-(9), wherein in the compound of formula (I) or a pharmaceutically acceptable salt thereof, R⁷ and R⁸ are the same and each is selected from hydrogen, alkyl, amido, alkylamido, dialkylamido, and R⁵.

Aspect (11) The method of any one of aspects (1)-(5) and (7)-(10), wherein in the compound of formula (I) or a pharmaceutically acceptable salt thereof, LINKER is selected from
—(CH₂)ₙ— that is optionally substituted with a hydroxy, wherein n is an integer of 1-12,

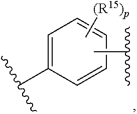

wherein each R¹⁵ is the same or different and is selected from alkyl, halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, R¹⁶ is a bond or —NH—, and p is 0 or an integer of 1-2,

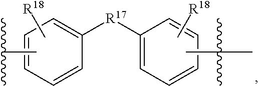

wherein R¹⁷ is selected from a bond; —CH═CH—, —N═N—, —CH₂—, —NHC(O)NH—, and —NHC(O)-Ph-C(O)NH—, and R¹⁸ is hydrogen, alkyl, or alkoxy, and

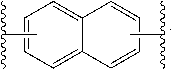

Aspect (12) The method of any one of aspects (1)-(5) and (7)-(10), wherein in the compound of formula (I) or a pharmaceutically acceptable salt thereof, LINKER is

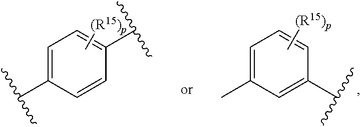

wherein
p is 0, or
p is 1 and R¹⁵ is selected from alkyl, halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, and
R¹⁶ is a bond or —NH—.

Aspect (13) The method of any one of aspects (1)-(12), wherein the compound of formula (I) is a pharmaceutically acceptable salt.

Aspect (14) The method of aspect (1) or (2), wherein the compound of formula (I) or a pharmaceutically acceptable salt thereof is

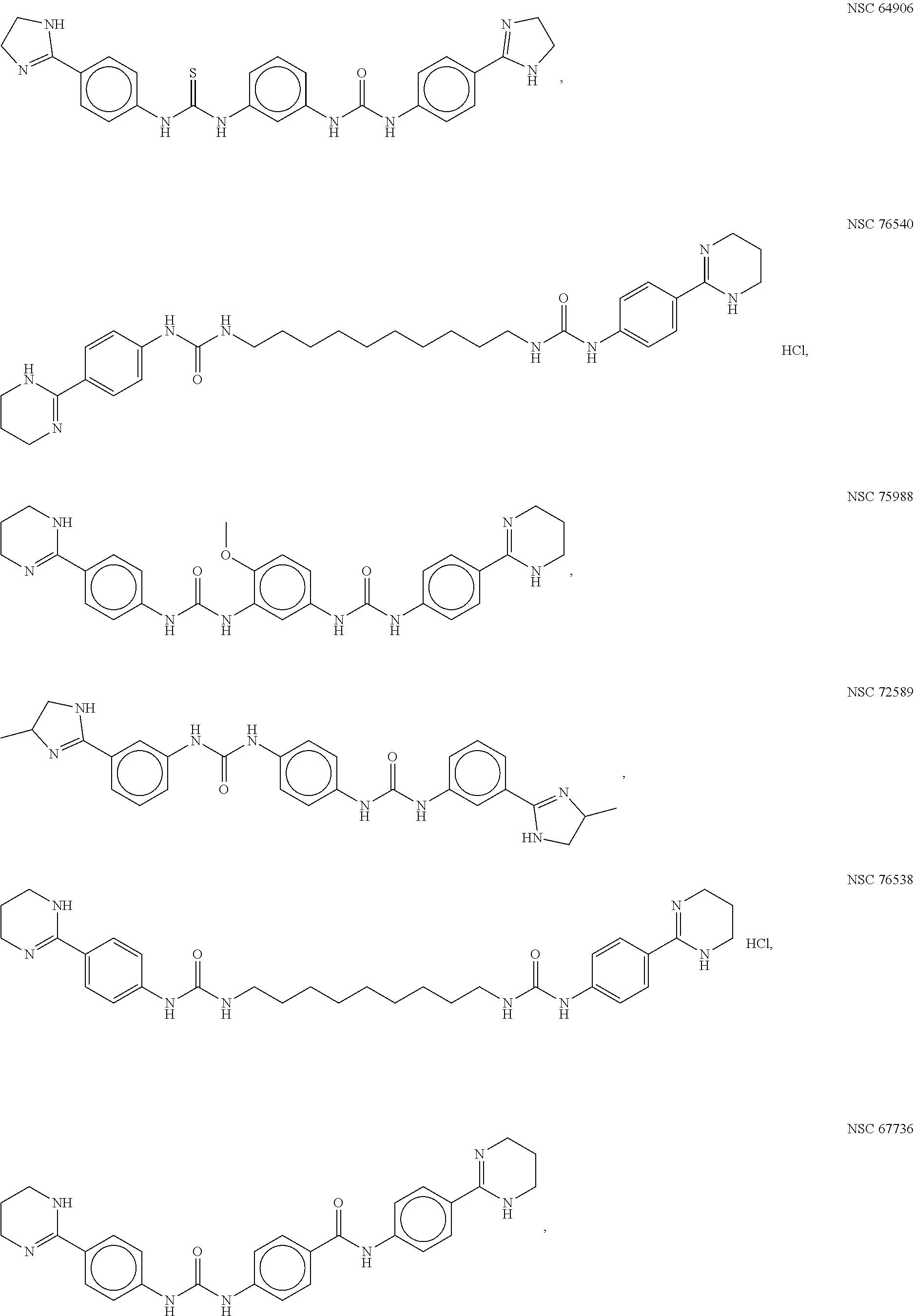

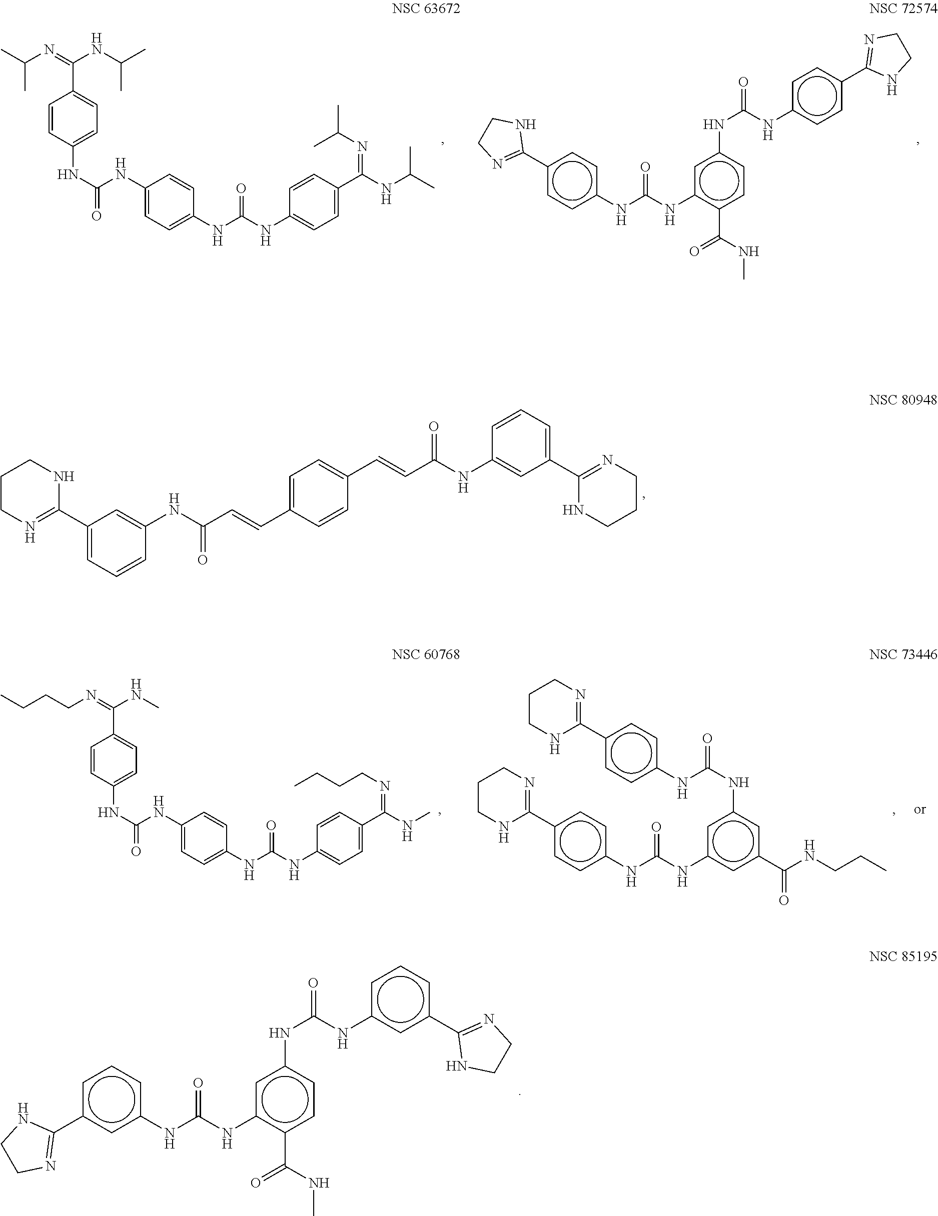

Aspect (15) The method of any one of aspects (1)-(14), wherein the disorder mediated by claudin-2 and/or claudin-15 is an intestinal disorder.

Aspect (16) The method of aspect (15), wherein the intestinal disorder is colitis (including ulcerative colitis), Crohn's disease, inflammatory bowel disease (IBD), enteritis, gastroenteritis, irritable bowel syndrome (IBS), celiac disease, enteropathies (autoimmune- or drug induced), pouchitis, or diarrhea.

Aspect (17) The method of any one of aspects (1)-(16), wherein the compound of formula (I) or a pharmaceutically acceptable salt thereof inhibits claudin-2 and/or claudin-15.

Aspect (18) A compound that is a 1,3- or 1,4-substituted phenyl bridged compound of formula (Ia)

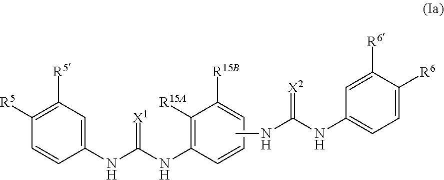

wherein
   X¹ and X² are the same or different and each is O or S;
one of R⁵ and R^{5′} is

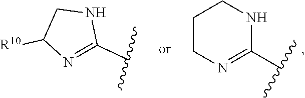

and the other is hydrogen;
one of R⁶ and R^{6′} is

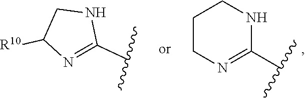

and the other is hydrogen;
wherein R⁵ and R⁶ are the same and R^{5′} and R^{6′} are the same;
R¹⁰ is hydrogen or alkyl;
R^{15A} and R^{15B} are the same or different and each is selected from hydrogen, alkyl, alkoxy, amino, alkylamido, 1,4-dihydroimidazolyl, and guanidinyl,
or a pharmaceutically acceptable salt thereof.

Aspect (19) The compound of aspect (18) or a pharmaceutically acceptable salt thereof, wherein X¹ and X² are both O.

Aspect (20) The compound of aspect (18) or a pharmaceutically acceptable salt thereof, wherein at least one of X¹ and X² is S.

Aspect (21) The compound of any one of aspects (18)-(20) or a pharmaceutically acceptable salt thereof, wherein
the compound of formula (Ia) is a 1,3-substituted phenyl bridged compound,
R⁵ and R⁶ are both

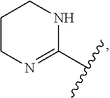 [SMILES: *C1=NCCCN1]

R^{5′} and R^{6′} are both hydrogen, and
R^{15A} is hydrogen.

Aspect (22) The compound of any one of aspects (18)-(20) or a pharmaceutically acceptable salt thereof, wherein
the compound of formula (Ia) is a 1,3-substituted phenyl bridged compound,
R⁵ and R⁶ are both

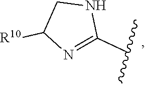

R^{5′} and R^{6′} are both hydrogen,
R¹⁰ is hydrogen, and
R^{15B} is hydrogen.

Aspect (23) The compound of any one of aspects (18)-(20) or a pharmaceutically acceptable salt thereof, wherein
the compound of formula (Ia) is a 1,4-substituted phenyl bridged compound,
R⁵ and R⁶ are both

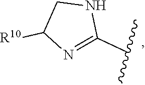

R^{5′} and R^{6′} are both hydrogen,
R¹⁰ is methyl, and
R^{15B} is hydrogen.

Aspect (24) The compound of aspect (18) selected from

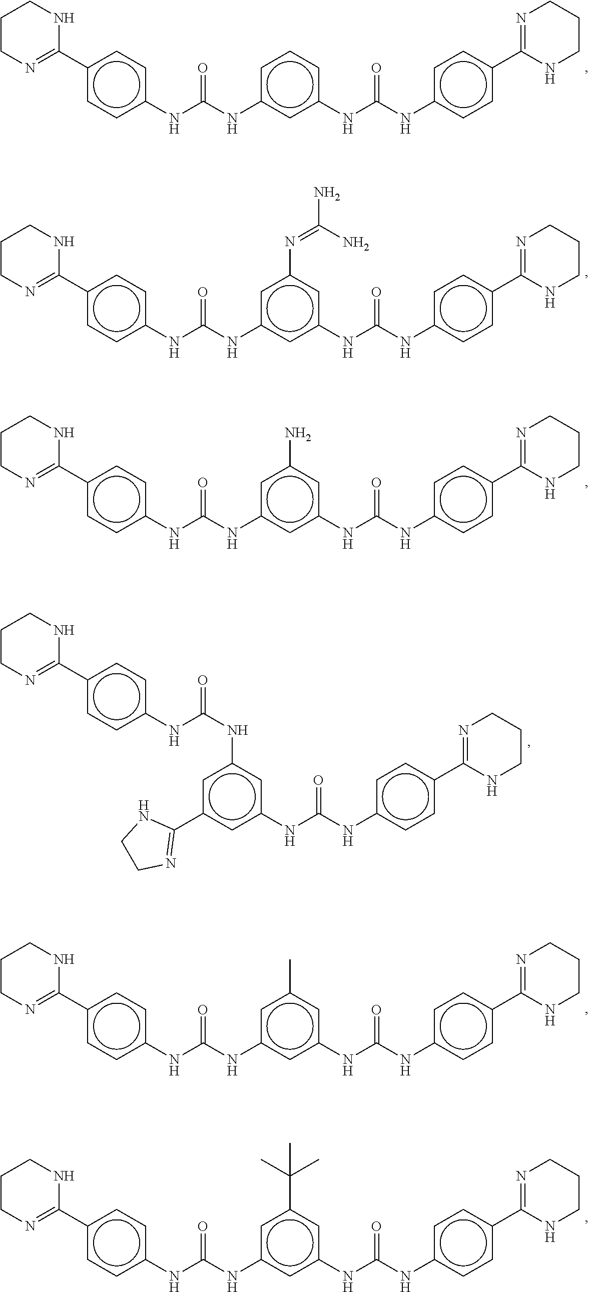

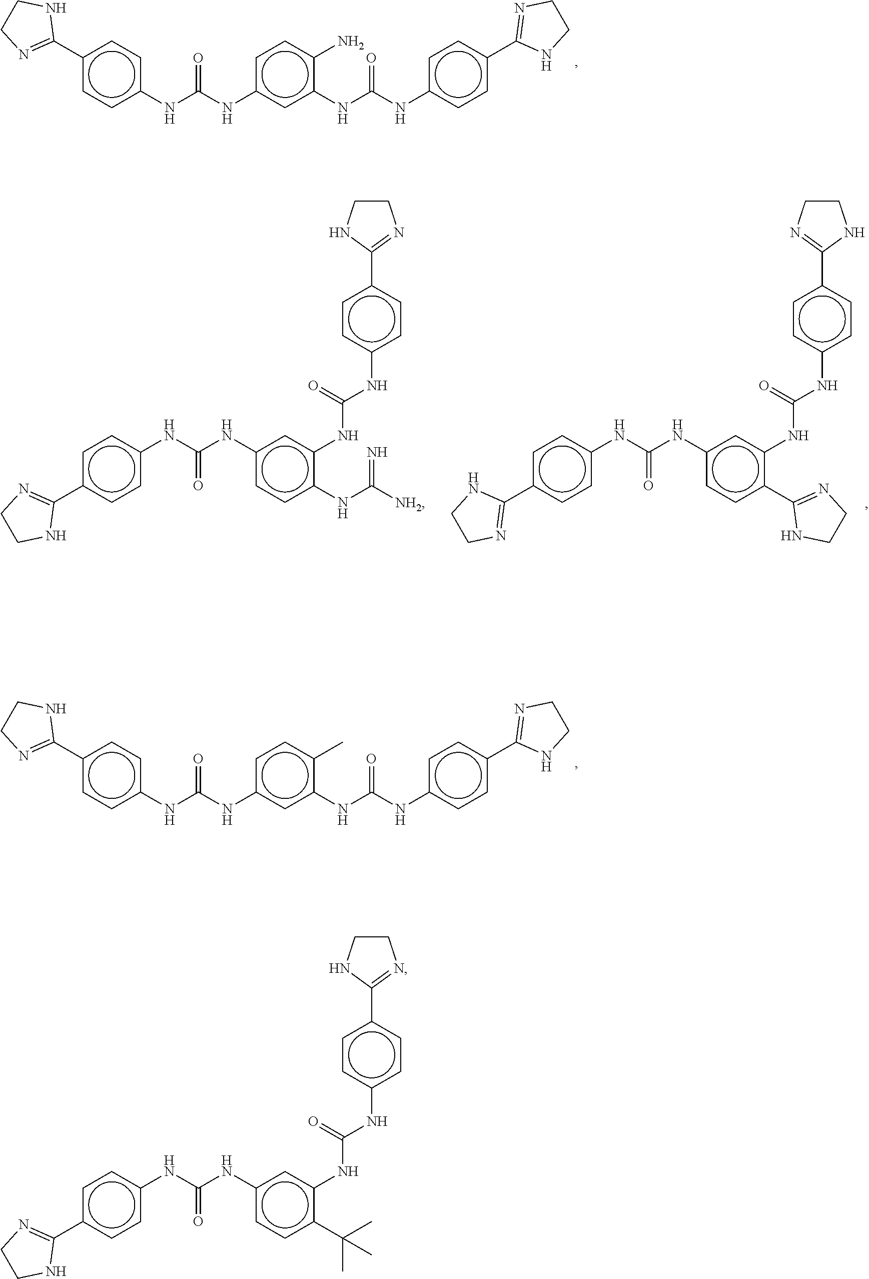

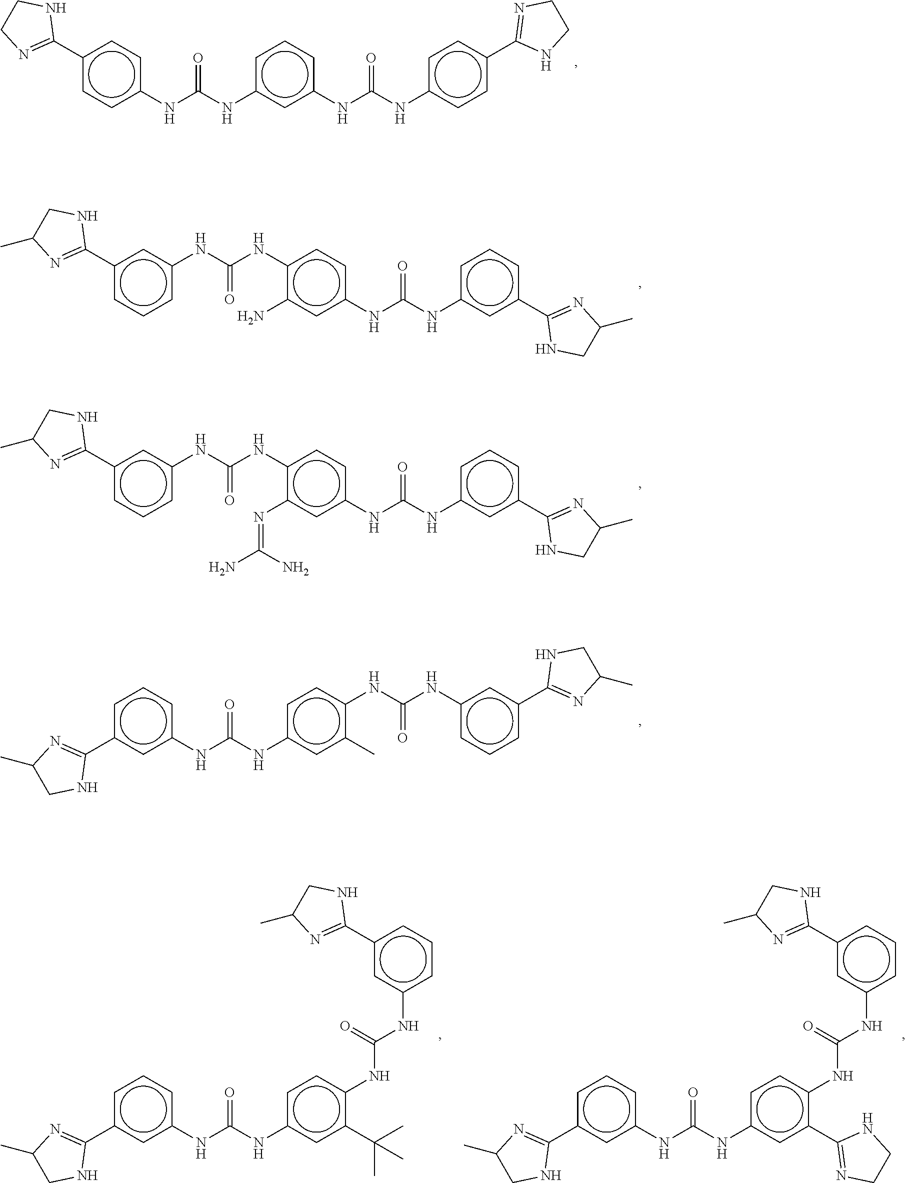

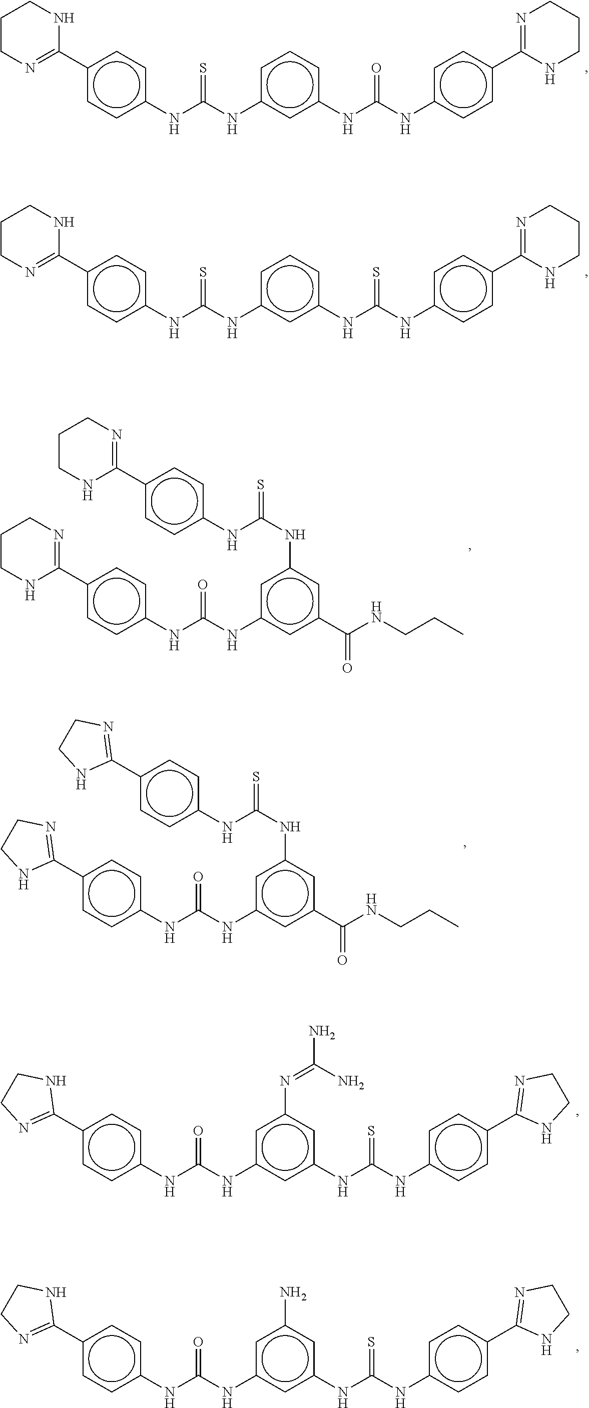

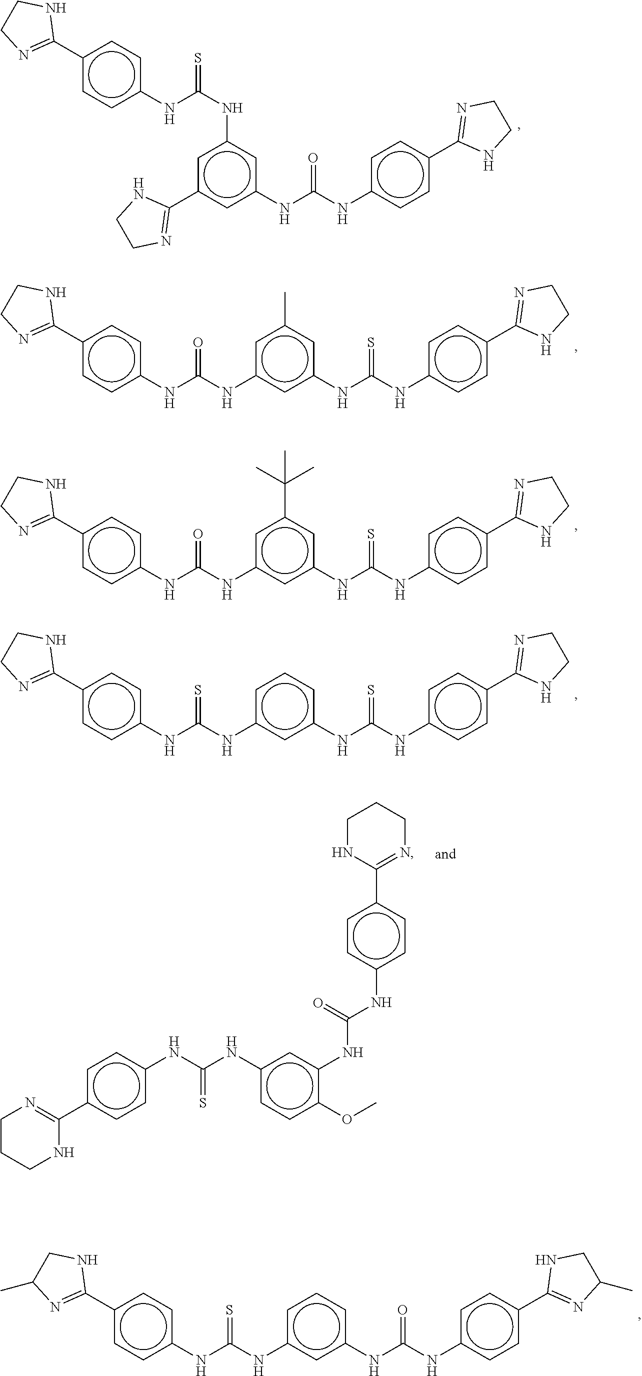

or a pharmaceutically acceptable salt thereof.

Aspect (25) A pharmaceutical composition comprising the compound of any one of aspects (18)-(24) or a pharmaceutically acceptable salt thereof and at least one carrier.

Aspect (26) A method of treating a disorder mediated by claudin-2 and/or claudin-15 in a subject comprising administering to the subject an effective amount of the compound of any one of aspects (18)-(24) or a pharmaceutically acceptable salt thereof to the subject.

Aspect (27) The method of aspect (26), wherein the disorder mediated by claudin-2 and/or claudin-15 is an intestinal disorder selected from colitis (including ulcerative colitis), Crohn's disease, enteritis, gastroenteritis, inflammatory bowel disease (IBD), irritable bowel syndrome (IBS), celiac disease, enteropathies (autoimmune- or drug induced), pouchitis, or diarrhea.

Aspect (28) A method of treating a disorder mediated by inhibiting claudin-2 and/or claudin-15 in a subject comprising administering to the subject an effective amount of a compound selected from

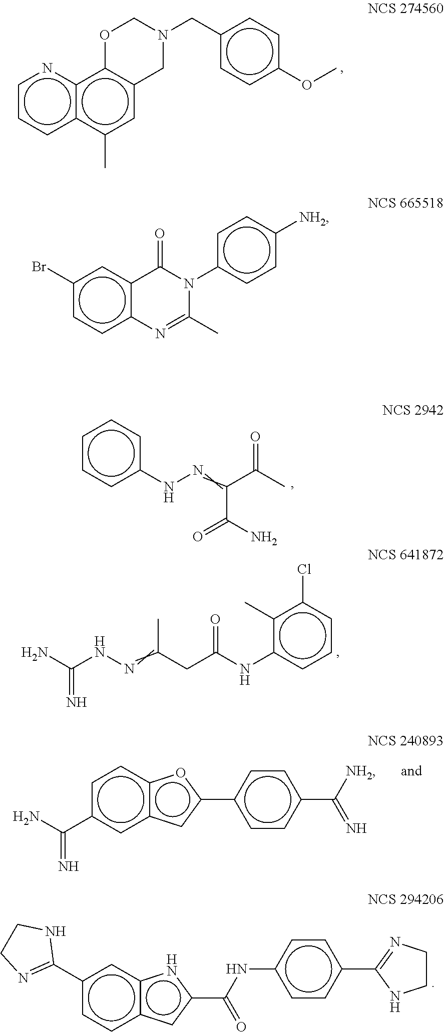

Aspect (29) A method of treating a disorder mediated by claudin-2 and/or claudin-15 in a subject comprising administering to the subject an effective amount of a compound of formula (II)

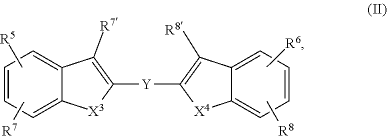

wherein
X³ and X⁴ are the same or different and each is O, S, or NH;
R⁵ and R⁶ are the same and are selected from

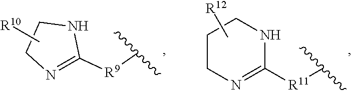

and —C(NHR¹³)NR⁴
wherein
   R⁹ and R¹¹ are the same or different and each is a bond, —CH₂—, —CH₂CH₂—, or —NH—;
   R¹⁰ and R¹² are the same or different and each is hydrogen, alkyl, or hydroxy; and
   R¹³ and R¹⁴ are the same or different and each is hydrogen or alkyl;
R⁷, R⁸, R^{7′}, and R^{8′} are the same or different and each is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
Y is

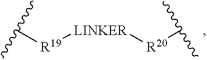

wherein
LINKER is selected from
   —CR²¹═CR²²—,
      —CR²¹2CR²²₂—, and

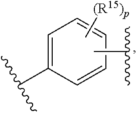

wherein each R¹⁵, R²¹, and R²² is the same or different and is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2; and
each of R¹⁹ and R²⁰ is independently selected from
(i) a bond,
(ii) —CR²¹═CR²²—,
(iii) —CR²¹2CR²²₂—, and

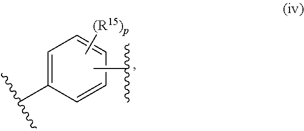

wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2;
or a pharmaceutically acceptable salt thereof to the subject.

Aspect (30) The method of aspect (29), wherein in the compound of formula (II) or a pharmaceutically acceptable salt thereof, X³ and X⁴ are both O.

Aspect (31) The method of aspect (29) or (30), wherein in the compound of formula (II) or a pharmaceutically acceptable salt thereof, R⁵ and R⁶ are both —C(NHR¹³)═NR¹⁴, wherein R¹³ and R¹⁴ are the same or different and each is hydrogen or alkyl.

Aspect (32) The method of aspect (31), wherein R¹³ and R¹⁴ are both hydrogen.

Aspect (33) The method of any one of aspects (29)-(32), wherein R⁷, R⁸, R^{7′}, and R^{8′} are the same or different and each is independently selected from hydrogen and alkyl.

Aspect (34) The method of any one of aspects (29)-(33), wherein R⁷, R⁸, R^{7′}, and R^{8′} are each hydrogen.

Aspect (35) The method of any one of aspects (29)-(34), wherein Y is of formula:

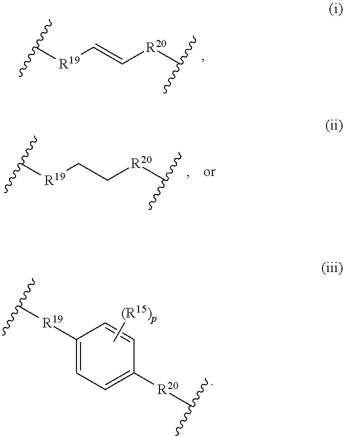

Aspect (36) The method of any one of aspects (29)-(35), wherein the compound of formula (II) is a pharmaceutically acceptable salt.

Aspect (37) The method of aspect (29), wherein the compound of formula (II) or a pharmaceutically acceptable salt thereof is

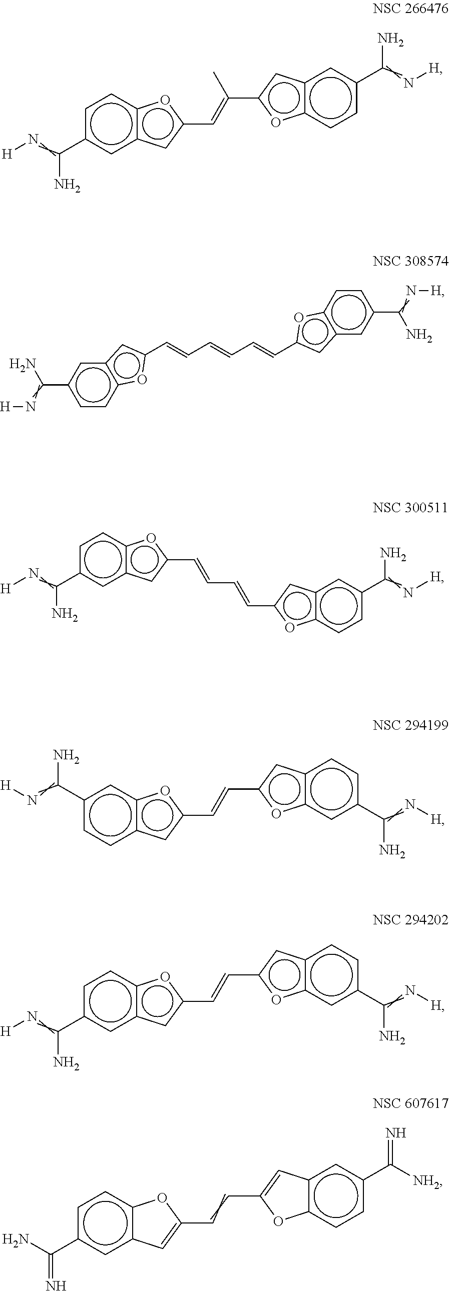

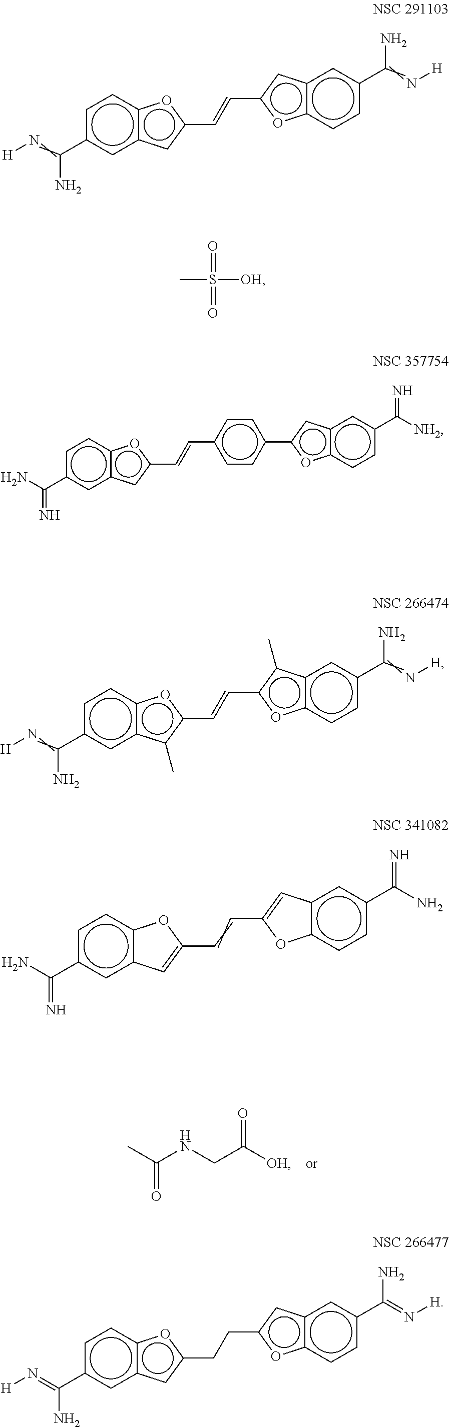

Aspect (38) The method of any one of aspects (29)-(37), wherein the disorder mediated by claudin-2 and/or claudin-15 is an intestinal disorder.

Aspect (39) The method of aspect (38), wherein the intestinal disorder is colitis, Crohn's disease, enteritis, gastroenteritis, inflammatory bowel disease (IBD), irritable bowel syndrome (IBS), celiac disease, enteropathies (autoimmune- or drug induced), pouchitis, or diarrhea.

Aspect (40) The method of any one of aspects (29)-(39), wherein the compound of formula (II) or a pharmaceutically acceptable salt thereof inhibits claudin-2 and/or claudin-15.

The following examples further illustrate the disclosure but, of course, should not be construed as in any way limiting its scope.

### Example 1

Exemplary compounds of formula (Ia) (e.g., compound 1) can be prepared in accordance with the reaction schemes set forth in FIG. 1 and FIG. 2. FIG. 1 is a reaction scheme of preparing building blocks 3 and 3a-1. Boc is a tert-butyloxycarbonyl protecting group; THE is tetrahydrofuran; CDI is 1,1′-carbonyldiimidazole; and DCM is dichloromethane. FIG. 2 is a reaction scheme of preparing compound 1. TEA is triethylamine; DIEA is N,N-diisopropylethylamine; and TFA is trifluoroacetic acid. Experimental protocols for the preparation of compounds set forth in FIG. 1 and FIG. 2 are provided below.

To a solution of compound 1 (70.0 g, 472 mmol, 1.00 eq) in EtOH (700 mL) was added NaOEt (3.22 g, 47.2 mmol, 0.10 eq) at 20-25° C. and stirred at 20-25° C. for 3 hours, then compound 1-a (35.08 g, 473 mmol, 39.5 mL, 1.00 eq) was added to the solution at 25-30° C., then warmed to 70-75° C. and stirred at 70-75° C. for 12 hours. The reaction was monitored with LCMS, and compound 1 was consumed and the desired MS was detected (M/Z+1=206.2). The reaction mixture was concentrated under vacuum and reaction mixture was quenched by addition water 300 mL at 25° C., and then diluted with HCl (2M) 150 mL until pH=2 and extracted with Ethyl acetate (250 mL*3). The aqueous layer was diluted with NaOH (6 M) 180 mL until pH=10, then extracted with ethyl acetate (200 mL*3). The mixture was dried over Na₂SO₄, filtered, and concentrated under reduced pressure to give a residue. The residue was dissolved in ethyl acetate (80 mL), and then petroleum ether (50 mL) was added. The resulting solution was stirred for 10 min and the solid was separated. The suspension was extracted and filtered under reduced pressure, and the filter residue was washed with petroleum ether (150 mL*3) and collected. Compound 2 (54.0 g, 263 mmol, 55.7% yield) was obtained as a yellow solid which was confirmed by ¹HNMR (400 MHz, DMSO_d6, δ: 8.18-8.26 (m, 2H), 7.97-8.04 (m, 2H), 3.35-3.42 (m, 5H), 1.67-1.73 (m, 2H)).

To a solution of compound 2 (54 g, 263.14 mmol, 1.00 eq) in THE (550 mL) was added TEA (39.99 g, 395 mmol, 55 mL, 1.50 eq) at 20-25° C., then the mixture cooled to 0° C. and (Boc)₂O (69.35 g, 317 mmol, 73 mL, 1.21 eq) was added to the solution at 0° C., then warmed to 20-25° C. for 12 hours. LCMS showed compound 2 was completely converted, and trace of desired MS (M/Z+1=306.2) was formed. The reaction mixture was poured into water (800 mL), extracted with ethyl acetate (400 mL*3). The combined organic layer was washed with saturated saltwater (800 mL), dried over Na₂SO₄, filtered, and concentrated under reduced pressure to give a residue. The reaction mixture was poured into water (800 mL) and extracted with ethyl acetate (400 mL*3). The combined organic layer was washed with saturated saltwater (800 mL), dried over Na₂SO₄, filtered, and concentrated under reduced pressure to give a residue. The residue was purified by column chromatography (SiO₂, dichloromethane:methanol=80:1 to 10:1). Thin layer chromatography (dichloromethane:methanol=20:1) showed the product (R_{f}=0.60), which was collected. Compound 2-a1 (36.2 g, 118 mmol, 45.1% yield) was obtained as a yellow solid which was confirmed by ¹HNMR (400 MHz, DMSO_d6, δ: 8.19-8.31 (m, 2H), 7.61-7.73 (m, 2H), 3.61-3.70 (m, 2H), 3.54-3.59 (m, 2H), 1.78-1.87 (m, 2H), 1.05-1.11 (m, 9H)).

To a solution of compound 2-a1 (36.2 g, 118 mmol, 1.00 eq) in MeOH (400 mL) was added Pd/C (3.60 g, 118 mmol, 10% purity, 1.00 eq) at 20-25° C. under N₂ atmosphere. The suspension was degassed and purged with H₂ three times. The mixture warmed to 30-40° C. and was stirred at 30-40° C. under H₂ (50 Psi) at H₂ for 48 hours. Thin layer chromatography (dichloromethane:methanol=20:1) showed compound 2-a1 (R_{f}=0.40) was consumed and one new main spot (R_{f}=0.00) formed. The reaction mixture was filtered and the filtrate was washed with MeOH (800 mL), and the resulting mixture was concentrated. The residue was purified by prep-HPLC (column: Phenomenex luna C18 250×50 mm×5 μm; mobile phase: [water (TFA)-ACN]; B %: 5%-35%, 18 min) and lyophilized. Compound 3 (13.5 g, 49.0 mmol, 82.7% yield) was obtained as a yellow solid which was confirmed by ¹HNMR (400 MHz, DMSO_d6, δ: 7.04-7.16 (m, 2H), 6.46-6.57 (m, 2H), 5.27-5.36 (m, 2H), 3.50-3.57 (m, 2H), 3.37-3.43 (m, 2H), 1.70-1.82 (m, 2H), 1.08-1.17 (m, 9H)).

A mixture of compound 3 (10.5 g, 38.1 mmol, 1 eq) in DCM (100 mL) at 20-25° C., then CDI (12.37 g, 76.3 mmol, 2.00 eq) in DCM (50 mL) was added to the solution at 20-25° C. The mixture was stirred at 20-25° C. for 1 hour under N₂ atmosphere. The reaction was monitored with LCMS, and the desired MS was detected (M/Z+1+MeOH=234.1). The reaction mixture was concentrated under vacuum. The residue was taken to the next step without purification. Compound 3-a1 (26 g, crude) was obtained as a gray solid, which was confirmed by next step.

To a solution of compound 5 (3.34 g, 56.5 mmol, 4.65 mL, 1.00 eq) in DCM (130 mL) was added TEA (5.71 g, 56.4 mmol, 7.85 mL, 1.00 eq) at 25-30° C., and the mixture was cooled to 0° C. Then compound 5a (13 g, 56.4 mmol, 1.00 eq) was added to the solution at 0° C., then warmed to 25-30° C., and stirred at 25-30° C. for 12 hours. The reaction was monitored with LCMS, and the desired MS was detected (M/Z+1=254.0). The reaction mixture was concentrated under vacuum, and the resulting residue was purified by column chromatography (SiO₂, petroleum ether:ethyl acetate=30:1 to 3:1). Thin layer chromatography (petroleum ether:ethyl acetate=3:1) showed the product (R_{f}=0.50), which was collected. Compound 6 (12.3 g, 48.6 mmol, 86.2% yield) was obtained as a yellow solid which was confirmed by ¹HNMR (400 MHz, DMSO_d6, δ: 9.15-9.21 (m, 1H), 9.04-9.08 (m, 2H), 8.93-8.99 (m, 1H), 3.24-3.34 (m, 3H), 1.53-1.66 (m, 2H), 0.84-0.97 (m, 3H)).

To a solution of compound 6 (11.8 g, 46.6 mmol, 1.00 eq) in MeOH (120 mL) was added Pd/C (1.40 g, 46.60 mmol, 10% purity, 1.00 eq) under N₂ atmosphere. The suspension was degassed and purged with H₂ three times. The mixture was stirred at 30-40° C. under H₂ (50 Psi) for 12 hours. Thin layer chromatography (petroleum ether:ethyl acetate=3:1) showed compound 6 (R_{f}=0.30) was consumed and one new spot (R_{f}=0.00) formed. The reaction mixture was filtered, and the resulting filtrate was washed with MeOH (300 mL) and concentrated. The residue was taken to the next step without purification. Compound 7 (8.60 g, 44.5 mmol, 95.5% yield) was obtained as a red solid which was confirmed by ¹HNMR (400 MHz, DMSO_d6, δ: 7.80-8.02 (m, 1H), 6.16-6.29 (m, 2H), 5.84-5.97 (m, 1H), 4.82 (s, 4H), 3.03-3.20 (m, 2H), 1.34-1.56 (m, 2H), 0.76-0.91 (m, 3H)).

To a solution of compound 7 (4.85 g, 14.7 mmol, 1.00 eq) in DMF (150 mL) was added compound 3-a1 (26 g, 86.28 mmol, 5.85 eq) and DIEA (7.42 g, 57.4 mmol, 10 mL, 3.89 eq) at 20-25° C. and stirred at 20-25° C. for 12 hours. The reaction was monitored with LCMS, and compound 7 was consumed and the desired MS was detected (M/Z+1=796.5). The reaction mixture was concentrated under vacuum, and the crude product was purified by reverse-phase HPLC (NH₃·H₂O condition). Compound 7-a (8.05 g, 10.1 mmol, 68.6% yield) was obtained as a red solid which was confirmed by ¹HNMR (400 MHz, DMSO_d6, δ: 8.94-9.01 (m, 2H), 8.85-8.92 (m, 2H), 8.34-8.41 (m, 1H), 7.43-7.51 (m, 6H), 7.23-7.40 (m, 5H), 3.56-3.62 (m, 4H), 3.45-3.50 (m, 4H), 3.16-3.23 (m, 2H), 1.75-1.86 (m, 4H), 1.48-1.59 (m, 2H), 1.08-1.13 (m, 18H), 0.85-0.94 (m, 3H)).

To a solution of compound 7-a (8.89 g, 11.2 mmol, 1.00 eq) in DCM (80 mL) was added TFA (30.8 g, 270 mmol, 20 mL, 24.2 eq) at 20-25° C. and stirred at 20-25° C. for 24 hours. The reaction was monitored with LCMS, and compound 7-a was consumed and the desired MS was detected (M/Z+1=596.5). The reaction mixture was concentrated under vacuum and the residue was purified by prep-HPLC (column: YMC Triart C18 250×50 mm×7 μm; mobile phase: [water (HCl)-ACN]; B %: 7%-37%, 20 min) and lyophilized. Compound 1 (3.82 g, 6.37 mmol, 57.0% yield, 99.3% purity) was obtained as a yellow solid which was confirmed by LCMS (M/Z+1=596.7), HPLC, ¹FNMR, and ¹HNMR (400 MHz, DMSO-d6, δ: 9.94-10.12 (m, 2H), 9.73-9.89 (m, 4H), 9.51-9.66 (m, 2H), 8.35-8.46 (m, 1H), 7.80-7.85 (m, 1H), 7.65-7.73 (m, 8H), 7.46-7.54 (m, 2H), 3.42-3.53 (m, 8H), 3.14-3.25 (m, 2H), 1.83-2.04 (m, 4H), 1.45-1.58 (m, 2H), 0.78-0.95 (m, 3H)).

### Example 2

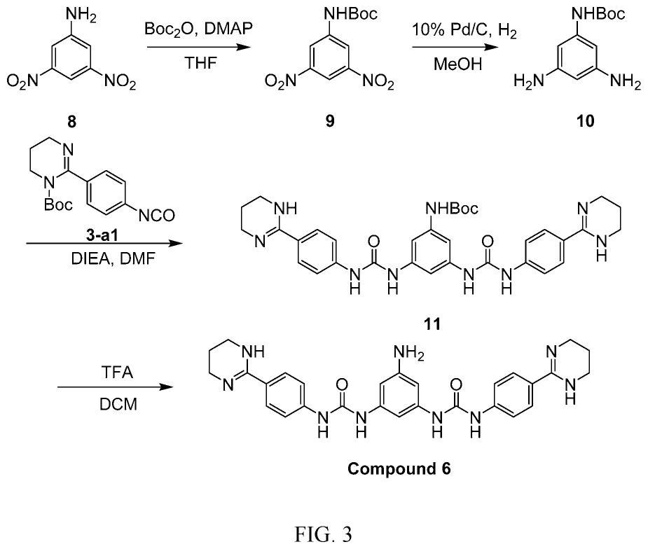

Exemplary compounds of formula (Ia) (e.g., compound 6) can be prepared in accordance with the reaction scheme set forth in FIG. 3. FIG. 3 is a reaction scheme of preparing compound 6. DMAP is 4-dimethylaminopyridine. Experimental protocols for the preparation of compounds set forth in FIG. 3 are provided below.

To a solution of compound 8 (1.00 g, 5.46 mmol, 1.00 eq) in THE (30 mL) was added Boc₂O (3.58 g, 16.4 mmol, 3.76 mL, 3.00 eq), DMAP (7 mg, 57.3 μmol, 0.10 eq), and TEA (1.38 g, 13.65 mmol, 1.90 mL, 2.5 eq) at 25° C. The mixture was stirred at 70° C. for one hour. Thin layer chromatography (petroleum ether:ethyl acetate=5:1) showed compound 8 (Rf=0.5) was consumed, and a major new spot (R_{f}=0.70) with lower polarity was detected. The mixture was evaporated to remove the solvents, and the resulting residue was dissolved in ethyl acetate (20 mL), washed with iN HCl (aq, 10 mL), NaHCO₃ (10 mL), and brine (10 mL), dried over Na₂SO₄, filtered, and concentrated under reduced pressure to give a residue. The residue was re-dissolved in EtOH (30 mL), and the resulting solution was treated with K₂CO₃ (2.48 g). The foregoing mixture was heated to 50° C. for one hour, and then concentrated to dryness. The resulting residue was dissolved in ethyl acetate (20 mL) and washed with iN HCl (10 mL) and brine (10 mL), dried over Na₂SO₄, and concentrated under vacuum. The residue was purified by column chromatography (SiO₂, petroleum ether:ethyl acetate=10:1 to 5:1). Compound 9 (1.31 g, 4.61 mmol, 84.4% yield) was obtained as white solid, which was confirmed by ¹HNMR (400 MHz, CDCl₃, δ: 8.69 (s, 1H), 8.64 (s, 2H), 7.03 (s, 1H), 1.57 (s, 10H)).

To a solution of compound 9 (1.31 g, 4.63 mmol, 1.00 eq) in MeOH (15 mL) was added Pd/C (131 mg, 4.63 mmol, 10.0% purity, 1.00 eq) at 25-30° C. under N₂ atmosphere. The suspension was degassed and purged with H₂ three times. The resulting mixture was stirred at 30-40° C. under H₂ (50 Psi) for 12 hours. Thin layer chromatography (petroleum ether:ethyl acetate=5:1) showed compound 9 (R_{f}=0.40) was consumed and one new spot (R_{f}=0.05) formed. The reaction mixture was filtered and the resulting filtrate was washed with MeOH (300 mL) and concentrated. The residue was taken to the next step without purification. Compound 10 (1.00 g, 4.48 mmol, 96.8% yield) was obtained as a pink solid which was confirmed by ¹HNMR (400 MHz, DMSO-d6, δ: 8.62-8.76 (m, 1H), 5.93-6.05 (m, 2H), 5.44-5.51 (m, 1H), 4.52-4.65 (m, 4H), 1.43-1.49 (m, 9H)).

To a solution of compound 10 (90 mg, 403 μmol, 1.00 eq) in DMF (5 mL) was added DIEA (200 mg, 1.55 mmol, 270 μL, 3.85 eq) and compound 3-a1 (700 mg, 2.32 mmol, 5.76 eq) from Example 1 at 20-25° C., and the mixture was stirred at 20-25° C. for 12 hours. The reaction mixture was concentrated under vacuum and the resulting residue was purified by prep-HPLC (column: Welch Xtimate C18 150×25 mm×5 μm; mobile phase: [water (NH₃H₂O)-ACN]; B %: 40%-70%, 8 min) and lyophilized. Compound 11 (160 mg, 193.72 μmol, 48.06% yield) was obtained as a white solid.

To a solution of compound 11 (160 mg, 194 μmol, 1.00 eq) in MeOH (3 mL) was added HCl/dioxane (4 M, 6 mL, 124 eq) at 15-20° C. and stirred at 15-20° C. for 24 hours. The reaction was monitored with LCMS, which showed that compound 11 was consumed and the desired MS was detected (M/Z+1=526.3). The reaction mixture was concentrated under vacuum and the crude product was triturated with DMSO (5 mL) at 15-20° C. for 30 min and filtered, and the filter cake was collected. The resulting crude product was triturated with H₂O (5 mL) at 15-20° C. for 30 min and filtered, and the filter cake was collected. Compound 6 (12.72 mg, 21.86 μmol, 11.3% yield, 96.6% purity, HCl) was obtained as a yellow solid which was confirmed by LCMS (M/Z+1=526.4), HPLC, ¹FNMR, and ¹HNMR (400 MHz, DMSO-d6, δ: 9.65-9.84 (m, 4H), 9.32-9.50 (m, 2H), 8.80-8.98 (m, 2H), 7.54-7.80 (m, 9H), 6.40-6.51 (m, 2H), 5.01-5.20 (m, 2H), 3.45-3.50 (m, 8H), 1.95-2.01 (m, 4H)).

### Example 3

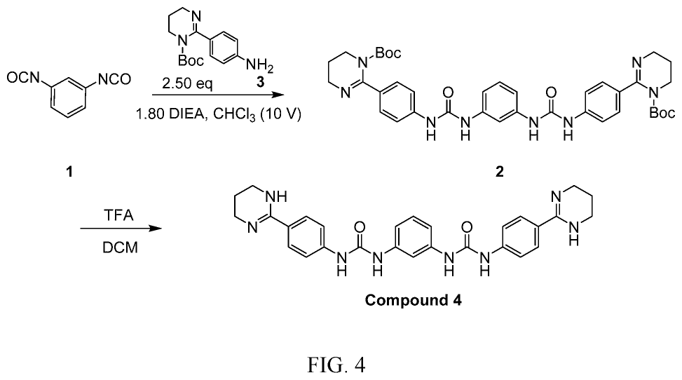

Exemplary compounds of formula (Ia) (e.g., compound 4) may be prepared in accordance with the reaction scheme set forth in FIG. 4. FIG. 4 is a reaction scheme of preparing compound 4. Experimental protocols for the preparation of compounds set forth in FIG. 4 are provided below.

To a solution of compound 1 in CHCl₃ (4 mL) was added compound 3 (125 mg, 780 μmol, 2.5 eq), as prepared in Example 1, and DIEA (74.2 mg, 574 μmol, 100 μL, 1.84 eq) at 25-30° C. The mixture was warmed to 50° C. and stirred at 50° C. for 12 hours. After 12 hours, the reaction mixture was concentrated under vacuum. The resulting residue containing compound 2 may be reacted with trifluoroacetic acid and dichloromethane to obtain compound 4. The reaction mixture was purified by prep-HPLC (column: Phenomenex luna C18 150×25 mm×10 μm; mobile phase: [water (FA)-ACN]; B %: 13%-43%, 9 min) and concentrated under vacuum. The reaction product was further purified by prep-HPLC (column: Phenomenex luna C18 150×25 mm×10 μm; mobile phase: [water (HCl)-ACN]; B %: 0%-25%, 9 min) and lyophilized. Compound 4 (9.43 mg, 17.12 μmol, 31.7% yield, 99.3% purity, HCl) was obtained as a yellow solid which was confirmed by LCMS (M/Z+1=511.4), HPLC, ¹FNMR, and ¹HNMR (400 MHz, DMSO-d6, δ: 9.64-9.94 (m, 6H), 9.24-9.46 (m, 2H), 7.49-7.82 (m, 9H), 6.95-7.30 (m, 3H), 3.42-3.55 (m, 8H), 1.90-2.06 (m, 4H)).

### Example 4

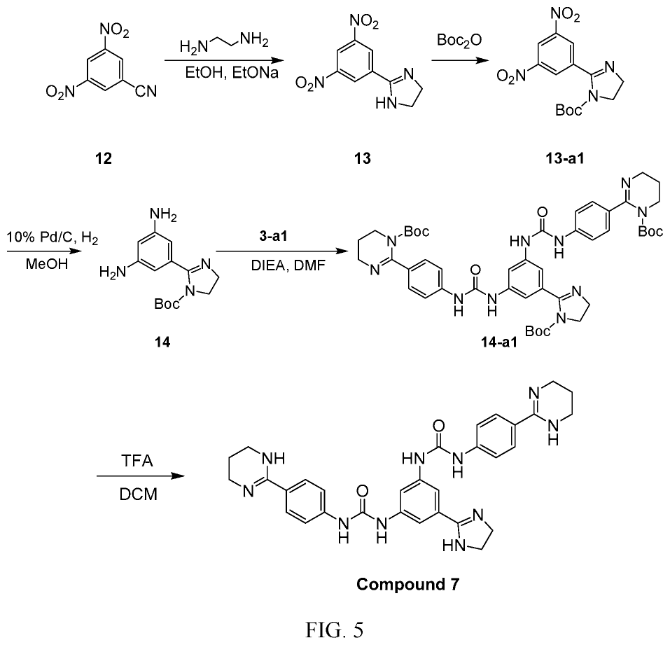

Exemplary compounds of formula (Ia) (e.g., compound 7) can be prepared in accordance with the reaction scheme set forth in FIG. 5. FIG. 5 is a reaction scheme of preparing compound 7. Experimental protocols for the preparation of compounds set forth in FIG. 5 are provided below.

To a solution of compound 12 (1.50 g, 7.77 mmol, 1.00 eq) in EtOH (10 mL) was added NaOEt (106 mg, 1.56 mmol, 0.2 eq) at 20-25° C. and stirred for 3 hours. Then ethylenediamine (467 mg, 7.77 mmol, 520 μL, 1.00 eq) was added and the reaction was stirred at 70-75° C. for 12 hours. Thin layer chromatography (dichloromethane:methanol=10:1) showed that compound 12 (R_{f}=0.80) was consumed and one new spot (R_{f}=0.10) formed. The reaction mixture was quenched by addition water (20 mL) at 25° C., and then diluted with HCl (10 mL) until pH=2 and extracted with ethyl acetate (20 mL*3). The aqueous layer was diluted with NaOH (6 M) 15 mL until pH=10, then extracted with ethyl acetate (20 mL*3). The organic layers were washed with brine (30 mL*3), dried over Na₂SO₄, filtered, and concentrated under reduced pressure to give a residue. The residue was taken to the next step without purification. Compound 13 (700 mg, 2.96 mmol, 38.2% yield) was obtained as a brown solid, which was confirmed by ¹HNMR (400 MHz, DMSO-d6, δ: 8.95-9.02 (m, 2H), 8.87-8.93 (m, 1H), 7.55-7.67 (m, 1H), 3.76-3.98 (m, 2H), 3.47-3.69 (m, 2H)).

To a solution of compound 13 (700 mg, 2.96 mmol, 1.00 eq) in THF (30 mL) was added TEA (872 mg, 8.62 mmol, 1.20 mL, 2.91 eq) and Boc₂O (779 mg, 3.57 mmol, 820 μL, 1.20 eq) at 20-25° C., and then the mixture was stirred at 20-25° C. for 36 hours. Thin layer chromatography (dichloromethane:methanol=20:1) showed that compound 13 (R_{f}=0.30) was consumed and one new spot (R_{f}=0.60, 0.70) formed. The reaction mixture was poured into water (100 mL) and extracted with ethyl acetate (40 mL*3). The combined organic layer was washed with saturated saltwater (90 mL), dried over Na₂SO₄, filtered, and concentrated under reduced pressure to give a residue. The residue was purified by column chromatography (SiO₂, petroleum ether:ethyl acetate=30:1 to 1:1). Compound 13-a1 (1.30 g, crude) was obtained as a yellow oil, which was confirmed by ¹HNMR (400 MHz, DMSO-d6, δ: 8.87-8.92 (m, 1H), 8.70-8.75 (m, 2H), 3.94-3.96 (m, 4H), 1.23-1.25 (m, 9H)).

To a solution of compound 13-a1 (1.30 g, 3.87 mmol, 1.00 eq) in MeOH (15 mL) was added Pd/C (130 mg, 3.87 mmol, 10% purity, 1.00 eq) at 20-25° C. under N₂ atmosphere. The suspension was degassed and purged with H₂ three times. The mixture warmed to 30-40° C. and was stirred at 30-40° C. under H₂ (50 Psi) for 12 hour. Thin layer chromatography (petroleum ether:ethyl acetate=1:1) showed that compound 13-a1 (R_{f}=0.60) was consumed and two new spots (R_{f}=0.10, 0.00) formed. The reaction mixture was filtered, and the filtrate was washed with MeOH (400 mL) and concentrated. The residue was purified by prep-HPLC (column: YMC Triart C18 250×50 mm×7 μm; mobile phase: [water (NH₄HCO₃)-ACN]; B %: 14%-44%, 20 min) and lyophilized. Compound 14 (250 mg, 905 μmol, 23.4% yield) was obtained as a white solid, which was confirmed by ¹HNMR (400 MHz, DMSO-d6, δ: 5.88-5.92 (m, 2H), 5.84-5.87 (m, 1H), 4.64-4.79 (m, 4H), 3.67-3.85 (m, 4H), 1.23 (s, 9H)).

To a solution of compound 14 (50 mg, 181 μmol, 1 eq) in DMF (2 mL) was added DIEA (89.04 mg, 689 μmol, 120 μL, 3.81 eq) and compound 3-a1 (140 mg, 465 μmol, 2.57 eq) from Example 1 at 20-25° C. and stirred at 20-25° C. for 24 hours. The reaction was monitored with LCMS, and the desired MS was detected (M/Z+1=879.5). The reaction mixture was filtered, and the mother liquor was collected. The resulting residue was purified by prep-HPLC (column: Welch Xtimate C18 150×25 mm×5 μm; mobile phase: [water (NH₃H₂O)-ACN]; B %: 37%-67%, 8 min) and lyophilized. Compound 14-a1 (130 mg, 148 μmol, 81.8% yield) was obtained as a yellow solid, which was confirmed by the next step.

To a solution of Compound 14-a1 (130 mg, 148 μmol, 1.00 eq) in DCM (5 mL) was added TFA (770 mg, 6.75 mmol, 0.50 mL, 45.6 eq) at 0° C., and the mixture warmed to 20° C. and stirred at 20° C. for 12 hours. The reaction was monitored with LCMS, and the desired MS was detected (M/Z+1=579.7). The reaction mixture was concentrated under vacuum, and the resulting residue was purified by prep-HPLC (column: Phenomenex luna C18 150×25 mm×10 μm; mobile phase: [water (FA)-ACN]; B %: 0%-22%, 10 min) and lyophilized. The resulting material was further purified by prep-HPLC (column: Phenomenex luna C18 150×25 mm×10 μm; mobile phase: [water (HCl)-ACN]; B %: 0%-25%, 10 min) and lyophilized. Compound 7 (34.84 mg, 56.4 μmol, 38.1% yield, 99.6% purity, HCl) was obtained as a white solid, which was confirmed by LCMS (M/Z+1=579.5), HPLC, ¹FNMR, and ¹HNMR (400 MHz, DMSO-d6, δ: 10.48-10.75 (m, 2H), 10.22-10.41 (m, 2H), 9.95-10.10 (m, 2H), 9.73-9.89 (m, 4H), 7.93-7.97 (m, 1H), 7.68-7.72 (m, 8H), 7.64-7.67 (m, 2H), 3.96-4.04 (m, 4H), 3.44-3.51 (m, 8H), 1.85-2.04 (m, 5H)).

### Example 5

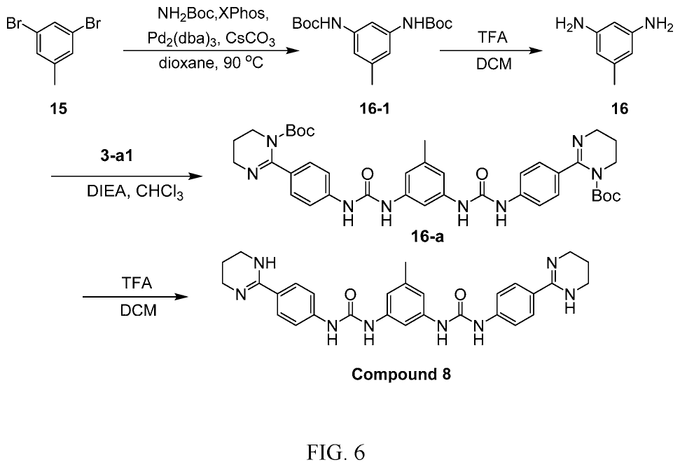

Exemplary compounds of formula (Ia) (e.g., compound 8) can be prepared in accordance with the reaction scheme set forth in FIG. 6. FIG. 6 is a reaction scheme of preparing compound 8. Experimental protocols for the preparation of compounds set forth in FIG. 6 are provided below.

To a solution of compound 15 (1.00 g, 4.00 mmol, 1.00 eq) in dioxane (12 mL) was added NH₂Boc (1.12 g, 9.60 mmol, 2.40 eq), Cs₂CO₃ (3.65 g, 11.2 mmol, 2.80 eq), Pd₂(dba)₃ (147 mg, 160 μmol, 4.01e-2 eq) and XPhos (232 mg, 400.95 μmol, 0.10 eq) at 25-30° C. The reaction mixture was warmed to 90° C. and heated at 90° C. for 4 hours. Then, NH₂Boc (982 mg, 8.38 mmol, 2.40 eq), Cs₂CO₃ (3.19 g, 9.78 mmol, 2.80 eq), Pd₂(dba)₃ (128 mg, 139 μmol, 0.04 eq), and XPhos (202 mg, 349 μmol, 0.1 eq) were added to the solution, and the reaction mixture was stirred at 90° C. for and additional 12 hour. Thin layer chromatography (petroleum ether:ethyl acetate=3:1) showed that compound 15 (R_{f}=0.30) was consumed and two new spots (R_{f}=0.20, 0.00) formed. The reaction mixture was filtered and concentrated under vacuum. The resulting residue was purified by column chromatography (SiO₂, petroleum ether:ethyl acetate=30:1 to 3:1). Compound 16-1 (664 mg, 2.06 mmol, 58.9% yield) was obtained as a yellow solid, which was confirmed by ¹HNMR (400 MHz, DMSO-d6, δ: 8.85-9.13 (m, 1H), 8.04-8.23 (m, 1H), 7.21-7.45 (m, 5H), 6.57-6.73 (m, 1H), 5.47-5.60 (m, 2H)).

To a solution of compound 16-1 (664 mg, 2.06 mmol, 1.00 eq) in DCM (5 mL) was added TFA (770 mg, 6.75 mmol, 0.50 mL, 3.28 eq) at 0° C., and the reaction mixture was warmed to 25-30° C. and stirred at 25-30° C. for 17 hours. Thin layer chromatography (petroleum ether:ethyl acetate=5:1) showed that compound 16-1 (R_{f}=0.40) was consumed and one major new spot (R_{f}=0.00) with larger polarity was detected. The reaction mixture was concentrated under vacuum, and the resulting residue was taken to the next step without purification. Compound 16 (480 mg, 2.03 mmol, 98.7% yield, TFA) was obtained as a red solid, which was confirmed by ¹HNMR (400 MHz, DMSO-d6, δ: 6.20-6.48 (m, 3H), 2.15-2.23 (m, 3H)).

To a solution of compound 16 (20 mg, 164 μmol, 1.00 eq) in CHCl₃ (4 mL) was added TEA (50.9 mg, 503 μmol, 70.00 μL, 3.07 eq) and compound 3-a1 (300 mg, 995 μmol, 6.08 eq) from Example 1 at 20-25° C. The reaction mixture was warmed to 45° C. and stirred at 45° C. for 12 hours. The reaction was monitored with LCMS, and compound 16 was consumed and the desired MS was detected (M/Z+1=725.5). The reaction mixture was concentrated under vacuum, and the resulting residue was purified by prep-HPLC (column: Phenomenex C18 150×30 mm×5 μm; mobile phase: [water (NH₄HCO₃)-ACN]; B %: 35%-65%, 8 min) and lyophilized. Compound 16-a (40 mg, 55.2 μmol, 33.7% yield) was obtained as a white solid, which was confirmed by next step.

To a solution of compound 16-a (40 mg, 55.2 μmol, 1.00 eq) in DCM (2 mL) was added TFA (770 mg, 6.75 mmol, 0.5 mL, 122 eq) at 0° C., and the reaction mixture was stirred at 20-25° C. for 14 hours. The reaction was monitored with LCMS, and compound 16-a was consumed and the desired MS was detected (M/Z+1=525.5). The reaction mixture was filtered, and the resulting residue was purified by prep-HPLC (column: Phenomenex luna C18 150×25 mm×10 μm; mobile phase: [water (FA)-ACN]; B %: 3%-33%, 10 min) and lyophilized. Then the residue was further purified by prep-HPLC (column: Phenomenex luna C18 150×25 mm×10 μm; mobile phase: [water (HCl)-ACN]; B %: 3%-33%, 10 min) and lyophilized. Compound 8 (9.76 mg, 17.01 μmol, 59.1% yield, 97.8% purity, HCl) was obtained as a white solid, which was confirmed by LCMS (M/Z+1=525.5), HPLC, ¹FNMR and ¹HNMR (400 MHz, DMSO-d6, δ: 9.94-10.08 (m, 2H), 9.77-9.87 (m, 4H), 9.42-9.53 (m, 2H), 7.59-7.78 (m, 8H), 7.48-7.55 (m, 1H), 6.91-6.99 (m, 2H), 3.42-3.51 (m, 8H), 2.20-2.29 (m, 3H), 1.90-2.02 (m, 4H)).

### Example 6

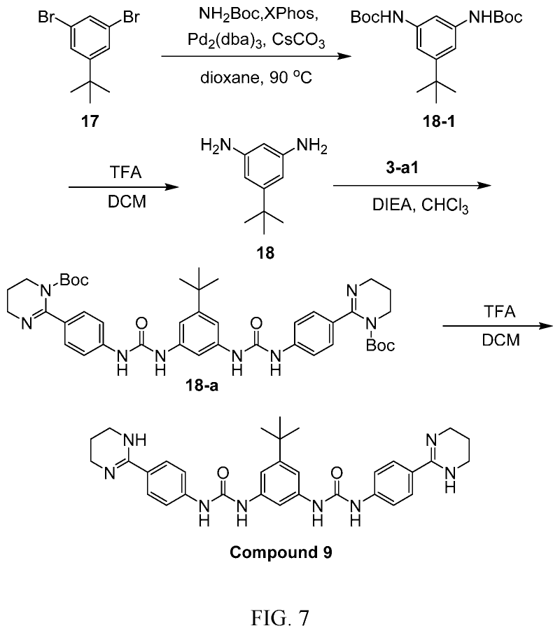

Exemplary compounds of formula (Ia) (e.g., compound 9) can be prepared in accordance with the reaction scheme set forth in FIG. 7. FIG. 7 is a reaction scheme of preparing compound 9. Experimental protocols for the preparation of compounds set forth in FIG. 7 are provided below.

To a solution of compound 17 (1.00 g, 3.42 mmol, 1.00 eq) in dioxane (12 mL) was added NH₂Boc (2.01 g, 17.1 mmol, 5.00 eq) Cs₂CO₃ (6.69 g, 20.5 mmol, 6.00 eq), XPhos (397 mg, 686 μmol, 0.20 eq), and Pd₂(dba)₃ (314 mg, 342 μmol, 0.10 eq) at 25° C., and the reaction mixture was warmed to 90° C. and stirred at 90° C. for 18 hours under N₂. Thin layer chromatography (petroleum ether:ethyl acetate=10:1) showed that compound 17 (R_{f}=1.0) was consumed, and a major new spot (R_{f}=0.70) with larger polarity was detected. The reaction mixture was poured into water (20 mL) and extracted with ethyl acetate (15 mL*3). The combined organic layer was washed with brine (15 mL), dried over Na₂SO₄, filtered, and concentrated. The resulting residue was purified by column chromatography (SiO₂, petroleum ether:ethyl acetate=20:1 to 10:1). Compound 18-1 (1.15 g, 3.15 mmol, 91.9% yield) was obtained as yellow solid, which was confirmed by ¹HNMR (400 MHz, CDCl3, δ: 7.23-7.31 (m, 1H), 6.92-7.01 (m, 2H), 6.31-6.48 (m, 2H), 1.42-1.45 (m, 18H), 1.19-1.22 (m, 9H)).

To a solution of compound 18-1 in DCM (2 mL), was added TFA (3.13 g, 27.4 mmol, 2.03 mL, 20.0 eq) at 0° C. The mixture was stirred at 25° C. for 1 hour and a large quantity of precipitate was formed. The mixture was concentrated under reduced pressure and the residue was taken to the next step without purification. Compound 18 (370 mg, 1.33 mmol, 96.9% yield, TFA) was obtained as a yellow solid.

To a solution of compound 18 (30 mg, 107 μmol, 1.00 eq, TFA) in CHCl₃ (4 mL) was added DIEA (59.36 mg, 459 μmol, 80 μL, 4.26 eq) and compound 3-a1 (300 mg, 995 μmol, 9.23 eq) from Example 1 at 20-25° C. The reaction mixture was warmed to 45° C. and stirred at 45° C. for 12 hours. The reaction was monitored with LCMS, and compound 18 was consumed and the desired MS was detected (M/Z+1=767.5). The reaction mixture was concentrated under vacuum, and the resulting residue was purified by prep-HPLC (column: Welch Xtimate C18 150×25 mm×5 μm; mobile phase: [water (NH₃H₂O)-ACN]; B %: 42%-72%, 8 min) and lyophilized. Compound 18-a (50 mg, 65.2 μmol, 60.5% yield) was obtained as a white solid, which was confirmed by ¹HNMR (400 MHz, DMSO-d6, δ: 8.63-8.79 (m, 4H), 7.51-7.55 (m, 1H), 7.43-7.50 (m, 4H), 7.32-7.38 (m, 4H), 7.09-7.17 (m, 2H), 3.56-3.62 (m, 4H), 3.45-3.49 (m, 4H), 1.75-1.85 (m, 4H), 1.25-1.30 (m, 9H), 1.08-1.14 (m, 18H)).

To a solution of compound 18-a (50 mg, 65.2 μmol, 1.00 eq) in DCM (2 mL) was added TFA (770 mg, 6.75 μmol, 0.5 mL, 104 eq) at 0° C. The reaction mixture was warmed to 20-25° C. and stirred at 20-25° C. for 12 hours. The reaction was monitored with LCMS, and the desired MS was detected (M/Z+1=567.4). The reaction mixture was concentrated under reduced pressure and the resulting residue was purified by prep-HPLC (column: Phenomenex luna C18 150×25 mm×10 μm; mobile phase: [water (FA)-ACN]; B %: 7%-37%, 10 min) and lyophilized. Compound 9 (22.12 mg, 35.7 μmol, 54.8% yield, 98.9% purity, HCl) was obtained as a white solid, which was confirmed by LCMS (M/Z+1=567.4), HPLC, ¹FNMR, and ¹HNMR (400 MHz, DMSO-d6, δ: 9.73-9.82 (m, 4H), 9.50-9.69 (m, 2H), 9.18-9.31 (m, 2H), 7.61-7.74 (m, 8H), 7.45-7.56 (m, 1H), 7.07-7.26 (m, 2H), 3.44-3.52 (m, 8H), 1.92-2.02 (m, 4H), 1.27 (s, 9H)).

### Example 7

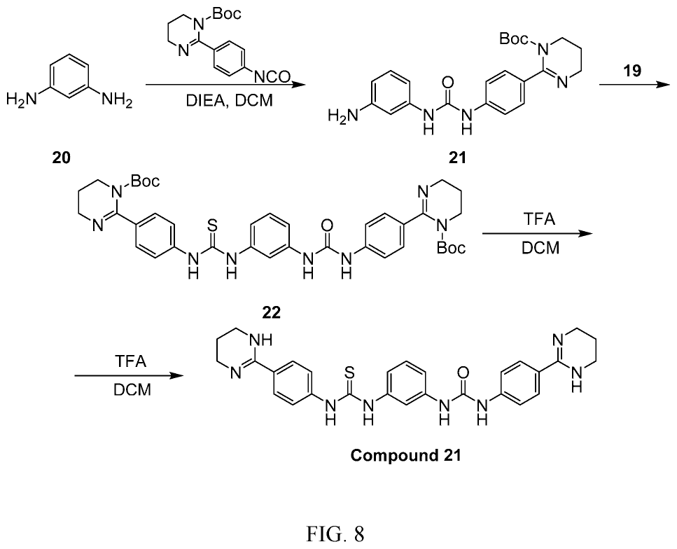

Exemplary compounds of formula (Ia) (e.g., compound 21) can be prepared in accordance with the reaction scheme set forth in FIG. 8. FIG. 8 is a reaction scheme of preparing compound 21. Experimental protocols for the preparation of compounds set forth in FIG. 8 are provided below.

To a solution of compound 3 (200 mg, 726 μmol, 1.00 eq) from Example 1 in DCM (2 mL) at 20-25° C., was added TCDI (129 mg, 723 μmol, 1.00 eq) in DCM (2.00 mL) at 20-25° C., and the reaction mixture was stirred at 20-25° C. for 1 hour. The reaction was monitored with LCMS, and the desired MS was detected (M/Z+1=318.2). The reaction mixture was concentrated under vacuum and the resulting residue was taken to the next step without purification. Compound 19 (230 mg, 724 μmol, 99.7% yield) was obtained as a yellow solid.

To a solution of compound 20 (233 mg, 2.15 mmol, 2.00 eq) in DCM (6 mL) was added DIEA (274 mg, 2.12 mmol, 370 μL, 1.97 eq) and compound 3-a1 (325 mg, 1.08 mmol, 1.00 eq) from Example 1 at 20-25° C., and the reaction was stirred at 20-25° C. for 12 hours. The reaction was monitored with LCMS, and the desired MS was detected (M/Z+1=410.2). The reaction mixture was filtered and the mother liquor was collected. The collected residue was purified by prep-HPLC (column: Phenomenex C18 250×50 mm×10 um; mobile phase: [water (ammonia hydroxide)-ACN]; B %: 21%-51%, 8 min) and lyophilized. Compound 21 (171 mg, 417 μmol, 38.7% yield) was obtained as a white solid, which was confirmed by ¹HNMR (400 MHz, DMSO-d6, δ: 8.66-8.72 (m, 1H), 8.34-8.44 (m, 1H), 7.41-7.48 (m, 2H), 7.30-7.36 (m, 2H), 6.86-6.94 (m, 1H), 6.75-6.79 (m, 1H), 6.53-6.59 (m, 1H), 6.17-6.23 (m, 1H), 4.99-5.07 (m, 2H), 3.55-3.62 (m, 2H), 3.44-3.51 (m, 2H), 1.74-1.85 (m, 2H), 1.10 (s, 9H)).

To a solution of compound 21 (100 mg, 244 μmol, 1.00 eq) in DCM (4 mL) was added DIEA (66.8 mg, 516 μmol, 90 μL, 2.12 eq) and compound 19 (100 mg, 315 μmol, 1.29 eq) at 20-25° C., and the reaction mixture was stirred at 20-25° C. for 12 hours. The reaction was monitored with LCMS, and the desired MS was detected (M/Z+1=727.4). The reaction mixture was concentrated under vacuum, and the resulting residue was purified by prep-HPLC (column: Waters xbridge 150×25 mm×10 um; mobile phase: [water (NH₄HCO₃)-ACN]; B %: 35%-65%, 11 min) and lyophilized. Compound 22 (100 mg, 137 μmol, 56.3% yield) was obtained as a yellow solid, which was confirmed by ¹HNMR (400 MHz, DMSO-d6, δ: 9.79-9.90 (m, 2H), 8.78-8.84 (m, 1H), 8.70-8.78 (m, 1H), 7.68 (s, 1H), 7.51-7.56 (m, 2H), 7.42-7.49 (m, 2H), 7.31-7.41 (m, 4H), 7.27 (s, 2H), 7.11-7.15 (m, 1H), 3.55-3.63 (m, 4H), 3.45-3.51 (m, 4H), 1.77-1.84 (m, 4H), 1.07-1.14 (m, 18H)).

To a solution of compound 22 (100 mg, 137 μmol, 1.00 eq) in DCM (4 mL) was added TFA (770 mg, 6.75 mmol, 0.50 mL, 49.1 eq) at 0° C., and the reaction mixture was warmed to 20° C. and stirred 20° C. for 12 hours. The reaction was monitored with LCMS, and the desired MS was detected (M/Z+1=527.3). The reaction mixture was concentrated under vacuum, and the resulting residue was purified by prep-HPLC (column: Phenomenex Luna C18 100×30 mm×5 μm; mobile phase: [water (FA)-ACN]; B %: 1%-20%, 8 min) and lyophilized. Then the reaction product was further purified by prep-HPLC (column: Phenomenex luna C18 150×25 mm×10 μm; mobile phase: [water (HCl)-ACN]; B %: 1%-30%, 10 min) and lyophilized. Compound 21 (6.28 mg, 11.06 μmol, 23.6% yield, 99.2% purity, HCl) was obtained as a yellow solid, which was confirmed by LCMS (M/Z+1=527.3), HPLC, ¹FNMR, and ¹HNMR (400 MHz, DMSO-d6, δ:10.78-10.91 (m, 1H), 10.54-10.68 (m, 1H), 9.85-9.94 (m, 3H), 9.76-9.84 (m, 2H), 9.49-9.60 (m, 1H), 7.84-7.97 (m, 2H), 7.60-7.76 (m, 7H), 7.17-7.34 (m, 3H), 3.41-3.55 (m, 8H), 1.89-2.05 (m, 4H)).

### Example 8

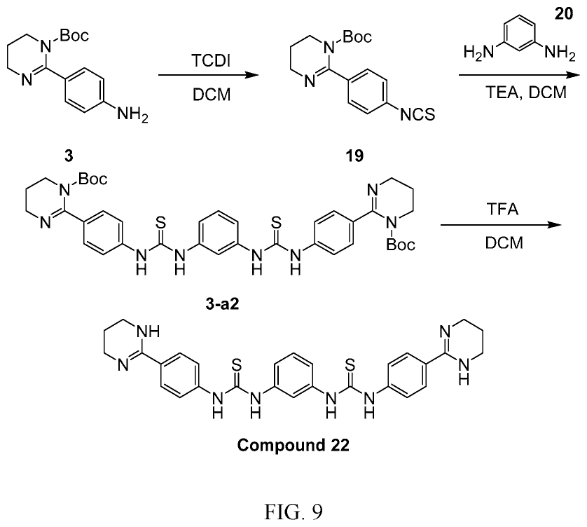

Exemplary compounds of formula (Ia) (e.g., compound 22) can be prepared in accordance with the reaction scheme set forth in FIG. 9. FIG. 9 is a reaction scheme of preparing compound 22. TCDI is 1,1′-thiocarbonyldiimidazole. Experimental protocols for the preparation of compounds set forth in FIG. 9 are provided below.

To a solution of compound 3 (200 mg, 726 μmol, 1.00 eq) from Example 1 in DCM (2 mL) at 20-25° C., was added TCDI (129 mg, 723 μmol, 1.00 eq) in DCM (2.00 mL) at 20-25° C., and the reaction mixture was stirred at 20-25° C. for 1 hour. The reaction was monitored with LCMS, and the desired MS was detected (M/Z+1=318.2). The reaction mixture was concentrated under vacuum and the resulting residue was taken to the next step without purification. Compound 19 (230 mg, 724 μmol, 99.7% yield) was obtained as a yellow solid.

To a solution of compound 19 (450 mg, 1.42 mmol, 1.00 eq) and DIEA (550 mg, 4.26 mmol, 741 μL, 3.00 eq) in DCM (10.0 mL) at 15-25° C., was added compound 20 (70.0 mg, 647 μmol, 2.50 eq) and the reaction mixture was stirred at 15-25° C. for 12 hours under N₂. The reaction was monitored with LCMS, and the desired MS was detected (M/Z+1=743.3). The crude product was purified by prep-HPLC (column: Phenomenex C18 250×50 mm×10 μm; mobile phase: [water (ammonia hydroxide v/v)-ACN]; B %: 33%-63%, 8 min). Compound 3-a2 was obtained as white solid, which was confirmed by LCMS.

To a solution of compound 3-a2 (80.0 mg, 107 μmol, 1.00 eq) in DCM (10.0 mL) was added dropwise TFA (13.0 mg, 114 μmol, 8.44 μL, 1.06 eq) at 0° C., and the reaction mixture was stirred at 25° C. for 12 hours. The reaction was monitored with LCMS, and the desired MS was detected (M/Z+1=543.4) was detected. The crude product was purified by prep-HPLC (column: Phenomenex Luna C18 150×25 mm×10 μm; mobile phase: [water(HCl)-ACN]; B %: 0%-25%, 10 min). Compound 22 (6.38 mg, 11.2 μmol, 10.4% yield, 95.5% purity) was obtained as yellow gum, which was confirmed by LCMS (M/Z+1=543.4), HPLC, ¹FNMR, and ¹HNMR (400 MHz, DMSO-d6, δ: 10.48 (d, 4H), 9.82 (s, 4H), 7.84 (d, 4H), 7.80 (s, 1H), 7.68 (d, 4H), 7.36 (s, 2H), 3.48 (s, 8H), 1.96-2.00 (m, 4H)).

### Example 9

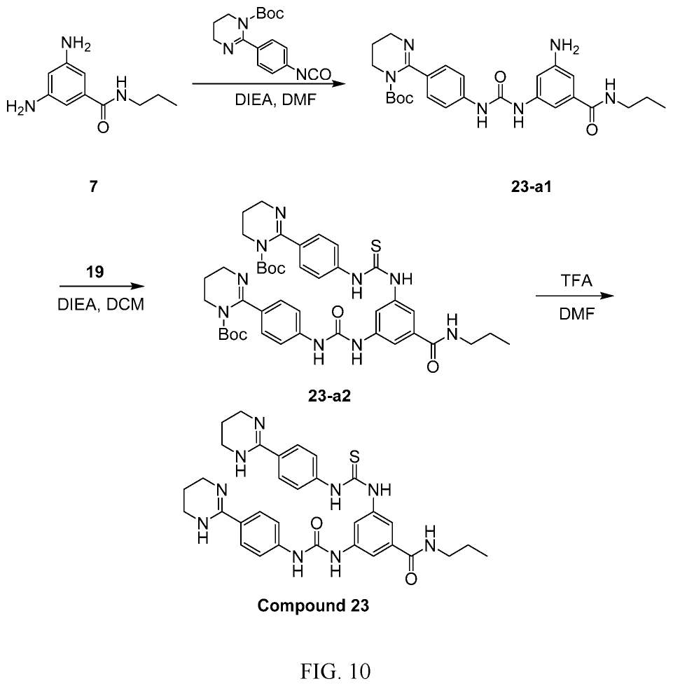

Exemplary compounds of formula (Ia) (e.g., compound 23) can be prepared in accordance with the reaction scheme set forth in FIG. 10. FIG. 10 is a reaction scheme of preparing compound 23. Experimental protocols for the preparation of compounds set forth in FIG. 10 are set forth below.

To a solution of compound 7 (1.03 g, 5.35 mmol, 2.02 eq) and DIEA (687 mg, 5.32 mmol, 925 μL, 2.00 eq) in DMF (12.0 mL) was added compound 3-a1 (800 mg, 2.65 mmol, 1.00 eq) from Example 1 drop-wise at 25° C. The reaction mixture was stirred under N₂ at 25° C. for 12 hours. The reaction was monitored with LCMS, and the desired MS was detected (M/Z+1=495.4). The crude product was purified by prep-HPLC (column: YMC Triart C18 250×50 mm×7 μm; mobile phase: [water(NH₄HCO₃)-ACN]; B %: 23%-53%, 20 min). Compound 23-a1 was obtained as white solid, which was confirmed by ¹HNMR (400 MHz, DMSO-d6, δ 8.80 (s, 1H), 8.56 (s, 1H), 8.16 (t, J=5.6 Hz, 1H), 7.44 (d, J=8.6 Hz, 2H), 7.36 (d, J=8.6 Hz, 2H), 6.92 (s, 2H), 6.60 (d, J=1.5 Hz, 1H), 5.24 (s, 2H), 3.60 (t, J=6.6 Hz, 2H), 3.48 (d, J=5.8 Hz, 2H), 3.12-3.20 (m, 2H), 1.76-1.82 (m, 2H), 1.44-1.54 (m, 2H), 1.10 (s, 9H), 0.88 (t, J=7.4 Hz, 3H)).

To a solution of compound 23-a1 (156 mg, 315 μmol, 1.00 eq) and DIEA (41.0 mg, 317 μmol, 55.2 μL, 1.01 eq) in DCM (2.00 mL), was added compound 19 (100 mg, 315 μmol, 1.00 eq) from Example 8 at 25° C., and the reaction mixture was stirred at 25° C. for 12 hours. The reaction was monitored with LCMS, and the desired MS was detected (M/Z+1=812.4) was detected. Compound 23-a2 was obtained a yellow solid, which was confirmed by ¹HNMR (400 MHz, DMSO-d6, δ 9.92 (s, 2H), 8.82-8.92 (m, 2H), 8.42 (t, J=5.6 Hz, 1H), 7.80 (s, 1H), 7.68 (s, 1H), 7.46-7.56 (m, 5H), 7.32-7.40 (m, 4H), 3.56-3.62 (m, 4H), 3.48 (m, 4H), 3.20 (d, J=6.8 Hz, 2H), 1.76-1.84 (m, 4H), 1.48-1.56 (m, 2H), 1.08-1.12 (m, 18H), 0.88 (t, J=7.4 Hz, 3H)).

To a solution of compound 23-a2 (120 mg, 147 μmol, 1.00 eq) in MeOH (3.00 mL) was added dropwise HCl/MeOH (4 M, 1.50 mL, 40.6 eq) at 0° C., and the reaction mixture was stirred at 25° C. for 12 hours. The reaction was monitored with LCMS, and the desired MS was detected (M/Z+1=612.5) was detected. The residue was purified by prep-HPLC (column: Phenomenex luna C18 150×25 mm×10 μm; mobile phase: [water(HCl)-ACN]; B %: 2%-32%, 10 min). Compound 23 was obtained a white solid, which was confirmed by LCMS (M/Z+1=612.5), HPLC, ¹FNMR, and ¹HNMR (400 MHz, DMSO-d6, δ: 10.8 (s, 1H), 10.4-10.4 (m, 1H), 9.76-9.92 (m, 5H), 9.56-9.64 (m, 1H), 8.44 (t, J=5.2 Hz, 1H), 7.92 (d, J=8.0 Hz, 2H), 7.82 (s, 1H), 7.66-7.76 (m, 7H), 7.58 (s, 1H), 3.48 (d, J=4.0 Hz, 8H), 3.21 (d, J=6.5 Hz, 2H), 1.98 (d, J=4.0 Hz, 4H), 1.48-1.58 (m, 2H), 0.90 (t, J=7.4 Hz, 3H)).

### Example 10

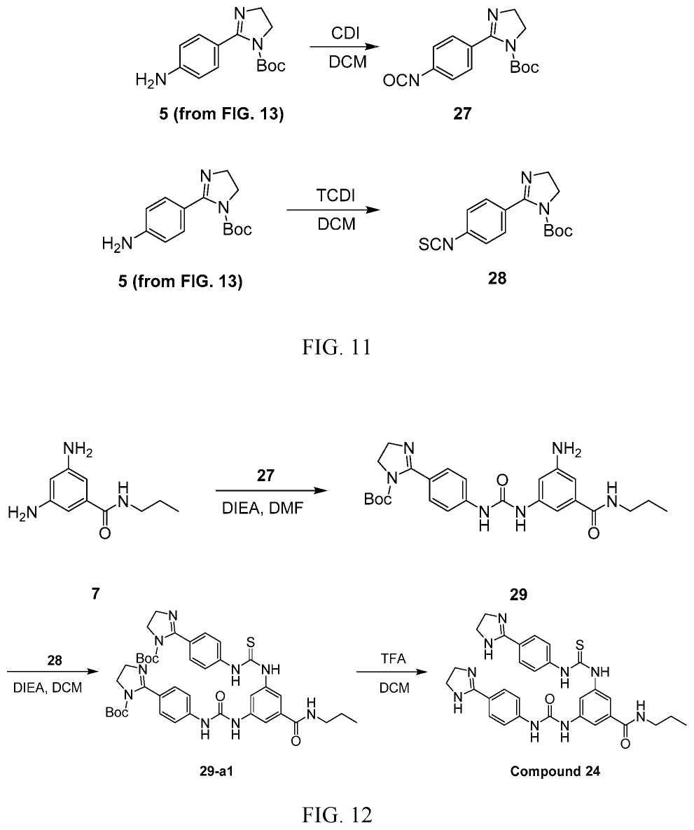

Exemplary compounds of formula (Ia) (e.g., compound 24) can be prepared in accordance with the reaction schemes set forth in FIG. 11 and FIG. 12. FIG. 11 is a reaction scheme of preparing building blocks 27 and 28. FIG. 12 is a reaction scheme of preparing compound 24. Experimental protocols for the preparation of compounds set forth in FIG. 11 and FIG. 12 are provided below.

To a solution of compound 5 (250 mg, 957 μmol, 1.00 eq) from Example 11 in DCM (5 mL) was added CDI (155 mg, 955.91 μmol, 1.00 eq) at 20° C. and the reaction mixture was stirred at 20° C. for 1 hour. The reaction was monitored by LCMS, and the desired MS was detected (M/Z+1+32=320.2). The reaction mixture was concentrated under vacuum and compound 27 (500 mg, crude) was obtained as a yellow solid, which was used directly in the next reaction.

To a solution of compound 5 (300 mg, 1.15 mmol, 1.00 eq) in DCM (6 mL) was added TCDI (205 mg, 115 mmol, 1.00 eq) at 20° C. and the reaction mixture was stirred at 20° C. for 1 hour. The reaction was monitored by LCMS, and the desired MS was detected (M/Z+1-56=248.0). The reaction mixture was concentrated under vacuum and compound 28 (500 mg, crude) was obtained as a yellow solid, which was used directly in the next reaction.

To a solution of compound 7 (300 mg, 1.55 mmol, 1.00 eq) in DMF (5 mL) was added DIEA (742 mg, 5.74 mmol, 1 mL, 3.70 eq) and compound 28 (377 mg, 1.24 mmol, 0.80 eq) at 20° C., and the reaction mixture was stirred at 20° C. for 12 hours. The reaction was monitored by LCMS, and the desired MS was detected (M/Z+1=497.3). The reaction mixture was filtered and the mother liquor was collected. The resulting residue was purified by prep-HPLC (column: Phenomenex C18 250×50 mm×10 μm; mobile phase: [water (ammonia hydroxide v/v)-ACN]; B %: 20%-50%, 8 min) and lyophilized. Compound 29 (210 mg, 423 μmol, 27.2% yield) was obtained as a white solid, which was confirmed by ¹HNMR (400 MHz, DMSO-d6, δ: 7.33-7.45 (m, 5H), 7.24-7.30 (m, 5H), 5.19-5.25 (m, 2H), 4.84-4.91 (m, 1H), 4.70-4.79 (m, 1H), 3.94-3.99 (m, 1H), 3.77-3.84 (m, 1H), 3.57-3.65 (m, 1H), 3.32-3.44 (m, 1H), 2.98-3.08 (m, 2H), 2.43-2.57 (m, 2H), 1.43-1.46 (m, 9H)).

To a solution of compound 29 (210 mg, 423 μmol, 1.00 eq) in DMF (5 mL) was added DIEA (223 mg, 1.72 mmol, 300 μL, 4.07 eq) and compound 27 (300 mg, 1.04 mmol, 2.47 eq) at 20-25° C., and the reaction mixture was stirred at 20-25° C. for 12 hours. The reaction was monitored by LCMS, and the desired MS was detected (M/Z+1=784.3). The reaction mixture was filtered and the mother liquor was collected. The resulting residue was purified by prep-HPLC (column: Phenomenex C18 250×50 mm×10 μm; mobile phase: [water (ammonia hydroxide v/v)-ACN]; B %: 33%-63%, 8 min) and lyophilized. Compound 29-a1 (321 mg, 409 μmol, 96.8% yield) was obtained as a white solid, which was used directly in the next reaction.

To a solution of compound 29-a1 (321 mg, 409 μmol, 1.00 eq) in DCM (4 mL) was added TFA (2.15 g, 18.8 mmol, 1.40 mL, 46.0 eq) at 0° C., and the reaction mixture was warmed to 15-20° C. and stirred at 15-20° C. for 12 hours. The reaction was monitored by LCMS, and the desired MS was detected (M/Z+1=584.4). The reaction mixture was concentrated under vacuum, and the resulting residue was purified by prep-HPLC (column: Welch Xtimate C18 150×25 mm×5 μm; mobile phase: [water (TFA)-ACN]; B %: 2%-32%, 10 min) and lyophilized. Compound 24 (32.96 mg, 53.7 μmol, 13.1% yield, 95.2% purity, HCl) was obtained as a yellow solid, which was confirmed by LCMS (M/Z+1=584.3), HPLC, ¹FNMR, and ¹HNMR (400 MHz, DMSO-d6, δ:10.86-10.99 (m, 1H), 10.64-10.71 (m, 1H), 10.44-10.54 (m, 2H), 10.31-10.40 (m, 2H), 9.96-10.10 (m, 1H), 9.59-9.71 (m, 1H), 8.40-8.50 (m, 1H), 7.89-7.99 (m, 6H), 7.80-7.86 (m, 1H), 7.68-7.76 (m, 3H), 7.53-7.62 (m, 1H), 3.92-4.04 (m, 8H), 3.13-3.25 (m, 2H), 1.44-1.60 (m, 2H), 0.84-0.92 (m, 3H)).

### Example 11

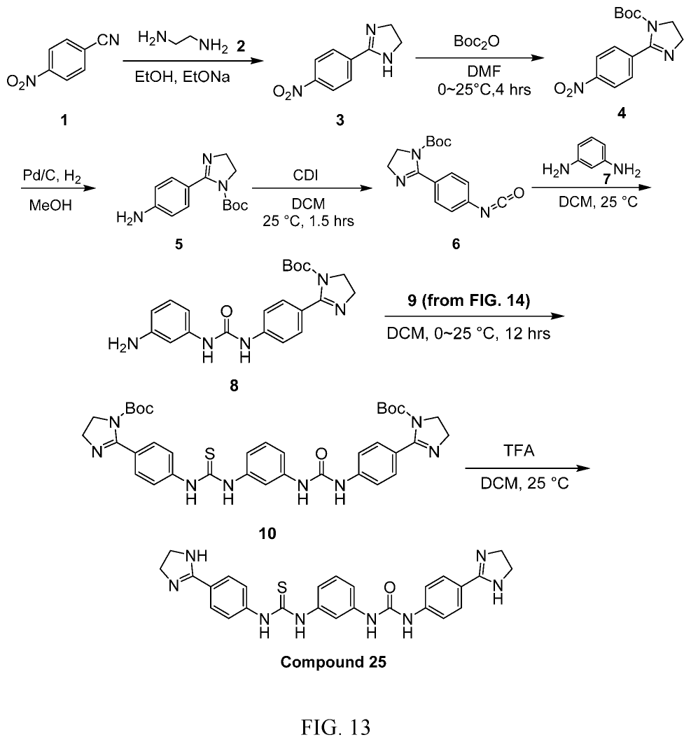

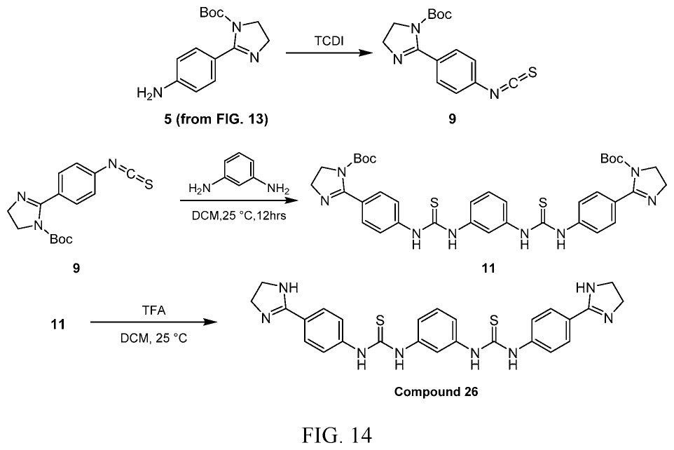

Exemplary compounds of formula (Ia) (e.g., compound 25) can be prepared in accordance with the reaction scheme set forth in FIG. 13. FIG. 13 is a reaction scheme of preparing compound 25. Experimental protocols for the preparation of compounds set forth in FIG. 13 are provided below.

To a mixture of compound 1 (200 g, 1.35 mol, 1 eq) in EtOH (2.4 L) was added sodium ethanolate (22.97 g, 337.57 mmol, 0.25 eq) at 20° C., and the resulting mixture was stirred at 20° C. for 3 hours. After three hours, ethane-1,2-diamine (126 g, 2.10 mol, 140.31 mL, 1.55 eq) was added to the mixture at 20° C., and the reaction was warmed to 75° C. and stirred at 75° C. for 12 hours. The reaction was monitored by LCMS, and the desired MS was detected (M/Z+1=149.1). The reaction mixture was quenched with H₂O (2500 mL) at 25° C., and was adjusted to pH=2 with 4 M HCl (500 mL) under 15° C., and the mixture was extracted with ethyl acetate (1000 mL*3). The aqueous layer was adjusted to pH=10 with 6 M NaOH (300 mL) under 15° C., and then extracted with ethyl acetate (2500 mL*3). The organic layers were washed with brine (1500 mL), dried over Na₂SO₄, filtered, and concentrated under reduced pressure to give a residue. The resulting residue was used directly in the next step. Compound 3 (200 g, crude) was obtained as yellow solid, which was confirmed by ¹HNMR (400 MHz, DMSO-d6, δ ppm 3.45 (br t, J=9.88 Hz, 2H) 3.86 (br t, J=9.94 Hz, 2H) 7.18 (br s, 1H) 8.06 (br d, J=8.50 Hz, 2H) 8.29 (br d, J=8.63 Hz, 2H)).

To a solution of compound 3 (100 g, 523 mmol, 1.00 eq) and DIEA (137.27 g, 1.06 mol, 185 mL, 2.03 eq) in DCM (1000 mL) was added Boc₂O (136.80 g, 626.80 mmol, 144 mL, 1.2 eq) at 25° C., and the reaction mixture was stirred for 3 hours at 25° C. Thin layer chromatography (petroleum ether:ethyl acetate=1:1) showed compound 3 (R_{f}=0.10) was consumed and a large new spot (R_{f}=0.20) was formed. The reaction was monitored by LCMS, and the desired MS was detected (M/Z+1=292). The residue was dissolved in ethyl acetate (200 mL) and petroleum ether (200 mL) was added drop wise to get white slurry. A yellow solid was collected after filtering, and the crude product was taken in next step. Compound 4 (124 g, 425.68 mmol, 81.38% yield) was obtained as yellow solid, which was confirmed by ¹HNMR (400 MHz, DMSO-d6, δ ppm 1.21 (s, 9H) 3.91 (s, 4H) 7.75 (d, J=8.88 Hz, 2H) 8.23-8.30 (m, 2H)).

To a solution of compound 4 (74 g, 254.03 mmol, 1 eq) in EtOH (800 mL) was added Pd/C (5 g, 254.03 mmol, 10% purity, 1 eq) under N₂ atmosphere at 25° C. The suspension was degassed and purged with H₂ three times. Then, the mixture was stirred at 40° C. under H₂ (50 Psi) for 6 hours. Thin layer chromatography (dichloromethane:MeOH=10:1) showed compound 4 (Rf=0.20) was consumed and a large new spot (Rf=0.05) was formed. The reaction mixture was concentrated under vacuum, and the resulting residue was purified by silica gel column chromatography (SiO₂, dichloromethane:MeOH=30:1 to 10:1, R_{f}=0.05). The crude product was further purified by prep-HPLC (NH₃H₂O conditions). Compound 5 (47.5 g, 167.23 mmol, 65.83% yield, 92.0% purity) was obtained as yellow solid, which was confirmed by ¹HNMR (400 MHz, DMSO-d6, δ ppm 1.26 (s, 9H) 3.67-3.74 (m, 2H) 3.78-3.85 (m, 2H) 5.42 (s, 2H) 6.46-6.54 (m, 2H) 7.16 (d, J=8.63 Hz, 2H)).

To a solution of compound 5 (10 g, 38.27 mmol, 1 eq) in DCM (100 mL) was added CDI (6.30 g, 38.85 mmol, 1.02 eq) at 25° C., and the reaction mixture was stirred for 2 hours at 25° C. The reaction was monitored by LCMS, and the desired MS was detected (M/Z+16=304). The reaction mixture was concentrated under vacuum and the crude product was used in the next reaction. Compound 6 (16 g, crude) was obtained as yellow oil, which was confirmed by LCMS (M/Z+16=304).

To a solution of benzene-1,3-diamine (7, 16.00 g, 147.92 mmol, 5 eq) and DIEA (7.79 g, 60.28 mmol, 10.5 mL, 2.04 eq) in DCM (20 mL) was dropwise added compound 6 (8.5 g, 29.58 mmol, 1 eq) in DCM (100 mL) at 25° C., and the resulting mixture was stirred for 6 hours at 25° C. The reaction was monitored by LCMS, and the desired MS was detected (M/Z+1=396). The reaction mixture was concentrated under vacuum, and the resulting crude product was purified by prep-HPLC (column: YMC Triart C18 250×50 mm×7 μm; mobile phase: [water (NH₃H₂O)-ACN]; B %: 29%-59%, 20 min). Compound 8 (6.5 g, 13.81 mmol, 46.67% yield, 84% purity) was obtained as yellow solid, which was confirmed by LCMS (M/Z+1=396).

To a solution of compound 8 (7 g, 17.70 mmol, 1 eq) and DIEA (4.60 g, 35.59 mmol, 6.2 mL, 2.01 eq) in DCM (70 mL) was added compound 9 (6.34 g, 20.90 mmol, 1.18 eq) from Example 12 at 25° C., and the resulting mixture was stirred for 16 hours at 25° C. The reaction was monitored by LCMS, and the desired MS was detected (M/Z+16=699). The reaction mixture was concentrated under vacuum, and the crude product was purified by prep-HPLC (column: YMC Triart C18 70×250 mm×7 um; mobile phase: [water (NH₃H₂O)-ACN]; B %: 40%-70%, 20 min). Compound 10 (4 g, 5.61 mmol, 31.69% yield, 98% purity) was obtained as yellow solid, which was confirmed by ¹HNMR (400 MHz, DMSO-d6, δ ppm 1.25 (br d, J=3.50 Hz, 18H) 3.66-4.01 (m, 8H) 7.00-7.72 (m, 12H) 8.81 (br d, J=11.51 Hz, 2H) 9.90 (br s, 2H)).

To a solution of compound 10 (5.5 g, 7.87 mmol, 1 eq) and TFA (77.00 g, 675.30 mmol, 50 mL, 85.80 eq) in DCM (50 mL) was added p-cresol (1.70 g, 15.74 mmol, 1.65 mL, 2 eq) dropwise at 25° C., and the resulting mixture was stirred for 12 hours at 25° C. The reaction was monitored by LCMS, and the desired MS was detected (M/Z+1=499). The reaction mixture was concentrated under vacuum, and the crude product was purified by prep-HPLC (column: Phenomenex luna C18 250×50 mm×10 μm; mobile phase: [water(HCl)-ACN]; B %: 2%-30%, 25 min). Compound 25 (800 mg, 1.47 mmol, 18.74% yield, 91.9% purity) was obtained as white solid, which was confirmed by LCMS (M/Z+1=499), HPLC, and ¹HNMR (400 MHz, DMSO-d6, δ ppm 3.97 (d, J=8.50 Hz, 8H) 7.20-7.32 (m, 3H) 7.66-7.74 (m, 3H) 7.92-8.00 (m, 6H) 9.66 (s, 1H) 10.11 (s, 1H) 10.43 (s, 2H) 10.53 (s, 2H) 10.77 (s, 1H) 11.05 (s, 1H)).

### Example 12

Exemplary compounds of formula (Ia) (e.g., compound 26) can be prepared in accordance with the reaction scheme set forth in FIG. 14. FIG. 14 is a reaction scheme of preparing compound 26. Experimental protocols for the preparation of compounds set forth in FIG. 14 are provided below.

To a solution of compound 5 (10 g, 38.27 mmol, 1 eq) from Example 11 in DCM (100 mL) was added di(imidazol-1-yl)methanethione (TCDI; 7.00 g, 39.28 mmol, 1.03 eq) dropwise at 25° C., and the resulting mixture was stirred for 2 hours at 25° C. The reaction was monitored by LCMS, and the desired MS was detected (M/Z+1=304). The reaction mixture was concentrated under vacuum, and the crude product was used directly in next step. Compound 9 (18.0 g, crude) was obtained as yellow solid, which was confirmed by LCMS (M/Z+1=304).

To a solution of compound 9 (1.00 g, 3.30 mmol, 1 eq) and DIEA (890.40 mg, 6.89 mmol, 1.20 mL, 2.09 eq) in DCM (5 mL) was added benzene-1,3-diamine (180 mg, 1.66 mmol, 5.05e-1 eq) dropwise in DCM (5 mL) at 25° C., and the resulting mixture was stirred for 12 hours at 25° C. The reaction was monitored by LCMS, and the desired MS was detected (M/Z+1=715). The reaction mixture was concentrated under vacuum, and the crude product was purified by prep-HPLC (column: YMC Triart C18 250×50 mm×7 μm; mobile phase: [water (NH₃H₂O)-ACN]; B %: 41%-71%). Compound 11 (445 mg, 591.34 μmol, 17.94% yield, 95% purity) was obtained as white solid, which was confirmed by ¹HNMR (400 MHz, DMSO-d6, δ ppm 1.24 (s, 18H) 3.77-3.92 (m, 8H) 7.25-7.34 (m, 3H) 7.41 (d, J=8.63 Hz, 4H) 7.55 (d, J=8.63 Hz, 4H) 7.72 (s, 1H) 9.89 (s, 2H) 9.97 (s, 2H)).

To a solution of compound 11 (100 mg, 139.88 μmol, 1 eq) in DCM (1 mL) was added TFA (1.54 g, 13.51 mmol, 1 mL, 96.55 eq) at 0° C., and the resulting mixture was stirred for 1 hour. The reaction was monitored by LCMS, and the desired MS was detected (M/Z+1=515). The reaction mixture was concentrated under vacuum, and the crude product was purified by prep-HPLC (column: Phenomenex luna C18 150×25 mm×10 μm; mobile phase: [water (HCl)-ACN]; B %: 0%-29%, 10 min). Compound 26 (35 mg, 64.47 μmol, 46.09% yield, 94.8% purity) was obtained yellow solid, which was confirmed by LCMS (M/Z+1=515), HPLC, and ¹HNMR (400 MHz, DMSO-d6, δ ppm 3.98 (s, 8H) 7.30-7.41 (m, 3H) 7.84 (s, 1H) 7.95 (s, 8H) 10.51 (s, 4H) 10.81 (s, 2H) 10.98 (s, 2H)).

### Example 13

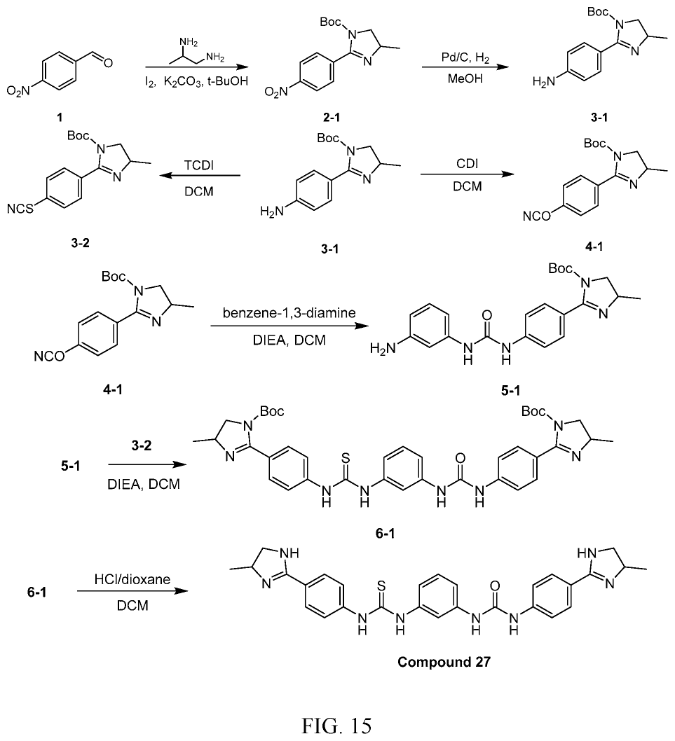

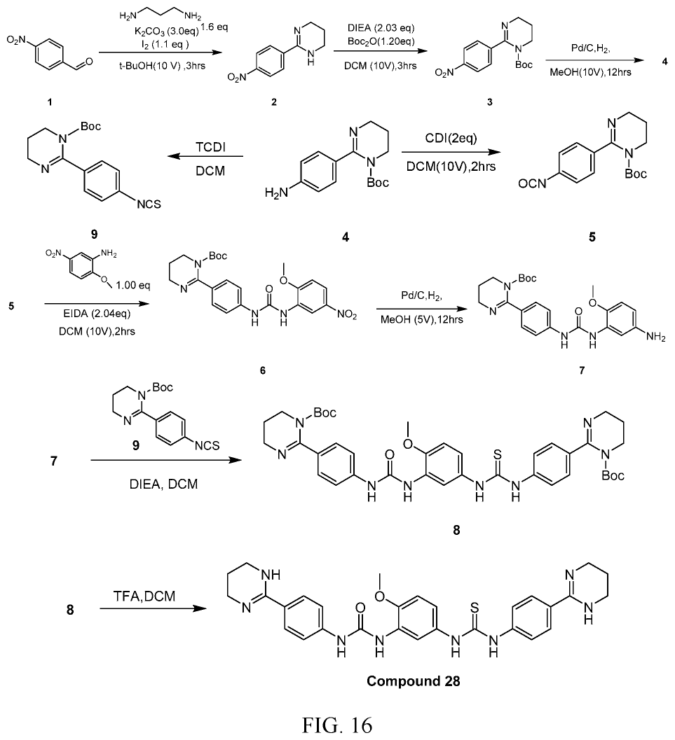

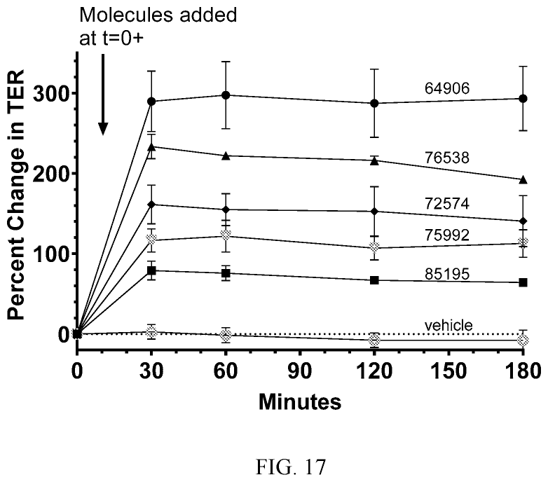

Exemplary compounds of formula (Ia) (e.g., compound 27) can be prepared in accordance with the reaction scheme set forth in FIG. 15. FIG. 15 is a reaction scheme of preparing compound 27. Experimental protocols for the preparation of compounds set forth in FIG. 15 are provided below.

To a solution of compound 1 (5 g, 33.0 mmol, 1.00 eq) in t-BuOH (70 mL) was added propane-1,2-diamine (3.85 g, 51.9 mmol, 4.44 mL, 1.57 eq) at 25° C. and the resulting mixture was stirred at 70° C. for 30 min. Then 12 (14.9 g, 58.9 mmol, 11.8 mL, 1.78 eq) and K₂CO₃ (19.5 g, 141 mmol, 4.27 eq) were added to the reaction at 70° C., and the mixture was stirred for 3 hours. Thin layer chromatography (dichloromethane:MeOH=10:1) showed that compound 1 (R_{f}=0.3) was consumed and a large new spot (R_{f}=0.3) was formed. The reaction mixture was cooled to 25° C. and quenched with Na₂S₂O₃ (50 mL) until the iodine color almost disappeared. The organic layer was separated and concentrated, and the resulting residue was dissolved with H₂O, the pH was adjusted to pH=2 with 4M HCl (20 mL), and extracted with ethyl acetate (50 mL*3). The aqueous layer was adjusted to pH=12 with 6 M NaOH (40 mL), and then extracted with ethyl acetate (100 mL*3). The organic layer was washed with brine (100 mL), dried over Na₂SO₄, filtered, and concerned under reduce pressure. Compound 2-1 (6.16 g, 29.4 mmol, 89.0% yield, 98.1% purity) was obtained as a yellow solid, which was confirmed by LCMS (M/Z+1=206), and was used in the next step without purification.

A solution of compound 2-1 (1.00 g, 3.28 mmol, 1.00 eq) in MeOH (10 mL) was degassed and purged with N₂ three times, and Pd/C (0.1 g, 3.28 mmol, 10% purity, 1.00 eq) was added at 20° C. The resulting mixture was stirred at 20° C. for 12 hours under H₂ (6.60 mg, 3.28 mmol, 15 psi, 1.00 eq) atmosphere. The reaction was monitored by LCMS, and the desired MS was detected (M/Z+1=276). Upon completion, the reaction was filtered and the resulting filtrate was concentrated. Compound 3-1 (0.75 g, 2.72 mmol, 83.1% yield) was obtained as yellow solid, which was confirmed by LCMS (M/Z+1=276), and was used in the next step without purification.

To a solution of compound 3-1 (200 mg, 726 μmol, 1.00e q) in DCM (2 mL) was added TCDI (133 mg, 748 μmol, 1.03 eq) at 20° C., and the resulting mixture was stirred at 25° C. for 2 hours. Thin layer chromatography (dichloromethane:methanol=10:1) showed that compound 3-1 was consumed and a main spot formed. The reaction mixture was concentrated under vacuum, and the crude product was used in the next step without purification. Compound 3-2 (200 mg, 630 μmol, 86.7% yield) was obtained as yellow oil.

To a solution of compound 3-1 (300 mg, 1.09 mmol, 1.00 eq) in DCM (3 mL) was added CDI (233 mg, 1.44 mmol, 1.32 eq) at 20° C., and the resulting mixture was stirred at 25° C. for 2 hours. Thin layer chromatography (dichloromethane:methanol=10:1) showed that compound 3-1 was consumed and a new spot (R_{f}=0.33) was formed. The reaction mixture was concentrated under vacuum, and the crude product was used in the next step without purification. Compound 4-1 (300 mg, 995 μmol, 91.3% yield) was obtained as yellow oil.

To a solution of compound 4-1 (300 mg, 995 μmol, 1.00 eq) in DCM (5 mL) was added DIEA (262 mg, 2.03 mmol, 353 μL, 2.04 eq) and benzene-1,3-diamine (538 mg, 4.97 mmol, 5.00 eq), and the resulting mixture was stirred at 25° C. for 6 hours. The reaction was monitored by LCMS, and the desired MS was detected (M/Z+1=410). Upon completion, the reaction was concentrated under vacuum, and the resulting residue was purified by prep-HPLC (column: Waters xbridge 150×25 mm×10 μm; mobile phase: [water (NH₄HCO₃)-ACN]; B %: 24%-54%, 9 min). Compound 5-1 (100 mg, 244 μmol, 24.5% yield) was obtained as a white solid, which was confirmed by LCMS (M/Z+1=410).

To a solution of compound 5-1 (100 mg, 244 μmol, 1.00 eq) in DCM (1 mL) was first added DIEA (63 mg, 487 μmol, 84.9 μL, 2.00 eq), and then compound 3-2 (77 mg, 242 μmol, 1.0 eq) was added at 20° C. The resulting mixture was stirred at 25° C. for 16 hours. The reaction was monitored by LCMS, and the desired MS was detected (M/Z+1=727). Upon completion, the reaction was concentrated under vacuum, and the resulting residue was purified by prep-HPLC (neutral condition with column: Waters xbridge 150×25 mm×10 μm; mobile phase: [water (NH₄HCO₃)-ACN]; B %: 38%-68%, 11 min). The crude product compound 6-1 (100 mg, 137.57 μmol, 56.3% yield) was obtained as a yellow solid, which was confirmed by LCMS (M/Z+1=727), and was used into the next step.

To a solution of compound 6-1 (170 mg, 233 μmol, 1.00 eq) in DCM (2 mL) was added HCl/dioxane (4 M, 584 μL, 10 eq) at 0° C., and the resulting mixture was stirred at 25° C. for 12 hours. The reaction was monitored by LCMS, and the desired MS was detected (M/Z+1=527). Upon completion, the reaction was concentrated under vacuum, and the resulting residue was purified by prep-HPLC (HCl condition with column: Welch Xtimate C18 150×25 mm×5 μm; mobile phase: [water (HCl)-ACN]; B %: 1%-31%, 8 min). The obtained product was further purified by prep-HPLC (HCl condition of column: Welch Ultimate C18 150×25 mm×5 μm; mobile phase: [water (TFA)-ACN]; B %: 8%-38%, 10 min). Compound 27 (2.1 mg, 18.99 μmol) was obtained as a gray solid, which was confirmed by LCMS (M/Z+1=527), HPLC, and ¹HNMR (400 MHz, DMSO-d6, δ 10.98 (s, 1H), 10.72 (s, 1H), 10.65 (s, 1H), 10.54 (s, 1H) 10.48 (s, 1H), 10.37 (s, 1H), 10.06 (s, 1H), 9.62 (s, 1H), 7.96-7.72 (m, 6H), 7.71-7.69 (m, 3H), 7.29-7.22 (m, 3H), 4.49-4.11 (m, 2H), 4.10-4.08 (m, 2H), 3.58-3.38 (m, 2H), 1.37-1.03 (m, 6H)).

### Example 14

Exemplary compounds of formula (Ia) (e.g., compound 28) can be prepared in accordance with the reaction scheme set forth in FIG. 16. FIG. 16 is a reaction scheme of preparing compound 28. Experimental protocols for the preparation of compounds set forth in FIG. 16 are provided below.

A solution of compound 1 (10.0 g, 66.2 mmol, 1.00 eq) and propane-1,3-diamine (5.40 g, 72.8 mmol, 6.08 mL, 1.10 eq) in t-BuOH (100 mL) was stirred at 70° C. for 0.5 hour, and then 12 (21.0 g, 82.7 mmol, 16.7 mL, 1.25 eq) and K₂CO₃ (27.5 g, 199 mmol, 3.01 eq) were added to the reaction. The resulting mixture was stirred at 70° C. for 3 hour. The reaction was monitored by LCMS, and the desired MS was detected (M/Z+1=206.4). Upon completion, the reaction solution was poured into a saturated Na₂SO₃ solution (300 mL), and the aqueous layer was checked with starch potassium iodide paper. The organic layer was extracted with DCM (150 mL*3) dried over Na₂SO₄, and concentrated. Compound 2 (14.0 g, crude) was obtained as a yellow solid, which was confirmed by ¹HNMR (400 MHz, CDCl₃, δ 8.22 (d, J=8.8 Hz, 2H), 7.85 (d, J=8.8 Hz, 2H), 3.55 (t, J=5.8 Hz, 5H), 1.90 (q, J=5.8 Hz, 2H)).

To a solution of compound 2 (14.0 g, 68.2 mmol, 1.00 eq) in DCM (150 mL) was added TEA (13.8 g, 137 mmol, 19.0 mL, 2.00 eq) and Boc₂O (25.6 g, 117 mmol, 27.0 mL, 1.72 eq), and the resulting mixture was stirred at 25° C. for 12 hours. The reaction was monitored by LCMS, and the desired MS was detected (M/Z+1=306.1). Upon completion, the reaction solution was concentrated under reduced pressure, and the resulting residue was purified by column chromatography (SiO₂, petroleum ether:ethyl acetate=20:1 to 1:1, R_{f}=0.3). Compound 3 (14.3 g, 46.8 mmol, 68.65% yield) was obtained as a yellow solid, which was confirmed by ¹HNMR (400 MHz, CDCl₃, δ 8.24 (d, J=8.6 Hz, 2H), 7.66 (d, J=8.6 Hz, 2H), 3.77 (t, J=6.4 Hz, 2H), 3.70 (t, J=5.8 Hz, 2H), 1.97 (q, J=6.0 Hz, 2H), 1.16 (s, 9H)).

To a solution of compound 3 (13.5 g, 44.2 mmol, 1.00 eq) in MeOH (150 mL) was added Pd/C (1.00 g, 4.42 mmol, 10% purity, 0.10 eq), and the resulting mixture was stirred at 25° C. for 5 hours under H₂. The reaction was monitored by LCMS, and the desired MS was detected (M/Z+1=276.1). Upon completion, the reaction mixture was filtered with celite, and concentrated under vacuum. The resulting product was used in the next reaction without further purification. Compound 4 (14.0 g, crude) was obtained as a white solid, which was confirmed by ¹HNMR (400 MHz, CDCl₃, δ 7.31 (d, J=8.4 Hz, 2H), 6.64 (d, J=8.4 Hz, 2H), 3.68 (t, J=6.6 Hz, 2H), 3.57 (t, J=5.8 Hz, 2H), 1.92 (td, J=6.4, 12.4 Hz, 2H), 1.18 (s, 9H)).

To a solution of compound 4 (4.00 g, 13.8 mmol, 95% purity, 1.00 eq) in DCM (60.0 mL) was slowly added CDI (4.50 g, 27.8 mmol, 2.01 eq), and the resulting mixture was stirred at 25° C. for 2 hours. Thin layer chromatography (dichloromethane:methanol=7:1) showed reactant was consumed and a new spot (R_{f}=0.70) was formed. The reaction solution was used directly for the next step. Compound 5 (4.16 g, crude) was obtained as a yellow liquid.

To a solution of 2-methoxy-5-nitro-aniline (2.23 g, 13.3 mmol, 1.00 eq) in DCM (100 mL) was added DIEA (3.49 g, 27.0 mmol, 4.70 mL, 2.03 eq), and then compound 5 (4.00 g, 13.3 mmol, 1.00 eq) was added dropwise. The resulting mixture was stirred at 25° C. for 12 hours. The reaction was monitored by LCMS, and the desired MS was detected (M/Z+1=470.1). Upon completion, the reaction solution was poured into water (150 mL), extracted with DCM (100 mL*3), the organic layer was washed with brine (100 ml*2), the organic layer dry with Na₂SO₄, filtered, and concentrated under reduced pressure to give a residue. The resulting residue was purified by prep-HPLC (basic conditions). Compound 6 (3.50 g, crude) was obtained as a yellow solid, which was confirmed by LCMS (M/Z+1=470.1).

To a solution of compound 6 (2.50 g, 5.32 mmol, 1.00 eq) in MeOH (60.0 mL) was added Pd/C (300 mg, 5.32 mmol, 10% purity, 1.00 eq), and the resulting mixture was stirred at 25° C. for 3 hours under H₂. The reaction was monitored by LCMS, and the desired MS was detected (M/Z+1=440.4). Upon completion, the reaction mixture was filtered and concentrated under vacuum. Compound 7 (3.00 g, crude) was obtained as a brown solid, which was confirmed by LCMS (M/Z+1=440.4).

To a solution of compound 7 (1.50 g, 3.41 mmol, 1.00 eq) in DCM (30.0 mL) was added DIEA (1.11 g, 8.61 mmol, 1.50 mL, 2.52 eq) and compound 9 (1.65 g, 5.20 mmol, 1.52 eq) from Example 12. The resulting mixture was stirred at 25° C. for 12 hours. The reaction was monitored by LCMS, and the desired MS was detected (M/Z+1=757.3). Upon completion, the reaction solution was concentrated under reduced pressure, and the resulting residue was purified by prep-HPLC (basic condition: column: YMC Triart C18 250×50 mm×7 μm; mobile phase: [water (NH₃H₂O)-ACN]; B %: %-%, 22 min). Compound 8 (700 mg, crude) was obtained as a yellow solid, which was confirmed by LCMS (M/Z+1=757.3).

To a solution of compound 8 (300 mg, 396 μmol, 1.00 eq) in DCM (3.00 mL) was added TFA (4.31 g, 37.8 mmol, 2.80 mL, 95.4 eq) and H₂O (1.40 g, 77.7 mmol, 1.40 mL, 196 eq). The resulting mixture was stirred at 25° C. for 6 hours. The reaction was monitored by LCMS, and the desired MS was detected (M/Z+1=557.4). Upon completion, the reaction mixture was concentrated under vacuum, and the resulting residue was purified by prep-HPLC (TFA condition: column: Welch Xtimate C18 150×25 mm×5 μm; mobile phase: [water (TFA)-ACN]; B %: 2%-32%, 50 min). Compound 28 (100 mg, 156 μmol, 39.4% yield, 87% purity) was obtained as a white solid, which was confirmed by LCMS (M/Z+1=557.4), HPLC, and ¹HNMR (400 MHz, CDCl₃, δ 10.98 (br s, 1H), 10.56 (s, 1H), 10.33 (s, 1H), 9.97-9.86 (m, 4H), 8.61 (s, 1H), 8.22 (d, J=2.4 Hz, 1H), 7.96 (br d, J=8.8 Hz, 2H), 7.75-7.67 (m, 6H), 7.13 (dd, J=2.4, 8.6 Hz, 1H), 7.01 (d, J=8.8 Hz, 1H), 3.87 (s, 3H), 3.47 (br d, J=3.2 Hz, 8H), 1.96 (br d, J=3.6 Hz, 4H)).

### Example 15

This example demonstrates the measurement of transepithelial electrical resistance by exemplary compounds of the disclosure.

Electrical measures are used to assess the flow of ions through the claudin channels. The most commonly used measurement in the tight junction field is transepithelial electrical resistance (TER). These assays can be performed using epithelial cells grown on permeable supports (in vitro) or using intact tissue from the GI tract mounted across apertures (ex vivo).

Two different Madin-Darby Canine Kidney (MDCK) I cell lines were used, which respectively express either claudin-2 or -15 when grown in the presence of doxycycline. These cells are useful in assessing the specificity of molecule effects on claudin-2 versus claudin-15. Caco-2 BBe epithelial cells, which normally express claudin-2 and some claudin-15, were also used to better understand the utility of these molecules in blocking claudins in the gastrointestinal (GI) tract. Caco-2 BBe and MDCK cells were grown on 0.33 cm² polycarbonate semi-permeable membranes with 0.4 μm pores (Corning Life Sciences, Corning, NY) and used 4 days (MDCK) or 3 weeks (Caco-2 BBe) after plating.

To measure TER, a two-electrode current clamp technique was used to measure TER across the layer of cells. This was done using a pair of recording electrodes (one on each side of the epithelial layer) to apply a steady transepithelial current step (ΔI) of 10 μA. A second pair of electrodes was used to measure the transepithelial voltage step (ΔV) needed to attain this current. Current and voltage were used to calculate TER using Ohm's law (ΔV=ΔI×TER). A higher TER means that there are fewer ions passing through the tight junction. In contrast, a decrease in TER could suggest damage of the epithelium or cell death.

Electrode bridges were prepared using 1% agarose in Hank's balanced saline solution (HBSS), 135 mM NaCl, 0.3 mM Na₂HPO₄, 0.4 mM MgSO₄, 0.5 mM MgCl₂, 0.3 mM KH₂PO₄, 1.3 mM CaCl₂), 10 mM HEPES, and 5 mM KOH, pH 7.4. Bridges were connected to calomel and Ag—AgCl electrodes and a current clamp (University of Iowa Bioengineering, Iowa City, IA). Experiments were performed at 37° C. with cells bathed in HBSS.

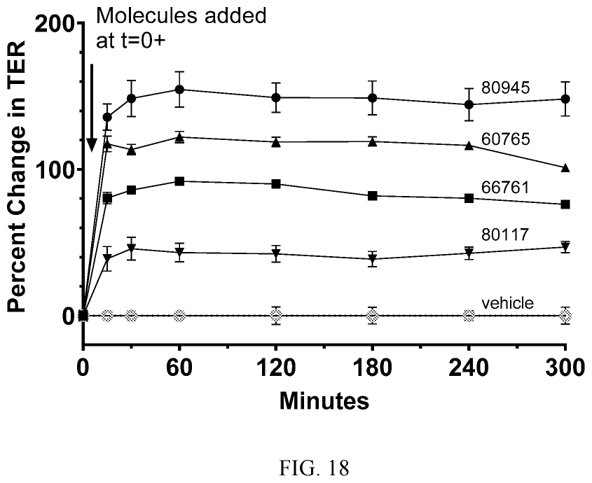

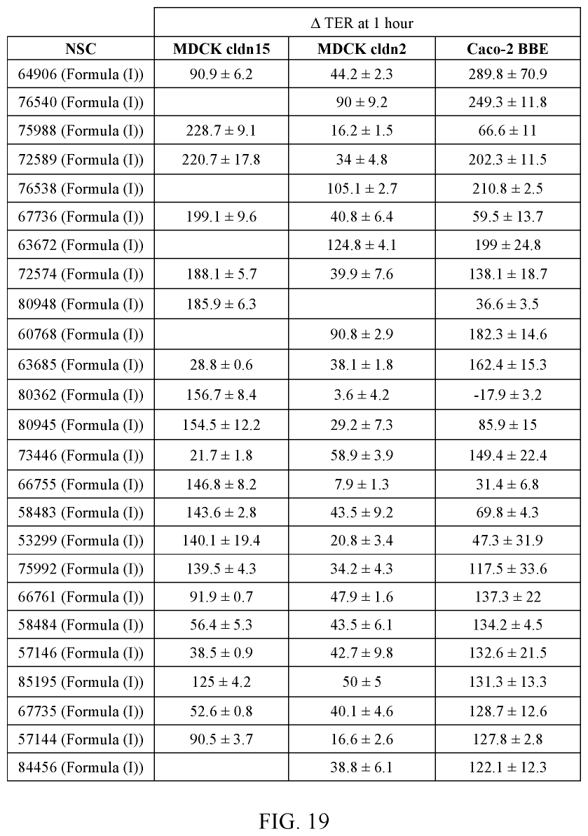

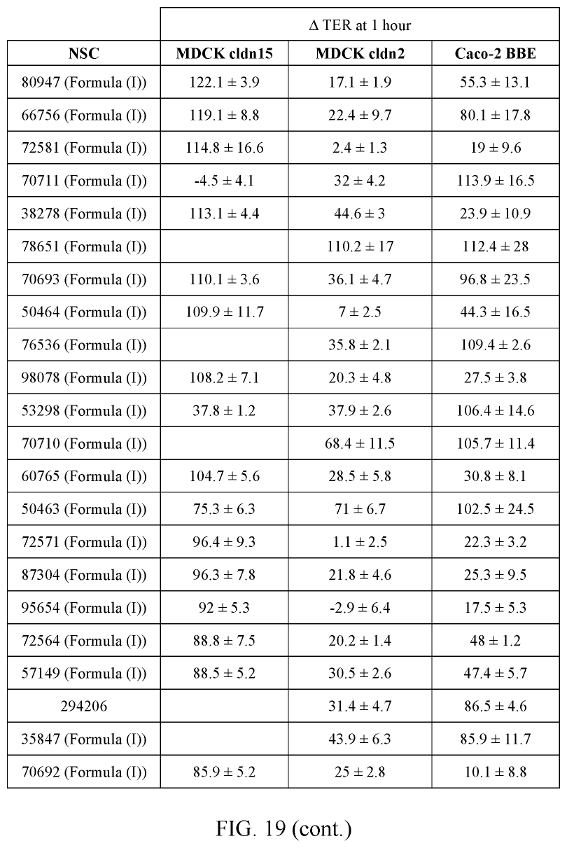

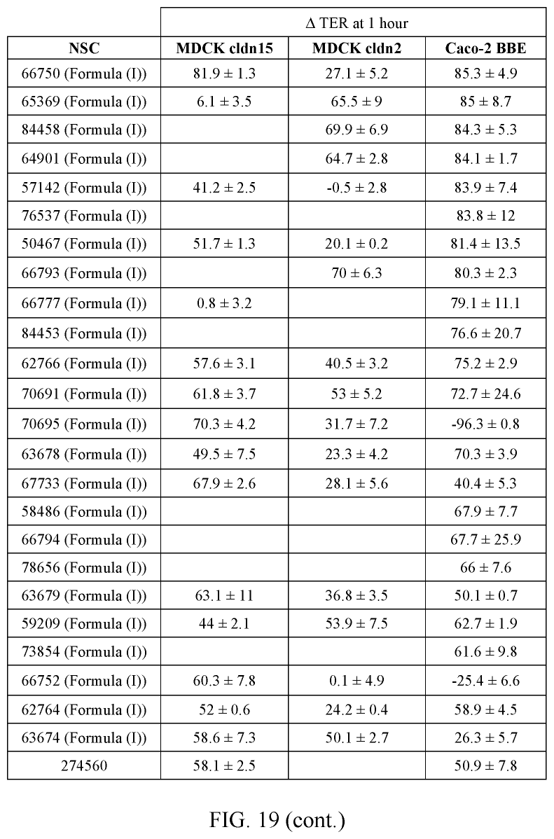

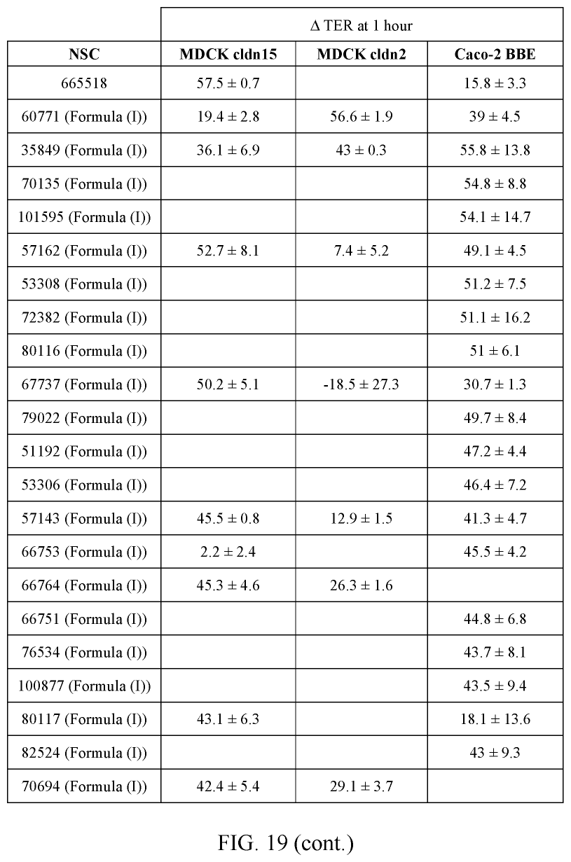

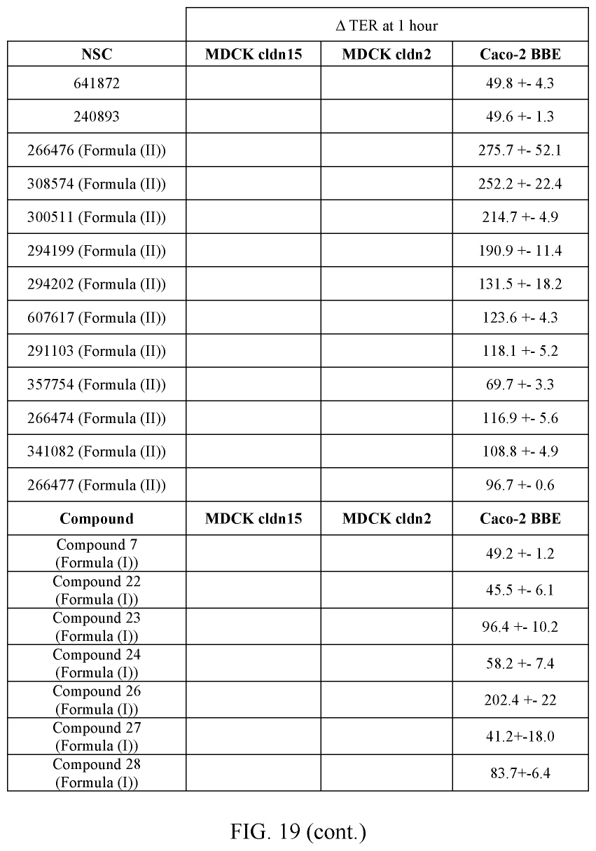

Molecules were solubilized in vehicle (dimethylsulfoxide (DMSO)) and applied to both sides of the epithelial monolayer after first measuring TER at t=0 (FIGS. 17 and 18). Final DMSO concentrations were <0.25%. Readings were performed for at least 3 hours after application to rule out epithelial cell injury, which would reduce TER. Initial screening was performed using 100 μM molecule concentration. FIG. 17 shows that TER was stably increased after molecules were added to apical and basolateral sides of Caco-2 BBe epithelial monolayers. FIG. 18 shows that TER was stably increased after molecules were added to apical and basolateral sides of MDCK I monolayers induced to express claudin-15. Additional data showing the change in TER after 1 hour contact time for MDCK cells (induced to express either claudin-2 or -15) and Caco-2 BBe cells are set forth in the table in FIG. 19.

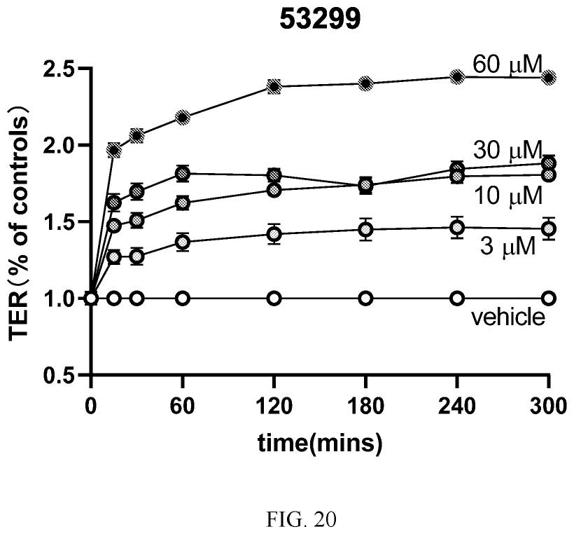

Follow up studies were also performed at lower concentrations for NSC 53299 (FIG. 20). Resistance measurements were normalized to values of resistance with vehicle but without test compound (vehicle). FIG. 20 evidences that while the inventive compounds were screened at concentrations of 100 μM, the claudin blockers are also effective at low micromolar concentrations.

### Example 16

This example demonstrates ion selectivity measurements of exemplary compounds of formula (I) in an aspect of the disclosure.

A second technique for studying the blockade of claudin pores is ion selectivity measurements. The general concept of these measurements is that if an ionic gradient exists across a semipermeable barrier, the reversal potentials (e.g., current where net current flux=0) depends on the relative permeabilities of the ions comprising the gradient. Larger disparities in permeabilites for the ions generating the gradient result in higher magnitudes of reversal potentials.

Reversal potentials (Vᵣₑᵥ) were measured by clamping transepithelial current to zero (I=0) before and after basolateral or apical replacement of HBSS with media in which 50% of NaCl was iso-osmotically replaced by mannitol. These NaCl dilution potentials measure Na⁺ permeability relative to Cl⁻ permeability. Alternatively, substitution of 135 mM NaCl with 135 mM XCl, allowed calculation of the permeability of monovalent cations methylamine (MA⁺), tetramethylammonium (TMA⁺), and tetraethylammonium (TEA⁺) relative to Na⁺. Relative permeabilities (PNa⁺/PCl⁻) or (PX⁺/PNa⁺) were determined using the Goldmann-Hodgkin-Katz voltage equation, measured Vᵣₑᵥ, and known composition of basolateral and apical solutions.

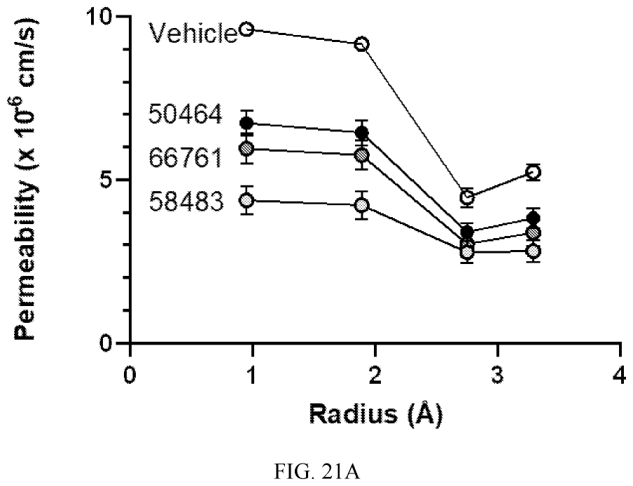

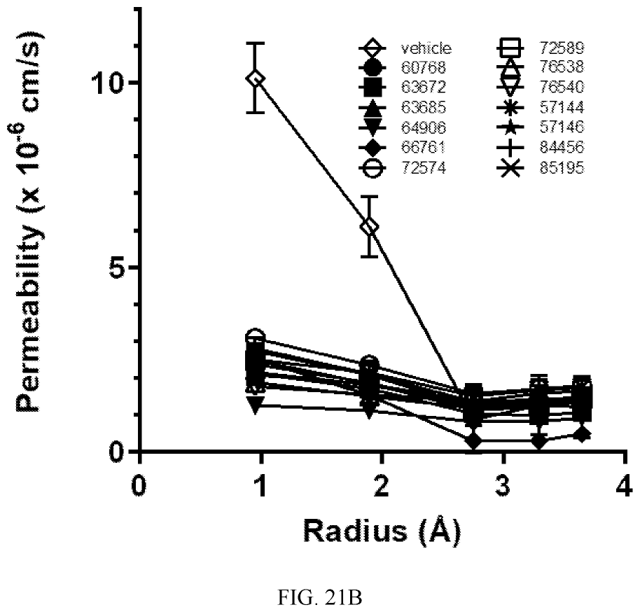

Ion selectivity measurements show that claudin-blocking molecules primarily reduce the permeability of cations with radii<2.5 Å when compounds of formula (I) were applied at 100 μM (FIGS. 21A and 21). FIG. 21A shows measurements in MDCK I cells induced to express claudin-15. FIG. 21B shows measurements in Caco-2 BBe cells. The tested compounds of formula (I) surprisingly had a dramatic ability to eliminate small cation permeability compared to vehicle only (control).

### Example 17

This example demonstrates the measurement of transmucosal barrier function of exemplary compounds of formula (I) in an aspect of the disclosure.

The same techniques to measure TER and ion selectivity also apply to intact mucosa isolated from mice. The only significant difference is the tissue was first stripped of its muscularis propria and mounted in chambers, known as Ussing chambers. The same techniques were used to measure ion selectivity. Identical solutions were used, but also bubbled with 100% oxygen.

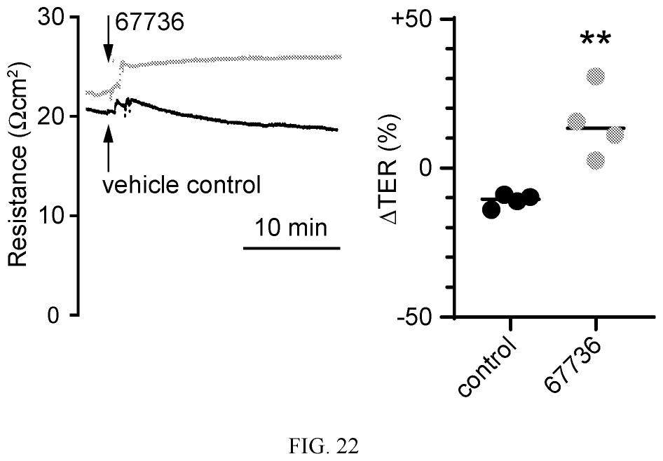

For TER measurements, duodenum segments were stripped of muscularis propria and mounted in Ussing chambers, and equilibrated in oxygenated HBSS for 15 min at 37° C. Vehicle control solution (DMSO) or the same solution containing 100 μM NSC 67736 were then added bilaterally. TER was recorded using current clamp pulses from 0 to 10 μA and Vᵣₑᵥ was recorded by clamping current to 0. Electrode bridges were made 3% agarose KCl. FIG. 22 shows that NSC 67736 increases barrier function in mouse mucosa.

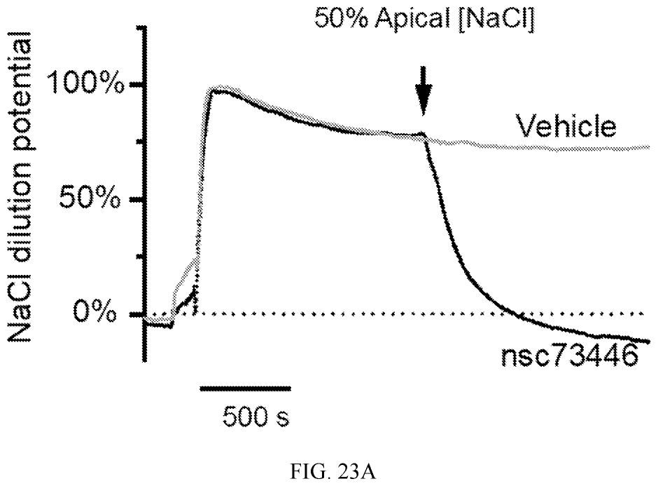

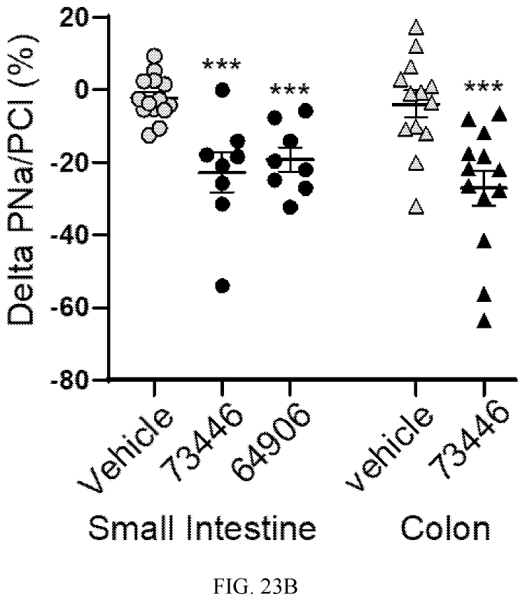

Small intestine or colon segments were stripped of muscularis propria, mounted in Ussing chambers, and equilibrated in oxygenated HBSS for 15 min at 37° C. Apical solution was changed to HBSS containing 50% [NaCl], which induced epithelial depolarization due to the claudin cation selectivity. Upon addition of claudin blocking molecules, reduced sodium permeability results in repolarization. Vehicle control solution (DMSO) had no significant effect to reduce the NaCl dilution potential. See FIG. 23A. The extent of repolarization is proportional to claudin channel blockade. The ion movements of PNa⁺ relative to PCl⁻ are shown in FIG. 23B. These demonstrate that NSC 73446 and NSC 64906 reduce Na⁺ permeability relative to Cl⁻ permeability in mouse small intestine and colon.

### Example 18

This example demonstrates the effect of a compound of formula (I) on water transport across human colonic epithelium, as evidenced by an organoid shrinkage model.

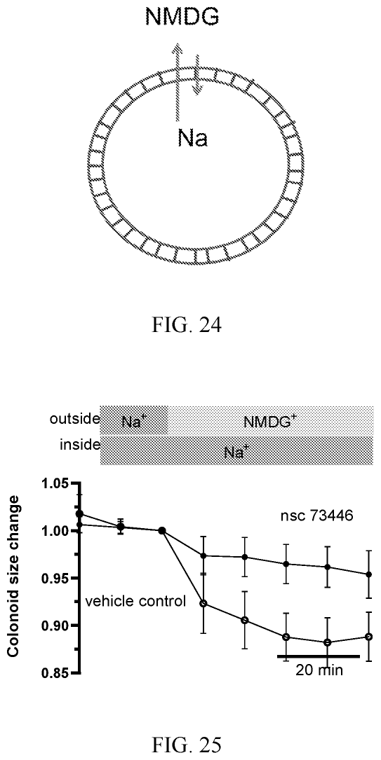

For this study, organoids derived from human colon biopsies (colonoids) were first equilibrated in steady-state with Na⁺ as the predominant extracellular cation. Extraluminal Na⁺ was then replaced with N-methyl-D-glucamine (NMDG⁺) by buffer exchange. The buffer exchange generates a large concentration gradient for Na⁺ to diffuse out of the organoid lumen. Due to its size, NMDG⁺ does not diffuse into the organoid lumen through claudin channels. See, for example, FIG. 24. As a result, the net Na⁺ flux across tight junctions creates an osmotic gradient for water to exit the organoid through tight junctions, causing organoids to shrink. The size of organoids is measured by time-lapse microscopy, and the rate and extent of shrinkage can be determined as an assay of claudin permeability. Reduced rate and extent of shrinkage indicates reduced permeability.

Colonoids derived from a patient with inflammatory bowel disease were first equilibrated in steady-state with Na⁺ containing media. Equilibrated colonoids were treated with NSC 73446 or a vehicle control and the extraluminal Na⁺ was then replaced with N-methyl-D-glucamine (NMDG⁺) by buffer exchange. The results of the time-lapse colonoid size are set forth in FIG. 25. As is apparent from the results set forth in FIG. 25, water transport across human epithelium was modulated by NSC 73446. More particularly, NSC 73446 minimizes the rate and extent of colonoid size decrease relative to the vehicle control, demonstrating that NSC 73446 reduces water transport out of the lumen of the colonoid.

### Example 19

This example provides all-atom molecular dynamics simulations to calculate ion permeability of claudin-15 to NaCl in the presence of compounds of formula (I).

Ion transport was simulated by applying a voltage bias across the claudin-15 pore. Repeat simulations at multiple voltages were performed to obtain the current-voltage (I-V) relationship. The channel conductance (G) was then estimated from current-voltage (I-V) curves (I=VG).

To minimize computational efforts, a reduced all-atom model of claudin-15 pores was developed to calculate the channel conductance to NaCl. The reduced all-atom model of the claudin-15 pores included a channel that only contained the ion conduction pore. The simulations systems contained three channels in a unit cell, which increased statistical significance of conductance data. This model was equilibrated with all-atom resolution in 200 mM of NaCl solution before applying a voltage bias. All simulations were performed using the program NAMD and CHARMM36 forcefield for protein, water, and ions. To include the compounds of formula (I) in the all-atom molecular dynamics simulations, atomic and inter-atomic interactions parameters, also known as forcefield parameters, of the molecules were needed. Thus, forcefield parameters for two molecules, namely, NSC 64906 and NSC 73446, were prepared in accordance with CHARMM format.

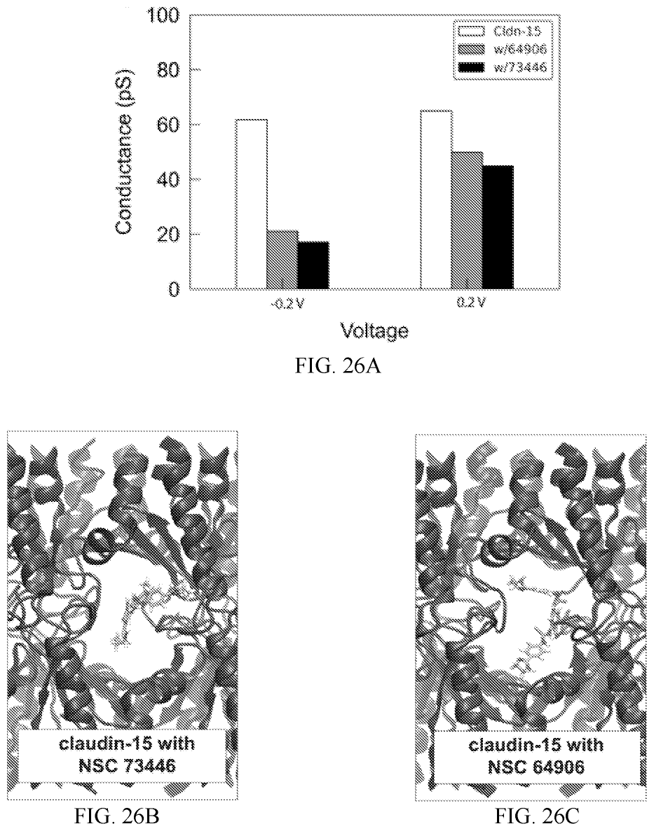

The conductance at voltages of +0.2 V and −0.2 V, using 50 ns of all-atom molecular dynamics simulation, were first calculated for claudin-15 channels in 200 mM NaCl in the absence of any additional molecules. Then, each of compounds of formula (I), i.e., NSC 64906 and NSC 73446, were added to the claudin-15 channel by placing them in the middle of the pore and equilibrating the system prior to application of the voltage. The system was then simulated under voltages of +0.2 V and −0.2 V, and the conductance of the channel to NaCl was calculated from the I-V curves. The results are set forth in FIGS. 26A-26C.

As shown in FIG. 26A, the conductivity of the claudin-15 channel to Na⁺ was reduced in the presence of NSC 64906 and NSC 73446. The reduction of current in the presence of NSC 64906 and NSC 73446 indicates that NSC 64906 and NSC 73446 block or partially block ion conduction through the pore. Without wishing to be bound by any particular theory, it is believed that NSC 64906 and NSC 73446 block the ion conduction by forming a complex in the pore of claudin-15, as evidenced by FIGS. 26B and 26C.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a disorder mediated by claudin-2 and/or claudin-15 in a subject comprising administering to the subject an effective amount of a compound of formula (I) 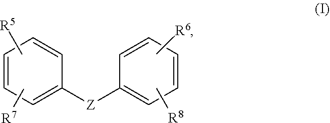
wherein
R⁵ and R⁶ are the same and are selected from 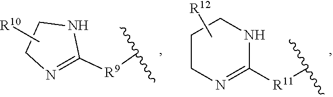
and —C(NHR¹³)═NR¹⁴;
wherein
R⁹ and R¹¹ are the same or different and each is a bond, —CH₂—, —CH₂CH₂—, or —NH—;
R¹⁰ and R¹² are the same or different and each is hydrogen, alkyl, or hydroxy; and
R¹³ and R¹⁴ are the same or different and each is alkyl;
R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—; and
Z is selected from 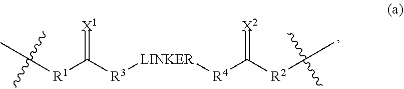
wherein
X¹ and X² are the same or different and each is O or S;
R¹ and R² are the same or different and each is —NH— or a bond;
R³ and R⁴ are the same or different and each is —NH—, —CH═CH—, —CH₂CH₂—, —NHC(O)—, —NHC(O)CH₂NHC(O)NH—, or a bond;
provided that R¹ and R³ are not both bonds and R² and R⁴ are not both bonds; and
LINKER is selected from
(i) —(CH₂)ₙ— that is optionally substituted with a hydroxy, wherein n is an integer of 1-12,
(ii) —O(CH₂)ₘO— that is optionally substituted, wherein m is an integer of 3-6,
(iii) —CH═CH—, 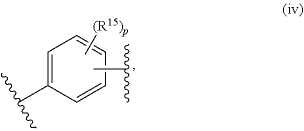
wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2, 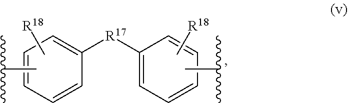
wherein R¹⁷ is selected from a bond, —CH═CH—, —N═N—, —CH₂—, —NHC(O)—, —C(O)NH—, —NHC(O)NH—, —NHC(O)—(CH₂)₁₋₄—C(O)NH—, —NHC(O)NH—(CH₂)₁₋₄—NHC(O)NH—, —NHC(O)-Ph-C(O)NH—, and —NHC(O)NH-Ph-NHC(O)NH—, and each R¹⁸ is the same or different and each is hydrogen, alkyl, alkoxy, 1,4-dihydroimidazolyl, or —R¹⁶—C(O)NH-Ph-R⁵, 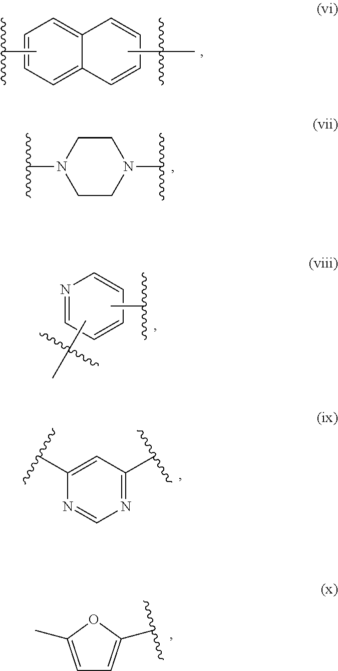
and (xi) a bond,
and 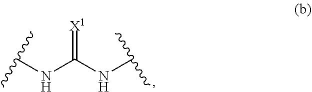
wherein X¹ is O or S;
or a pharmaceutically acceptable salt thereof to the subject.

2. The method of claim 1, wherein the compound of formula (I) is a compound of formula (I-1) 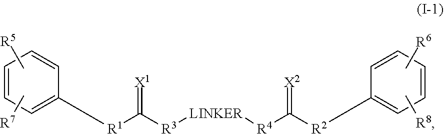
wherein
X¹ and X² are the same or different and each is O or S;
R¹ and R² are the same or different and each is —NH— or a bond;
R³ and R⁴ are the same or different and each is —NH—, —CH═CH—, —CH₂CH₂—, —NHC(O)—, —NHC(O)CH₂NHC(O)NH—, or a bond;
provided that R¹ and R³ are not both bonds and R² and R⁴ are not both bonds;
R⁵ and R⁶ are the same and are selected from 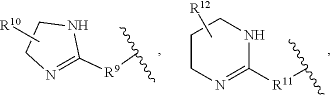
and —C(NHR¹³)=¹⁴;
wherein
R⁹ and R¹¹ are the same or different and each is a bond, —CH₂—, —CH₂CH₂—, or —NH—;
R¹⁰ and R¹² are the same or different and each is hydrogen, alkyl, or hydroxy; and
R¹³ and R¹⁴ are the same or different and each is alkyl;
R⁷ and R⁸ are the same or different and each is selected from hydrogen, alkyl, halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, and R¹⁶ is a bond or —NH—; and
LINKER is selected from
—(CH₂)ₙ— that is optionally substituted with a hydroxy, wherein n is an integer of 1-12,
—O(CH₂)ₘO— that is optionally substituted, wherein m is an integer of 3-6,
—CH═CH—, 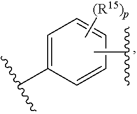
wherein each R¹⁵ is the same or different and is selected from alkyl, halo, nitro, amino, hydroxy, alkoxy, alkylthio (—S-alkyl), amido, alkylamido, dialkylamido, phenylamido, carboxylate, —R¹⁶—C(O)NH-Ph-R⁵, and R⁵, wherein R¹⁶ is a bond or —NH—, and p is 0 or an integer of 1-2, 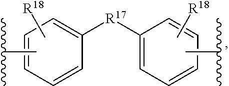
wherein R¹⁷ is selected from a bond, —CH═CH—, —N═N—, —CH₂—, —NHC(O)—, —C(O)NH—, —NHC(O)NH—, —NHC(O)—(CH₂)₁₋₄—C(O)NH—, —NHC(O)NH—(CH₂)₁₋₄—NHC(O)NH—, —NHC(O)-Ph-C(O)NH—, and —NHC(O)NH-Ph-NHC(O)NH—, and each R¹⁸ is the same or different and each is hydrogen, alkyl, alkoxy, 1,4-dihydroimidazolyl, or —R¹⁶—C(O)NH-Ph-R⁵, 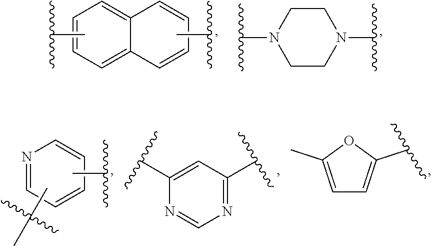
and a bond,
or a pharmaceutically acceptable salt thereof to the subject.

3. The method of claim 1, wherein in the compound of formula (I) or a pharmaceutically acceptable salt thereof, X¹ and X² are both O.

4. The method of claim 1, wherein in the compound of formula (I) or a pharmaceutically acceptable salt thereof, R¹ and R² are each —NH—.

5. The method of claim 1, wherein in the compound of formula (I) or a pharmaceutically acceptable salt thereof, R³ and R⁴ are each —NH— or a bond.

6. The method of claim 1, wherein the compound of formula (I) is a compound of formula (I-2) 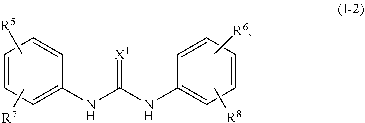
wherein
X¹ is O or S;
R⁵ and R⁶ are the same and are selected from 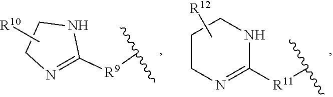
and —C(NHR¹³)═NR¹⁴;
wherein
R⁹ and R¹¹ are the same or different and each is a bond, —CH₂—, —CH₂CH₂—, or —NH—;
R¹⁰ and R¹² are the same or different and each is hydrogen, alkyl, or hydroxy; and
R¹³ and R¹⁴ are the same or different and each is alkyl; and
R⁷ and R⁸ are the same or different and each is selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein R¹⁶ is a bond or —NH—;
or a pharmaceutically acceptable salt thereof to the subject.

7. The method of claim 1, wherein in the compound of formula (I) or a pharmaceutically acceptable salt thereof, R⁵ and R⁶ are both

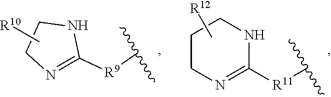

8. The method of claim 7, wherein in the compound of formula (I) or a pharmaceutically acceptable salt thereof, R⁹ and R¹¹ are both a bond.

9. The method of claim 8, wherein in the compound of formula (I) or a pharmaceutically acceptable salt thereof, R¹⁰ and R¹² are both hydrogen.

10. The method of claim 1, wherein in the compound of formula (I) or a pharmaceutically acceptable salt thereof, R⁷ and R⁸ are the same and each is selected from hydrogen, alkyl, amido, alkylamido, dialkylamido, and R⁵.

11. The method of claim 1, wherein in the compound of formula (I) or a pharmaceutically acceptable salt thereof, LINKER is selected from
—(CH₂)ₙ— that is optionally substituted with a hydroxy, wherein n is an integer of 1-12, 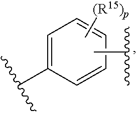
wherein each R¹⁵ is the same or different and is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, R¹⁶ is a bond or —NH—, and p is 0 or an integer of 1-2, 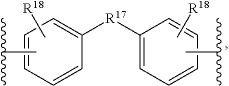
wherein R¹⁷ is selected from a bond; —CH═CH—, —N═N—, —CH₂—, —NHC(O)NH—, and —NHC(O)-Ph-C(O)NH—, and R¹⁸ is hydrogen, alkyl, or alkoxy, and

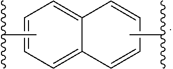

12. The method of claim 1, wherein in the compound of formula (I) or a pharmaceutically acceptable salt thereof, LINKER is 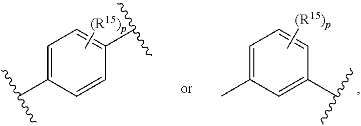
wherein
p is 0, or
p is 1 and R¹⁵ is selected from alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, and
R¹⁶ is a bond or —NH—.

13. The method of claim 1, wherein the compound of formula (I) is a pharmaceutically acceptable salt.

14. The method of claim 1, wherein the compound of formula (I) or a pharmaceutically acceptable salt thereof is

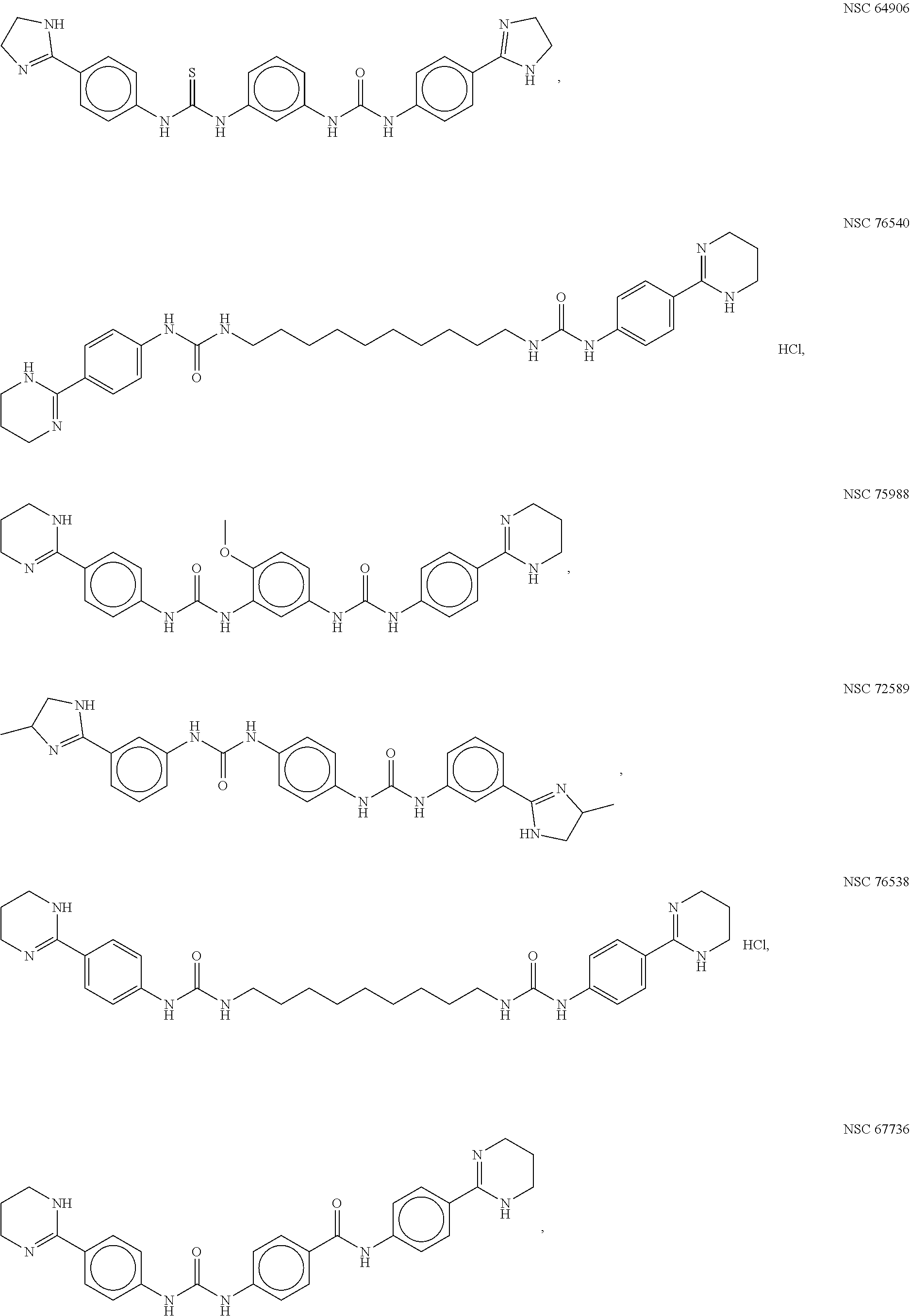

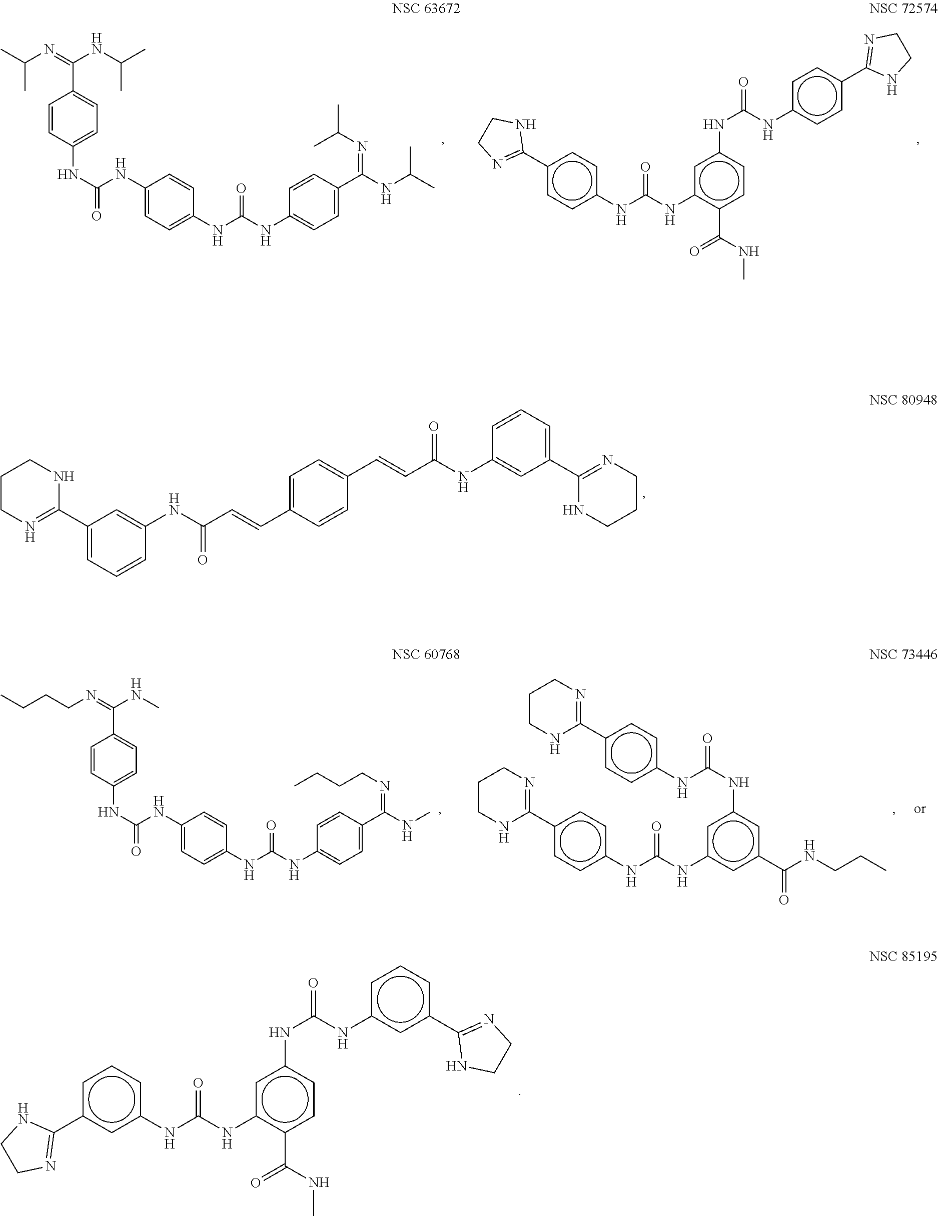

15. The method of claim 1, wherein the disorder mediated by claudin-2 and/or claudin-15 is an intestinal disorder.

16. The method of claim 15, wherein the intestinal disorder is colitis, Crohn's disease, enteritis, gastroenteritis, inflammatory bowel disease (IBD), irritable bowel syndrome (IBS), celiac disease, enteropathies, pouchitis, or diarrhea.

17. The method of claim 1, wherein the compound of formula (I) or a pharmaceutically acceptable salt thereof inhibits claudin-2 and/or claudin-15.

18. A compound that is a 1,3- or 1,4-substituted phenyl bridged compound of formula (Ia) 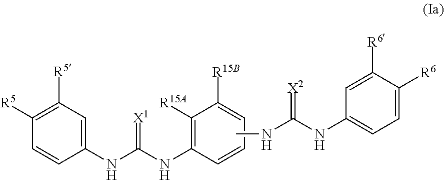
wherein
X¹ and X² are the same or different and each is O or S;
one of R⁵ and R^{5′} is 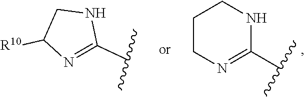
and the other is hydrogen;
one of R⁶ and R^{6′} is 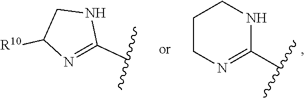
and the other is hydrogen;
wherein R⁵ and R⁶ are the same and R^{5′} and R^{6′} are the same;
R¹⁰ is hydrogen or alkyl;
R^{15A} and R^{15B} are the same or different and each is selected from hydrogen, alkyl, alkoxy, amino, alkylamido, 1,4-dihydroimidazolyl, and guanidinyl, or a pharmaceutically acceptable salt thereof.

19. The compound of claim 18 or a pharmaceutically acceptable salt thereof, wherein X¹ and X² are both O.

20. The compound of claim 18 or a pharmaceutically acceptable salt thereof, wherein at least one of X¹ and X² is S.

21. The compound of claim 18 or a pharmaceutically acceptable salt thereof, wherein
the compound of formula (Ia) is a 1,3-substituted phenyl bridged compound,
R⁵ and R⁶ are both 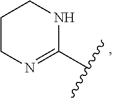 [SMILES: *C1=NCCCN1]
R^{5′} and R^{6′} are both hydrogen, and
R^{15A} is hydrogen.

22. The compound of claim 18 or a pharmaceutically acceptable salt thereof, wherein
the compound of formula (Ia) is a 1,3-substituted phenyl bridged compound,
R⁵ and R⁶ are both 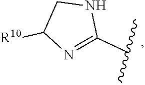
R^{5′} and R^{6′} are both hydrogen,
R¹⁰ is hydrogen, and
R^{15B} is hydrogen.

23. The compound of claim 18 or a pharmaceutically acceptable salt thereof, wherein
the compound of formula (Ia) is a 1,4-substituted phenyl bridged compound,
R⁵ and R⁶ are both 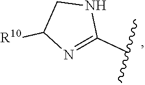
R^{5′} and R^{6′} are both hydrogen,
R¹⁰ is methyl, and
R^{15B} is hydrogen.

23. The compound of claim 18 selected from 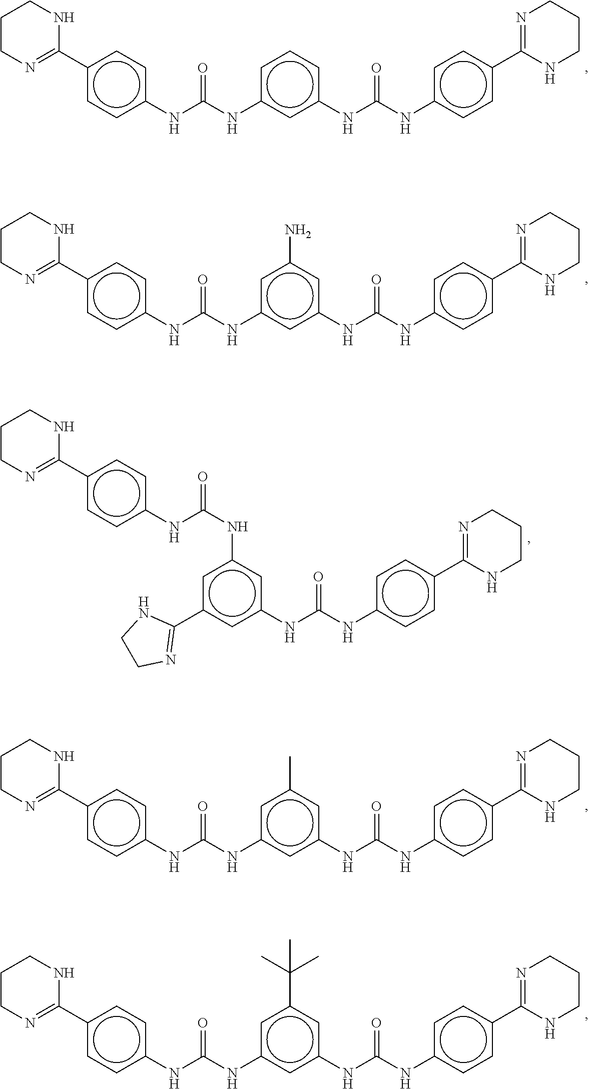 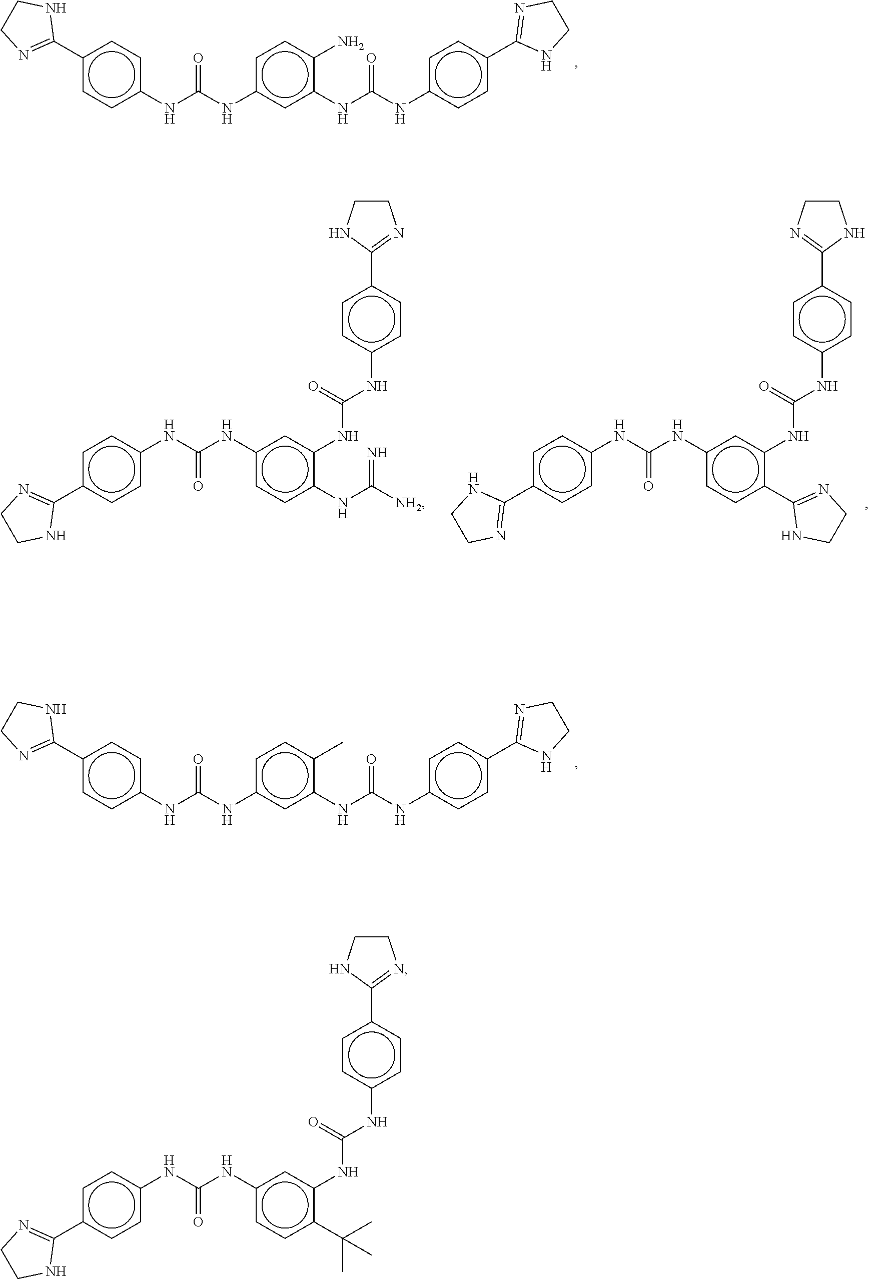 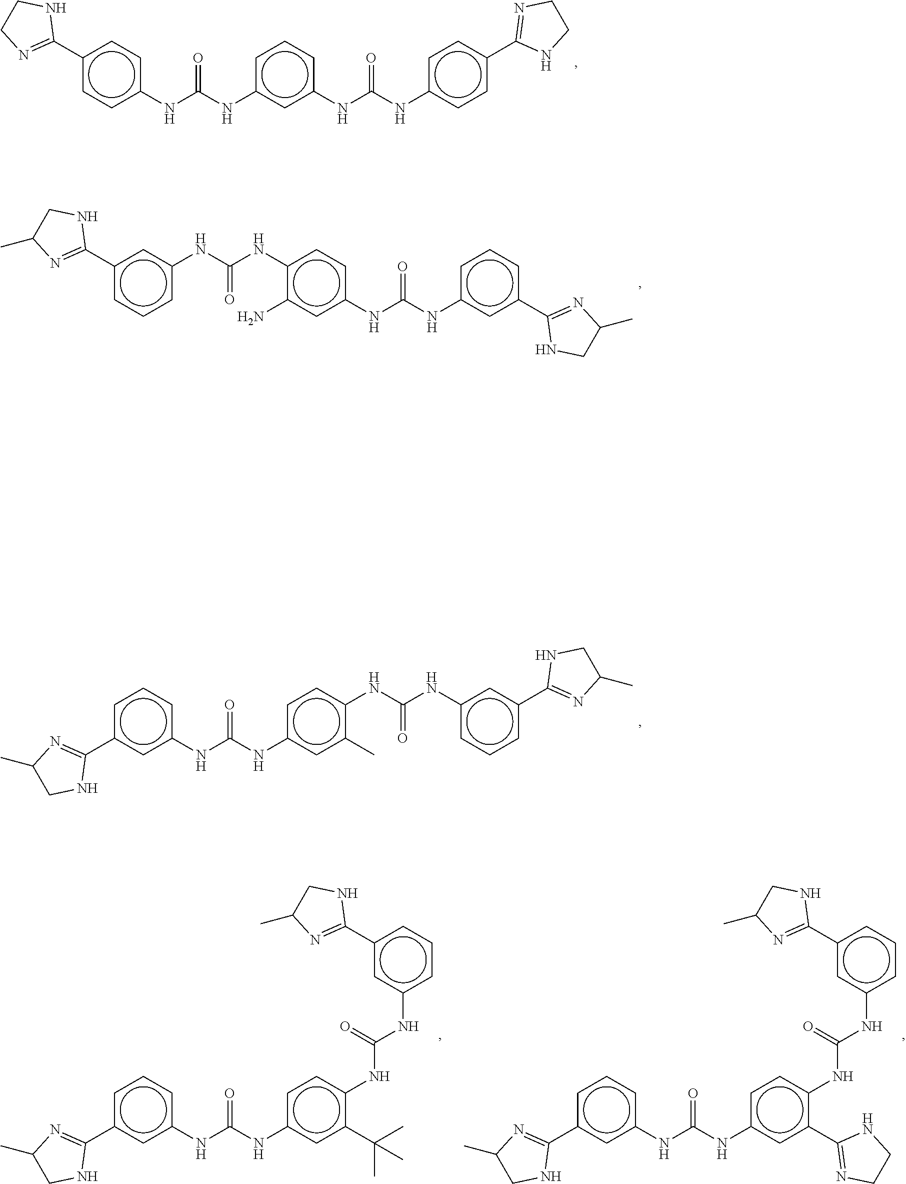 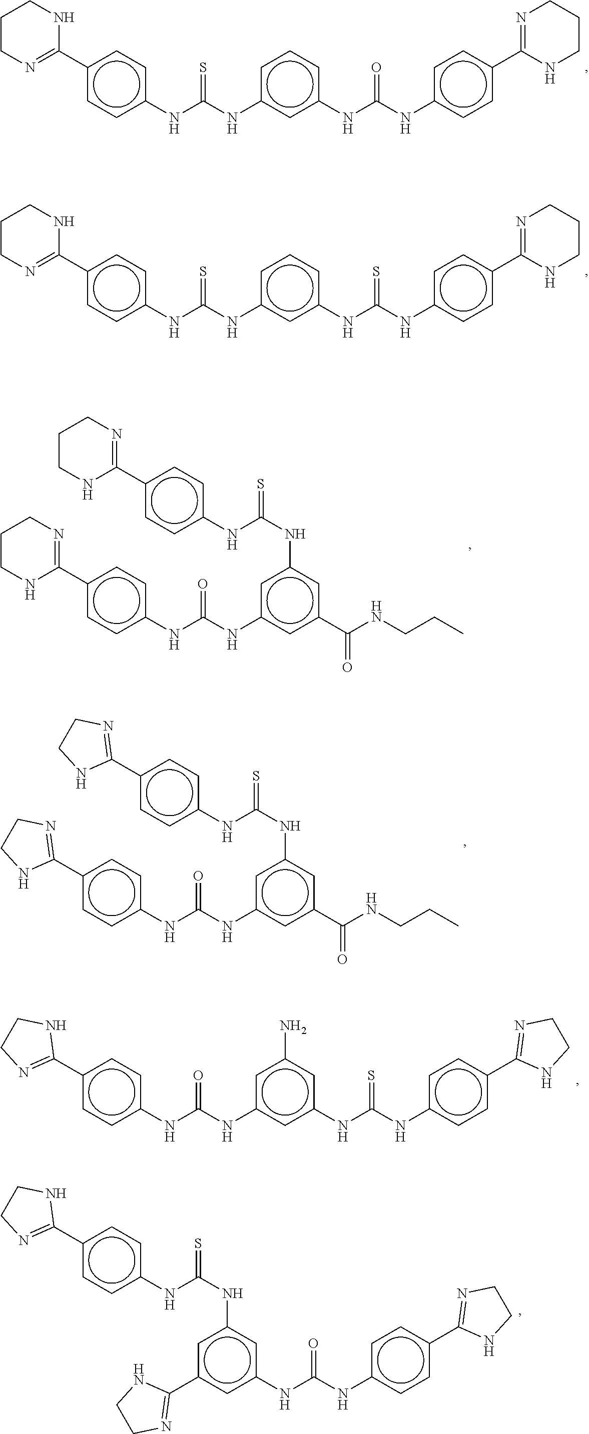 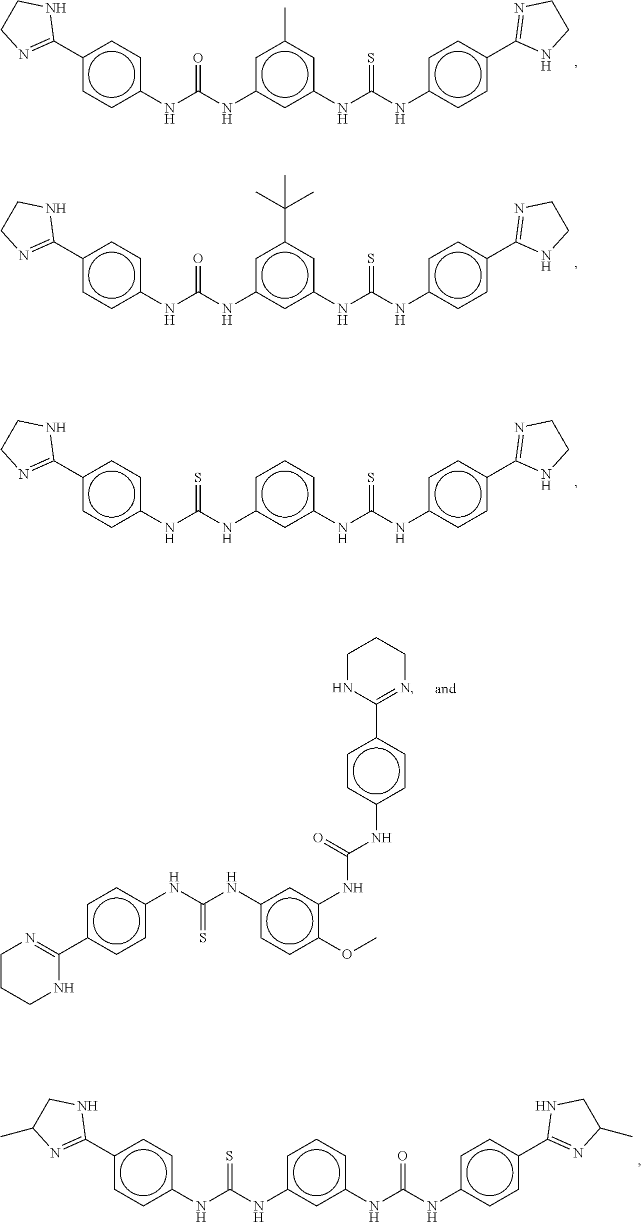
or a pharmaceutically acceptable salt thereof.

25. A pharmaceutical composition comprising the compound of claim 18 or a pharmaceutically acceptable salt thereof and at least one carrier.

26. A method of treating a disorder mediated by claudin-2 and/or claudin-15 in a subject comprising administering to the subject an effective amount of the compound of claim 18 or a pharmaceutically acceptable salt thereof to the subject.

27. The method of claim 26, wherein the disorder mediated by claudin-2 and/or claudin-15 is an intestinal disorder selected from colitis, Crohn's disease, enteritis, gastroenteritis, inflammatory bowel disease (IBD), irritable bowel syndrome (IBS), celiac disease, enteropathies, pouchitis, or diarrhea.

28. A method of treating a disorder mediated by inhibiting claudin-2 and/or claudin-15 in a subject comprising administering to the subject an effective amount of a compound selected from

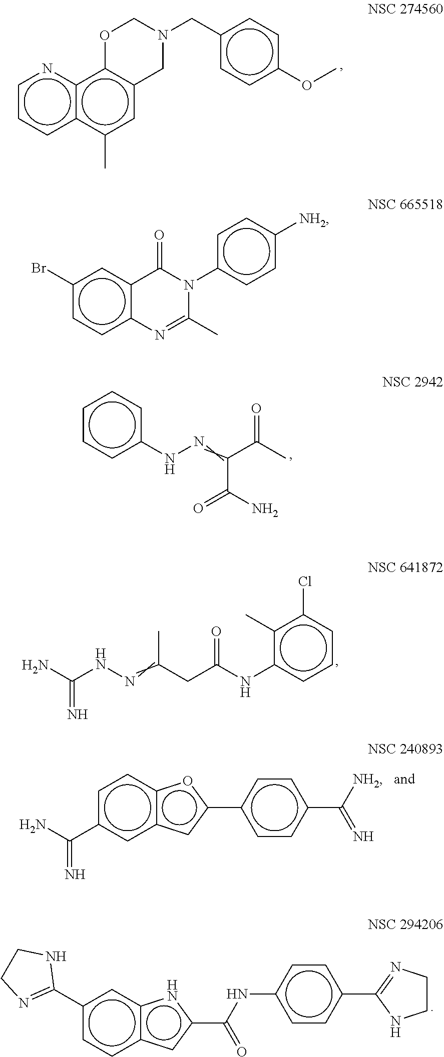

29. A method of treating a disorder mediated by claudin-2 and/or claudin-15 in a subject comprising administering to the subject an effective amount of a compound of formula (II) 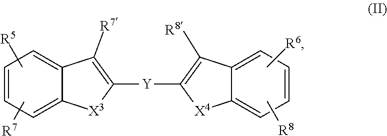
wherein
X³ and X⁴ are the same or different and each is O, S, or NH;
R⁵ and R⁶ are the same and are selected from 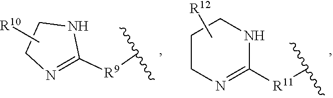
and —C(NHR¹³)═NR¹⁴;
wherein
R⁹ and R¹¹ are the same or different and each is a bond, —CH₂—, —CH₂CH₂—, or —NH—;
R¹⁰ and R¹² are the same or different and each is hydrogen, alkyl, or hydroxy; and
R¹³ and R¹⁴ are the same or different and each is hydrogen or alkyl;
R⁷, R⁸, R^{7′}, and R^{8′} are the same or different and each is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, wherein each R¹⁶ independently is a bond or —NH—; and
Y is 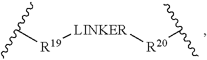
wherein
LINKER is selected from
(a) —CR²¹═CR²²—,
(b) —CR²¹₂CR²²₂—, and 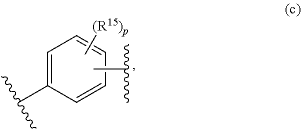
wherein each R¹⁵, R²¹, and R²² is the same or different and is independently selected from hydrogen, alkyl, halo, nitro, amino, hydroxy, alkoxy, alkylthio, amido, alkylamido, dialkylamido, phenylamido, carboxylate, —R¹⁶—C(O)NH-Ph-R⁵, and R⁵, and each p independently is 0 or an integer of 1-2; and
each of R¹⁹ and R²⁰ is independently selected from
(i) a bond,
(ii) —CR²¹═CR²²—,
(iii) —CR²¹2CR²²₂—, and 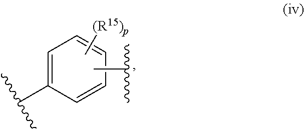
or a pharmaceutically acceptable salt thereof to the subject.

30. The method of claim 29, wherein in the compound of formula (II) or a pharmaceutically acceptable salt thereof, X³ and X⁴ are both O.

31. The method of claim 29, wherein in the compound of formula (II) or a pharmaceutically acceptable salt thereof, R⁵ and R⁶ are both —C(NHR¹³)NR¹⁴, wherein R¹³ and R¹⁴ are the same or different and each is hydrogen or alkyl.

32. The method of claim 31, wherein R¹³ and R¹⁴ are both hydrogen.

33. The method of claim 29, wherein R⁷, R⁸, R^{7′}, and R^{8′} are the same or different and each is independently selected from hydrogen and alkyl.

34. The method of claim 29, wherein R⁷, R⁸, R^{7′}, and R^{8′} are each hydrogen.

35. The method of claim 29, wherein Y is of formula:

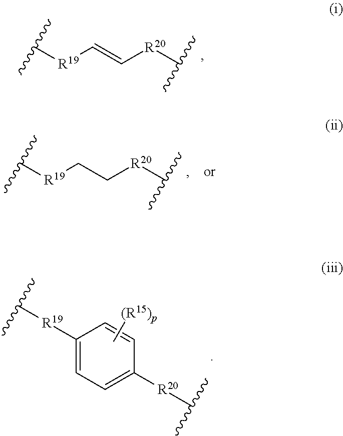

36. The method of claim 29, wherein the compound of formula (II) is a pharmaceutically acceptable salt.

37. The method of claim 29, wherein the compound of formula (II) or a pharmaceutically acceptable salt thereof is

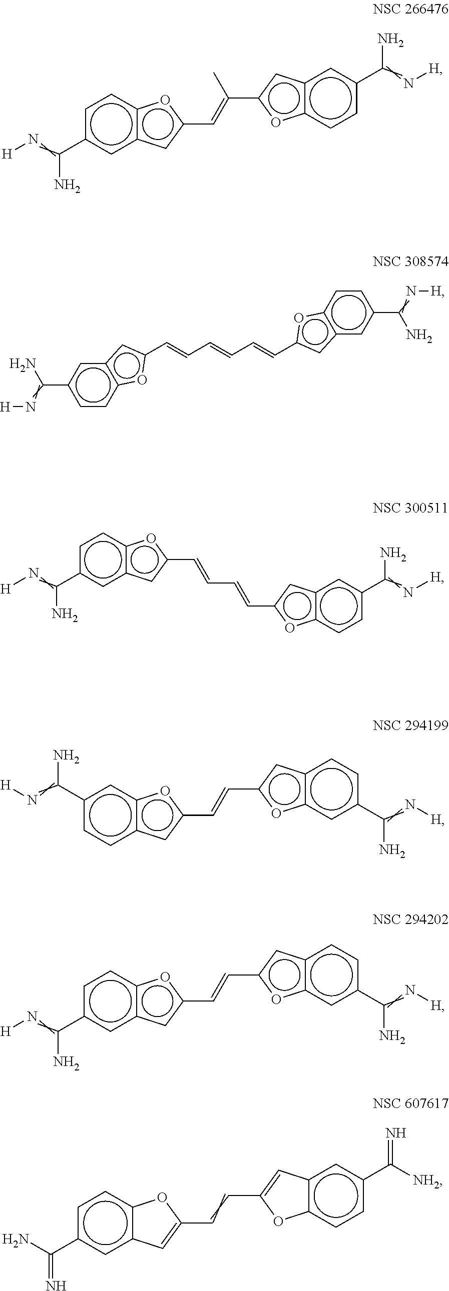

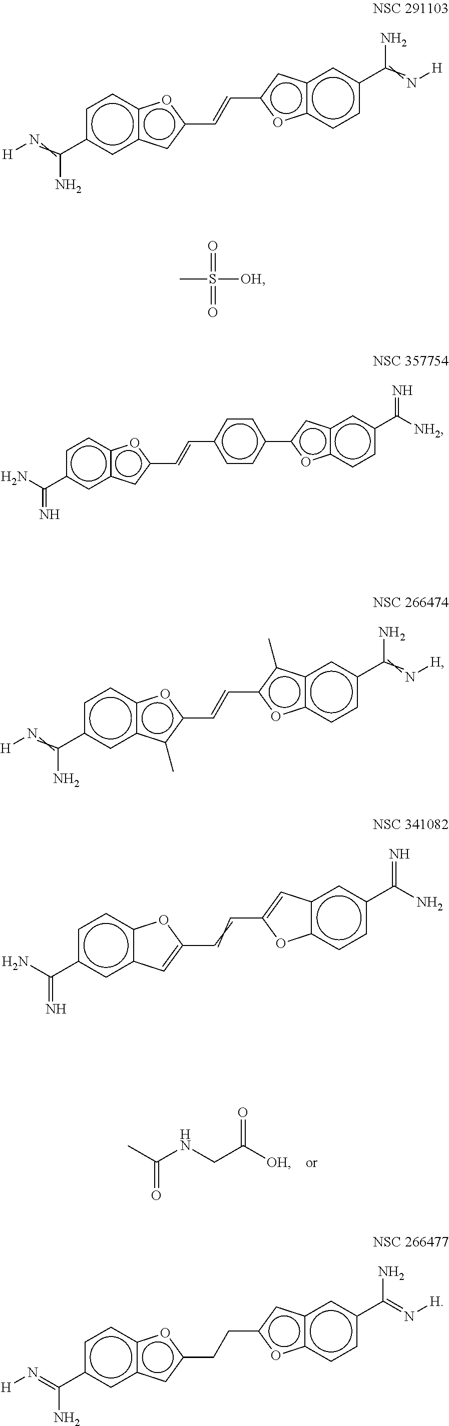

38. The method of claim 29, wherein the disorder mediated by claudin-2 and/or claudin-15 is an intestinal disorder.

39. The method of claim 38, wherein the intestinal disorder is colitis, Crohn's disease, enteritis, gastroenteritis, inflammatory bowel disease (IBD), irritable bowel syndrome (IBS), celiac disease, enteropathies, pouchitis, or diarrhea.

40. The method of claim 29, wherein the compound of formula (II) or a pharmaceutically acceptable salt thereof inhibits claudin-2 and/or claudin-15.
